(19) United States
(12) Patent Application Publication
(10) Pub. No.: US 2022/0235088 A1
(43) Pub. Date: Jul. 28, 2022

(54) INHIBITORS OF ADENYLATE-FORMING ENZYME MENE

(71) Applicants: Memorial Sloan-Kettering Cancer Center, New York, NY (US); The Research Foundation for The State University of New York, Albany, NY (US)

(72) Inventors: Derek Shieh Tan, New York, NY (US); Christopher E. Evans, New York, NY (US); Peter J. Tonge, Newbury, Berkshire (GB); Melissa Lynn Boby, New York, NY (US); Yuanyuan Si, Champaign, IL (US)

(73) Assignees: Memorial Sloan-Kettering Cancer Center, New York, NY (US); The Research Foundation for The State University of New York, Albany, NY (US)

(21) Appl. No.: 17/429,319

(22) PCT Filed: Feb. 7, 2020
(86) PCT No.: PCT/US20/17140

§ 371 Date: Aug. 6, 2021

Related U.S. Application Data
(60) Provisional application No. 62/802,650, filed on Feb. 7, 2019.

(51) Int. Cl. C07H 19/167; C07H 19/173

(52) U.S. Cl. CPC C07H 19/167; C07H 19/173

(57) ABSTRACT

Provided herein are compounds of Formula (I) and pharmaceutically acceptable salts or tautomers thereof which may inhibit adenylate-forming enzymes. Also provided are pharmaceutical compositions, kits, uses, and methods involving the inventive compounds for the treatment and/or prevention of an infectious disease (e.g., bacterial infection (e.g., tuberculosis, methicillin-resistant Staphylococcus aureus)).

### RELATED APPLICATIONS

This application is a national stage filing under 35 U.S.C. § 371 of international PCT application PCT/US2020/017140, filed Feb. 7, 2020, which claims priority under 35 U.S.C. § 119(e) to U.S. Provisional Patent Application, U.S. Ser. No. 62/802,650, filed Feb. 7, 2019, the contents of both which are incorporated herein by reference in their entireties.

### GOVERNMENT SUPPORT

This invention was made with government support under GM100477, GM102864 and CA008748 awarded by the National Institutes of Health. The government has certain rights in the invention.

### BACKGROUND OF THE INVENTION

The spread of drug-resistant pathogens such as multi- and extensively drug-resistant Mycobacterium tuberculosis and methicillin-resistant Staphylococcus aureus (MRSA) is a major threat to human health and places a significant burden on healthcare systems (Brown, E. D. et al. Nature 2016, 529, 336; Lewis, K. Nat. Rev. Drug Discovery 2013, 12, 371; CDC “Antibiotic Resistance Threats in the United States, 2013,” www.cdc.gov/drugresistance/threat-report-2013/; WHO “Antimicrobial resistance: Global report on surveillance 2014,” www.who.int/drugresistance/documents/surveillancereport/en/; The Pew Charitable Trusts “A Scientific Roadmap for Antibiotic Discovery,” 2016; www.pewtrusts.org/en/research-and-analysis/reports/2016/05/a-scientific-roadmap-for-antibiotic-discovery). Thus, the development of novel antibiotics that circumvent existing resistance mechanisms is urgently needed. The biological redox cofactor menaquinone is the sole electron-carrier in the electron transport chain of Gram positive bacteria, all bacteria growing anaerobically, and mycobacteria (Meganathan, R. et al. EcoSal Plus 2009, 3, doi: 10.5582/ddt.2016.01041; Meganathan, R. Vitam. Horm. 2001, 61, 173; Furt; F. et al. Plant J. 2010, 64, 38). Although higher mammals use menaquinone as a cofactor in many enzymes such as γ-glutamyl carboxylase, this quinone is acquired from the diet or from gut flora rather than through de novo biosynthesis (Dowd, P. et al. Annu. Rev. Nutr. 1995, 15, 419; Danziger, J. Clin. J. Am. Soc. Nephrol. 2008, 3, 1504; Nakagawa, K. et al. Nature 2010, 468, 117). Thus, inhibitors that target the bacterial menaquinone biosynthesis pathway are a promising avenue for future antibiotics that target drug resistant pathogens (Paudel, A. et al. Drug Discoveries Ther. 2016, 10, 123).

Menaquinone is derived from chorismate through a series of at least nine distinct enzyme-catalyzed transformations (FIG. 1) (Meganathan, R. Vitam. Horm. 2001, 61, 173; Jiang, M. et al. Biochemistry 2008, 47, 3426; Begley, T. P. et al. “Cofactor biosynthesis: A mechanistic perspective.” In Biosynthesis; Springer, 1998, pp 93). Inhibitors have been developed against most enzymes in the pathway including MenD (Fang, M. et al. Biochemistty 2010, 49, 2672; Fang, M. et al. Biochemistry 2011, 50, 8712) MenC (Pulaganti, M. et al. Appl. Biochem. Biotechnol. 2014, 172, 1407), MenE (Tian, Y. et al. Biochemistry 2008, 47, 12434), MenB (Li, X. el al. ACS Med Chem. Lett. 2011, 2, 818; Li. X. et al. Bioorg. Med. Chem. Lett. 2010, 20, 6306), and MenA (Kurosu, M. el al. Med. Chem. 2009, 5, 197; Dhiman, R. K. et al. Mol. Microbial. 2009, 72, 85; Kurosu, M. et al. Molecules 2010, 15, 1531; Li. K. et al. “J. Med. Chem. 2014, 57, 3126). Many of these compounds have significant antimicrobial activity, consistent with genetic studies which have demonstrated the essentiality of men genes in bacteria such as Bacillus subtilis and M. tuberculosis (Sassetti, C. M. et al. Mol. Microbiol. 2003, 48, 77; Kobayashi, K. et al. Proc. Natl. Acad. Sci. USA 2003, 100, 4678; Akerley, B. J. et al. Proc. Natl. Acad. Sci. USA 2002, 99, 966; Chen, W. H. et al. Nucleic Acids Res. 2017, 45, D940).

MenE, the acyl-CoA synthetase in the menaquinone biosynthetic pathway, has been the critical focus of efforts in this field. MenE, a member of the ANL (acyl-CoA synthetase, non-ribosomal peptide synthetase adenylation domain, luciferase) family (Gulick, A. M. ACS Chem. Biol. 2009, 4, 811), carries out a two-step reaction involving the initial activation of o-succinylbenzoate (OSB) by adenylation to form a tightly-bound OSB-adenosine monophosphate (AMP) intermediate, followed by a subsequent thioesterification with CoA to form OSB-CoA (FIG. 1) (Meganathan, R.; et al. J. Bacterial. 1979, 140, 92; Kwon, O. et al. J. Bacterial. 1996, 178, 6778). Previous efforts to inhibit MenE have focused on the use of acyl-AMS (5′-O-(N-acylsulfamoyl) adenosine) analogues (Lu, X. el al. Bioorg Med. Chem. Lett. 2008, 18, 5963; Lu, X. et al. ChemBioChem 2012, 13, 129; Matarlo, J. S. et al. Biochemistry 2015, 54, 6514; Evans, C. E. et al. Org Lett 2016, 18, 6384), inspired by natural products such as ascamycin (Isono, K. et al. Journal of Antibiotics 1984, 37, 670; Ueda, H, et al. Biochimica et Biophysica Acta, Protein Structure and Molecular Enzymology 1991, 1080, 126). Using this approach, OSB-AMS (1), a tight-binding, low nanomolar inhibitor of the S. aureus, M. tuberculosis, and E. coli MenE enzymes (Lu, X. et al. ChemBioChem 2012, 13, 129) were developed. While effective in biochemical assays, OSB-AMS has significant pharmacological liabilities that limit its further preclinical development. Both the carboxylate and sulfamate are depmtonated at physiologic pH, and this is the likely source of its poor cellular permeability (Yoshimura, F. et al. Antimicroh. Agents Chemother. 1985, 27, 84; Richter, M. F. et al. Nature 2017, 545, 299). Negative charge is also associated with high plasma protein binding and rapid renal clearance (Varma, M. V. S. et al. J. Med. Chem. 2009, 52,4844; Charifson, P. S. J. Med. Chem. 2014, 57, 9701). Finally, some acyl sulfamates may be hydrotytically unstable in vivo (Tiwari, D. et al. Sci. Transl. Med. 2018, 10, eaal1803). The free carboxylate of OSB is necessary for potent binding to MenE (Matarlo, J. S. et al. Biochemistry 2015, 54, 6514), but it is reported that one is able to replace the OSB motif with a difluoroindanediol that is neutral at physiologic pH (Matarlo, J. S. et al. Biochemistry 2015, 54, 6514; Evans, C. E. et al. Org Lett 2016, 18, 6384). Certain difluoroindanediol compounds were prepared in Evans, C. E. et al. Org. Lett. 2016, 18, 6384-6387, the contents of which are incorporated herein by reference in their entirety. Thus, subsequent efforts have focused on replacing the acyl-sulfamate moiety to avoid the pharmacological liabilities associated with this linker.

### SUMMARY OF THE INVENTION

Previous attempts to identify alternative bioisosteres of the native acyiphosphate motif have met with limited success, with most substitutions resulting in significant loss of biochemical activity against the target adenylate-forming enzyme (Soares da Costa, T. P. et al. J. Biol. Chem. 2012, 287, 17823; Somu, R. V. et al. J. Med. Chem. 2006, 49, 31; Vannada, J. et al. Org Lett 2006, 8, 4707; Qiao, C. H. et al. J. Med. Chem. 2007, 50, 6080; Engelhart, C. A. et al. J. Org. Chem. 2013, 78, 7470; Dawadi, S. et al. ACS Med. Chem. Lett. 2018, 9, 386; Callahan, B. P. et al. Bioorg. Med. Chem. Len. 2006, 16, 3802; Bisseret, P. et al. Tetrahedron Lett. 2007, 48, 6080). Thus, a virtual library of OSB-AMS linker analogues was designed and used computational docking to prioritize compounds for synthesis, leading to the identification of several novel linker analogues, the most promising of which was further characterized through X-ray crystallography to elucidate its mechanism of binding for comparison to the computational prediction.

Reported herein is the development of new acyl-AMS analogues containing an phenylene, pyridinylene, or pyrimidinylene linker. In order to overcome pharmacological liabilities and potential metabolic instability of previous acyl-AMS analogues bearing an acylsulfamate moiety which becomes charged at physiological pH, the acylsulfamate linker was replaced with a phenylene, pyridinylene, or pyrimidinylene linker which remains neutral. These analogues were capable of inhibiting E. coli MenE, showing that replacement of the acyl-sulfamate moiety found in most inhibitors of adenylate-forming enzymes is feasible and provides a path forward for the development of new MenE inhibitors, new adenylate-forming enzyme inhibitors, and new antibiotics. Detailed herein is the use of phenylene, pyridinylene, or pyrimidinylene linker based acyl-AMS analogues as inhibitors of menaquinone biosynthesis (e.g., inhibition of MenE) demonstrating their propensity for use as antimicrobials, such as antibacterial (e.g., for use against E. coli, M. tuberculosis, B. onthracis, S. aureus, Y. pestis. and P. aeruginosa), antifungals, antivirals, and antiparasitics. Also provided herein are pharmaceutical compositions, methods of treatment and/or prevention, uses, methods of synthesis of the analogues, and kits.

In one aspect, the present disclosure provides compounds of Formula (I):

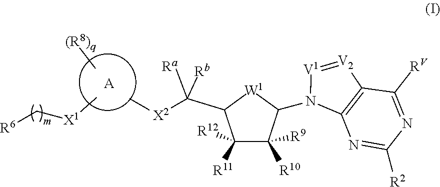

or a pharmaceutically acceptable salt or tautomer thereof, wherein Ring A, R^{V}, R², R⁹, R¹⁰, R¹¹, R¹², R^{a}, R^{b}, R⁶, R⁸, V¹, V², W¹, X¹. X², q and in are as defined herein.

In another aspect, the present disclosure provides compounds of Formula (I-A):

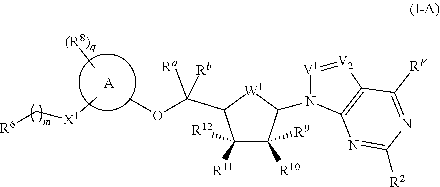

or a pharmaceutically acceptable salt, stereoisomer, or tautomer thereof, wherein Ring A, R^{V}, R², R⁹, R¹⁰, R¹¹, R¹², R^{a}, R^{b}, R⁶, R⁸, V¹, V², W¹, X¹, q and m are as defined herein.

In another aspect, the present disclosure provides compounds of Formula (I-B):

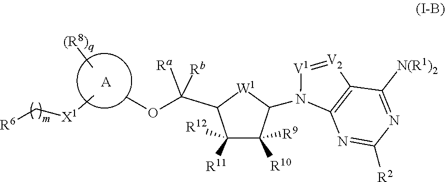

or a pharmaceutically acceptable salt, stereoisomer, or tautomer thereof, wherein Ring A, R¹, R², R⁹, R¹⁰, R¹¹, R¹², R^{a}, R^{b}, R⁶, R⁸, V¹, V², W¹, X¹, q and m are as defined herein.

In another aspect, the present disclosure provides compounds of Formula (II):

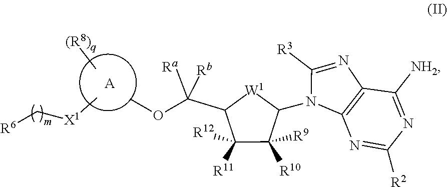

or a pharmaceutically acceptable salt, stereoisomer, or tautomer thereof, wherein Ring A, R², R³, R⁹, R¹⁰R¹¹, R¹², R^{a}, R^{b}, R⁶, R⁸, W¹, X¹, q and m are as defined herein.

In another aspect, the present disclosure provides compounds of Formula (III):

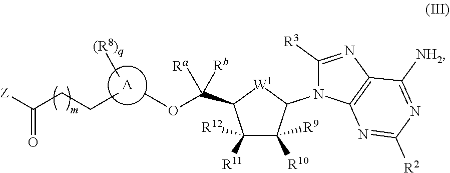

or a pharmaceutically acceptable salt, stereoisomer, or tautomer thereof, wherein Ring A, R², R³, R⁹, R¹⁰, R¹¹, R¹², R^{a}, R^{b}, R⁸, W¹, Z, q and m are as defined herein.

In another aspect, the present disclosure provides compounds of Formula (IV):

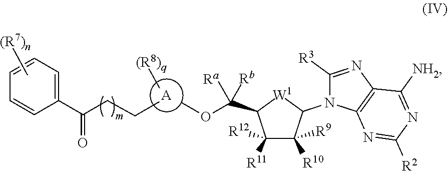

or a pharmaceutically acceptable salt, stereoisomer, or tautomer thereof, wherein Ring A, R², R³, R⁹, R¹⁰, R¹¹, R¹², R^{a}, R^{b}, R⁸, R⁷, W¹, q, m, and n are as defined herein.

In another aspect, the present disclosure provides compounds of Formula (IV-A):

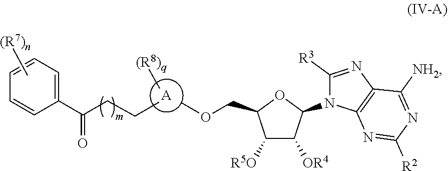

or a pharmaceutically acceptable salt, stereoisomer, or tautomer thereof, wherein Ring A, R², R³, R⁴, R⁵, R⁸, R⁷, q, m, and n are as defined herein.

In another aspect, the present disclosure provides compounds of Formula (IV-B):

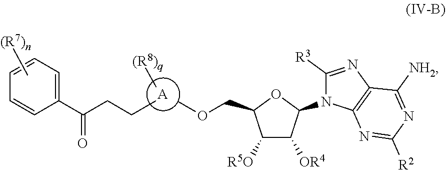

or a pharmaceutically acceptable salt, stereoisomer, or tautomer thereof, wherein Ring A, R², R³, R⁴, R⁵, R⁸, R⁷, q, and n are as defined herein.

In another aspect, the present disclosure provides compounds of Formula (IV-C):

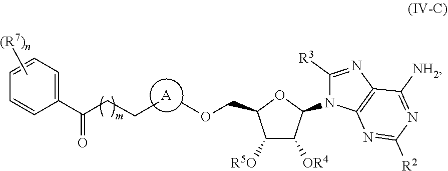

or a pharmaceutically acceptable salt, stereoisomer, or tautomer thereof, wherein Ring A, R², R³, R⁴, R⁵, R⁷, m, and n are as defined herein.

In another aspect, the present disclosure provides compounds of Formula (lV-D):

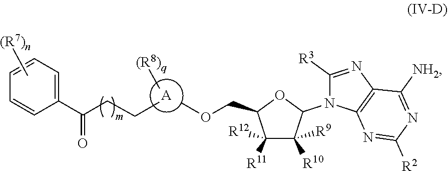

or a pharmaceutically acceptable salt or tautomer thereof, wherein Ring A, R², R³, R⁹, R¹⁰, R¹¹, R¹², R⁷, q, m, and n are as defined herein.

In another aspect, the present disclosure provides compounds of Formula (V):

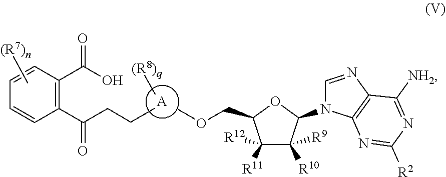

or a pharmaceutically acceptable salt, stereoisomer, or tautomer thereof, wherein Ring A, R², R⁹, R¹⁰, R¹¹, R¹², R⁷, R⁸, q, and n are as defined herein.

In another aspect, the present disclosure provides compounds of Formula (V-A):

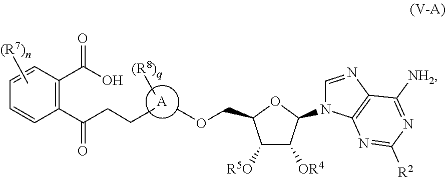

or a pharmaceutically acceptable salt, stereoisomer, or tautomer thereof, wherein Ring A, R², R⁴, R⁵, R⁸, R⁷, q, and n are as defined herein.

In another aspect, the present disclosure provides compounds of Formula (V-B):

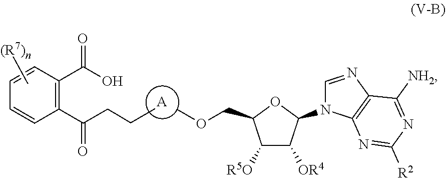

or a pharmaceutically acceptable salt, stereoisomer, or tautomer thereof, wherein Ring A, R², R⁴, R⁵, R⁷, and n are as defined herein.

In another aspect, the present disclosure provides compounds of Formula (V-C):

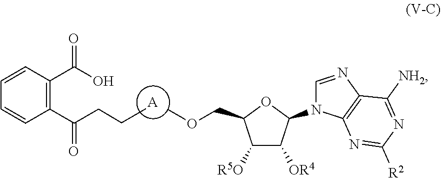

or a pharmaceutically acceptable salt, stereoisomer, or tautomer thereof, wherein Ring A, R², R⁴, and R⁵ are as defined herein.

In another aspect, the present disclosure provides compounds of Formula (VI):

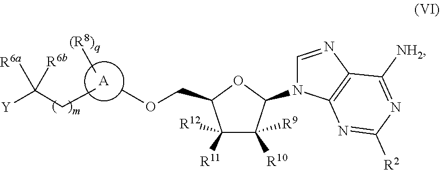

or a pharmaceutically acceptable salt, stereoisomer, or tautomer thereof, wherein Ring A, R², R⁹, R¹⁰, R¹¹, R¹², R^{6a}, R^{6b}, R⁸, q, m, and Y are as defined herein.

In another aspect, the present disclosure provides compounds of Formula (VI-A):

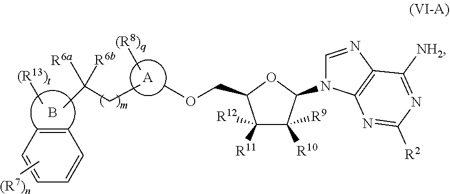

or a pharmaceutically acceptable salt, stereoisomer, or tautomer thereof, wherein Ring A, Ring B, R², R⁹, R¹⁰, R¹¹, R¹², R^{6a}, R^{6b}, R⁷, R⁸, R¹³, q, m, n, and t are as defined herein.

In another aspect, the present disclosure provides compounds of Formula (VI-B):

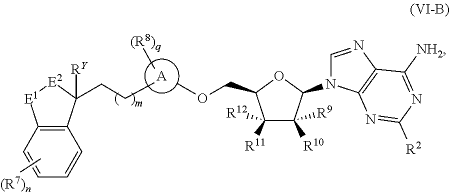

or a pharmaceutically acceptable salt, stereoisomer, or tautomer thereof, wherein R², R⁹, R¹⁰, R¹¹, R¹², R⁷, R⁸, R^{Y}, E¹, E², q, m, and n are as defined herein.

In another aspect, the present disclosure provides compounds of Formula (VI-C):

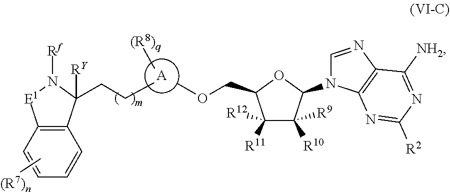

or a pharmaceutically acceptable salt, stereoisomer, or tautomer thereof, wherein R², R⁹, R¹⁰, R¹¹, R¹², R⁷, R⁸, R^{f}, R^{Y}, E¹, q, m, and n are as defined herein.

In another aspect, the present disclosure provides compounds of Formula (VI-D):

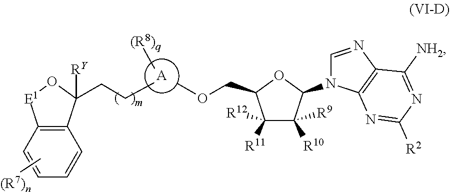

or a pharmaceutically acceptable salt, stereoisomer, or tautomer thereof, wherein R², R⁹, R¹⁰, R¹¹, R¹², R⁷, R⁸, R^{Y}, E¹, q, m, and n are as defined herein.

In another aspect, the present disclosure provides compounds of Formula (VI-E):

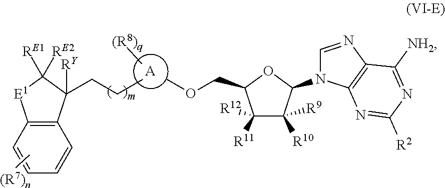

or a pharmaceutically acceptable salt, stereoisomer, or tautomer thereof, wherein R², R⁹, R¹⁰, R¹¹, R¹², R⁷, R⁸, R^{E1}, R^{E2}, R^{Y}, E¹, q, m, and n are as defined herein.

In another aspect, the present disclosure provides compounds of Formula (VI-F):

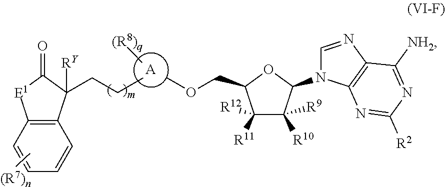

or a pharmaceutically acceptable salt, stereoisomer, or tautomer thereof, wherein R², R⁹, R¹⁰, R¹¹, R¹², R⁷, R⁸, R^{Y}, E¹, q, m, and n are as defined herein.

In another aspect, the present disclosure provides compounds of Formula (VI-G):

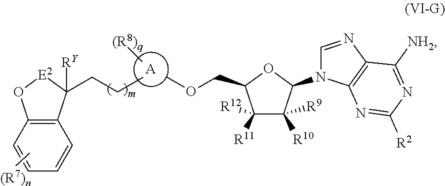

or a pharmaceutically acceptable salt, stereoisomer, or tautomer thereof, wherein R², R⁹, R¹⁰, R¹¹, R¹², R⁷, R⁸, R^{Y}, E², q, m, and n are as defined herein.

In another aspect, the present disclosure provides compounds of Formula (VI-H):

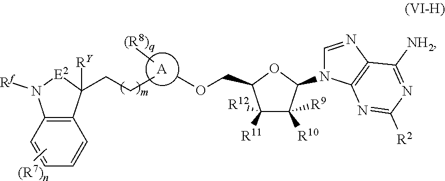

or a pharmaceutically acceptable salt, stereoisomer, or tautomer thereof, wherein R², R⁹, R¹⁰, R¹¹, R¹², R⁷, R⁸, R^{f}, R^{Y}, E², q, m, and n are as defined herein.

In another aspect, the present disclosure provides compounds of Formula (VI-I):

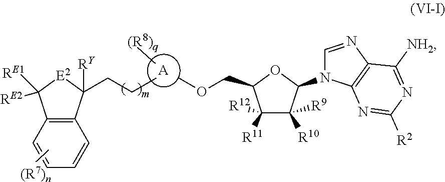

or a pharmaceutically acceptable salt, stereoisomer, or tautomer thereof, wherein R², R⁹, R¹⁰, R¹¹, R¹², R⁷, R⁸, R^{E1}, R^{E2}, R^{Y}, E², q, m, and n are as defined herein.

In another aspect, the present disclosure provides compounds of Formula (VI-J):

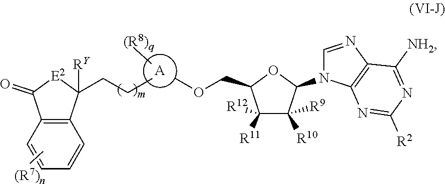

or a pharmaceutically acceptable salt, stereoisomer, or tautomer thereof, wherein R², R⁹, R¹⁰, R¹¹, R¹², R⁷, R⁸, R^{Y}, E², q, m, and n are as defined herein.

As described herein, the present disclosure provides exemplary compounds including but not limited to:

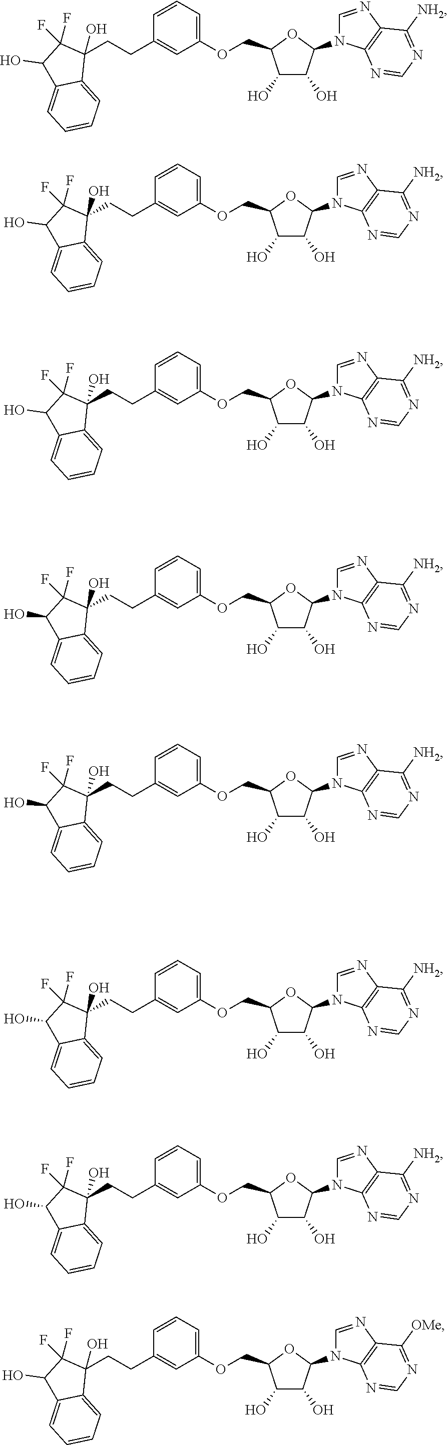

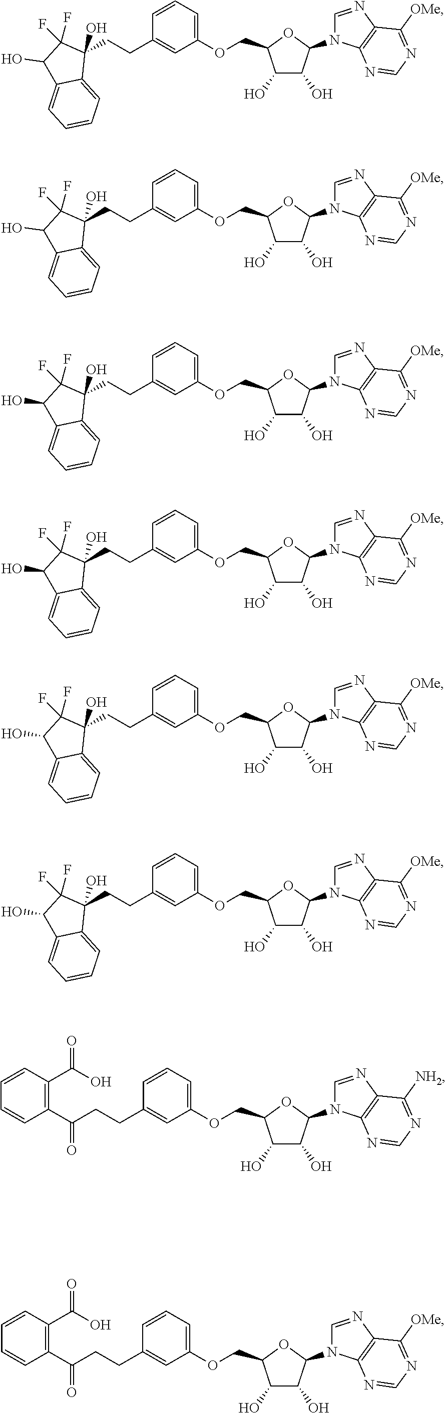

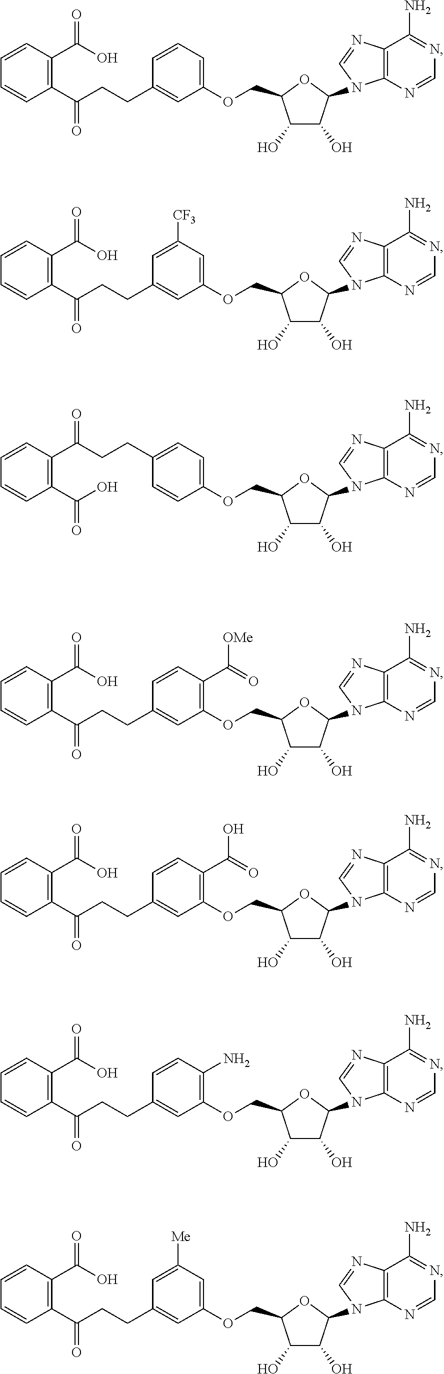

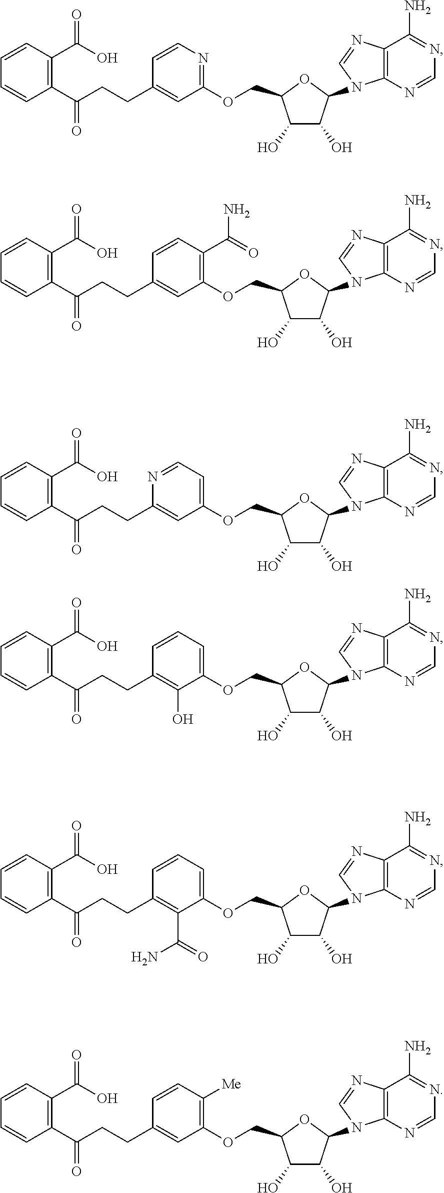

In another aspect, the present disclosure provides pharmaceutical compositions including a compound described herein, and optionally a pharmaceutically acceptable excipient. In certain embodiments, the pharmaceutical compositions described herein include an effective amount of a compound described herein. In certain embodiments, the pharmaceutical compositions described herein include an additional pharmaceutical agent. The pharmaceutical composition may be useful for treating andior preventing an infectious disease. In some embodiments, the infectious disease is a bacterial infection (e.g., a gram positive bacterial infection, a gram negative bacterial infection, an E. coli infection, a M. tuberculosis infection (e.g., multi-drug-resistant tuberculosis (MDR-TB) infection or extensively drug-resistant tuberculosis (XDR-TB) infection), a B. anthracis infection, a S. aureus infection (e.g., a methicillin-resistant Staphylococcus aureus (MRSA) infection, vancomycin-intermediate Staphylococcus (wrens (VISA) infection, a vancomycin-resistant Stophylococrns aureus (VRSA) infection), a Y. pestis infection, a P. aertiginosa infection). In some embodiments, the disease is a viral infection, a parasitic infection, or a fungal infection.

The pharmaceutical compositions described herein may be useful for treating or preventing tuberculosis.

The present disclosure describes methods for administering to a subject in need thereof (e.g., a subject with an infection) an effective amount of a compound, or a pharmaceutical composition thereof, as described herein. In certain embodiments, a method described herein further comprises administering to the subject an additional pharmaceutical agent (e.g., another antimicrobial agent).

In yet another aspect, the present disclosure provides compounds for use in the treatment or prevention of an infectious disease in a subject. In some embodiments, the present disclosure provides compounds for use in the treatment or prevention of a bacterial infection. In some embodiments, the present disclosure provides compounds for use in the treatment or prevention of tuberculosis.

In another aspect, the present disclosure provides methods for treating and/or preventing a disease. Exemplary diseases which may be treated include bacterial infections (e.g., Mycobacterium tuberculosis infection), fungal infections, viral infections, and fungal infections. In certain embodiments, the bacterial infection may be caused by a gram positive bacteria or a gram negative bacteria. In some embodiments, the bacterial infection is caused by antibiotic resistant organisms. In some embodiments, the bacterial infection is tuberculosis.

Another aspect of the disclosure relates to methods of inhibiting menaquinone biosynthesis (e.g., inhibiting MenE). In certain embodiments, the disclosure provides methods of inhibiting IvIenE.

In yet another aspect, the present disclosure provides compounds, and pharmaceutical compositions thereof, as described herein for use in any method of the disclosure.

Another aspect of the present disclosure relates to kits comprising a container with a compound, or pharmaceutical composition thereof, as described herein. The kits described herein may include a single dose or multiple doses of the compound or pharmaceutical composition. The kits may be useful in any method of the disclosure. In certain embodiments, the kit further includes instructions for using the compound or pharmaceutical composition. A kit described herein may also include information (e.g. prescribing information) as required by a regulatory agency, such as the U.S. Food and Drug Administration (FDA).

The details of certain embodiments of the invention are set forth in the Detailed Description of Certain Embodiments, as described below. Other features, objects, and advantages of the invention will be apparent from the Definitions, Examples, Figures, and Claims,

### BRIEF DESCRIPTION OF THE DRAWINGS

The accompanying drawings, which constitute a part of this application, illustrate several embodiments of the invention and together with the description, serve to explain the principles of the invention.

FIG. 1 shows the classical menaquinone biosynthesis pathway and structure of MenE inhibitor OSB-AMS. At least nine enzymes catalyze the formation of menaquinone from chorismate. The fifth enzyme, MenE, is an acyl-CoA synthetase which catalyzes the ATP-dependent ligation of CoA to o-succinylbenzoate (OSB) via an OSB-AMP intermediate. OSB-AMS (1) is a stable analogue of OSB-AMP in which the mixed carboxyphosphate anhydride is replaced with sulfamate.

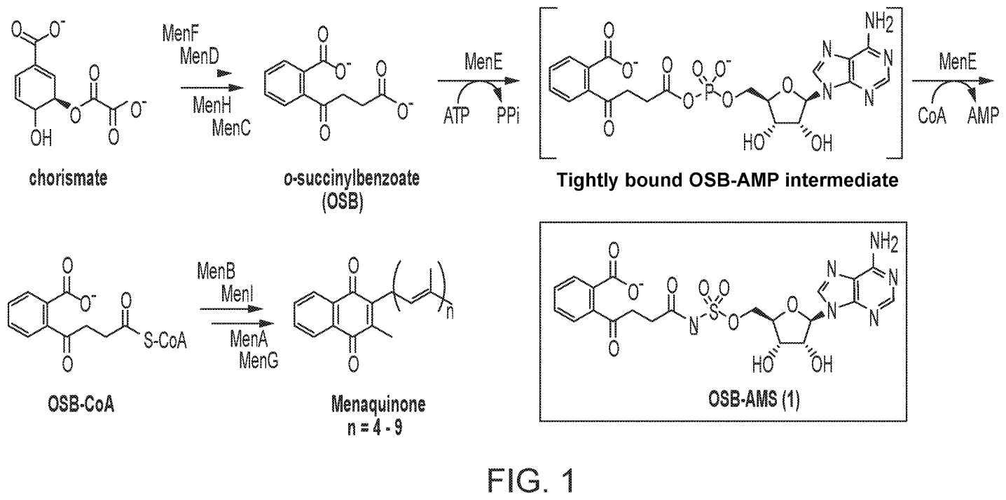

FIG. 2A shows the X-ray co-crystal structure of OSB-AMS (1) bound in the active site of MenE with key binding interactions (PDB: 5C5H).

FIG. 2B shows the X-ray co-crystal structure of the in-phenyl ether analog (5) of OSB-AMS (1) bound in the active site of MenE with key binding interactions (PDB: 5C5H).

FIG. 3A shows an overlay of structures of E, coli wild-type MenE with m-phenyl ether analogue 5 bound, E. coli MenE (R195K) with OSB-AMS (1) bound (PDB; 5C5H), and S. aureus MenE apo structures (PDB: 3LPI., chains A and B). Alignment of the large N-terminal domains (E. coli residues 1-351, rmsd 0.49 A; S. aureus residues 1-396, rmsd 1.64 Å relative to 5C5H) reveals a 22° rotation of the small C-terminal domain about the E. coli hinge residue D352 in the structure with analogue 5 compared to that with OSB-AMS (1), while larger 144° and 151° rotations of the C-terminal domains are observed in the S. aureus apo structures. The E. coli lynchpin residue K437 is observed in the structure with OSB-AMS (1), but is disordered in the structure with ,n-phenyl ether analogue 5.

FIG. 3B shows the active site of wild-type MenE with in-phenyl ether analogue 5 bound, revealing binding interactions and 1.4 Å shift of ribose motif compared to OSB-AMS (1).

FIG. 3C shows the putative active-site interactions of m-phenyl ether analogue 5 with wild-type MenE.

### DETAILED DESCRIPTION OF CERTAIN EMBODIMENTS

Provided herein are compounds which may inhibit adenylate-forming enzymes. In certain embodiments, the compounds of the invention inhibit o-succinylbenzoate-CoA synthetase (MenE). The compounds may interact with MenE so as to disrupt the activity of MenE in converting o-succinylbenzoate (OSB) to o-succinylbenzoate-CoA (OSB-CoA). MenE catalyzes two transformations in tandem. The first reaction combines OSB and ATP to form the intermediate OSB-AMP and pyrophosphate as a by-product. In the second reaction, CoA is conjugated to OSB to form OSB-CoA, and AMP is released. In some embodiments, a compound described herein affects the ability of MenE to form OSB-AMP, i.e., it inhibits the first transformation. in some embodiments, a compound described herein affects the ability of MenE to form OSB-CoA, i.e.. it inhibits the second transformation. In some embodiments, a compound may inhibit both the first and second transformation.

In the biosynthesis of menaquinone, OSB-CoA is subsequently converted to 1,4-dihydroxy-2-napthoyl-CoA (DHNA-CoA). and ultimately to menaquinone. Thus, a compound of the invention may inhibit menaquinone biosynthesis. In some embodiments, a compound provided inhibits menaquinone biosynthesis by inhibiting MenE. In some embodiments, a compound provided inhibits menaquinone biosynthesis by inhibiting the formation of OSB-CoA.

Provided herein are compounds for the treatment and/or prevention of diseases, including bacterial infections. The compounds may inhibit a particular enzyme (e.g., MenE) of an organism (e.g., Mycobacterium tuberculosis, Staphylococcus aureus) responsible for a bacterial infection (e.g., Mycobacterium tuberculosis infection, Staphylococcus aureus (e.g., MRSA)). Further, the compounds may be used to treat or prevent a bacterial infection (e.g., tuberculosis). The present disclosure provides compounds, pharmaceutical compositions, synthesis methods, methods of treatment, uses, and kits useful for treating or preventing an infectious disease. In certain embodiments, the infectious disease is tuberculosis. In certain embodiments, the infectious disease is caused by methicillin-resistant Staphylococcus aureus (MRSA).

In some aspects, compounds of the present disclosure are of Formula (I):

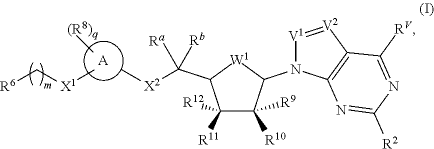

or a pharmaceutically acceptable salt, stereoisomer, or tautomer thereof, wherein:
V¹ is ═CR³— or ═N—;
V² is ═CH— or ═N—;
R^{V} is —N(R¹)₂, —OR¹, halogen, or hydrogen;
R¹ is hydrogen, optionally substituted alkyl, optionally substituted heteroalkyl, optionally substituted carbocyclyl, optionally substituted heterocyclyl, optionally substituted aryl, optionally substituted heteroaryl, or optionally substituted acyl, or two R¹ are joined to form an optionally substituted heterocyclic ring;
each of R² and R³ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, —N(R^{e})₂, —N₃, —SO₂H, —SO₃H; —SR^{e}, —SSR^{e}, —OC(═O)R^{e}, —OCO₂R^{e}, —OC(═O)N(R^{e})₂, —C(═O)N(R^{e})₂, —NC(═O)N(R^{e})₂, —OC(═O)O(R^{e})₂, —SO₂R^{e}, —SO₂OR^{e}, —OSO₂R^{e}, —S(═O)R^{e}, or —OS(═O)R^{e};
W¹ is —O—, —CR^{e}₂—, —NR^{e}—, or —S—;
each of R⁹, R¹⁰, R¹¹, and R¹² is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR⁴, —OR⁵, —OR^{e}, —N(R^{e})₂, —N₃, —SO₂H, —SO₃H; —SR^{e}, —OC(═O)R^{e}, —C(═O)OR^{e}, —C(═O)N(R^{e})₂, —N(R^{e})C(═O)R^{e}, or —SO₂R^{e}, or two occurrence of any R⁹, R¹⁰, R¹¹, and R¹² are joined to form an optionally substituted carbocyclic ring or an optionally substituted heterocyclic ring;
each of R⁴ and R⁵ is independently hydrogen, optionally substituted C₁₋₆ alkyl, optionally substituted acyl, or an oxygen protecting group, or R⁴ and R⁵ are joined to form an optionally substituted heterocyclic ring;
each of R^{a} and R^{b} is independently hydrogen, halogen, optionally substituted C₁₋₆ alkyl, —OR^{e}, or —N(R^{e})₂;
X¹ is a bond, —C(R^{e})₂—, —O—, or —NR^{e}—;
X² is a bond, —C(R^{e})₂—, —O—, or —NR^{e}—;
R⁶ is of the formula:

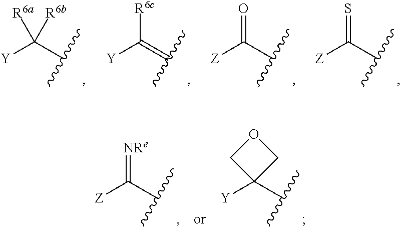

Ring A is phenylene, pyridinylene, or pyrimidinylene;
each of Y and Z is independently optionally substituted alkyl, optionally substituted alkenyl, optionally substituted alkynyl, optionally substituted heteroalkyl, optionally substituted heteroalkenyl, optionally substituted heteroalkynyl, optionally substituted alkoxy, optionally substituted amino, —OR^{e}, —N(R^{e})₂, optionally substituted carbocyclyl, optionally substituted heterocyclyl, optionally substituted aryl, or optionally substituted heteroaryl;
each of R^{6a}, R^{6b}, and R^{6c} is independently hydrogen, halogen, optionally substituted C₁₋₆ alkyl, —OR^{e}, or —N(R^{e})₂;
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, an oxygen protecting group when attached to an oxygen atom, a nitrogen protecting group when attached to a nitrogen atom, or a sulfur protecting group when attached to a sulfur atom, or two R^{e} are joined to form an optionally substituted carbocyclic, an optionally substituted aryl, an optionally substituted heterocyclic, or optionally substituted heteroaryl ring;
each occurrence of R^{e} is independently halogen, optionally substituted alkyl, haloalkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —COOH, COOR^{e}, —CONH₂, —CON(R^{e})₂, —NO₂, —CN, —OR^{e}, or —N(R^{e})₂, or two R⁸ are joined to form an optionally substituted carbocyclyl ring, optionally substituted heterocyclyl ring, optionally substituted aryl, or optionally substituted heteroaryl ring; and
m is 0, 1, 2, 3, 4, 5, or 6; and
q is 0, 1, 2. 3, or 4.

### Nucleosbase, Purine, and Heterocycle Moiety

In some embodiments, V¹ is ═CR³—. In some embodiments, V¹ is ═CH—. In certain embodiments, V¹ is ═N—. In some embodiments, V¹ is ═CR³— wherein R³ is —F. In certain embodiments, R³ is —Cl, —Br, or —F. In certain embodiments, In some embodiments, V¹ is ═CR³— wherein R³ is —OR^{e} (e.g. —OH, —OMe, —O(C₁₋₆ alkyl)). In certain embodiments, In some embodiments, V¹ is ═CR³— wherein R³ is —N(R^{e})₂ (e.g., —NH₂, —NMe₂, —NH(C₁₋₆ alkyl)). In certain embodiments, V¹ is ═CR³—, wherein R³ is optionally substituted C₁₋₆ alkyl. In certain embodiments, V¹ is ═CR³—, wherein R³ is optionally substituted methyl. In certain embodiments, V¹ is R³—, wherein R³ is optionally substituted ethyl, propyl, or butyl. In certain embodiments, R³ is optionally substituted carbocyclyl (e.g., cyclopropyl, cyclohexyl). In certain embodiments, R³ is optionally substituted aryl (e.g.. phenyl).

In certain embodiments, R³ is hydrogen. In certain embodiments, R³ is halogen. In certain embodiments, R³ is —F. In certain embodiments, R³ is —Cl, —Br, or —F. In certain embodiments. R³ is —NO_{2.} In certain embodiments, R³ is —CN. In certain embodiments, R³ is —OR^{e} (e.g. —OH, —OMe, —O(C₁₋₆ alkyl)) In certain embodiments, R³ is —OR^{e}, and R^{e} is an oxygen protecting group. In certain embodiments, R³ is —N(R^{e})₂ (e.g., —NH₂, —NMe₂, —NH(C₁₋₆ alkyl)). In certain embodiments, R³ is —NHR^{e}, and R^{e} is a nitrogen protecting group. In certain embodiments, R³ is optionally substituted acyl (e.g., —C(═O)(R^{e}), —C(═O)O(R^{e}), —C(═O)NH(R^{e}), —C(═O)N(R^{e})₂). In some embodiments, R³ is —C(═O)OMe. In some embodiments, R³ is —C(═O)OH.

In certain embodiments, R³ is optionally substituted alkyl. e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R³ is optionally substituted C₁₋₆ alkyl. In certain embodiments, R³ is unsubstituted C₁₋₆ alkyl. In certain embodiments, R³ is unsubstituted methyl. In certain embodiments, R³ is unsubstituted ethyl, propyl. or butyl. In certain embodiments, R³ is unsubstituted C₁₋₆ alkyl. in certain embodiments, R³ is substituted methyl. In certain embodiments, R³ is substituted ethyl, propyl, or butyl. In certain embodiments, R³ is optionally substituted alkenyl, e.g., optionally substituted C₂₋₆ alkenyl. In certain embodiments, R³ is vinyl, allyl, or prenyl. In certain embodiments, R³ is optionally substituted alkynyl, e.g., C₂₋₆ alkynyl.

In certain embodiments, R³ is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R³ is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R³ is optionally substituted aryl, e.g., optionally substituted phenyl. in certain embodiments, R³ is optionally substituted heteroaryl, e.g., optionally substituted 5-6 membered heteroaryl, or optionally substituted 9-10 membered bicyclic heteroaryl. In certain embodiments, R³ is optionally substituted aralkyl, e.g., optionally substituted benzyl. in certain embodiments. R³ is optionally substituted heteroaralkyl, e.g., methyl substituted with a 5-6-membered heteroaryl ring.

In some embodiments, V² is In certain embodiments, V¹ is ═N—.

In some embodiments, V¹ is ═CR³— and V² is ═N—. In certain embodiments, V¹ is ═CH— and V² is ═N—.

In certain embodiments, R^{V} is —N(R¹)₂ (e.g., —NH₂, —NMe₂, —NH(C₁₋₆ alkyl)). In certain embodiments, R^{V} is —NHR¹, and R¹ is a nitrogen protecting group. In some embodiments, R^{V} is —OR¹ (e.g., —O(C₁₋₆ alkyl)). In some embodiments, R^{V} is —OCH₃. In some embodiments, R^{V} is —OH. In some embodiments, R^{V} is halogen. In certain embodiments, R^{V} is —F, —Br, —Cl, or —I. In certain embodiments, R^{V} is —F. In certain embodiments, R^{V} is hydrogen.

In certain embodiments, there is one instance of R¹. In some embodiments there are two instances of R¹. In certain embodiments, each instance of R¹ is independently selected, wherein all instances of R¹ are different. In certain embodiments, each instance of R¹ is independently selected, wherein some instances of R^{I} are different. In certain embodiments, all instances of R¹ are the same.

In some embodiments, R¹ is hydrogen. In some embodiments, R¹ is an optionally substituted C₁₋₄ alkyl. In certain embodiments, R¹ is unsubstituted methyl. In some embodiments, R¹ is unsubstituted ethyl. In some embodiments, R¹ is unsubstituted propyl. In certain embodiments, R¹ is unsubstituted isopropyl. In some embodiments, R¹ is unsubstituted propyl. In some embodiments, R¹ is unsubstituted butyl, sec-butyl, iso-butyl, or tert-butyl. In certain embodiments, R¹ is substituted methyl. In some embodiments, R¹ is substituted ethyl. In some embodiments, R¹ is substituted propyl. In certain embodiments, R¹ is substituted isopropyl. In some embodiments, R¹ is substituted propyl. In some embodiments, R¹ is substituted butyl, sec-butyl, iso-butyl, or tert-butyl. In some embodiments, R¹ is an optionally substituted C₅₋₈ alkyl. In certain embodiments, R¹ is a halogen-substituted alkyl (e.g., trifluoromethyl, difluoromethyl, monofluoromethyl, —CH₂—CH₂F). in certain embodiments R¹ is halogen. In certain embodiments, R¹ is an alkyl substituted with one or more instances of —NO₂, —CN, —OR^{e}, —N(R^{e})₂, —SR^{e}, —C(═O)R^{e}, —C(═O)OR^{e}, or —C(═O)NR^{e}. In some embodiments, R¹ is —CH₂CH₂NH₂. In some embodiments, R¹ is —CH₂CH₂OH. In some embodiments, R¹ is an optionally substituted C₃₋₆ carbocyclyl (e.g., cyclopropyl, cyclobutyl, cyclopentyl, cyclohexyl). In some embodiments, R¹ is a C₇₋₁₄ carbocyclyl. In certain embodiments, R¹ is a monocyclic carbocyclyl. In some embodiments, R¹ is a bicyclic carbocyclyl. In certain embodiments, R¹ is an optionally substituted C₅₋₆ heterocyclyl (e.g., tetrahydrofuranyl, tetrahydropyranyl, pyrrolidinyl, piperidinyl, piperazinyl, morpholinyl). In some embodiments, R¹ is an optionally substituted C₇₋₁₄ heterocyclyl. In some embodiments, R¹ is an optionally substituted aryl. In certain embodiments, R¹ is an optionally substituted phenyl. In certain embodiments, R¹ is an optionally substituted naphthyl. In some embodiments, R¹ is optionally substituted monocyclic heteroaryl (e.g., pyridinvl, pyrimidinyl, pyrazinyl, pyrrolyl, (uranyl, thiophenyl, imidaolyl). In certain embodiments, R¹ is optionally substituted bicyclic heteroaryl (e.g., indenyl, indolyl, quinolinyl, isoquinolinyl). In some embodiments, R¹ is optionally substituted acyl (e.g., formyl, acetyl, propionyl, benzoyl, acryloyl, trifluoroacetyl). In certain embodiments, R¹ is a nitrogen protecting group. In certain embodiments, R¹ is an oxygen protecting group.

In certain embodiments. R² is hydrogen. In certain embodiments, R² is halogen. In certain embodiments, R² is —F. In certain embodiments, R² is —Cl, —Br, or —F. In certain embodiments, R² is —NO₂. In certain embodiments, R² is —CN. In certain embodiments, R² is —OR^{e} (e.g. —OH, —OMe, —O(C₁₋₆ alkyl)) In certain embodiments, R² is —OR^{e}, and R^{e} is an oxygen protecting group. In certain embodiments, R² is —N(R^{c})₂ (e.g., —NH₂, —NMe₂, —NH(C₁₋₆ alkyl)). In certain embodiments, R² is —NHR^{e}, and R^{e} is a nitrogen protecting group. In certain embodiments, R² is optionally substituted acyl (e.g., —C(═O)(R^{e}), —C(═O)O(R^{e}), —C(═O)NH(R^{e})—, —C(═O)N(R^{e})₂). In some embodiments, R² is —C(═O)OMe. In some embodiments, R² is —C (═O)OH.

In certain embodiments. R² is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R² is optionally substituted C₁₋₆ alkyl. In certain embodiments, R² is unsubstituted C₁₋₆ alkyl. In certain embodiments, R² is unsubstituted methyl, In certain embodiments, R² is unsubstituted ethyl, propyl, or butyl. In certain embodiments, R² is substituted methyl. In certain embodiments, R² is substituted ethyl, propyl, or butyl. In certain embodiments, R² is optionally substituted alkenyl, e.g., optionally substituted C₂₋₆ alkenyl. In certain embodiments, R² is vinyl, allyl, or prenyl. In certain embodiments, R² is optionally substituted alkynyl, e.g., C₂₋₆ alkynyl.

In certain embodiments, R² is optionally substituted carbocyclyl, e,g., optionally substituted C₃₋₆ carbocyclyl, optionally substituted C₃₋₄ carbocyclyl, optionally substituted C₄₋₅ carbocyclyl. or optionally substituted C₅₋₆ carbocyclyl. In certain embodiments R² is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R² is optionally substituted aryl, e.g., optionally substituted phenyl. In certain embodiments, R² is optionally substituted heteroaryl, e.g., optionally substituted 5-6 membered heteroaryl, or optionally substituted 9-10 membered bicyclic heteroaryl. In certain embodiments, R² is optionally substituted aralkyl, e.g., optionally substituted benzyl. In certain embodiments, R² is optionally substituted heteroaralkyl, e.g., methyl substituted with a 5-6-membered heteroaryl ring.

In some embodiments, R² is

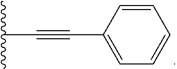 [SMILES: *C#Cc1ccccc1]

In certain embodiments. R² is

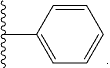 [SMILES: *c1ccccc1]

In some embodiments, R² is

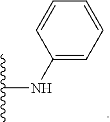 [SMILES: *Nc1ccccc1]

In certain embodiments, R² is

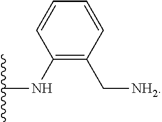 [SMILES: *Nc1ccccc1CN]

In some embodiments, R² is

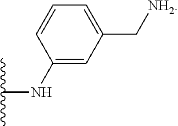 [SMILES: *Nc1cccc(CN)c1]

In certain embodiments, R² is

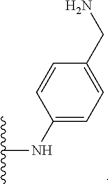 [SMILES: *Nc1ccc(CN)cc1]

### Ribose, Sugar, and Heterocycle Moiety

In some embodiments, W¹ is —O—. In certain embodiments, W¹ is —CR^{e}₂—. In certain embodiments, W¹ is —CH₂—. In certain embodiments, W¹ is —CF₂—. In some embodiments, W¹ is —NR^{e}—. In some embodiments, W¹ is —NR^{e}—, and R^{e} is H. In some embodiments, W¹ is —NR^{e}—, and R^{e} is —CH₃. In certain embodiments, W¹ is —S—.

In certain embodiments, R⁹ is hydrogen. In certain embodiments, R⁹ is halogen. In certain embodiments, R⁹ is —F. In certain embodiments, R⁹ is —Cl, —Br, or —F. In certain embodiments, R⁹ is —NO₂. In certain embodiments, R⁹ is —CN. In certain embodiments, R⁹ is —OR^{e} (e.g. —OH, —OMe, —O(C₁₋₆ alkyl)). In certain embodiments, R⁹ is —OH. In certain embodiments, R⁹ is —OR⁴. In certain embodiments, R⁹ is —OR⁵. In certain embodiments, R⁹ is —OR^{e}, and R^{e} is an oxygen protecting group. In certain embodiments, R⁹ is —N(R^{e})₂ (e.g., —NH₂, —NMe₂, —NH(C₁₋₆ alkyl)). In certain embodiments, R⁹ is —NHR^{e}, and R^{e} is a nitrogen protecting group. In certain embodiments, R⁹ is optionally substituted acyl (e.g., —C(═O)(R^{e}), —C(═O)O(R^{e}), —C(═O)NH(R^{e}), —C(═O)N(R^{e})₂). In some embodiments, R⁹ is —C(═O)OMe. In some embodiments, R⁹ is —C(═O)OH.

In certain embodiments, R⁹ is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R⁹ is optionally unsubstituted C₁₋₆ alkyl. In certain embodiments, R⁹ is unsubstituted C₁₋₆ alkyl. In certain embodiments, R⁹ is unsubstituted methyl. In certain embodiments, R⁹ is unsubstituted ethyl, propyl, or butyl. In certain embodiments, R⁹ is optionally substituted C₁₋₆ alkyl. In certain embodiments, R⁹ is substituted methyl (e.g., —CF₃, —CHF₂, —CH₂F). In certain embodiments, R⁹ is substituted ethyl, propyl, or butyl. In certain embodiments, R⁹ is optionally substituted alkenyl, e.g., optionally substituted C₂₋₆ alkenyl. In certain embodiments, R⁹ is vinyl, allyl, or prenyl. In certain embodiments, R⁹ is optionally substituted alkynyl, e.g., C₂₋₆ alkynyl.

In certain embodiments, R⁹ is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆, carbocyclyl. In certain embodiments R⁹ is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R⁹ is optionally substituted aryl, e.g., optionally substituted phenyl. in certain embodiments. R⁹ is optionally substituted heteroaryl, e.g.. optionally substituted 5-6 membered heteroaryl, or optionally substituted 9-10 membered bicyclic heteroaryl. In certain embodiments, R⁹ is optionally substituted aralkyl, e.g., optionally substituted benzyl. In certain embodiments, R⁹ is optionally substituted heteroaralkyl, e.g., methyl substituted with a 5-6-membered heteroaryl ring.

In certain embodiments, R¹⁰ is hydrogen. In certain embodiments, R¹⁰ is halogen. In certain embodiments, R¹⁰ is —F. In certain embodiments, R¹⁰ is —Cl, —Br, or —F. In certain embodiments, R¹⁰ is —NO₂. In certain embodiments, R¹⁰ is —CN. In certain embodiments, R¹⁰ is —OR^{e} (e.g. —OH, —OMe, —O(C₁₋₆ alkyl)). In certain embodiments, R¹⁰ is —OH. In certain embodiments, R¹⁰ is —OR⁴. In certain embodiments, R¹⁰ is —OR⁵. In certain embodiments, R¹⁰ is —OR^{e}, and R^{e} is an oxygen protecting group. In certain embodiments, R¹⁰ is —N(R^{e})₂ (e.g., —NH₂, —NMe₂, —NH(C₁₋₆ alkyl)). In certain embodiments, R¹⁰ is —NHR^{e}, and R^{e} is a nitrogen protecting group. In certain embodiments, R¹⁰ is optionally substituted acyl (e.g., —C(═O)(R^{e}), —C(═O)O(R^{e}), —C(═O)NH(R^{e}), —C(═O)N(R^{e})₂). In some embodiments, R¹⁰ is —C(═O)OMe. In some embodiments, R¹⁰ is —C(═O)OH.

In certain embodiments, R¹⁰ is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R¹⁰ is unsubstituted C₁₋₆ alkyl. In certain embodiments, R¹⁰ is unsubstituted methyl. In certain embodiments, R¹⁰ is unsubstituted ethyl, propyl, or butyl. In certain embodiments, R¹⁰ is optionally substituted C₁₋₆ alkyl. In certain embodiments, R¹⁰ is substituted methyl (e.g., —CF₃, —CHF₂, —CH₂F). In certain embodiments, R¹⁰ is substituted ethyl, propyl, or butyl. In certain embodiments, R¹⁰ is optionally substituted alkenyl, e.g., optionally substituted C₂₋₆ alkenyl. In certain embodiments. R¹⁰ is vinyl, allyl, or prenyl. In certain embodiments, R¹⁰ is optionally substituted alkynyl, e.g., C₂₋₆ alkynyl.

In certain embodiments, R¹⁰ is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R¹⁰ is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R¹⁰ is optionally substituted aryl, e.g., optionally substituted phenyl. In certain embodiments, R¹⁰ is optionally substituted heteroaryl, e.g., optionally substituted 5-6 membered heteroaryl, or optionally substituted 9-10 membered bicyclic heteroaryl. In certain embodiments, R¹⁰ is optionally substituted aralkyl, e.g., optionally substituted benzyl. In certain embodiments, R¹⁰ is optionally substituted heteroaralkyl, e.g., methyl substituted with a 5-6-membered heteroaryl ring.

In some embodiments, at least one of R⁹ or R¹⁰ is hydrogen. In some embodiments, at least one of R⁹ or R¹⁰ is —OR⁴. In some embodiments, at least one of R⁹ or R¹⁰ is —F.

In certain embodiments, R¹¹ is hydrogen. In certain embodiments, R¹¹ is halogen. In certain embodiments, R¹¹ is —F. In certain embodiments, R¹¹ is —Cl, —Br, or —F. In certain embodiments, R¹¹ is —NO₂. In certain embodiments, R¹¹ is —CN. In certain embodiments, R¹¹ is —OR⁴. In certain embodiments, R¹¹ is —OR⁵. In certain embodiments, R¹¹ is —OR^{e} (e.g. —OH, —OMe, —O(C₁₋₆ alkyl)). In certain embodiments, R¹¹ is —-OH. In certain embodiments, R¹¹ is —OR^{e}, and R^{e} is an oxygen protecting group. in certain embodiments, R¹¹ is —N(R^{e})₂ (e.g., —NH₂, —NMe₂, —NH(C₁₋₆ alkyl)). In certain embodiments, R¹¹ is —NHR^{e}, and R^{e} is a nitrogen protecting group. In certain embodiments, R¹¹ is optionally substituted acyl (e.g., —C(═O)(R^{e}), —C(═O)O(R^{e}), —C(═O)NH(R^{e}), —C(═O)N(R^{e})₂). In some embodiments, R¹¹ is —C(═O)OMe. In some embodiments, R¹¹ is —C(═O)OH.

In certain embodiments, R¹¹ is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R¹¹ is optionally substituted C₁₋₆ alkyl. In certain embodiments, R¹¹ is substituted methyl (e.g., —CF₃, —CHF₂, —CH₂F). In certain embodiments, R¹¹ is substituted ethyl, propyl, or butyl. In certain embodiments, R¹¹ is unsubstituted C₁₋₆ alkyl. In certain embodiments, R¹¹ is unsubstituted methyl. In certain embodiments, R¹¹ is unsubstituted ethyl, propyl, or butyl. In certain embodiments, R¹¹ is optionally substituted alkenyl. e.g., optionally substituted C₂₋₆ alkenyl. In certain embodiments, R¹¹ is vinyl, allyl, or prenyl. In certain embodiments, R¹¹ is optionally substituted alkynyl, e.g., C₂₋₆ alkynyl.

In certain embodiments, R¹¹ is optionally substituted carbocyclyl, e.g.. optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R¹¹ is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R¹¹ is optionally substituted aryl, e.g, optionally substituted phenyl. In certain embodiments, R¹¹ is optionally substituted heteroaryl, e.g., optionally substituted 5-6 membered heteroaryl, or optionally substituted 9-10 membered bicyclic heteroaryl. In certain embodiments. R¹¹ is optionally substituted aralkyl, e.g., optionally substituted benzyl. in certain embodiments, R¹¹ is optionally substituted heteroaralkyl, e.g., methyl substituted with a 5-6-membered heteroaryl ring.

In certain embodiments, R¹² is hydrogen. In certain embodiments, R¹² is halogen. In certain embodiments, R¹² is —F. In certain embodiments, R¹² is —Cl, —Br, or —F. In certain embodiments, R¹² is —NO₂. In certain embodiments, R¹¹ is —CN. In certain embodiments, R¹² is —OR¹¹ (e.g. —OH, —OMe, —O(C₁₋₆ alkyl)). In certain embodiments R¹² is —OH. In certain embodiments, R¹² is —OR⁴. In certain embodiments, R¹² is —OR⁵. In certain embodiments, R¹² is —OR^{e}, and R^{e} is an oxygen protecting group. In certain embodiments, R¹² is —N(R^{e})₂ (e.g., —NH₂, —NMe₂, —NH(C₁₋₆ alkyl)). In certain embodiments, R¹² is —NHR^{e}, and R^{e} is a nitrogen protecting group. In certain embodiments, R¹² is optionally substituted acyl (e.g., —C(═O)(R^{e}), —C(═O)N(R^{e}), —C(═O)NH(R^{e}), —C(═O)N(R^{e})₂). In some embodiments, R¹² is —C(═O)OMe. In some embodiments, R¹² is —C(═O)OH.

In certain embodiments, R¹² is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments, R¹² is optionally substituted C₁₋₆ alkyl. In certain embodiments, R¹²is substituted methyl (e.g., —CHF₂, —CH₂F). In certain embodiments, R¹² is substituted ethyl, propyl, or butyl. In certain embodiments, R¹² is unsubstituted C₁₋₆ alkyl. In certain embodiments, R¹² is unsubstituted methyl. In certain embodiments, R¹² is unsubstituted ethyl, propyl, or butyl. In certain embodiments. R¹² is optionally substituted alkenyl, e.g., optionally substituted C₂₋₆ alkenyl. In certain embodiments, R¹² is vinyl, allyl, or prenyl. In certain embodiments, R¹² is optionally substituted alkynyl, e.g., C₂₋₆ alkynyl.

In certain embodiments, R¹² is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R¹² is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R¹² is optionally substituted aryl, e.g., optionally substituted phenyl. In certain embodiments, R¹² is optionally substituted heteroaryl, e.g., optionally substituted 5-6 membered heteroaryl, or optionally substituted 9-10 membered bicyclic heteroaryl. In certain embodiments, R¹² is optionally substituted aralkyl, e.g., optionally substituted benzyl. In certain embodiments, R¹² is optionally substituted heteroaralkyl, e.g., methyl substituted with a 5-6-membered heteroaryl ring.

In some embodiments, at least one of R¹¹ or R¹² is hydrogen. In some embodiments, at least one of R¹¹ or R¹² is —OR⁵. In some embodiments, at least one of R¹¹ or R¹² is —F.

In certain embodiments, two occurrences of R⁹, R¹⁰, R¹¹, and R¹² groups are joined to form an optionally substituted carbocyclic ring. In certain embodiments, two occurrences of R⁹, R¹⁰, R¹¹, and R¹² groups are joined to form an optionally substituted C₃-C₆ heterocyclyl ring (e.g., cyclopropyl, cyclobutyl, cyclopentyl, cyclohexyl). In certain embodiments, two occurrences of R⁹, R¹⁰, R¹¹, and R¹² groups are joined to form an optionally substituted heterocyclic ring. In certain embodiments, two occurrences of R⁹, R¹⁰, R¹¹, and R¹² groups are joined to form an optionally substituted C₃-C₆ heterocyclyl ring (e.g., piperidinyl, piperazinyl, morpholinyl, pyrrolidinyl).

In some embodiments, R⁹ is —OR⁴, R¹⁰ is H, R¹¹is —OR⁵, and R¹² is H. In certain embodiments, R¹⁰ is —OR⁴, R⁹ is H, R¹² is —OR⁵, and R¹¹ is H. In some embodiments, R⁹ is —OH, R¹⁰ is H, R¹¹ is —OH, and R¹² is H. In certain embodiments, R¹⁰ is —OH, R⁹ is H, R¹² is —OH, and R¹¹ is H.

As generally defined herein, each of R⁴ and R⁵ is independently hydrogen, optionally substituted C₁₋₆ alkyl, optionally substituted acyl, or an oxygen protecting group, or R⁴ and R⁵ are joined to form an optionally substituted heterocyclic ring. The carbon to which R⁴ is attached may be in either the (R) or (S) configuration. The carbon to which R⁵ is attached may be in either the (R) or (S) configuration.

In certain embodiments, at least one of R⁴ and R⁵ is hydrogen. In certain embodiments, at least one of R⁴ and R⁵ is optionally substituted C₁₋₆ alkyl. In certain embodiments, at least one of R⁴ and R⁵ is unsubstituted C₁₋₆ alkyl. In certain embodiments, at least one of R⁴ and R⁵ is methyl. In certain embodiments, at least one of R⁴ and R⁵ is ethyl, propyl, or butyl. In certain embodiments, at least one of R⁴ and R⁵ is acyl (e.g., —C(═O)(R^{e}). —C(═O)O(R^{e}), —C(═O)NH(R^{e}), —C(═O)N(R^{e})₂). In certain embodiments, at least one of R⁴ and R⁵ is an oxygen protecting group. in some embodiments, at least one of R⁴ and R⁵ is silyl (e.g., TMS, TBDMS, TIPS). In some embodiments, at least one of R⁴ and R⁵ is acetyl (Ac), benzyl (Bn), benzoyl (Bz), or methoxymethyl ether (MOM).

In certain embodiments, both R⁴ and R⁵ are hydrogen. In certain embodiments, both R⁴ and R⁵ are optionally substituted C₁₋₆ alkyl. In certain embodiments, both R⁴ and R⁵ are unsubstituted C₁₋₆ alkyl. In certain embodiments, both R⁴ and R⁵ are methyl. In certain embodiments, both R⁴ and R⁵ are ethyl, propyl, or butyl. In certain embodiments, both R⁴ and R⁵ are acyl (e.g., —C(═O)(R^{e}), —C(═O)O(R^{e}), —C(═O)NH(R^{e}), —C(═O)N(R^{e})₂). In certain embodiments, both R⁴ and R⁵ are oxygen protecting groups. In some embodiments, both R⁴ and R⁵ are silyl (e.g., TMS, TBDMS, TIPS). In some embodiments, both R⁴ and R⁵ are acetyl (Ac), benzyl (Bn), benzoyl (Bz), or methoxymethyl ether (MOM).

In certain embodiments, R⁴ is hydrogen. In certain embodiments, R⁴ is optionally substituted C₁₋₆ alkyl, In certain embodiments, R⁴ is unsubstituted C₁₋₆ alkyl. In certain embodiments, R⁴ is methyl. In certain embodiments, R⁴ is ethyl, propyl, or butyl. In certain embodiments, R⁴ is acyl (e.g., —C(═O)(R^{e}), —C(═O)O(R^{e}), —C(═O)NH(R^{e}), —C(═O)N(R^{e})₂). In certain embodiments, R⁴ is an oxygen protecting group. In some embodiments, R⁴ is silyl (e.g., TMS, TBDMS, TIPS). In some embodiments, R⁴ is acetyl (Ac), benzyl (Bn), benzoyl (Bz), or methoxymethyl ether (MOM).

In certain embodiments. R⁵ is hydrogen. In certain embodiments, R⁵ is optionally substituted C₁₋₆ alkyl. In certain embodiments, R⁵ is unsubstituted C₁₋₆ alkyl. In certain embodiments, R⁵ is methyl. In certain embodiments, R⁵ is ethyl, propyl, or butyl. In certain embodiments, R⁵ is acyl (e.g., —C(═O)(R^{e}), —C(═O)O(R^{e}), —C(═O)N(R^{e})₂). In certain embodiments, R⁵ is an oxygen protecting group. In some embodiments, R⁵ is silyl (e.g., TMS, TBDMS, TIPS). In some embodiments, R⁵ is acetyl (Ac), benzyl (Bn), benzoyl (Bz), or methoxymethyl ether (MOM).

In certain embodiments, R⁴ and R⁵ are joined to form an optionally substituted heterocyclic ring. In certain embodiments, R⁴ and R⁵ are taken together to form a cyclic acetal (e.g., —C(CH₃)₂—).

As generally defined herein, each of R^{a} and R^{b} is independently hydrogen, halogen, optionally substituted C₁₋₆ alkyl, —OR^{e}, or —N(R^{e})₂. The carbon to which R^{a} and R^{b} is attached may be in either the (R) or (S) configuration. In certain embodiments, at least one of R^{a} and R^{b} is hydrogen. In certain embodiments, at least one of R^{a} and R^{b} is halogen. In some embodiments, at least one of R^{a} and R^{b} is —F. In some embodiments, at least one of R¹ and R^{b} is —Cl, —Br, or —I. In certain embodiments, at least one of R^{a} and R^{b} is optionally substituted C₁₋₆ alkyl. In certain embodiments, at least one of R^{a} and R^{b} is unsubstituted C₁₋₆ alkyl. In certain embodiments, at least one of R^{a} and R^{b} is methyl. In certain embodiments, at least one of R^{a} and R^{b} is ethyl, propyl, or butyl.

In certain embodiments, R^{a} is hydrogen. In certain embodiments, is halogen. In some embodiments, R^{a} is —F. In some embodiments, at least one of R^{a} is —Cl, —Br, or —I. In certain embodiments, R^{a} is optionally substituted C₁₋₆ alkyl. In certain embodiments, R^{a} is unsubstituted C₁₋₆ alkyl. In certain embodiments, R^{a} is methyl. In certain embodiments, R^{a} is ethyl, propyl, or butyl. In certain embodiments, R^{a} is —OR^{e}, e.g., —OH. In certain embodiments, R^{a} is —N(R^{e})₂. In certain embodiments, R^{a} is —NHR^{e}, e.g., —NH₂.

In certain embodiments, R^{b} is hydrogen. In certain embodiments, R^{b} is halogen. In some embodiments, R^{b} is —F. in some embodiments, at least one of R^{b} is —Cl, —Br, or —I. In certain embodiments, R^{b} is optionally substituted C₁₋₆ alkyl. In certain embodiments, R^{b} is unsubstituted C₁₋₆ alkyl. In certain embodiments, R^{b} is methyl. In certain embodiments, R^{b} is ethyl, propyl, or butyl. In certain embodiments, R^{b} is —OR^{e}, e.g., —OH. In certain embodiments, R^{b} is —N(R^{e})₂. In certain embodiments, R^{b} is —NHR^{e}, e.g., —NH₂.

In certain embodiments, both R^{a} and R^{b} are hydrogen. In certain embodiments, both R^{a} and R^{b} are halogen. In some embodiments, both R^{a} and R^{b} are —F. In some embodiments, both R^{a} and R^{b} are —Cl, —Br, or —I. In certain embodiments, both R^{a} and R^{b} are optionally substituted C₁₋₆ alkyl. In certain embodiments, both R^{a} and R^{b} are unsubstituted C₁₋₆ alkyl. In certain embodiments, both R^{a} and R^{b} are methyl. In certain embodiments, both R^{a} and R^{b} are ethyl, propyl, or butyl.

### Ring A Linker

In some embodiments, X² is a bond. In certain embodiments, X² is —O—. In certain embodiments, X² is —NH—. In certain embodiments, X² is —NR^{e}—, and R^{e} is unsubstituted alkyl. In certain embodiments, X² is —NR^{e}—, and R^{e} is methyl. In certain embodiments, X² is —NR^{e}—, and R^{e} is ethyl, propyl, or butyl. In certain embodiments, X² is —NR^{e}—, and R^{e} is a nitrogen protecting group. In certain embodiments, X² is —C(R^{e})₂—. In certain embodiments, X² is —C(R^{e})₂—, and R^{e} is hydrogen. In certain embodiments, X² is —C(R^{e})₂—, and R^{e} is C₁₋₆ alkyl.

In certain embodiments. Ring A is phenyl. In certain embodiments, Ring A is pyridinyl. In certain embodiments, Ring A is pyrimidinyl.

In certain embodiments, there are no instances of R⁸. In certain embodiments, there is a single instance of R⁸. In certain embodiments, there are multiple instances of R⁸. In certain embodiments, each instance of R⁸ is independently selected, wherein all instances of R⁸ are different. In certain embodiments, each instance of R⁸ is independently selected, wherein some instances of R⁸ are different. In certain embodiments, all instances of R⁸ are the same.

In certain embodiments, at least one instance of R⁸ is hydrogen. In certain embodiments, each instance of R⁸ is hydrogen. In certain embodiments, at least one instance of le is halogen (e.g., —F, —Cl, —Br, —I). In certain embodiments, at least one instance of R⁸ is F. In certain embodiments, at least one instance of R⁸ is Cl. In some embodiments, R⁸ is —O(R^{e}). In some embodiments, R⁸ is —OH. In certain embodiments, R⁸ is —O(C₁₋₆ alkyl). In some embodiments, R⁸ is —OCH₃. In some embodiments, R⁸ is —N(R^{e})₂. In certain embodiments, R⁸ is —NH₂. In some embodiments, R⁸ is —N(CH₃)₂. In some embodiments, R⁸ is —CN. In certain embodiments, R⁸ is optionally substituted acyl (e.g., —C(═O)CH₃, —C(═O)CH₂CH₃, —C(═O)CF₃). In certain embodiments, R⁸ is —C(═O)OH. In certain embodiments, R⁸ is —C(═O)CH₃. In certain embodiments, R⁸ is —C(═O)NH₂. In certain embodiments, R⁸ is —C(O)OH. In certain embodiments, R⁸ is —C(═O)N(CH₃)₂. In certain embodiments, at least one instance of R⁸ is optionally substituted C₁-C₆ alkyl (e.g., optionally substituted methyl (e.g., trifluoromethyl), optionally substituted ethyl, optionally substituted propyl). In certain embodiments, at least one instance of R⁸ is a halo-C₁-C₆ alkyl (e.g., trifluoromethyl, difluoromethyl, fluoromethyl). In certain embodiments, at least one instance of R⁸ is trifluoromethyl. In certain embodiments, R⁸ is optionally substituted alkenyl (e.g., optionally substituted vinylene). In certain embodiments, R⁸ is optionally substituted alkynyl (e.g., optionally substituted ethynyl). In certain embodiments, R⁸ is optionally substituted C₃-C₆ carbocyclyl ring (e.g., cyclopropyl, cyclopentyl, cyclohexyl). In certain embodiments, R⁸ is an optionally substituted C₃-C₆ heterocyclyl ring (e.g., piperidinyl, piperazinyl, morpholinyl, pyrrolidinyl). In certain embodiments, R⁸ is an optionally substituted aryl (e.g., phenyl, naphthyl). In certain embodiments, R⁸ is an optionally substituted heteroaryl (e.g., pyridinyl, pyrimidinyl, isoquinolinyl, thienopyrimidinyl). In certain embodiments, R⁸ is a nitrogen protecting group, oxygen protecting group, or sulfur protecting group.

In certain embodiments, two R⁸ groups are joined to form an optionally substituted carbocyclyl. In certain embodiments, two R⁸ groups are joined to form an optionally substituted C₃-C₆ carbocyclyl (e.g., cyclopropyl, cyclobutyl, cyclopentyl, cyclohexyl). In certain embodiments, two R⁸ groups are joined to form an optionally substituted heterocyclyl. In certain embodiments, two R⁸ groups are joined to form an optionally substituted C₃-C₆ heterocyclyl (e.g., piperidinyl, piperazinyl, morpholinyl, pyrrolidinyl). In certain embodiments, two R⁸ groups are joined to form an optionally substituted aryl. In certain embodiments, two R⁸ groups are joined to form an optionally substituted aryl (e.g., phenyl, naphthyl). In certain embodiments, two R⁸ groups are joined to form an optionally substituted heteroaryl ring. In certain embodiments, two R⁸ groups form an optionally substituted pyridinyl. In certain embodiments, two R⁸ groups form an optionally substituted pyrimidinyl. In certain embodiments, two R⁸ groups form an optionally substituted isoquinolinyl. In certain embodiments, two R⁸ groups form an optionally substituted thienopyrimidinyl.

In certain embodiments, q is 0. In certain embodiments, q is 1. In some embodiments, q is 2. In certain embodiments q is 3. In certain embodiments, q is 4.

In certain embodiments, the Ring A linker is substituted with R⁸ ortho to X². In certain embodiments, the Ring A linker is substituted with R⁸ meta to X². In some embodiments, the Ring A linker is substituted with R⁸ para to X². In some embodiments, q is 1 and R⁸ is —CF₃ ortha to X². In some embodiments, q is 1 and R⁸ is —CF₃ meta to X². In some embodiments, q is 1 and R⁸ is —CF₃ para to X². In some embodiments, q is 1 and R⁸ is —CH₃ ortho to X². In some embodiments, q is 1 and R⁸ is —CH₃ meta to X². In some embodiments, q is 1 and R⁸ is —CH₃ para to X². In some embodiments, q is 1 and R⁸ is —OH ortho to X². In some embodiments, q is 1 and R⁸ is —OH meta to X². In some embodiments, q is 1 and R⁸ is —OH porn to X². In some embodiments, q is 1 and R⁸ is —OCH₃ ortho to X². In some embodiments, q is 1 and R⁸ is —OCH₃ meta to X². In some embodiments, q is 1 and R⁸ is —OCH₃ para to X². In some embodiments, q is 1 and R⁸ is —NH₂ ortho to X². In some embodiments, q is 1 and R⁸ is —NH₂ meta to X². In some embodiments, q is 1 and R⁸ is —NH₂ para to X². In some embodiments, q is 1 and R⁸ is —NMe₂ ortho to X². In some embodiments, q is 1 and R⁸ is —NMe₂ meta to X². In some embodiments, q is 1 and R⁵ is —NMe₂ para to X². In some embodiments, q is 1 and R⁸ is —CN ortho to X². In some embodiments, q is 1 and R⁸ is —CN meta to X². In some embodiments, q is 1 and R⁸ is —CN para to X². In some embodiments. q is 1 and R⁸ is —Cl ortho to X². In some embodiments, q is 1 and R⁸ is —Cl meta to X². In some embodiments, q is 1 and R⁸ is —Cl para to X². in some embodiments, q is 1 and R⁸ is —F ortho to X². In some embodiments, q is 1 and R⁸ is —F meta to X². In some embodiments, q is 1 and R⁸ is —F para to X². In some embodiments, q is 1 and R⁸ is —COOH ortho to X². In some embodiments, q is 1 and R⁸ is —COOH meta to X². In some embodiments, q is 1 and R⁸ is —COOH para to X². In some embodiments, q is 1 and R⁸ is —COOCH₃ ortho to X². In some embodiments, q is 1 and R⁸ is —COOCH₃ meta to X². In some embodiments, q is 1 and R⁸ is —COOCH₃ para to X². In some embodiments, q is 1 and R⁸ is —COONH₂ ortho to X². In some embodiments, q is 1 and R⁸ is —COONH₂ meta to X². In some embodiments, q is 1 and R⁸ is —COONH₂ para to X². In some embodiments, q is 1 and R⁸ is —COONMe₂ ortho to X². In some embodiments, q is 1 and R⁸ is —COONMe₂ meta to X². In some embodiments, q is 1 and R⁸ is —COONMe₂ para to X².

In some embodiments, X¹ is attached to Ring A para to X². In some embodiments, X¹ is attached to Ring A meta to X². In some embodiments, X¹ is attached to Ring A ortho to X².

In some embodiments, Ring A is phenylene.

In some embodiments, Ring A is pyridinylene. In some embodiments, the pyridine nitrogen of Ring A is meta to X². In some embodiments, the pyridine nitrogen of Ring A is para to X². In some embodiments, the pyridine nitrogen of Ring A is ortho to X².

### Bridging Linker

In some embodiments, X¹ is a bond. In certain embodiments, X¹ is —O—. In certain embodiments, X¹ is —NH—. In certain embodiments, X¹ is —NR^{e}—, and R¹ is unsubstituted C₁₋₆ alkyl. In certain embodiments, X¹ is and —NR^{e}— is methyl. In certain embodiments, X¹ is —NR^{e}—, and R^{e} is ethyl, propyl, or butyl. In certain embodiments, X¹ is —NR^{e}—, and R^{e} is a nitrogen protecting group. In certain embodiments, X¹ is —C(R^{e})₂—. In certain embodiments, X¹ is —C(R^{e})₂— and R^{e} is hydrogen. In certain embodiments, X¹ is —C(R^{e})₂— and R^{e} is C₁₋₆ alkyl.

In certain embodiments, X¹ is bound to the Ring A linker orrho to X². In certain embodiments, X¹ is bound to the Ring A linker meta to X². In certain embodiments, X¹ is bound to the Ring A linker para to X².

In certain embodiments, m is 0. In some embodiments, in is 1. In certain embodiments, in is 2. In some embodiments, in is 3, 4, 5, or 6.

In some embodiments, X¹ is a bond and m is 2. In some embodiments, X¹ is a bond and m is 1. In some embodiments, X¹ is —CH₂— and m is 1.

R⁶, Z and Y

As generally defined herein, R⁶ is of the formula:

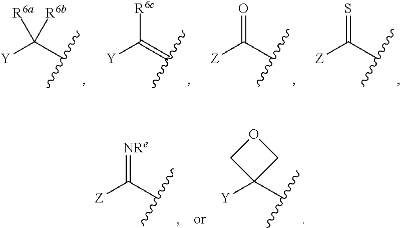

In certain embodiments, R⁶ is of formula:

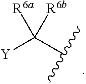

In some embodiments, R⁶ is of the formula:

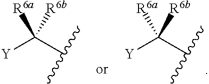

In certain embodiments, R⁶ is of the formula:

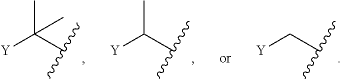

In certain embodiments, R⁶ is of the formula:

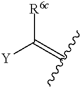

In some embodiments, R⁶ is of the formula:

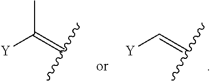

In certain embodiments, R⁶ is of the formula:

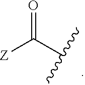

In some embodiments, R⁶ is of the fommla:

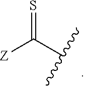

In certain embodiments. R⁶ is of the formula:

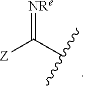

In some embodiments, R⁶ is of the formula:

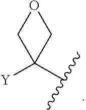

In certain embodiments, R⁶ is of formula:

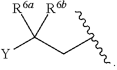

In some embodiments, R⁶ is of formula:

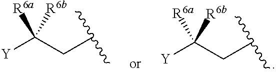

In certain embodiments, R⁶ is of formula:

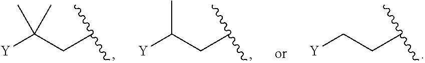

In certain embodiments, R⁶ is of formula:

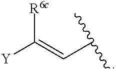

In some embodiments, R⁶ is of formula:

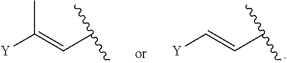

In certain embodiments, R⁶ is of formula:

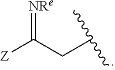

In some embodiments, R⁶ is of formula:

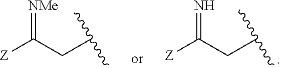

In certain embodiments, R⁶ is of formula:

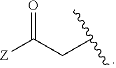

In some embodiments, R⁶ is of formula:

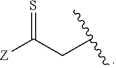

In certain embodiments, R⁶ is of formula:

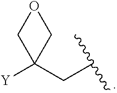

In some embodiments, X¹ is a bond, m is 2, and R⁶ is

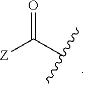

In some embodiments, X¹ is a bond, m is 1, and R⁶ is

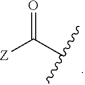

In some embodiments, X¹ is —CH₂—, m is 1, and R⁶ is

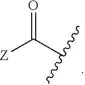

In some embodiments, X¹ is a bond, m is 2, and R⁶ is

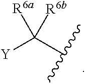

In some embodiments, X¹ is a bond, m is I, and R⁶ is

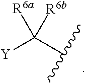

In some embodiments, X¹ is CH₂—, m is 1, and R⁶ is

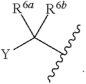

In certain embodiments, Y is optionally substituted alkyl (e.g., optionally substituted C₁₋₆ alkyl), optionally substituted alkenyl (e.g., optionally substituted C₁₋₆ alkenyl), or optionally substituted alkynyl (e.g., optionally substituted C₁₋₆ alkynyl). In certain embodiments, Y is optionally substituted heteroalkyl (e.g., optionally substituted C₁₋₆ heteroalkyl), optionally substituted heteroalkenyl (e.g., optionally substituted C₁₋₆ heteroalkenyl), or optionally substituted heteroalkynyl (e.g., optionally substituted C₁₋₆ heteroalkynyl). In certain embodiments, Y is optionally substituted alkoxy (e.g., optionally substituted C₁₋₆ alkoxy), optionally substituted amino, —OR^{e}, or —N(R^{e})₂. In certain embodiments, Y is optionally substituted carbocyclyl (e.g., optionally substituted monocyclic 3- to 7-membered carbocyclyl). In certain embodiments. Y is optionally substituted aryl (e.g., optionally substituted 6- to 14-membered aryl, e.g.. optionally substituted phenyl). In certain embodiments, Y is optionally substituted heteroaryl (e.g., optionally substituted tnonocyclic 5- or 6-membered heteroaryl, wherein 1, 2, 3, or 4 atoms in the heteroaryl ring system are independently oxygen, nitrogen, or sulfur). In certain embodiments, Y is optionally substituted heterocyclyl, optionally substituted 6-membered heteroaryl. in certain embodiments, Y is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl. In certain embodiments, Y is optionally substituted 6-membered heteroaryl, e.g., optionally substituted pyridyl. In certain embodiments, Y comprises a substituted five-membered carbocyclyl or a substituted five-membered heterocyclyl. In certain embodiments, Y comprises a fused bicyclic comprising a five-membered carbocyclyl ring fused to an optionally substituted aryl ring or a five-membered heterocyclyl ring fused to an optionally substituted aryl ring.

In some embodiments, Y is of formula:

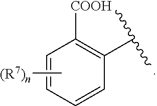

In some embodiments, Y is of formula:

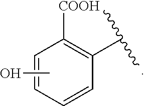

In certain embodiments. Y is of formula:

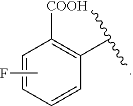

In certain embodiments, Y is of formula:

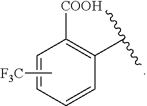

In certain embodiments, Y is of formula:

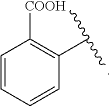 [SMILES: *c1ccccc1C(=O)O]

In some embodiments, Y is of formula:

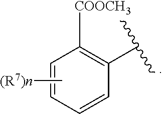

In some embodiments, Y is of formula:

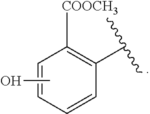

In some embodiments, Y is of formula:

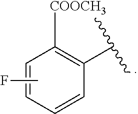

In some embodiments, Y is of formula:

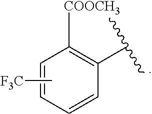

In some embodiments, Y is of formula:

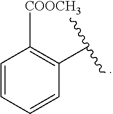 [SMILES: *c1ccccc1C(=O)OC]

In some embodiments, Y is of formula:

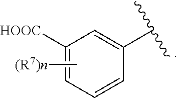

In some embodiments, Y is of formula:

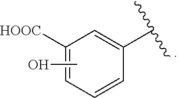

In certain embodiments, Y is of formula:

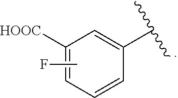

In certain embodiments, Y is of formula:

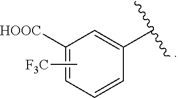

In certain embodiments, Y is of formula:

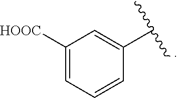 [SMILES: *c1cccc(C(=O)O)c1]

In some embodiments, Y is of formula:

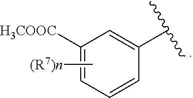

In some embodiments, Y is of formula:

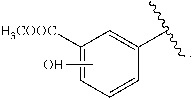

In certain embodiments, Y is of formula:

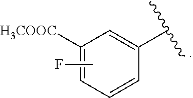

In certain embodiments, Y is of formula:

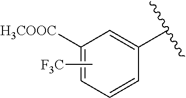

In certain embodiments, Ne is of formula:

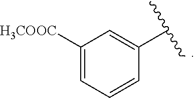

In certain embodiments, Y is of formula:

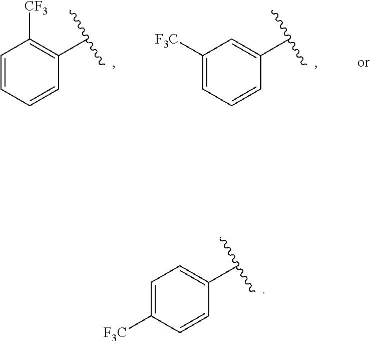 [SMILES: *c1ccccc1C(F)(F)F ; *c1cccc(C(F)(F)F)c1 ; *c1ccc(C(F)(F)F)cc1]

In some embodiments, Y is of fonnula:

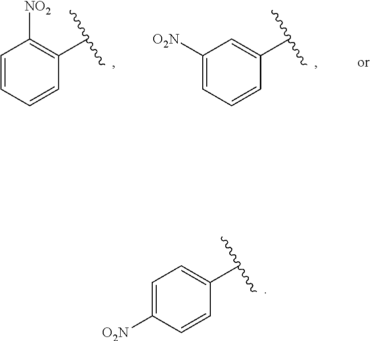 [SMILES: *c1ccccc1[N+](=O)[O-] ; *c1cccc([N+](=O)[O-])c1 ; *c1ccc([N+](=O)[O-])cc1]

In certain embodiments, Y is of formula

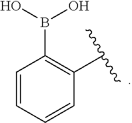 [SMILES: *c1ccccc1B(O)O]

In some embodiments, Y is of formula

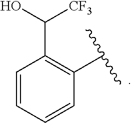 [SMILES: *c1ccccc1C(O)C(F)(F)F]

In certain embodiments. Y is of formula

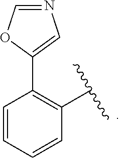 [SMILES: *c1ccccc1-c1cnco1]

In some embodiments, Y is of formula

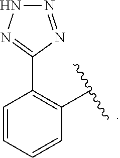 [SMILES: *c1ccccc1-c1nn[nH]n1]

In certain embodiments, Y is of formula

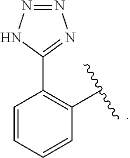 [SMILES: *c1ccccc1-c1nnn[nH]1]

In some embodiments, Y is of formula

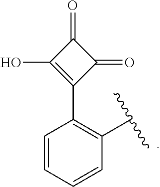 [SMILES: *c1ccccc1-c1c(O)c(=O)c1=O]

In certain embodiments, Y is of formula:

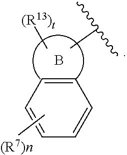

In certain embodiments, is of formula:

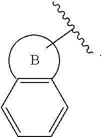

In certain embodiments, Y is of formula:

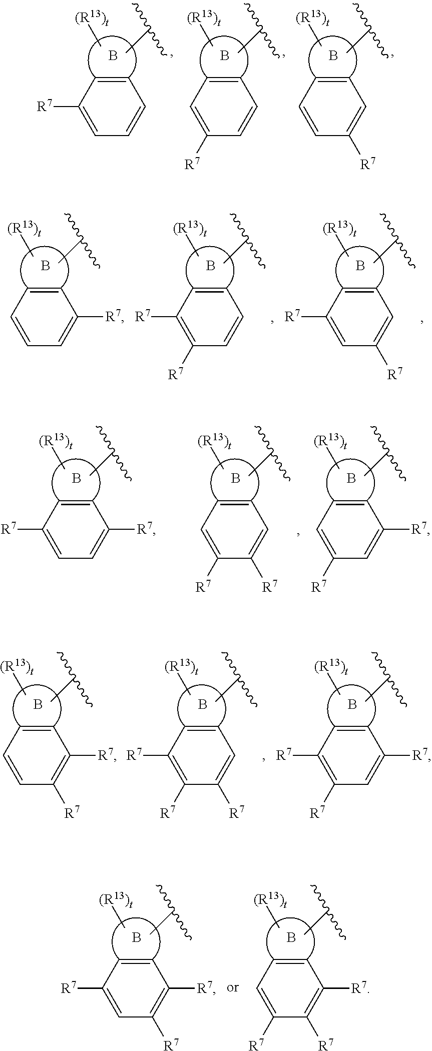

In certain embodiments, Ring B is an optionally substituted carbocyclic ring (e.g., an optionally substituted 5- to 6-membered carbocyclic ring). In certain embodiments, Ring B is a optionally substituted heterocyclic ring (e.g., an optionally substituted 5- to 6-membered heterocyclic ring, comprising 0 to 3 heteroatoms independently selected from O, N, and S). In certain embodiments, Ring B is an optionally substituted aryl ring (e.g., an optionally substituted phenyl ring). In certain embodiments, Ring B is an optionally substituted heteroaryl ring (e.g., an optionally substituted 5- to 6-membered heteroaryl ring, comprising 0 to 3 heteroatoms independently selected from O, N, and S).

In certain embodiments, Y is of formula:

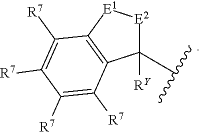

In certain embodiments, Y is of formula:

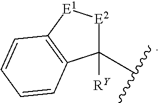

In certain embodiments, Y is of formula:

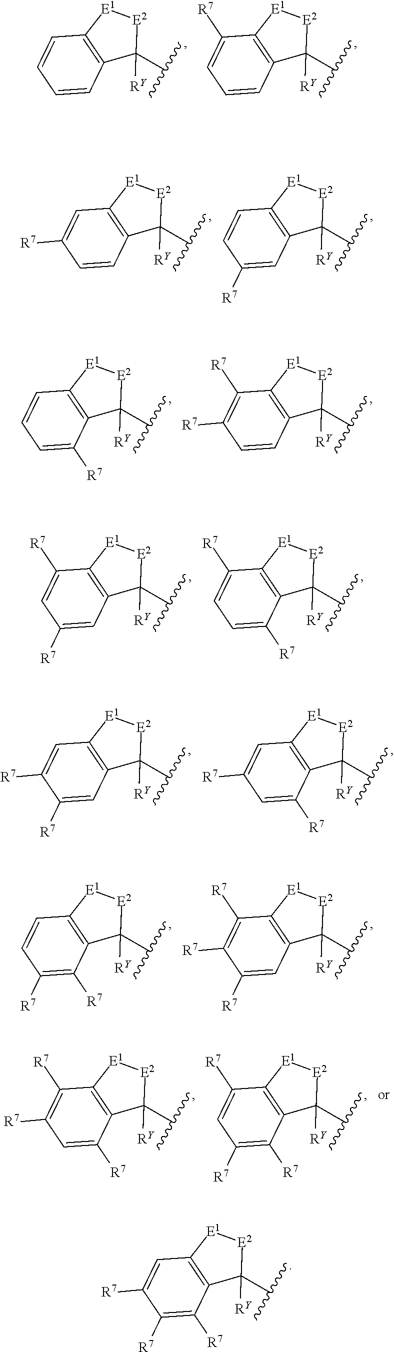

In certain embodiments, Y is of formula:

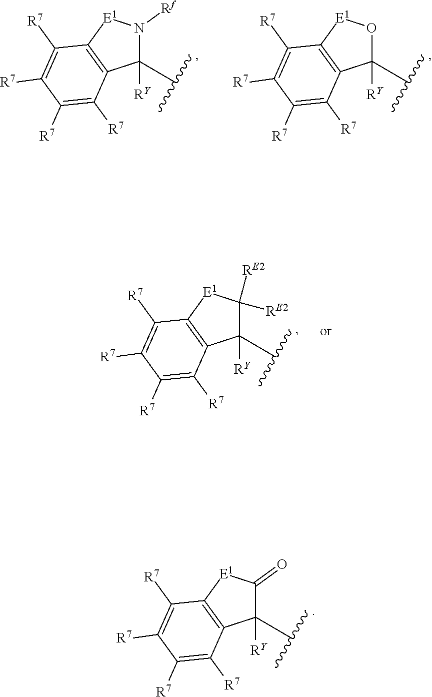

In certain embodiments, Y is of formula:

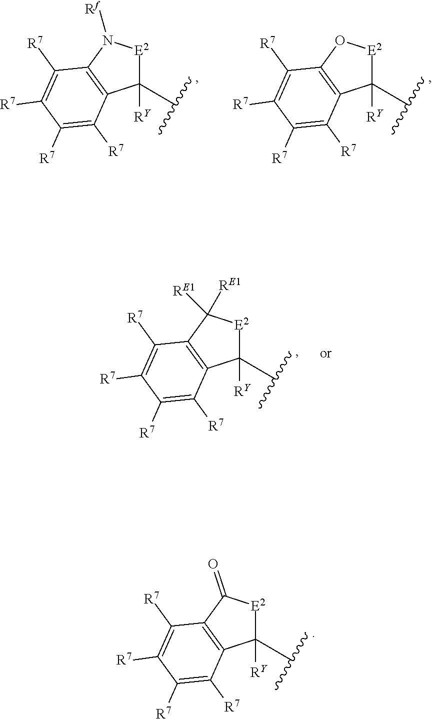

In certain embodiments, Y is of formula:

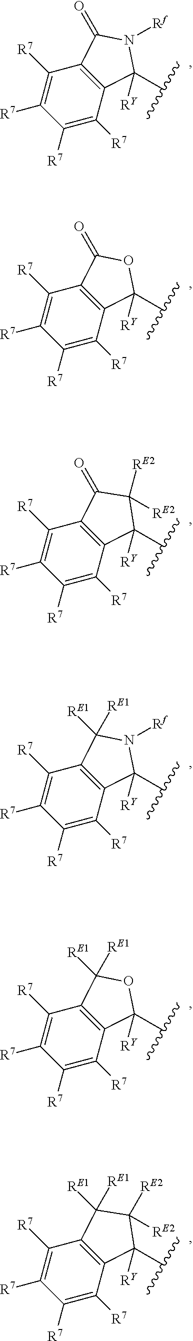

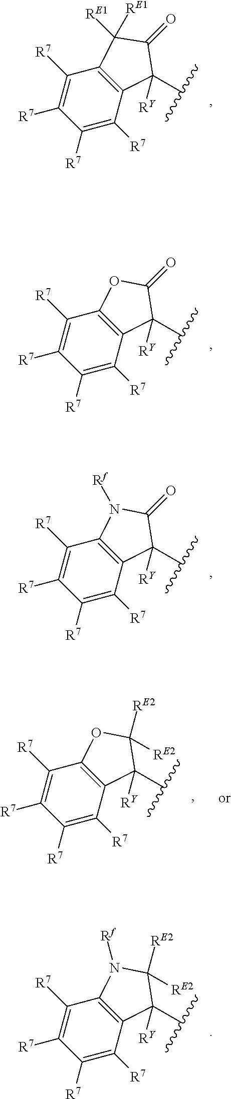

In certain embodiments, Y is of formula:

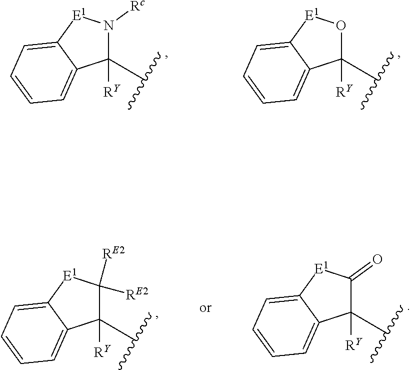

In certain embodiments, Y is of formula:

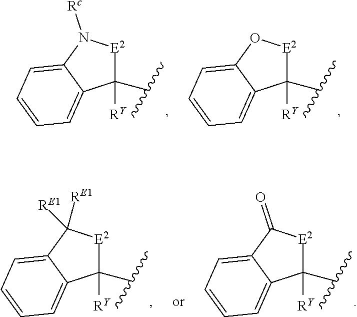

In certain embodiments, Y is:

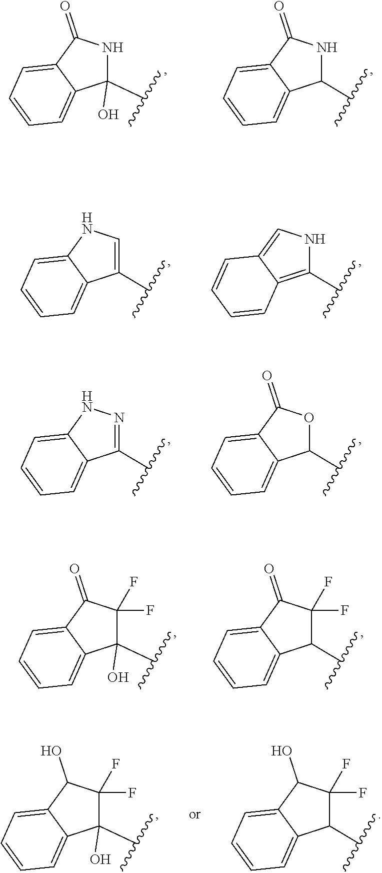

In certain embodiments, Y is:

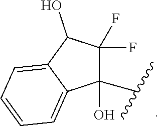 [SMILES: *C1(O)c2ccccc2C(O)C1(F)F]

In certain embodiments, Y is of one of the following formulae:

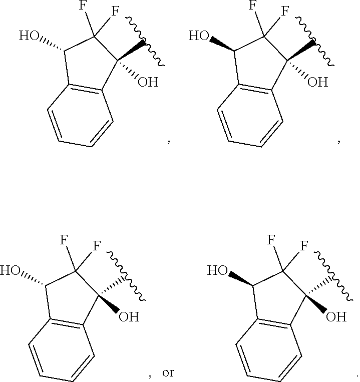 [SMILES: *[C@@]1(O)c2ccccc2[C@H](O)C1(F)F ; *[C@@]1(O)c2ccccc2[C@@H](O)C1(F)F ; *[C@]1(O)c2ccccc2[C@H](O)C1(F)F ; *[C@]1(O)c2ccccc2[C@@H](O)C1(F)F]

In certain embodiments, Z is optionally substituted alkyl (e.g., optionally substituted C₁₋₆ alkyl), optionally substituted alkenyl (e.g., optionally substituted C₁₋₆ alkenyl), or optionally substituted alkynyl (e.g, optionally substituted C₁₋₆ alkynyl). In certain embodiments, Z is optionally substituted heteroalkyl (e.g., optionally substituted C₁₋₆ heteroalkyl), optionally substituted heteroalkenyl (e.g., optionally substituted C₁₋₆ heteroalkenyl), or optionally substituted heteroalkynyl (e.g., optionally substituted C₁₋₆ heteroalkynyl). In certain embodiments, Z is optionally substituted alkoxy (e.g., optionally substituted C₁₋₆ alkoxy), optionally substituted amino, —OR^{e}, or —N(R^{e})₂. In certain embodiments, Z is optionally substituted carbocyclyl (e.g., optionally substituted monocyclic 3-, to 7-membered carbocyclyl). In certain embodiments, Z is optionally substituted aryl (e.g., optionally substituted 6- to 14-membered aryl, e.g.. optionally substituted phenyl). In certain embodiments, Z is optionally substituted heteroaryl (e.g., optionally substituted monocyclic 5- or 6-membered heteroaryl, wherein 1, 2, 3, or 4 atoms in the heteroaryl ring system are independently oxygen, nitrogen, or sulfur). In certain embodiments, Z is optionally substituted heterocyclyl, optionally substituted 6-membered heteroaryl. In certain embodiments, Z is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl. In certain embodiments, Z is optionally substituted 6-membered heteroaryl, e.g., optionally substituted pyridyl. In certain embodiments, Z comprises a substituted five-membered carbocyclyl or a substituted five-membered heterocyclyl. In certain embodiments, Z comprises a fused bicyclic comprising a five-membered carbocyclyl ring fused to an optionally substituted aryl ring or a five-membered heterocyclyl ring fused to an optionally substituted aryl ring.

In some embodiments, Z is of formula:

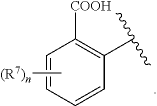

In some embodiments, Z is of formula:

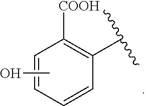

In certain embodiments, Z is of formula:

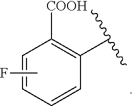

In certain embodiments, Z is of formula:

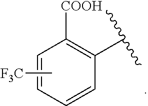

In certain embodiments, Z is of formula:

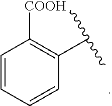 [SMILES: *c1ccccc1C(=O)O]

In some embodiments, Z is of formula:

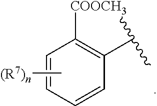

In some embodiments, Z is of formula:

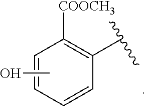

In some embodiments, Z is of formula:

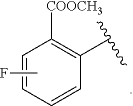

In some embodiments. Z is of formula:

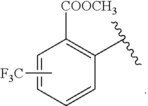

In some embodiments, Z is of formula:

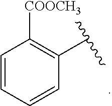 [SMILES: *c1ccccc1C(=O)OC]

In some embodiments, Z is of formula:

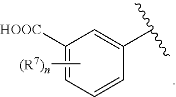

In some embodiments, Z is of formula:

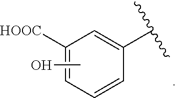

In certain embodiments, Z is of formula:

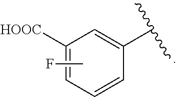

In certain embodiments, Z is of formula:

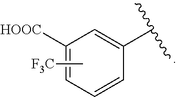

In certain embodiments, Z is of formula:

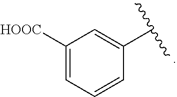 [SMILES: *c1cccc(C(=O)O)c1]

In some embodiments, Z is of formula:

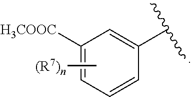

In some embodiments, Z is of formula:

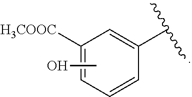

In certain embodiments, Z is of formula:

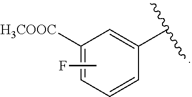

In certain embodiments, Z is of formula:

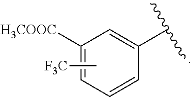

In certain embodiments, Z is of formula:

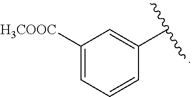

In certain embodiments, Z is of formula:

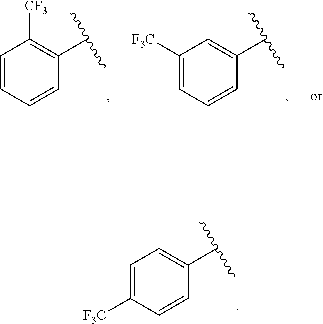 [SMILES: *c1ccccc1C(F)(F)F ; *c1cccc(C(F)(F)F)c1 ; *c1ccc(C(F)(F)F)cc1]

In some embodiments, Z is of formula:

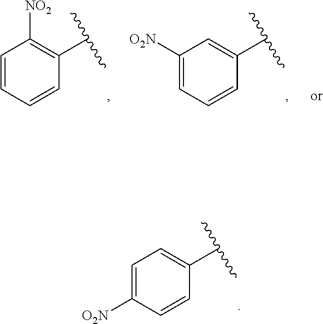 [SMILES: *c1ccccc1[N+](=O)[O-] ; *c1cccc([N+](=O)[O-])c1 ; *c1ccc([N+](=O)[O-])cc1]

In certain embodiments, Z is of formula:

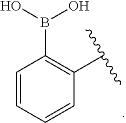 [SMILES: *c1ccccc1B(O)O]

In some embodiments, Z is of formula:

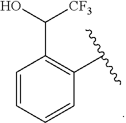 [SMILES: *c1ccccc1C(O)C(F)(F)F]

In certain embodiments, Z is of formula:

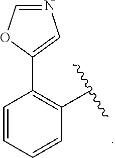 [SMILES: *c1ccccc1-c1cnco1]

In some embodiments, Z is of formula:

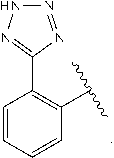 [SMILES: *c1ccccc1-c1nn[nH]n1]

In certain embodiments, Z is of formula:

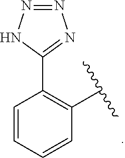 [SMILES: *c1ccccc1-c1nnn[nH]1]

In some embodiments, Z is of formula:

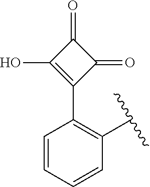 [SMILES: *c1ccccc1-c1c(O)c(=O)c1=O]

In certain embodiments, Z is of formula:

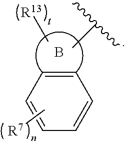

In certain embodiments, Z is of formula:

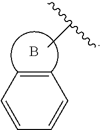

In certain embodiments, Z is of formula:

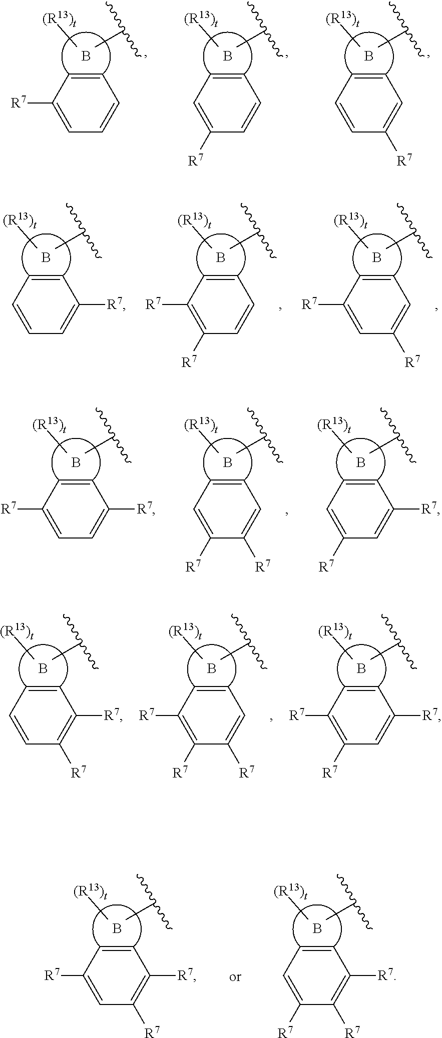

In certain embodiments, Ring B is an optionally substituted carbocyclic ring (e.g., an optionally substituted 5- to 6-membered carbocyclic ring). In certain embodiments, Ring B is a optionally substituted heterocyclic ring (e.g., an optionally substituted 5- to 6-membered heterocyclic ring, comprising 0 to 3 heteroatoms independently selected from O, N, and S). In certain embodiments, Ring B is an optionally substituted aryl ring (e.g., an optionally substituted phenyl ring). In certain embodiments, Ring B is an optionally substituted heteroaryl ring (e.g., an optionally substituted 5- to 6-membered heteroaryl ring, comprising 0 to 3 heteroatoms independently selected from O, N, and S).

In certain embodiments, Z is of formula:

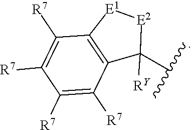

In certain embodiments, Z is of formula:

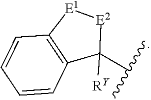

In certain embodiments, Z is of formula:

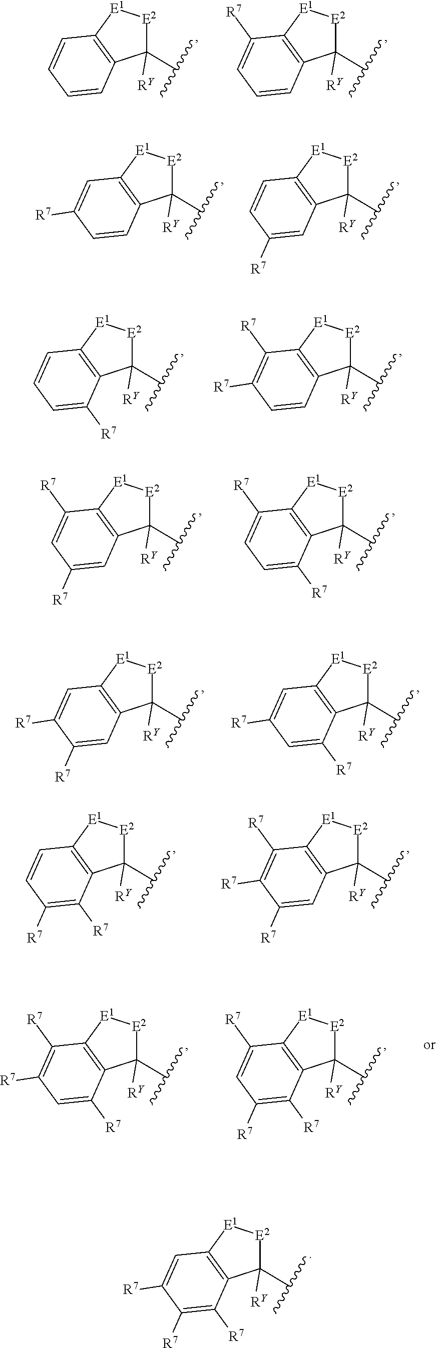

In certain embodiments, Z is of formula:

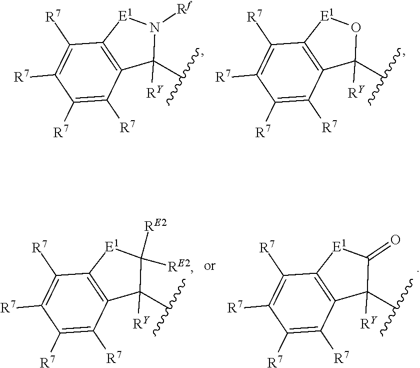

In certain embodiments, Z is of formula:

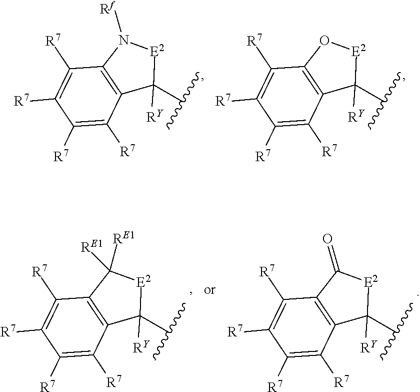

In certain embodiments, Z is of formula:

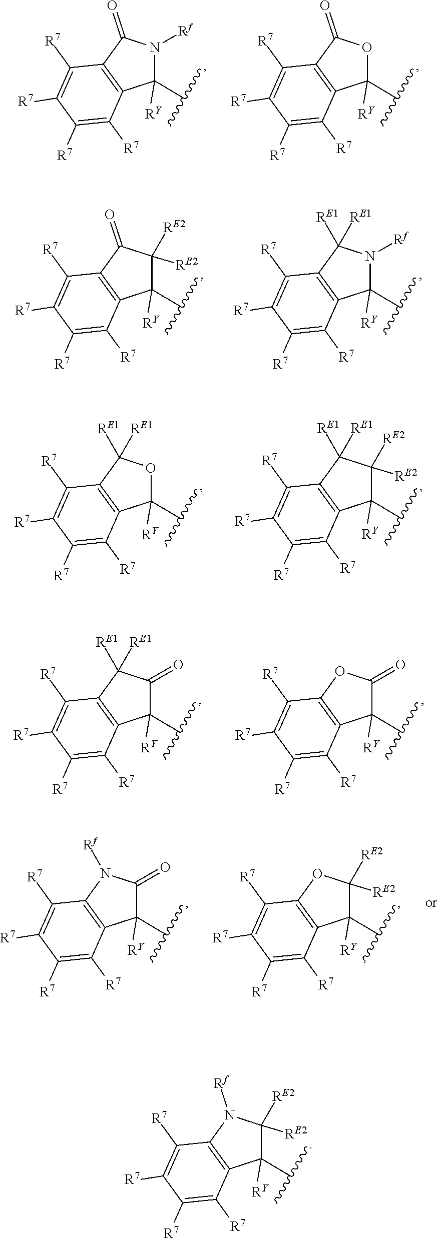

In certain embodiments, Z is of formula:

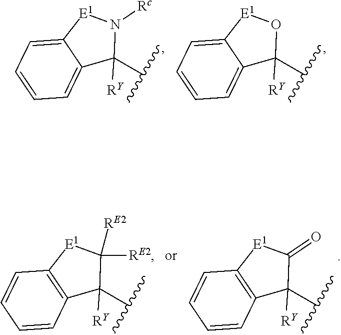

In certain embodiments, Z is of formula:

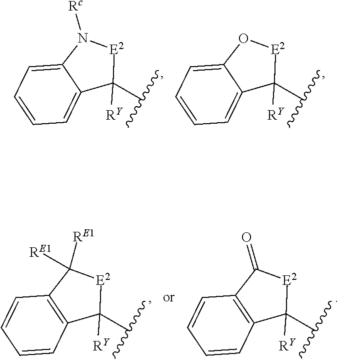

In certain embodiments, Z is of formula:

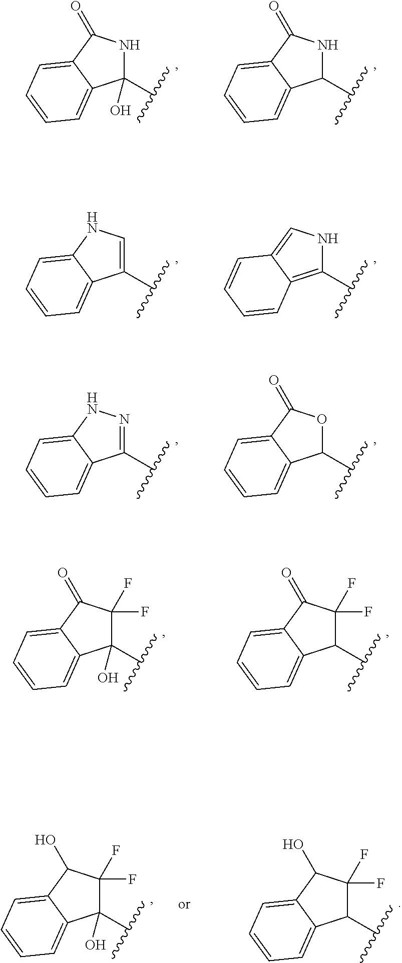

In certain embodiments, Z is of formula:

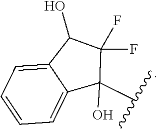 [SMILES: *C1(O)c2ccccc2C(O)C1(F)F]

In certain embodiments, Z is of formula:

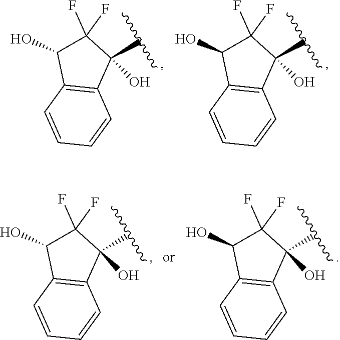 [SMILES: *[C@@]1(O)c2ccccc2[C@H](O)C1(F)F ; *[C@@]1(O)c2ccccc2[C@@H](O)C1(F)F ; *[C@]1(O)c2ccccc2[C@H](O)C1(F)F ; *[C@]1(O)c2ccccc2[C@@H](O)C1(F)F]

As generally defined herein, each of R^{6a} and R^{6b}, as contained in R⁶, is independently hydrogen, halogen, optionally substituted C₁₋₆ alkyl, —OR^{e}, or —N(R^{e})₂. The carbon to which R^{6a} and R^{6b} is attached may be in either the (R) or (S) configuration. In certain embodiments, at least one of R^{6a} and R^{6b} is hydrogen. In certain embodiments, at least one of R^{6a} and R^{6b} is halogen. In some embodiments, at least one of R^{6a} and R^{6b} is —F. In some embodiments, at least one of R^{6a} and R^{6b} is —Cl, —Br, or —I. In certain embodiments, at least one of R^{6a} and R^{6b} is optionally substituted C₁₋₆ alkyl. In certain embodiments, at least one of R^{6a} and R^{6b} is unsubstituted C₁₋₆ alkyl. In certain embodiments, at least one of R^{6a} and R^{6b} is methyl. In certain embodiments, at least one of R^{6a} and R^{6b} is ethyl, propyl, or butyl.

In certain embodiments, both R^{6a} and R^{6b} are hydrogen. In certain embodiments, both R^{6a} and R^{6b} are halogen. In some embodiments, both R^{6a} and R^{6b} are —F. In some embodiments. both R^{6a} and R^{6b} are —Cl, —Br, or —I. In certain embodiments, both R^{6a} and R^{6b} are optionally substituted C₁₋₆ alkyl. In certain embodiments, both R^{6a} and R^{6b} are unsubstituted C₁₋₆ alkyl. In certain embodiments, both R^{6a} and R^{6b} are methyl. In certain embodiments, both R^{6a} and R^{6b} are ethyl, propyl, or butyl.

In certain embodiments, R^{6a} is hydrogen. In certain embodiments, R^{6a} is halogen. In some embodiments, R^{6a} is —F. In some embodiments, at least one of R^{6a} is —Cl, —Br, or —I. In certain embodiments, R^{6a} is optionally substituted C₁₋₆ alkyl. In certain embodiments, R^{6a} is unsubstituted C₁₋₆ alkyl. In certain embodiments, R^{6a} is methyl. In certain embodiments, R^{6a} is ethyl, propyl, or butyl. In certain embodiments, R^{6a} is —OR^{e}, e.g., —OH. In certain embodiments, R^{6a} is —N(R^{e})₂. In certain embodiments, R^{6a} is —NHR^{e}, e.g., —NH₂.

In certain embodiments. R^{6b} is hydrogen. In certain embodiments, R^{6b} is halogen. In some embodiments, R^{6b} is —F. In some embodiments, at least one of R^{6b} is —Cl, —Br, or —I. In certain embodiments, R^{6b} is optionally substituted C₁₋₆ alkyl. In certain embodiments, R^{6b} is unsubstituted C₁₋₆ alkyl. In certain embodiments, R^{6b} is methyl. In certain embodiments, R^{6b} is ethyl, propyl, or butyl. In certain embodiments, R^{6b} is —OR^{e}, e.g., —OH. In certain embodiments, R^{6b} is —N(R^{e})₂. In certain embodiments, R^{6b} is —NHR^{e}, e.g., —NH₂.

In certain embodiments, R^{6e} is hydrogen. In certain embodiments, R^{6e} is halogen. In some embodiments, R^{6e} is —F. In some embodiments, at least one of R^{6e} is —Cl, —Br, or —I. In certain embodiments, R^{6e} is optionally substituted C₁₋₆ alkyl. In certain embodiments, R^{6e} is unsubstituted C₁₋₆ alkyl. In certain embodiments, R^{6e} is methyl. In certain embodiments, R^{6e} is ethyl, propyl, or butyl. In certain embodiments, R^{6e} is —OR^{e}, e.g., —OH. In certain embodiments, R^{6e} is —N(R^{e})₂. In certain embodiments, R^{6e} is —NHR^{e}, e.g., —NH₂.

### R^{e}

In certain embodiments, there are no instances of R^{e}. In certain embodiments, there is a single instance of R^{e}. In certain embodiments, there are multiple instances of R^{e}. In certain embodiments, each instance of R^{e} is independently selected, wherein all instances of R^{e} are different. In certain embodiments, each instance of R^{e} is independently selected, wherein some instances of R^{e} are different. In certain embodiments, all instances of R^{e} are the same.

In certain embodiments, at least one instance of R^{e} is hydrogen. In certain embodiments, each instance of R^{e} is hydrogen. In certain embodiments, R^{e} is optionally substituted acyl (e.g., —C(═O)CH₃, —C(═O)CH₂CH₃, —C(═O)CF₃). In certain embodiments, at least one instance of R^{e} is optionally substituted C₁-C₆ alkyl (e.g., optionally substituted methyl (e.g., trifluoromethyl), optionally substituted ethyl, optionally substituted propyl). In certain embodiments, R^{e} is optionally substituted alkenyl (e.g., optionally substituted vinylene). In certain embodiments, R^{e} is optionally substituted alkynyl (e.g., optionally substituted ethynyl). In certain embodiments, R^{e} is optionally substituted C₃-C₆ carbocyclyl ring (e.g., cyclopropyl, cyclopentyl, cyclohexyl). In certain embodiments, R^{e} is an optionally substituted C₃-C₆ heterocyclyl ring (e.g., piperidinyl, piperazinyl, morpholinyl, pyrrolidinyl). In certain embodiments, R^{e} is an optionally substituted aryl (e.g., phenyl, naphthyl). In certain embodiments, R^{e} is an optionally substituted heteroaryl (e.g., pyridinyl, pyrimidinyl, isoquinolinyl, thienopyrimidinyl). In certain embodiments, R^{e} is a nitrogen protecting group, oxygen protecting group, or sulfur protecting group.

In certain embodiments, two R^{e} groups are joined to form an optionally substituted carbocyclic ring. In certain embodiments, two R^{e} groups are joined to form an optionally substituted C₃-C₆ carbocyclyl ring (e.g., cyclopropyl, cyclobutyl, cyclopentyl, cyclohexyl). In certain embodiments, two R^{e} groups are joined to form an optionally substituted aryl ring. In certain embodiments, two R^{e} groups form an optionally substituted phenyl. In certain embodiments, two R^{e} groups form an optionally substituted naphthalenyl.

In certain embodiments, two R^{e} groups are joined to form an optionally substituted heterocyclic ring. In certain embodiments, two R^{e} groups are joined to form an optionally substituted C₃-C₆ heterocyclyl ring (e.g., piperidinyl, piperazinyl, morpholinyl, pyrrolidinyl). In certain embodiments, two R^{e} groups are joined to form an optionally substituted heteroaryl ring. In certain embodiments, two R^{e} groups form an optionally substituted pyridinyl. In certain embodiments, two R^{e} groups form an optionally substituted pyrimidinyl. In certain embodiments, two R^{e} groups form an optionally substituted isoquinolinyl. In certain embodiments, two R^{e} groups form an optionally substituted thienopyrimidinyl.

In certain embodiments, the compound is of the formula:

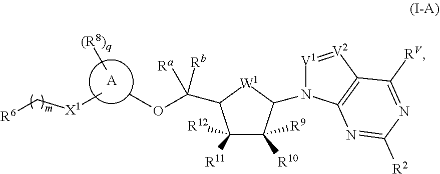

or a pharmaceutically acceptable salt, stereoisomer, or tautomer thereof.

In certain embodiments, the compound is of the formula:

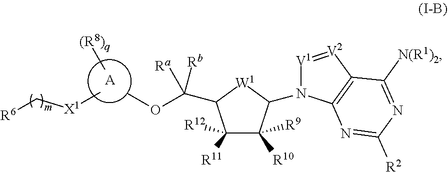

or a pharmaceutically acceptable salt, stereoisomer, or tautomer thereof.

In certain embodiments, the compound is of the formula:

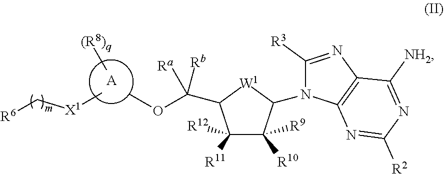

or a pharmaceutically acceptable salt, stereoisomer, or tautomer thereof.

In certain embodiments, the compound is of the formula:

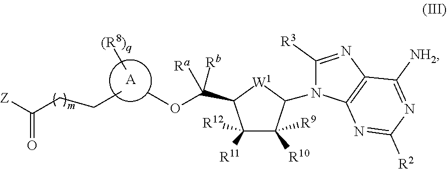

or a pharmaceutically acceptable salt, stereoisomer, or tautomer thereof

In certain embodiments, the compound is of the formula:

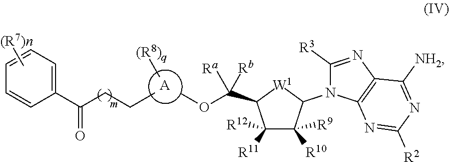

or a pharmaceutically acceptable salt, stereoisomer, or tautomer thereof, wherein:
each occurrence of R⁷ is independently is halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —COOR^{e}—CON(R^{e})₂, —NO₂, —CN, —OR^{e}, or —N(R^{e})₂, or two —R⁷ are joined to form an optionally substituted carbocyclyl, optionally substituted heterocyclyl, optionally substituted aryl, or optionally substituted heteroaryl ring: and
n is 0, 1, 2, 3, 4, or 5,
   and wherein R², R³, R⁹, R¹⁰, R¹¹, R¹², R⁷, R⁸, q, m, and n are as defined herein.

In certain embodiments, the compound is of the formula:

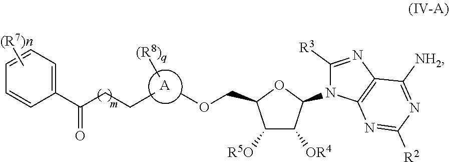

or a pharmaceutically acceptable salt, stereoisomer, or tautomer thereof.

In certain embodiments, the compound is of the formula:

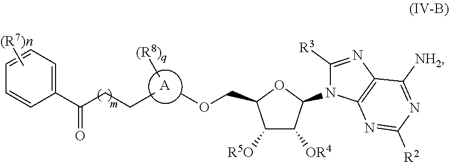

or a pharmaceutically acceptable salt, stereoisomer, or tautomer thereof.

In certain embodiments, the compound is of the formula:

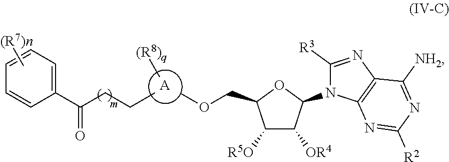

or a pharmaceutically acceptable salt, stereoisomer, or tautomer thereof.

In certain embodiments, the compound is of the formula:

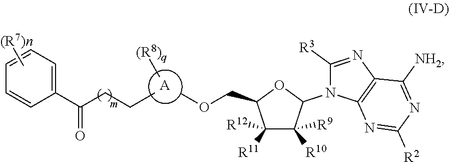

or a pharmaceutically acceptable salt, stereoisomer, or tautomer thereof.

In certain embodiments, there are no instances of R⁷. In certain embodiments, there is a single instance of R⁷. In certain embodiments, there are multiple instances of R.⁷. In certain embodiments, each instance of R⁷ is independently selected, and all instances of R⁷ are different. In certain embodiments, each instance of R⁷ is independently selected, and some instances of R⁷ are different. In certain embodiments, all instances of R⁷ are the same. In certain embodiments, when n is two, all instances of R⁷ are the same. In certain embodiments. when n is three, all instances of R⁷ are the same. In certain embodiments, when n is four, all instances of R⁷ are the same.

In some embodiments, R⁷ is —Cl, —Br, or —I. In some embodiments, R⁷ is —F. In certain embodiments, R⁷ is optionally substituted alkyl. In certain embodiments, R⁷ is optionally substituted C₁₋₆ alkyl. In certain embodiments, R⁷ is unsubstituted C₁₋₆ alkyl. In certain embodiments, R⁷ is methyl. In certain embodiments, R⁷ is ethyl, propyl, or butyl. In certain embodiments, R⁷ is substituted methyl. In certain embodiments, R⁷ is —CF₃. In certain embodiments, R⁷ is —CHF₂ or —CH₂F. In certain embodiments, R⁷ is optionally substituted alkenyl, e.g., optionally substituted C₂₋₆ alkenyl. In certain embodiments, R⁷ is vinyl, allyl, or prenyl. In certain embodiments, R⁷ is optionally substituted alkynyl, e.g., C₂₋₆ alkynyl.

In certain embodiments, R⁷ is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R⁷ is optionally substituted heterocyclyl, e.g.. optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R⁷ is optionally substituted aryl, e.g., optionally substituted phenyl. In certain embodiments, R⁷ is optionally substituted heteroaryl, e.g., optionally substituted 5-6 membered heteroaryl, or optionally substituted 9-10 membered bicyclic heteroaryl. In certain embodiments, R⁷ is optionally substituted aralkyl, e.g., optionally substituted benzyl. In certain embodiments, R⁷ is optionally substituted heteroaralkyl, e.g., methyl substituted with a 5-6-membered heteroaryl ring.

In certain embodiments, R⁷ is —NO₂. In certain embodiments, R⁷ is —CN. In certain embodiments, R⁷ is —OR^{e} (e.g., —OH, —OMe, —O(C₁₋₆ alkyl)). In certain embodiments, R⁷ is —OR^{e}, and R^{e} is an oxygen protecting group. In certain embodiments, R⁷ is —N(R^{e})₂ (e.g., —NH₂, —NMe₂, or —NH(C₁₋₆ alkyl)). In certain embodiments, R⁷ is —N(R^{e})₂, and R^{e} is a nitrogen protecting group, In certain embodiments, R⁷ is optionally substituted acyl (e.g., —C(═O)(R^{e}), —C(═O)O(R^{e}), —C(═O)NH(R^{e}), —C(═O)N(R^{e})₂). In some embodiments, R⁷ is —C(═O)OMe. In some embodiments, R⁷ is —C(═O)OH.

In certain embodiments, n is 0. In some embodiments, n is 1. In certain embodiments, n is 2. In some embodiments, n is 3.4, or 5.

In certain embodiments, n is 1 and R⁷ is ortho to the carbonyl. In certain embodiments, n is 1 and R⁷ is meta to the carbonyl. In certain embodiments, n is 1 and R⁷ is para to the carbonyl.

In some embodiments, a compound is of the formula:

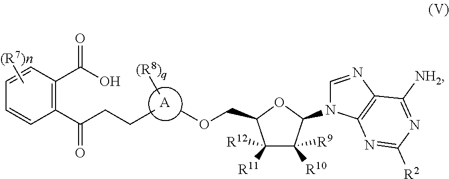

or a pharmaceutically acceptable salt, stereoisomer, or tautomer thereof.

In certain embodiments, a compound is of one of the following formulae:

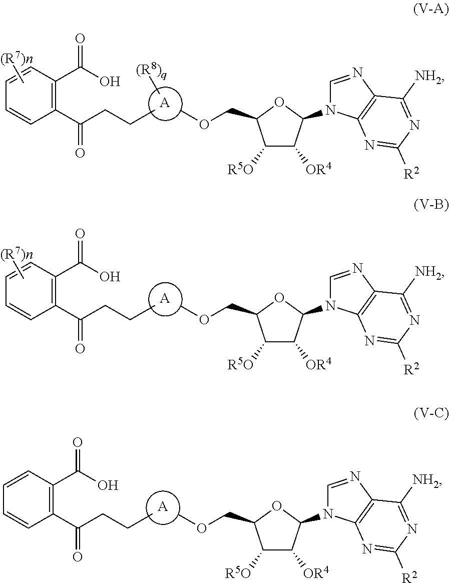

or a pharmaceutically acceptable salt, stereoisomer, or tautomer thereof.

In some aspects, the compound of Formula (I) is of Formula (VI):

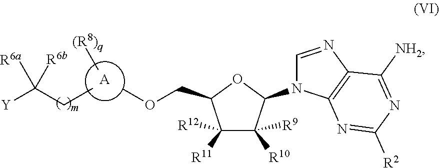

or a pharmaceutically acceptable salt, stereoisomer, or tautomer thereof.

In some aspects, the compound of Formula (I) is of Formula (VI-A):

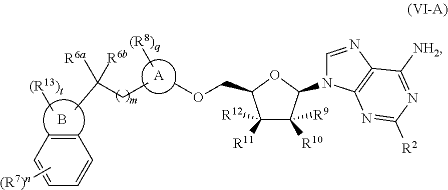

or a pharmaceutically acceptable salt, stereoisomer, or tautomer thereof, wherein:
Ring B is an optionally substituted carbocyclyl, optionally substituted heterocyclyl, optionally substituted aryl, or optionally substituted heteroaryl ring;
each occurrence of R¹³ is independently is hydrogen, halogen, optionally substituted alkyl, optionally substituted alkenyl, optionally substituted alkynyl, optionally substituted carbocyclyI, optionally substituted heterocyclyl, optionally substituted aryl, optionally substituted heteroaryl, optionally substituted acyl, —COOR^{e}, —CON(R^{e})₂, —NO₂, —CN, —OR^{e}, or —N(R^{e})₂; and
t is 0, 1, 2, 3, 4, or 5,
   and wherein R², R⁹, R¹⁰, R¹¹, R¹², R⁷, R⁸, R^{6a}, R^{6b}, q, in, and n are as defined herein.

In certain embodiments, Ring B is an optionally substituted carbocyclic ring (e.g.. an optionally substituted 5- to 6-membered carbocyclic ring). In certain embodiments, Ring B is a optionally substituted heterocyclic ring (e.g., an optionally substituted 5- to 6-membered heterocyclic ring, comprising 0 to 3 heteroatoms independently selected from O, N, and S). In certain embodiments, Ring B is an optionally substituted aryl ring (e.g., an optionally substituted phenyl ring). In certain embodiments, Ring B is an optionally substituted heteroaryl ring (e.g., an optionally substituted 5- to 6-membered heteroaryl ring, comprising 0 to 3 heteroatoms independently selected from O, N, and S).

In certain embodiments, there are no instances of R¹³. In certain embodiments, there is a single instance of R¹³. In certain embodiments, there are multiple instances of R¹³. In certain embodiments, each instance of R¹³ is independently selected, and all instances of R¹³ are different. In certain embodiments, each instance of R¹³ is independently selected, and some instances of R¹³ are different. In certain embodiments, all instances of R¹³ are the same.

In some embodiments, R¹³ is —Cl, —Br, or —I. in some embodiments, R¹³ is —F. In certain embodiments, R¹³ is optionally substituted alkyl. In certain embodiments, R¹³ is unsubstituted C₁₋₆ alkyl. In certain embodiments, R¹³ is methyl. In certain embodiments, R^{—}is ethyl, propyl, or butyl. In certain embodiments, R¹³ is —CF₃. In certain embodiments, R¹³ is optionally substituted alkenyl, e.g., optionally substituted C₂₋₆ alkenyl. In certain embodiments, R¹³ is vinyl, allyl, or prenyl. In certain embodiments, R¹³ is optionally substituted alkynyl, C₂₋₆ alkynyl.

In certain embodiments, R¹³ is optionally substituted carbocyclyl, e.g., optionally substituted C₃₋₆ carbocyclyl, optionally substituted C₃₋₄ carbocyclyl, optionally substituted C₄₋₅ carbocyclyl, or optionally substituted C₅₋₆ carbocyclyl. In certain embodiments R¹³ is optionally substituted heterocyclyl, e.g., optionally substituted 3-6 membered heterocyclyl, optionally substituted 3-4 membered heterocyclyl, optionally substituted 4-5 membered heterocyclyl, or optionally substituted 5-6 membered heterocyclyl.

In certain embodiments, R¹³ is optionally substituted aryl, e.g., optionally substituted phenyl. In certain embodiments, R¹³ is optionally substituted heteroaryl, e.g., optionally substituted 5-6 membered heteroaryl, or optionally substituted 9-10 membered bicyclic heteroaryl. In certain embodiments, R¹³ is optionally substituted aralkyl, e.g., optionally substituted benzyl. In certain embodiments, R¹³ is optionally substituted heteroaralkyl, e.g., methyl substituted with a 5-6-membered heteroaryl ring.

In certain embodiments, R¹¹ is —NO₂. In certain embodiments, R¹³ is —CN. In certain embodiments, R¹³ is —OR^{e} (e.g., —OH, —OMe, —O(C₁₋₆ alkyl)). In certain embodiments, R¹³ is —OR^{e}, and R^{e} is an oxygen protecting group. In certain embodiments, R¹³ is —N(R^{e})₂ (e.g., —NH₂, —NMe₂, or —NH(C₁₋₆ alkyl)). In certain embodiments, R¹³ is —N(R^{e}, and R^{e} is a nitrogen protecting group. In certain embodiments, R¹³ is optionally substituted acyl (e.g., —C(═O)(R^{e}), —C(═O)O(R^{e}), —C(═O)N(R^{e})₂). In some embodiments, R¹³ is —C(═O)OMe. In some embodiments, R¹³ is —C(═O)OH.

In some embodiments, t is 0. In certain embodiments, t is 1. In certain embodiments, t is 2. In certain embodiments, t is 3. In certain embodiments, t is 1, 2, or 3. In some embodiments, t is 4 or 5.

In some aspects, the compound of Formula (I) is of Formula (VI-B):

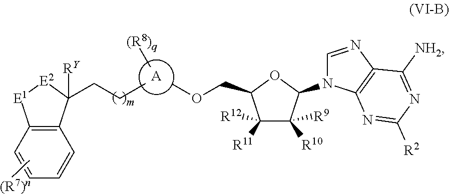

or a pharmaceutically acceptable salt, stereoisomer, or tautomer thereof, wherein:
E¹ is —C(═O)—, —C(═S)—, —C(═NR^{f})—, —C(R^{F1})₂—, —O—, or —NR^{f}—; and each R^{F1} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl. —OR^{e}, —SR^{e}, or —N(R^{e})₂;
E² is —C(═O)—, —C(50 S)—, —(═NR^{f})—, —(R^{E2})₂—, —O—, or —NR^{f}—; and each R^{E2} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, —SR^{e}, or —N(R^{e})₂; and
R^{Y} is hydrogen, halogen, optionally substituted alkyl, optionally substituted alkenyl, optionally substituted alkynyl, —OR^{e}, or —N(R^{e})₂.

As generally defined herein, E¹ is —C(═O)—, —C(═S)—, —C(═NR^{f})—, —C(R^{E1})₂—, —O—, or —NR^{f}—; and each R^{E1} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, —SR^{e}, or —N(R^{e})₂. When E¹ is —C(R^{E1})₂—, the carbon to which both R^{E1} are attached may be of either the (R)- or (S)-configuration.

In certain embodiments, E¹ is —C(═O)—. In certain embodiments, E¹ is —C(═S)—. In certain embodiments, E¹ is —C(═NR^{f})— (e.g., —C(═NH)—). In certain embodiments, E¹ is —C(R^{E1})₂— (e.g., —CH₂—, —CH(R^{E1})—). In some embodiments, E¹ is —CH(OR^{e})— (e.g., —CH(OH)—). In some embodiments, E¹ is —C(R^{E1})₂, wherein at least one occurrence of R^{E1} is halogen. In some embodiments, E¹ is —CF₂—. In certain embodiments. E¹ is —O—. In certain embodiments, E¹ is —NR^{f}— (e.g., —NH—, —NMe-).

As generally defined herein, E² is —C(═O)—, —C(═S)—, —C(═NR^{f})—, —C(R^{E2})₂—, —O—, or —NR^{f}—; and each R^{E2} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, or —N(R^{e})₂. When E² is —C(R^{E2})₂—, the carbon to which both R^{E2} are attached may be of either the (R) or (S) configuration.

In certain embodiments, E² is —C(═O)—. In certain embodiments, E² is —C(═S)—. In certain embodiments, E² is —C(═NR^{f})— (e.g., —C(═NH)—). In certain embodiments, E² is —C(R^{E2})₂— (e.g., —CH₂—, —CH(R^{E2})—). In some embodiments, E² is —CH(OR^{e})— (e.g., —CH(OH)—). In some embodiments, E² is —C(R^{E2})₂, wherein at least one occurrence of R^{E2} is halogen. In some embodiments, E² is —CF₂—. In certain embodiments, E² is —O—. In certain embodiments, E² is —NR^{f}— (e.g., —NH—, —NMe-).

R^{Y} is hydrogen, halogen, optionally substituted alkyl, optionally substituted alkenyl, optionally substituted alkynyl, —OR^{e}, or —N(R^{e})₂. The carbon to which R^{Y} is attached may be of either the (R)- or (S)-configuration.

In some embodiments, R^{f} is hydrogen. In certain embodiments, R^{f} is substituted C₁₋₆ alkyl. In certain embodiments, R^{f} is unsubstituted C₁₋₆ alkyl. In certain embodiments, E¹ and/or E² is —NR^{f}—, and R^{f} is methyl. In certain embodiments, E¹ and/or E² is —NR^{f}—, and R^{f} is ethyl, propyl, or butyl. In certain embodiments, E¹ and/or E² is —NR^{f}—, and R^{f} is optionally substituted acyl (e.g., —C(═O)(R^{e}), —C(═O)(R^{e})O(R^{e}), —C(═O)NH(R^{e}), —C(═O)N(R^{e})₂). In certain embodiments. E¹ and/or E² is —NR^{f}—, and R^{f} is a nitrogen protecting group.

In certain embodiments, R^{Y} is hydrogen. In certain embodiments, R^{Y} is halogen. In certain embodiments, R^{Y} is —F. In certain embodiments, R^{Y} is —Cl, —Br, or —F. In certain embodiments, R^{Y}, is —NO₂. In certain embodiments, R^{Y} is —CN. In certain embodiments, R^{Y} is —OR^{e} (e.g. —OH, —OMe, —O(C₁₋₆ alkyl)) In certain embodiments, R^{Y} is —OR^{e}, and R^{e} is an oxygen protecting group. In certain embodiments, R^{Y} is —N(R^{e})₂ (e.g., —NH₂, —NMe₂, —NH(C₁₋₆ alkyl)). In certain embodiments, R^{Y} is —NHR^{e}, and R^{e} is a nitrogen protecting group.

In certain embodiments, R^{Y} is optionally substituted alkyl, e.g., optionally substituted C₁₋₆ alkyl, optionally substituted C₁₋₂ alkyl, optionally substituted C₂₋₃ alkyl, optionally substituted C₃₋₄ alkyl, optionally substituted C₄₋₅ alkyl, or optionally substituted C₅₋₆ alkyl. In certain embodiments. R^{Y} is methyl. In certain embodiments. R^{Y} is ethyl, propyl, or butyl. In certain embodiments, R^{Y} is optionally substituted alkenyl, e.g., optionally substituted C₂₋₆ alkenyl. In certain embodiments, R^{Y} is vinyl, allyl, or prenyl. In certain embodiments, R^{Y} is optionally substituted alkynyl, e.g., C₂₋₆ alkynyl.

In some embodiments, a compound is of one of the following formulae:

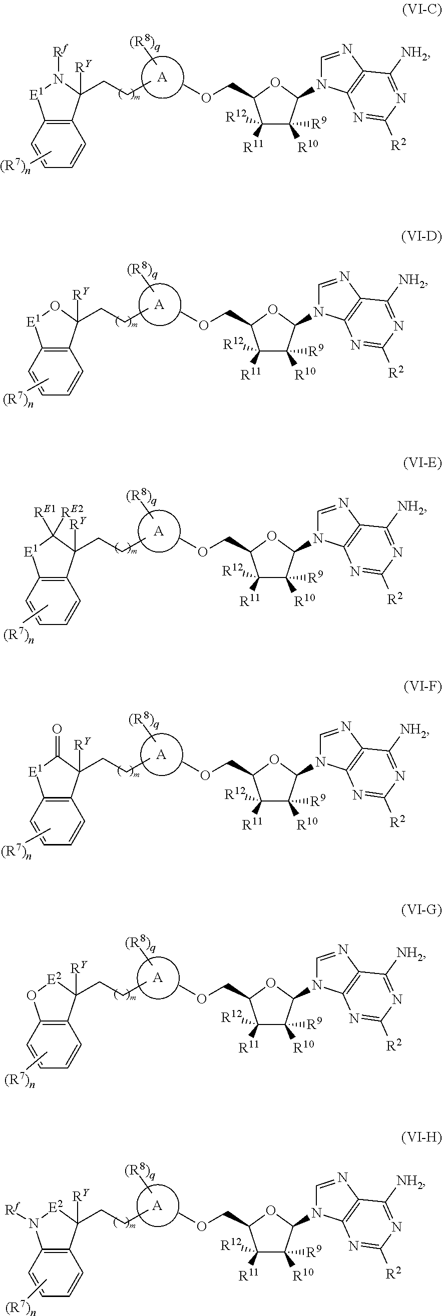

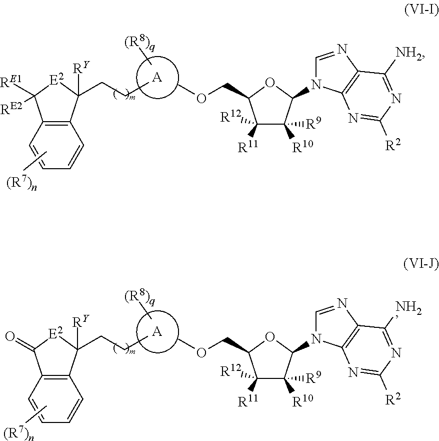

or a pharmaceutically acceptable salt, stereoisomer, or tautomer thereof.

In certain embodiments, the compound of Formula (I) is:

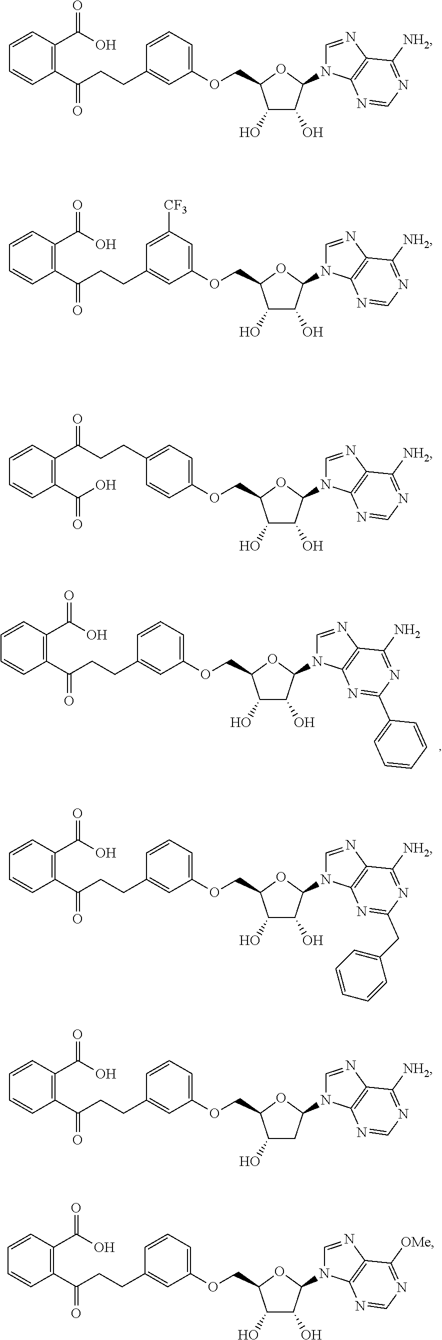

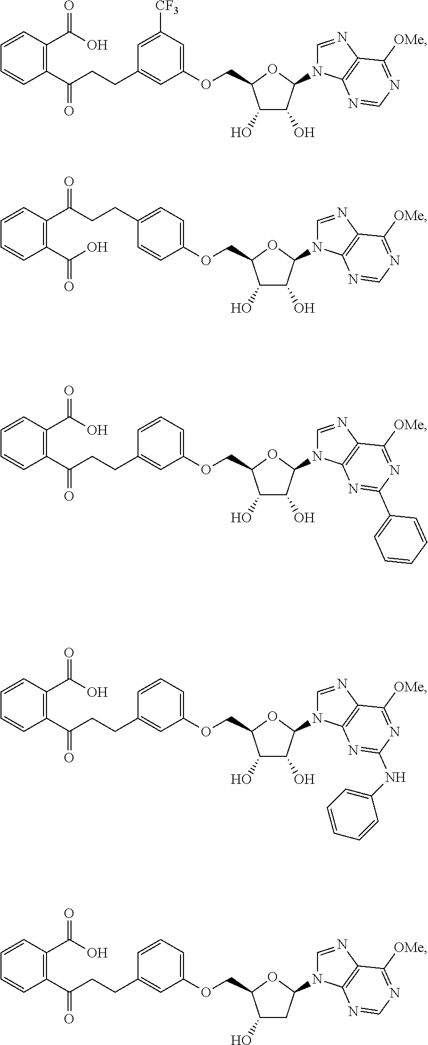 [SMILES: COc1ncnc2c1ncn2[C@@H]1O[C@H](COc2cc(CCC(=O)c3ccccc3C(=O)O)cc(C(F)(F)F)c2)[C@@H](O)[C@H]1O ; COc1ncnc2c1ncn2[C@@H]1O[C@H](COc2ccc(CCC(=O)c3ccccc3C(=O)O)cc2)[C@@H](O)[C@H]1O ; COc1nc(-c2ccccc2)nc2c1ncn2[C@@H]1O[C@H](COc2cccc(CCC(=O)c3ccccc3C(=O)O)c2)[C@@H](O)[C@H]1O ; COc1nc(Nc2ccccc2)nc2c1ncn2[C@@H]1O[C@H](COc2cccc(CCC(=O)c3ccccc3C(=O)O)c2)[C@@H](O)[C@H]1O ; COc1ncnc2c1ncn2[C@H]1C[C@H](O)[C@@H](COc2cccc(CCC(=O)c3ccccc3C(=O)O)c2)O1]

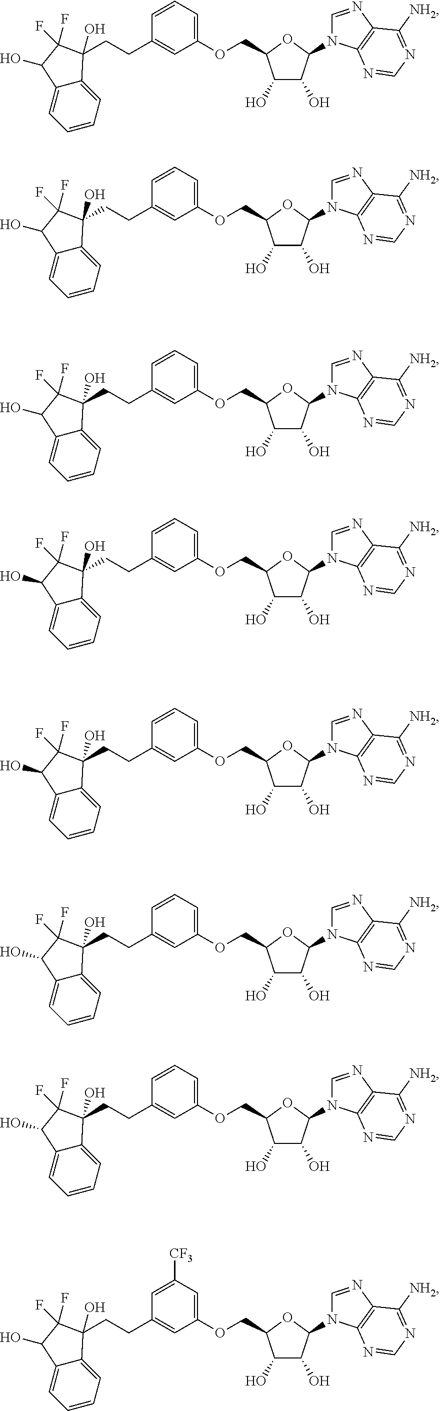

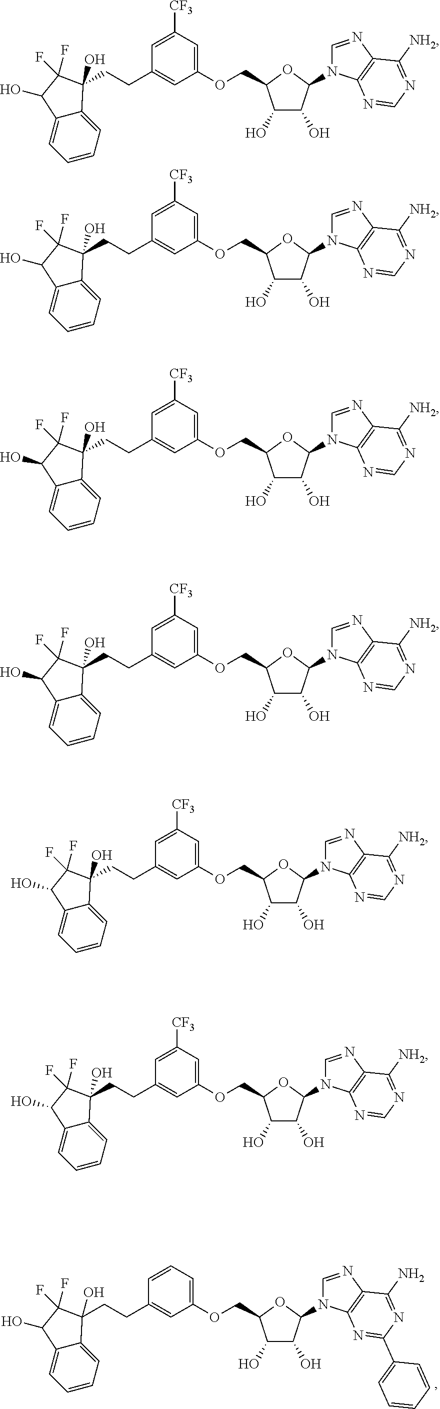

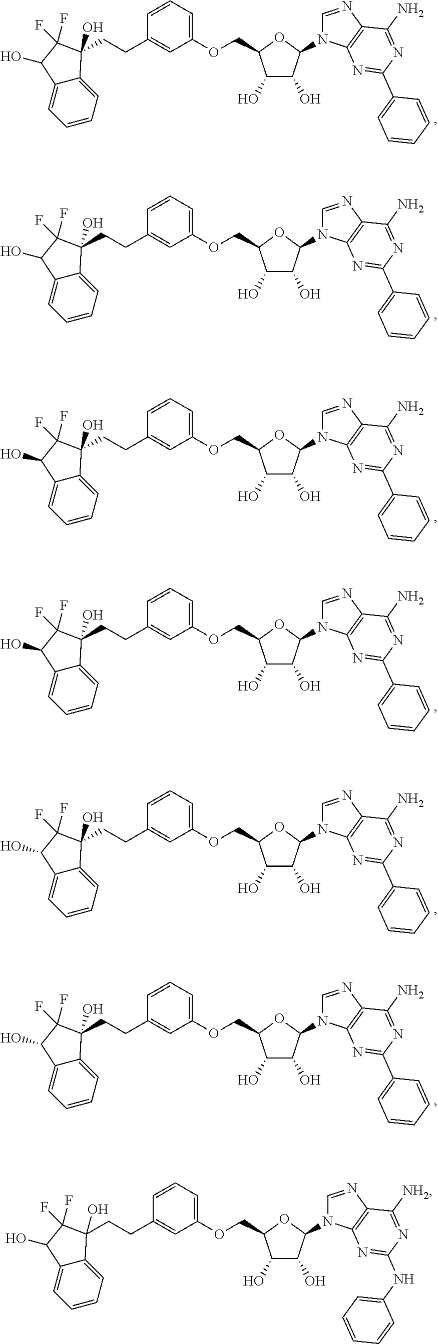

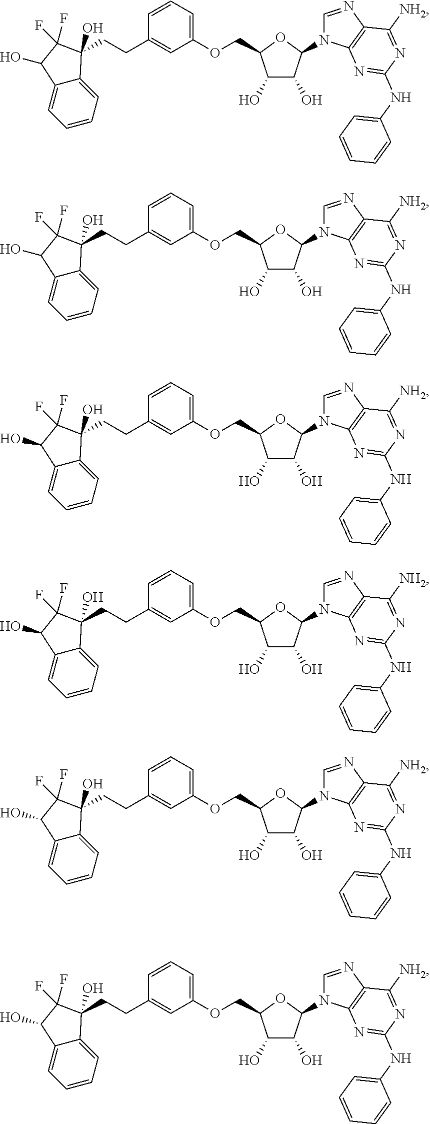

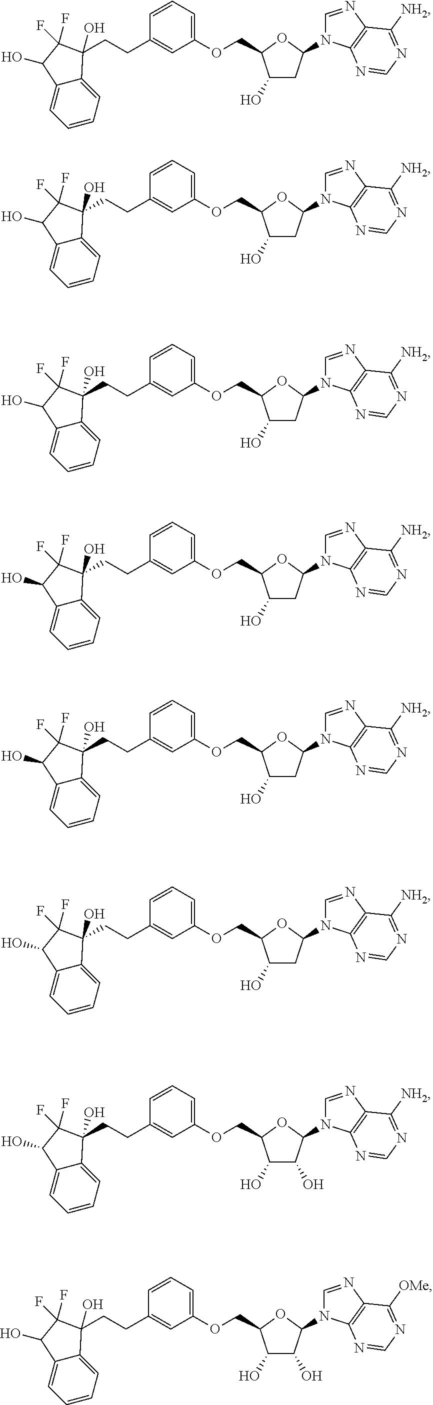

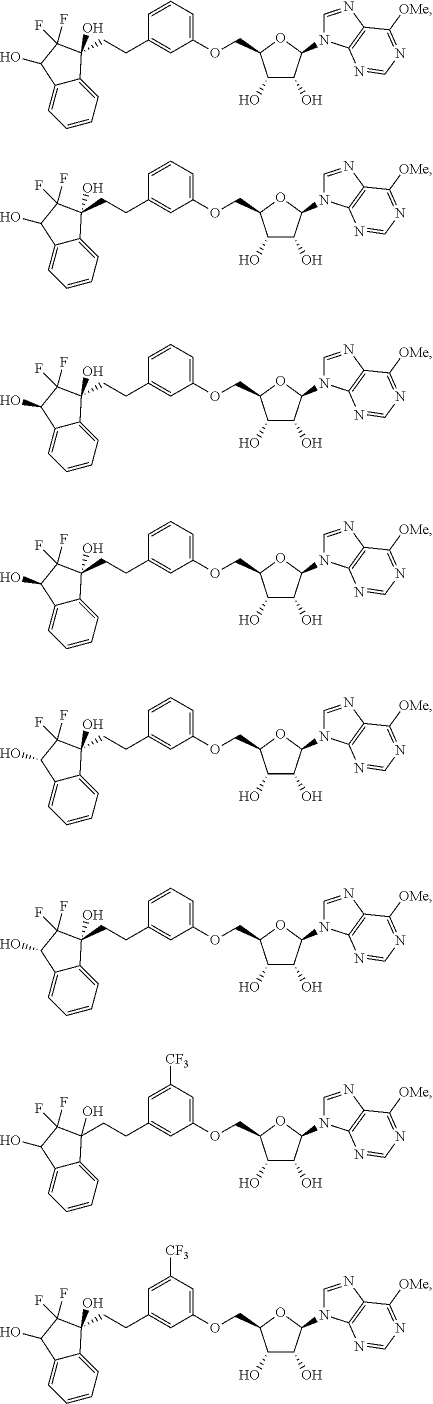

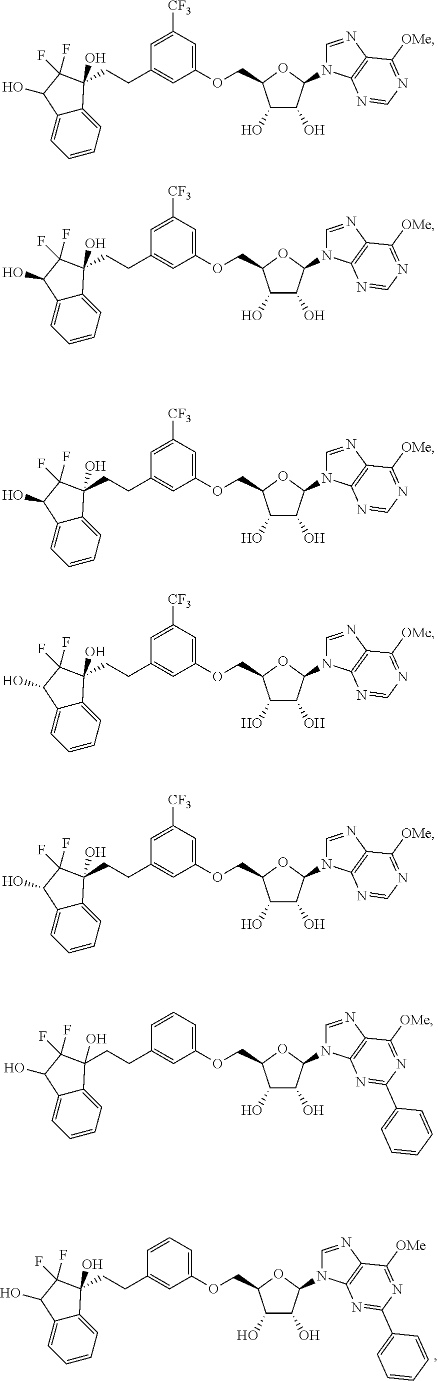

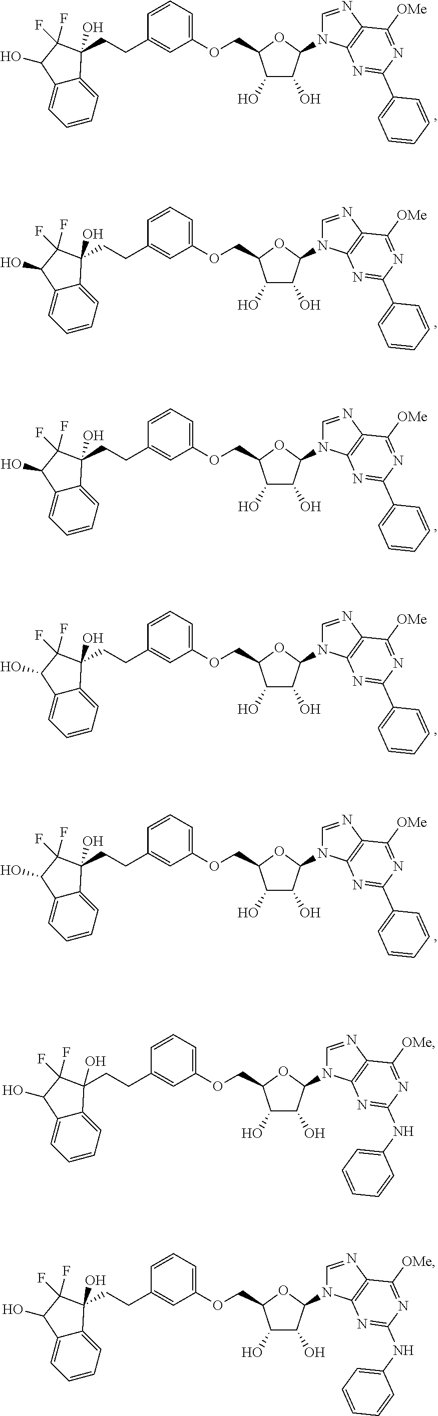

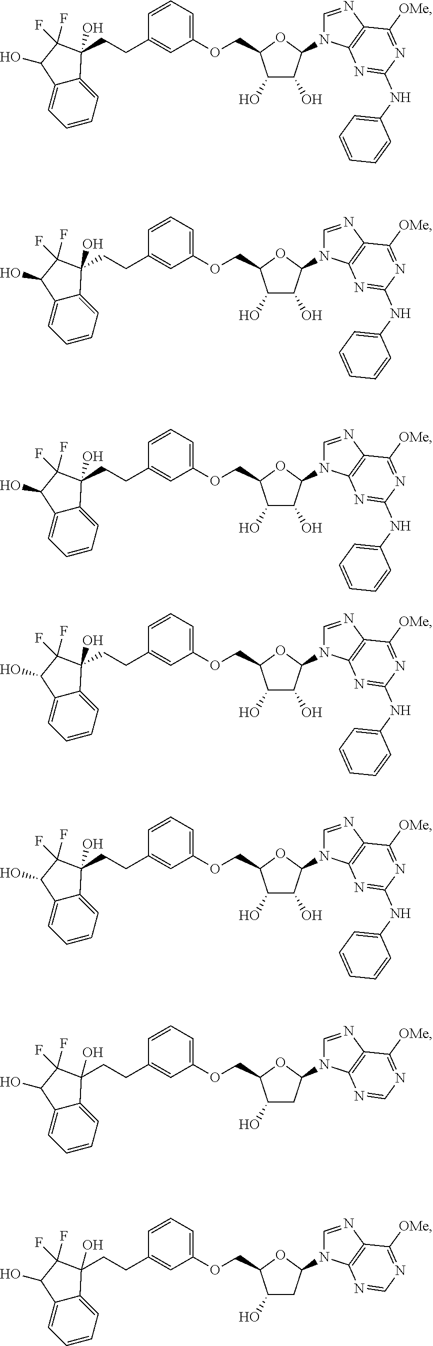

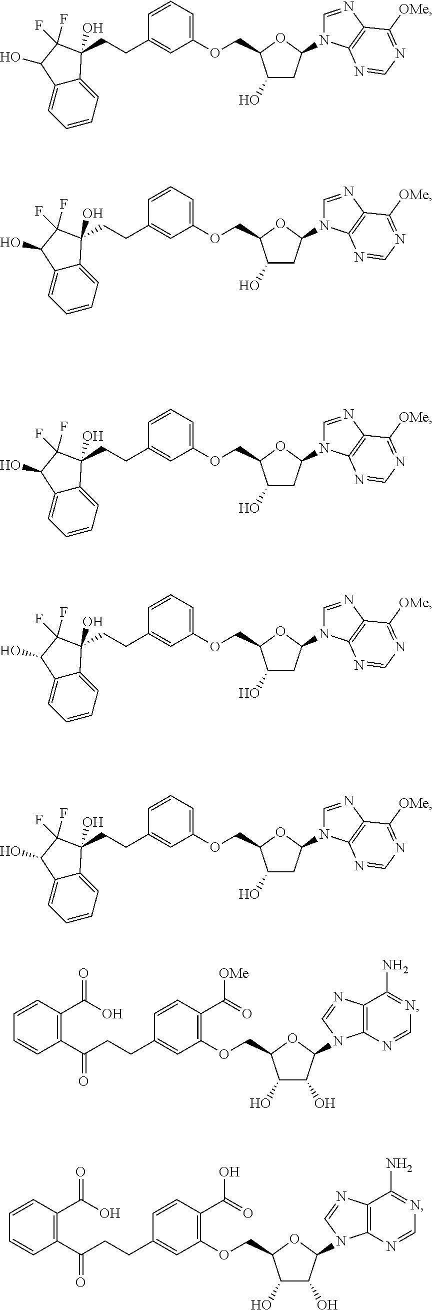

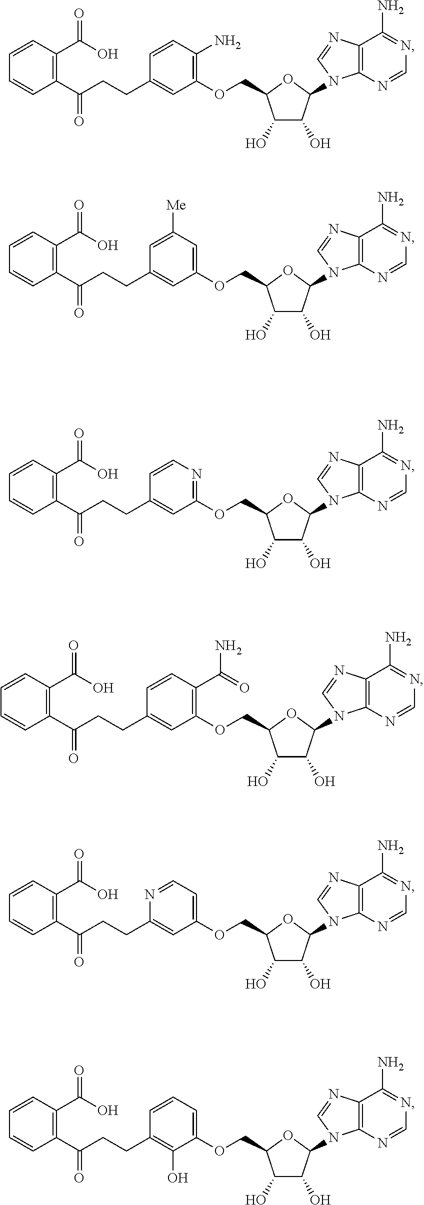 [SMILES: Nc1ccc(CCC(=O)c2ccccc2C(=O)O)cc1OC[C@H]1O[C@@H](n2cnc3c(N)ncnc32)[C@H](O)[C@@H]1O ; Cc1cc(CCC(=O)c2ccccc2C(=O)O)cc(OC[C@H]2O[C@@H](n3cnc4c(N)ncnc43)[C@H](O)[C@@H]2O)c1 ; Nc1ncnc2c1ncn2[C@@H]1O[C@H](COc2cc(CCC(=O)c3ccccc3C(=O)O)ccn2)[C@@H](O)[C@H]1O ; NC(=O)c1ccc(CCC(=O)c2ccccc2C(=O)O)cc1OC[C@H]1O[C@@H](n2cnc3c(N)ncnc32)[C@H](O)[C@@H]1O ; Nc1ncnc2c1ncn2[C@@H]1O[C@H](COc2ccnc(CCC(=O)c3ccccc3C(=O)O)c2)[C@@H](O)[C@H]1O ; Nc1ncnc2c1ncn2[C@@H]1O[C@H](COc2cccc(CCC(=O)c3ccccc3C(=O)O)c2O)[C@@H](O)[C@H]1O]

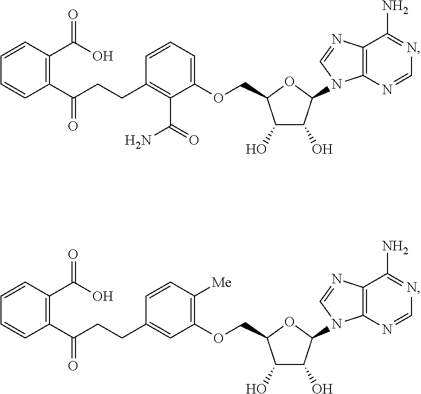 [SMILES: NC(=O)c1c(CCC(=O)c2ccccc2C(=O)O)cccc1OC[C@H]1O[C@@H](n2cnc3c(N)ncnc32)[C@H](O)[C@@H]1O ; Cc1ccc(CCC(=O)c2ccccc2C(=O)O)cc1OC[C@H]1O[C@@H](n2cnc3c(N)ncnc32)[C@H](O)[C@@H]1O]

or a pharmaceutically acceptable salt, stereoisomer, or tautomer thereof.

In certain embodiments, the compound of Formula (I) is:

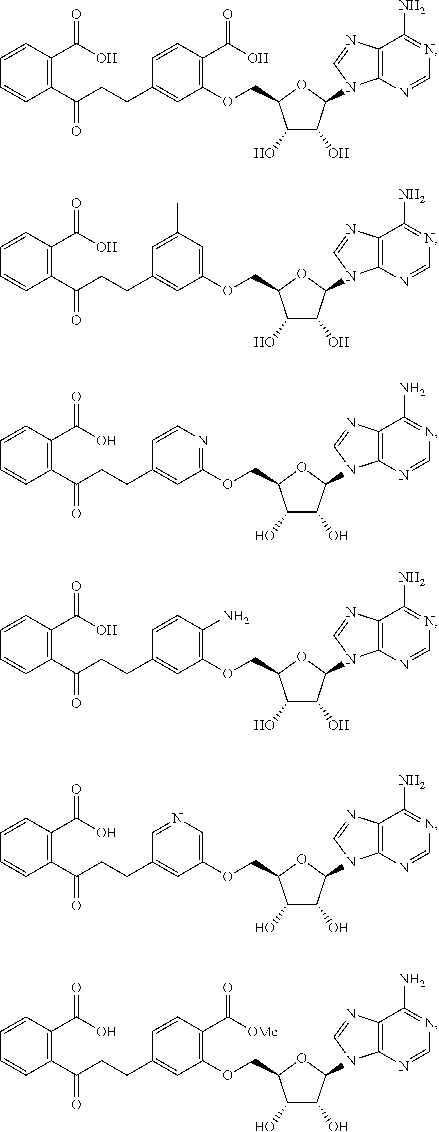

or a pharmaceutically acceptable salt, stereoisomer, or tautomer thereof.

In certain embodiments. a compound of Formula (I) may contain the moieties described in Tables A, B, C, and D below. Non-limiting examples of moieties appear in Tables A to D. The variables as expressed in Tables A, B, C, and D are as described herein. The moieties from Tables A, B, C, and D may be combined into a compound along with the variables as defined herein to generate compound of Formula (I).

TABLE A
Exemplary Purine and Heterocycle Moieties

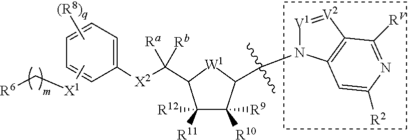

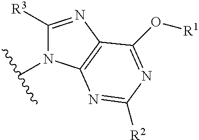

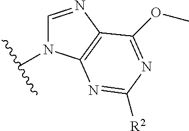

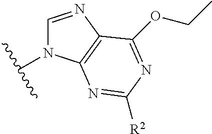

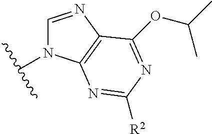

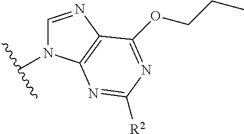

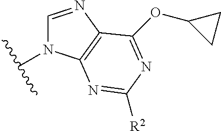

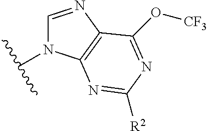

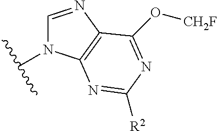

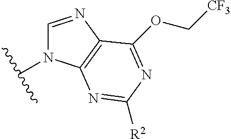

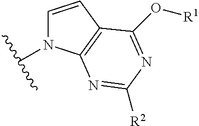

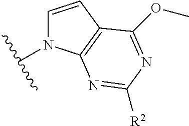

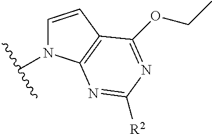

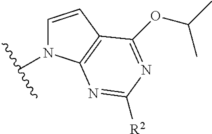

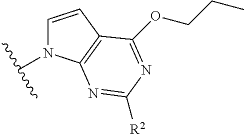

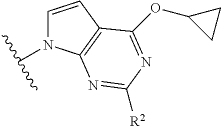

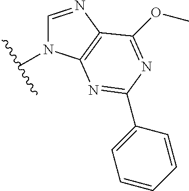 [SMILES: *n1cnc2c(OC)nc(-c3ccccc3)nc21]

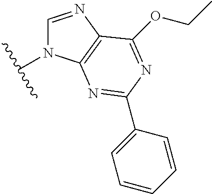 [SMILES: *n1cnc2c(OCC)nc(-c3ccccc3)nc21]

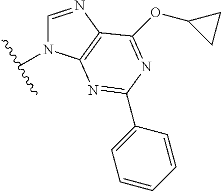 [SMILES: *n1cnc2c(OC3CC3)nc(-c3ccccc3)nc21]

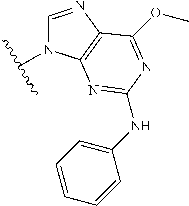 [SMILES: *n1cnc2c(OC)nc(Nc3ccccc3)nc21]

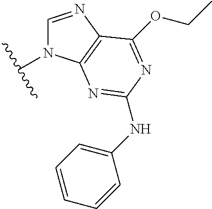 [SMILES: *n1cnc2c(OCC)nc(Nc3ccccc3)nc21]

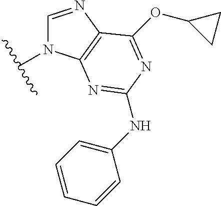 [SMILES: *n1cnc2c(OC3CC3)nc(Nc3ccccc3)nc21]

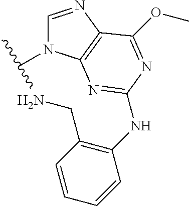 [SMILES: *n1cnc2c(OC)nc(Nc3ccccc3CN)nc21]

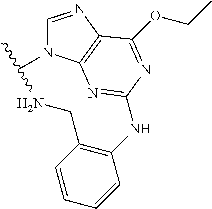 [SMILES: *n1cnc2c(OCC)nc(Nc3ccccc3CN)nc21]

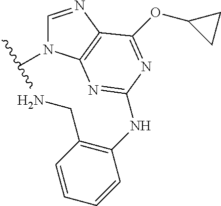 [SMILES: *n1cnc2c(OC3CC3)nc(Nc3ccccc3CN)nc21]

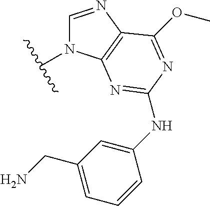 [SMILES: *n1cnc2c(OC)nc(Nc3cccc(CN)c3)nc21]

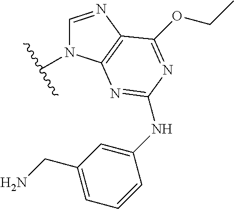 [SMILES: *n1cnc2c(OCC)nc(Nc3cccc(CN)c3)nc21]

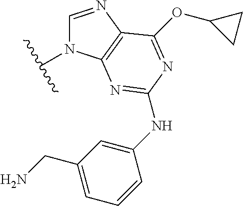 [SMILES: *n1cnc2c(OC3CC3)nc(Nc3cccc(CN)c3)nc21]

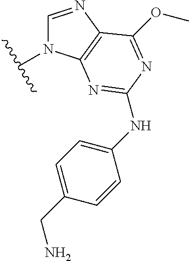 [SMILES: *n1cnc2c(OC)nc(Nc3ccc(CN)cc3)nc21]

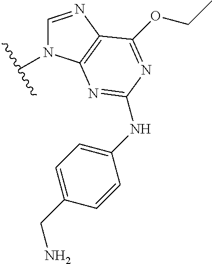 [SMILES: *n1cnc2c(OCC)nc(Nc3ccc(CN)cc3)nc21]

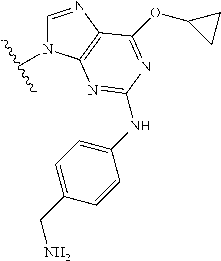 [SMILES: *n1cnc2c(OC3CC3)nc(Nc3ccc(CN)cc3)nc21]

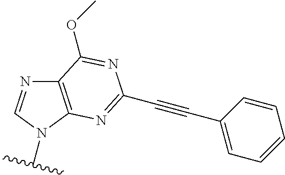 [SMILES: *n1cnc2c(OC)nc(C#Cc3ccccc3)nc21]

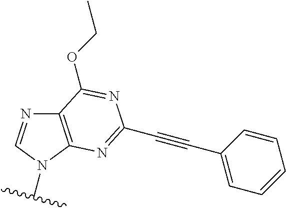 [SMILES: *n1cnc2c(OCC)nc(C#Cc3ccccc3)nc21]

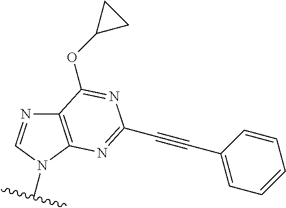 [SMILES: *n1cnc2c(OC3CC3)nc(C#Cc3ccccc3)nc21]

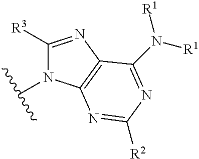

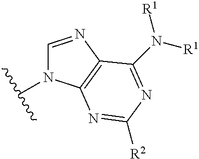

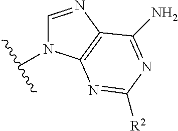

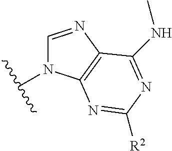

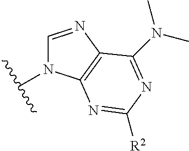

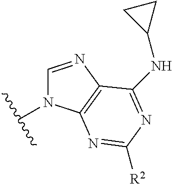

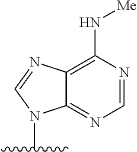 [SMILES: *n1cnc2c(NC)ncnc21]

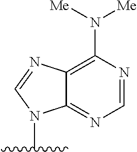 [SMILES: *n1cnc2c(N(C)C)ncnc21]

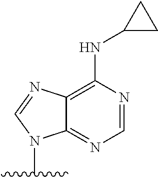 [SMILES: *n1cnc2c(NC3CC3)ncnc21]

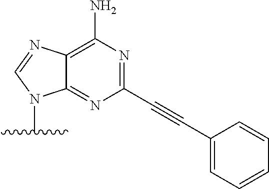 [SMILES: *n1cnc2c(N)nc(C#Cc3ccccc3)nc21]

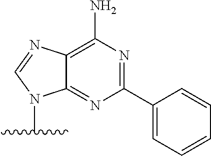 [SMILES: *n1cnc2c(N)nc(-c3ccccc3)nc21]

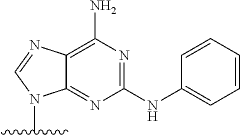 [SMILES: *n1cnc2c(N)nc(Nc3ccccc3)nc21]

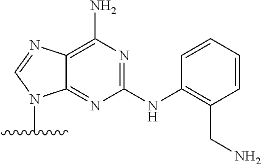 [SMILES: *n1cnc2c(N)nc(Nc3ccccc3CN)nc21]

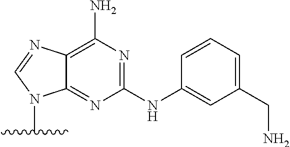 [SMILES: *n1cnc2c(N)nc(Nc3cccc(CN)c3)nc21]

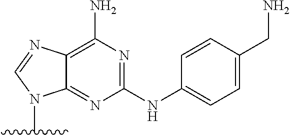 [SMILES: *n1cnc2c(N)nc(Nc3ccc(CN)cc3)nc21]

TABLE B
Exemplary Ribose and Heterocyclic Moieties

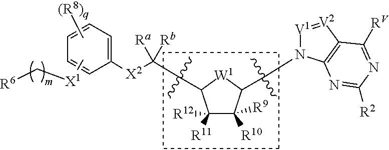

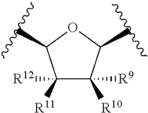

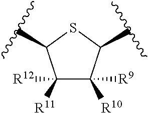

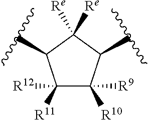

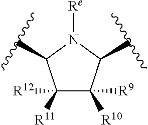

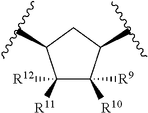

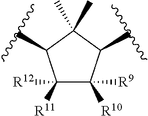

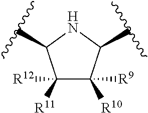

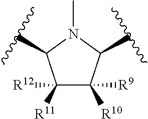

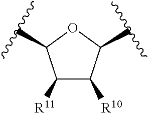

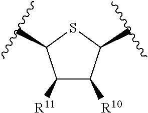

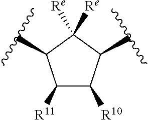

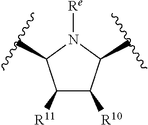

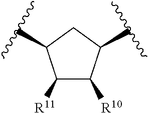

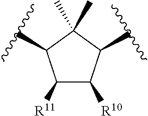

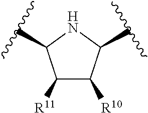

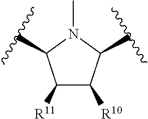

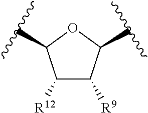

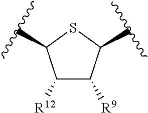

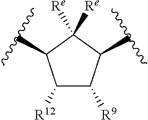

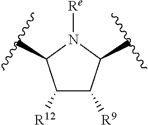

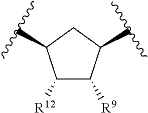

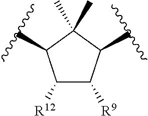

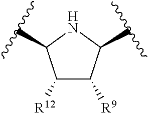

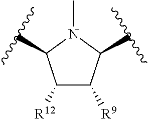

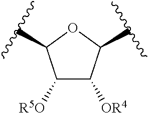

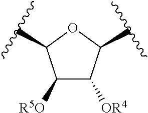

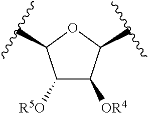

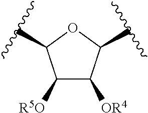

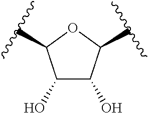 [SMILES: *[C@@H]1O[C@H](*)[C@@H](O)[C@H]1O]

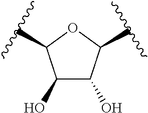 [SMILES: *[C@@H]1O[C@H](*)[C@H](O)[C@H]1O]

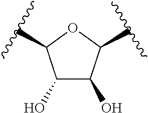 [SMILES: *[C@@H]1O[C@H](*)[C@@H](O)[C@@H]1O]

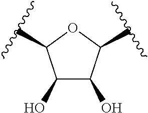 [SMILES: *[C@@H]1O[C@H](*)[C@H](O)[C@@H]1O]

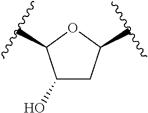 [SMILES: *[C@H]1C[C@H](O)[C@@H](*)O1]

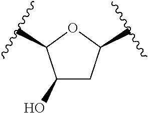 [SMILES: *[C@H]1C[C@@H](O)[C@@H](*)O1]

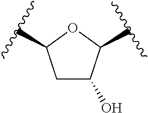 [SMILES: *[C@@H]1C[C@@H](O)[C@H](*)O1]

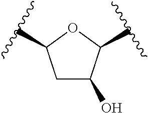 [SMILES: *[C@@H]1C[C@H](O)[C@H](*)O1]

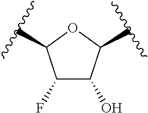 [SMILES: *[C@@H]1O[C@H](*)[C@@H](F)[C@H]1O]

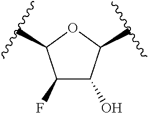 [SMILES: *[C@@H]1O[C@H](*)[C@H](F)[C@H]1O]

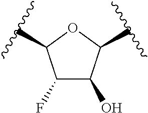 [SMILES: *[C@@H]1O[C@H](*)[C@@H](F)[C@@H]1O]

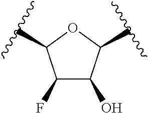 [SMILES: *[C@@H]1O[C@H](*)[C@H](F)[C@@H]1O]

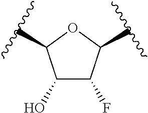 [SMILES: *[C@@H]1O[C@H](*)[C@@H](O)[C@H]1F]

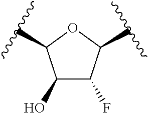 [SMILES: *[C@@H]1O[C@H](*)[C@H](O)[C@H]1F]

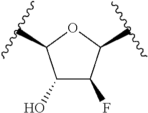 [SMILES: *[C@@H]1O[C@H](*)[C@@H](O)[C@@H]1F]

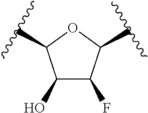 [SMILES: *[C@@H]1O[C@H](*)[C@H](O)[C@@H]1F]

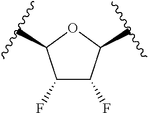 [SMILES: *[C@@H]1O[C@H](*)[C@@H](F)[C@H]1F]

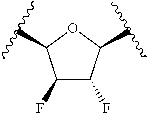 [SMILES: *[C@@H]1O[C@H](*)[C@H](F)[C@H]1F]

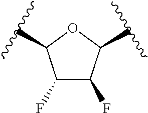 [SMILES: *[C@@H]1O[C@H](*)[C@@H](F)[C@@H]1F]

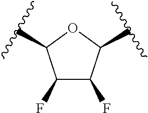 [SMILES: *[C@@H]1O[C@H](*)[C@H](F)[C@@H]1F]

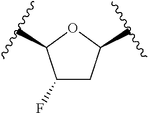 [SMILES: *[C@H]1C[C@H](F)[C@@H](*)O1]

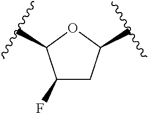 [SMILES: *[C@H]1C[C@@H](F)[C@@H](*)O1]

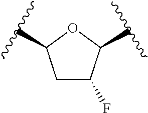 [SMILES: *[C@@H]1C[C@@H](F)[C@H](*)O1]

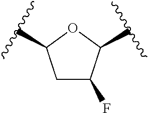 [SMILES: *[C@@H]1C[C@H](F)[C@H](*)O1]

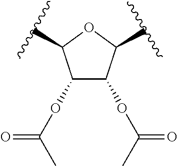 [SMILES: *[C@@H]1O[C@H](*)[C@@H](OC(C)=O)[C@H]1OC(C)=O]

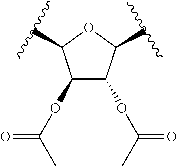 [SMILES: *[C@@H]1O[C@H](*)[C@H](OC(C)=O)[C@H]1OC(C)=O]

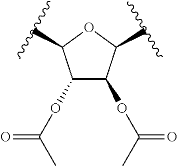 [SMILES: *[C@@H]1O[C@H](*)[C@@H](OC(C)=O)[C@@H]1OC(C)=O]

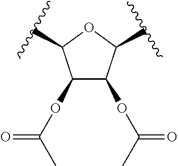 [SMILES: *[C@@H]1O[C@H](*)[C@H](OC(C)=O)[C@@H]1OC(C)=O]

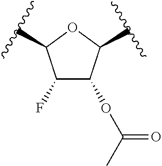 [SMILES: *[C@@H]1O[C@H](*)[C@@H](F)[C@H]1OC(C)=O]

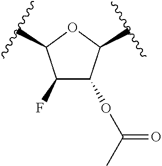 [SMILES: *[C@@H]1O[C@H](*)[C@H](F)[C@H]1OC(C)=O]

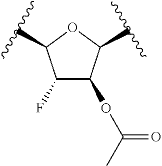 [SMILES: *[C@@H]1O[C@H](*)[C@@H](F)[C@@H]1OC(C)=O]

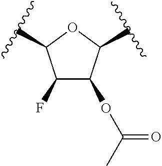 [SMILES: *[C@@H]1O[C@H](*)[C@H](F)[C@@H]1OC(C)=O]

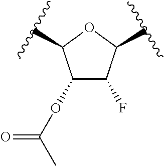 [SMILES: *[C@@H]1O[C@H](*)[C@@H](OC(C)=O)[C@H]1F]

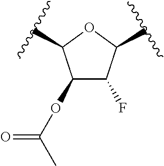 [SMILES: *[C@@H]1O[C@H](*)[C@H](OC(C)=O)[C@H]1F]

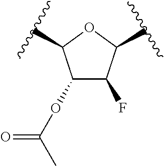 [SMILES: *[C@@H]1O[C@H](*)[C@@H](OC(C)=O)[C@@H]1F]

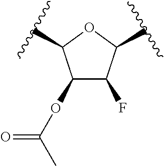 [SMILES: *[C@@H]1O[C@H](*)[C@H](OC(C)=O)[C@@H]1F]

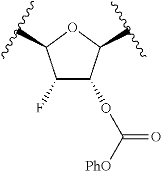 [SMILES: *[C@@H]1O[C@H](*)[C@@H](F)[C@H]1OC(=O)Oc1ccccc1]

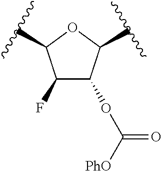 [SMILES: *[C@@H]1O[C@H](*)[C@H](F)[C@H]1OC(=O)Oc1ccccc1]

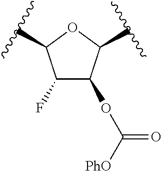 [SMILES: *[C@@H]1O[C@H](*)[C@@H](F)[C@@H]1OC(=O)Oc1ccccc1]

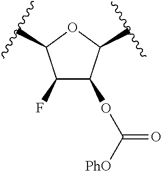 [SMILES: *[C@@H]1O[C@H](*)[C@H](F)[C@@H]1OC(=O)Oc1ccccc1]

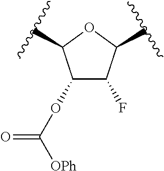 [SMILES: *[C@@H]1O[C@H](*)[C@@H](OC(=O)Oc2ccccc2)[C@H]1F]

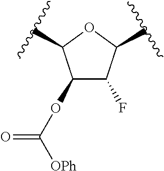 [SMILES: *[C@@H]1O[C@H](*)[C@H](OC(=O)Oc2ccccc2)[C@H]1F]

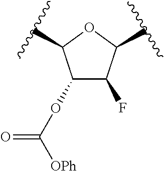 [SMILES: *[C@@H]1O[C@H](*)[C@@H](OC(=O)Oc2ccccc2)[C@@H]1F]

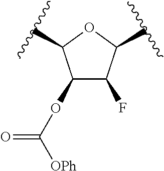 [SMILES: *[C@@H]1O[C@H](*)[C@H](OC(=O)Oc2ccccc2)[C@@H]1F]

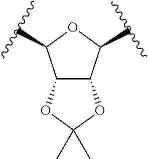 [SMILES: *[C@@H]1O[C@H](*)[C@H]2OC(C)(C)O[C@H]21]

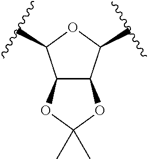 [SMILES: *[C@@H]1O[C@H](*)[C@@H]2OC(C)(C)O[C@@H]21]

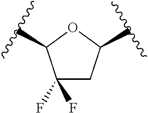

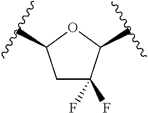

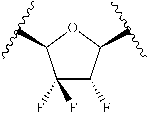

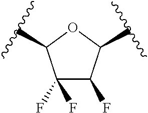

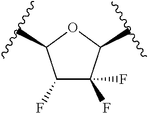

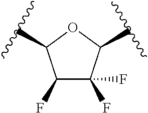

TABLE C
Exemplary Linker Moieties

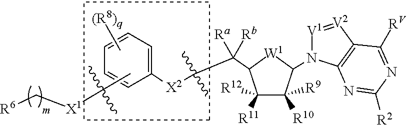

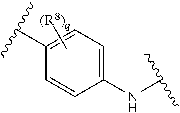

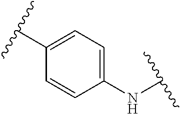 [SMILES: *Nc1ccc(*)cc1]

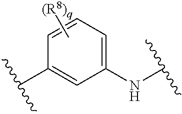

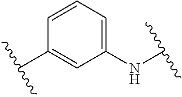 [SMILES: *Nc1cccc(*)c1]

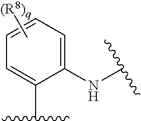

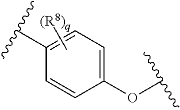

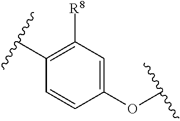

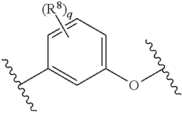

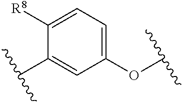

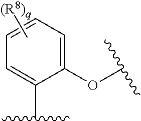

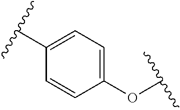 [SMILES: *Oc1ccc(*)cc1]

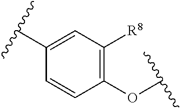

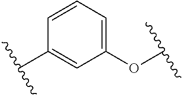 [SMILES: *Oc1cccc(*)c1]

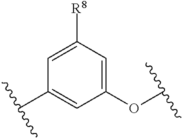

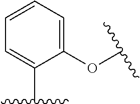 [SMILES: *Oc1ccccc1*]

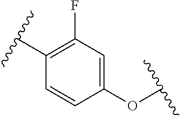 [SMILES: *Oc1ccc(*)c(F)c1]

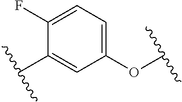 [SMILES: *Oc1ccc(F)c(*)c1]

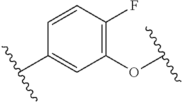 [SMILES: *Oc1cc(*)ccc1F]

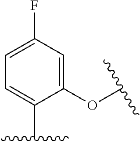 [SMILES: *Oc1cc(F)ccc1*]

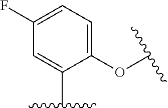 [SMILES: *Oc1ccc(F)cc1*]

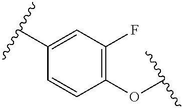 [SMILES: *Oc1ccc(*)cc1F]

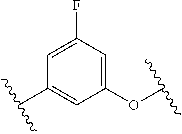 [SMILES: *Oc1cc(*)cc(F)c1]

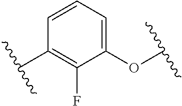 [SMILES: *Oc1cccc(*)c1F]

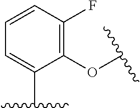 [SMILES: *Oc1c(*)cccc1F]

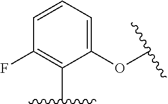 [SMILES: *Oc1cccc(F)c1*]

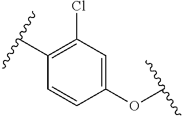 [SMILES: *Oc1ccc(*)c(Cl)c1]

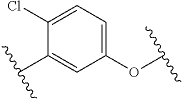 [SMILES: *Oc1ccc(Cl)c(*)c1]

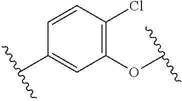 [SMILES: *Oc1cc(*)ccc1Cl]

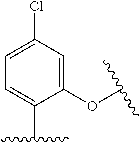 [SMILES: *Oc1cc(Cl)ccc1*]

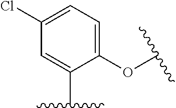 [SMILES: *Oc1ccc(Cl)cc1*]

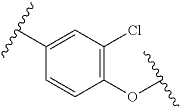 [SMILES: *Oc1ccc(*)cc1Cl]

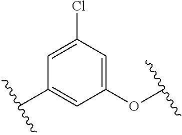 [SMILES: *Oc1cc(*)cc(Cl)c1]

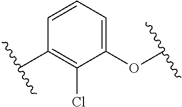 [SMILES: *Oc1cccc(*)c1Cl]

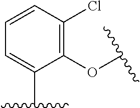 [SMILES: *Oc1c(*)cccc1Cl]

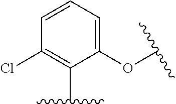 [SMILES: *Oc1cccc(Cl)c1*]

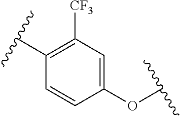 [SMILES: *Oc1ccc(*)c(C(F)(F)F)c1]

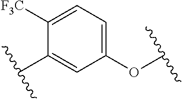 [SMILES: *Oc1ccc(C(F)(F)F)c(*)c1]

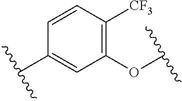 [SMILES: *Oc1cc(*)ccc1C(F)(F)F]

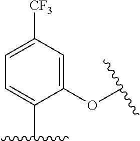 [SMILES: *Oc1cc(C(F)(F)F)ccc1*]

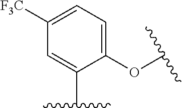 [SMILES: *Oc1ccc(C(F)(F)F)cc1*]

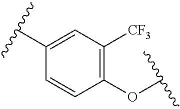 [SMILES: *Oc1ccc(*)cc1C(F)(F)F]

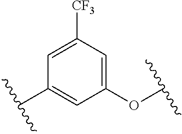 [SMILES: *Oc1cc(*)cc(C(F)(F)F)c1]

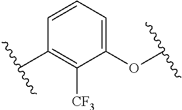 [SMILES: *Oc1cccc(*)c1C(F)(F)F]

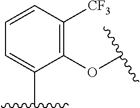 [SMILES: *Oc1c(*)cccc1C(F)(F)F]

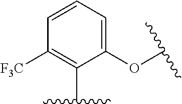 [SMILES: *Oc1cccc(C(F)(F)F)c1*]

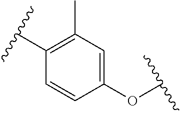 [SMILES: *Oc1ccc(*)c(C)c1]

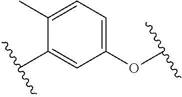 [SMILES: *Oc1ccc(C)c(*)c1]

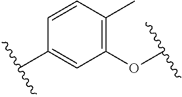 [SMILES: *Oc1cc(*)ccc1C]

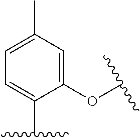 [SMILES: *Oc1cc(C)ccc1*]

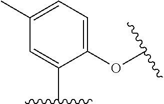 [SMILES: *Oc1ccc(C)cc1*]

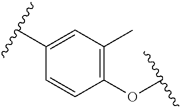 [SMILES: *Oc1ccc(*)cc1C]

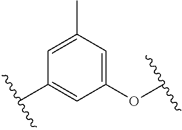 [SMILES: *Oc1cc(*)cc(C)c1]

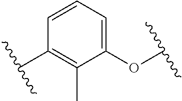 [SMILES: *Oc1cccc(*)c1C]

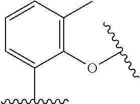 [SMILES: *Oc1c(*)cccc1C]

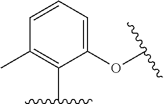 [SMILES: *Oc1cccc(C)c1*]

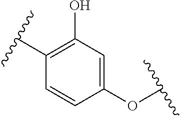 [SMILES: *Oc1ccc(*)c(O)c1]

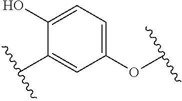 [SMILES: *Oc1ccc(O)c(*)c1]

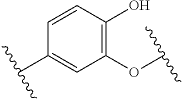 [SMILES: *Oc1cc(*)ccc1O]

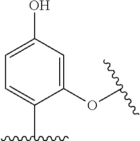 [SMILES: *Oc1cc(O)ccc1*]

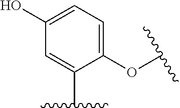 [SMILES: *Oc1ccc(O)cc1*]

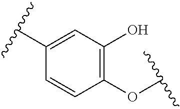 [SMILES: *Oc1ccc(*)cc1O]

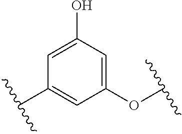 [SMILES: *Oc1cc(*)cc(O)c1]

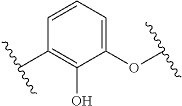 [SMILES: *Oc1cccc(*)c1O]

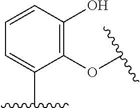 [SMILES: *Oc1c(*)cccc1O]

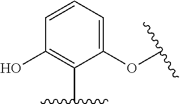 [SMILES: *Oc1cccc(O)c1*]

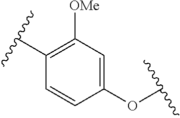 [SMILES: *Oc1ccc(*)c(OC)c1]

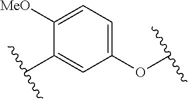 [SMILES: *Oc1ccc(OC)c(*)c1]

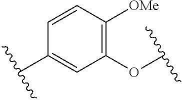 [SMILES: *Oc1cc(*)ccc1OC]

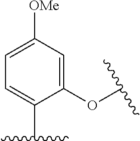 [SMILES: *Oc1cc(OC)ccc1*]

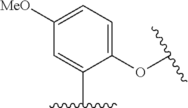 [SMILES: *Oc1ccc(OC)cc1*]

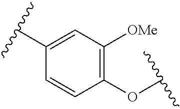 [SMILES: *Oc1ccc(*)cc1OC]

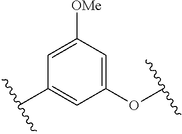 [SMILES: *Oc1cc(*)cc(OC)c1]

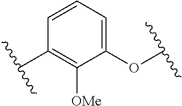 [SMILES: *Oc1cccc(*)c1OC]

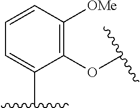 [SMILES: *Oc1c(*)cccc1OC]

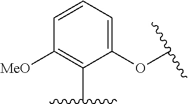 [SMILES: *Oc1cccc(OC)c1*]

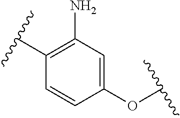 [SMILES: *Oc1ccc(*)c(N)c1]

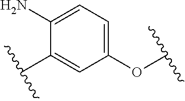 [SMILES: *Oc1ccc(N)c(*)c1]

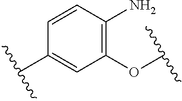 [SMILES: *Oc1cc(*)ccc1N]

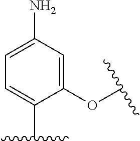 [SMILES: *Oc1cc(N)ccc1*]

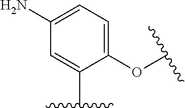 [SMILES: *Oc1ccc(N)cc1*]

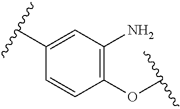 [SMILES: *Oc1ccc(*)cc1N]

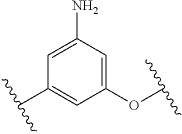 [SMILES: *Oc1cc(*)cc(N)c1]

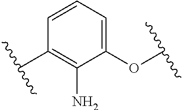 [SMILES: *Oc1cccc(*)c1N]

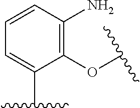 [SMILES: *Oc1c(*)cccc1N]

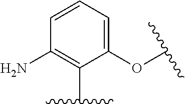 [SMILES: *Oc1cccc(N)c1*]

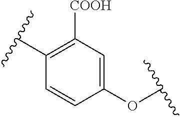 [SMILES: *Oc1ccc(*)c(C(=O)O)c1]

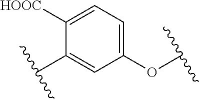 [SMILES: *Oc1ccc(C(=O)O)c(*)c1]

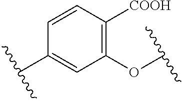 [SMILES: *Oc1cc(*)ccc1C(=O)O]

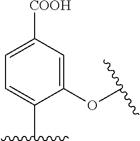 [SMILES: *Oc1cc(C(=O)O)ccc1*]

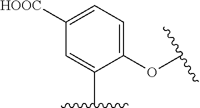 [SMILES: *Oc1ccc(C(=O)O)cc1*]

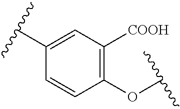 [SMILES: *Oc1ccc(*)cc1C(=O)O]

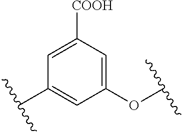 [SMILES: *Oc1cc(*)cc(C(=O)O)c1]

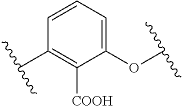 [SMILES: *Oc1cccc(*)c1C(=O)O]

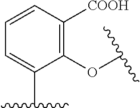 [SMILES: *Oc1c(*)cccc1C(=O)O]

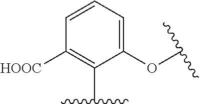 [SMILES: *Oc1cccc(C(=O)O)c1*]

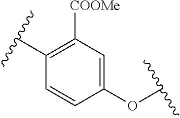 [SMILES: *Oc1ccc(*)c(C(=O)OC)c1]

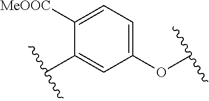 [SMILES: *Oc1ccc(C(=O)OC)c(*)c1]

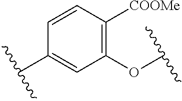 [SMILES: *Oc1cc(*)ccc1C(=O)OC]

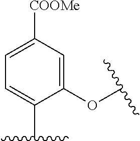 [SMILES: *Oc1cc(C(=O)OC)ccc1*]

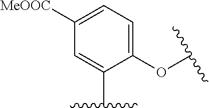 [SMILES: *Oc1ccc(C(=O)OC)cc1*]

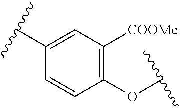 [SMILES: *Oc1ccc(*)cc1C(=O)OC]

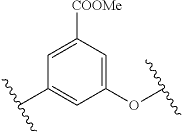 [SMILES: *Oc1cc(*)cc(C(=O)OC)c1]

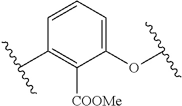 [SMILES: *Oc1cccc(*)c1C(=O)OC]

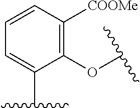 [SMILES: *Oc1c(*)cccc1C(=O)OC]

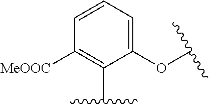 [SMILES: *Oc1cccc(C(=O)OC)c1*]

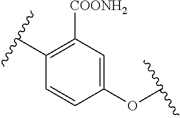

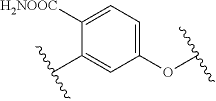

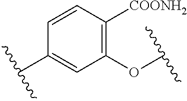

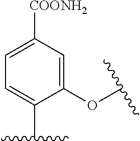

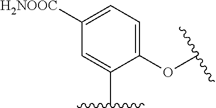

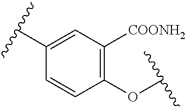

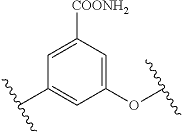

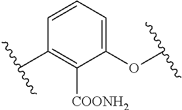

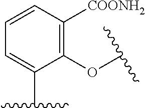

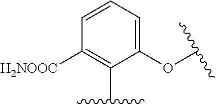

TABLE D
Exemplary R⁶ Moieties

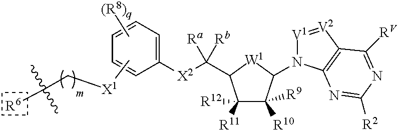

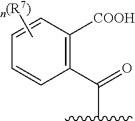

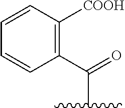 [SMILES: *C(=O)c1ccccc1C(=O)O]

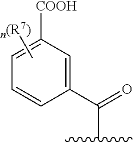

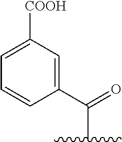 [SMILES: *C(=O)c1cccc(C(=O)O)c1]

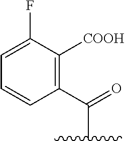 [SMILES: *C(=O)c1cccc(F)c1C(=O)O]

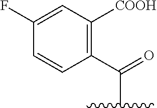 [SMILES: *C(=O)c1ccc(F)cc1C(=O)O]

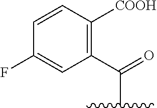 [SMILES: *C(=O)c1cc(F)ccc1C(=O)O]

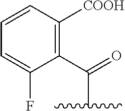 [SMILES: *C(=O)c1c(F)cccc1C(=O)O]

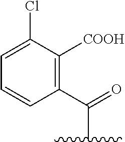 [SMILES: *C(=O)c1cccc(Cl)c1C(=O)O]

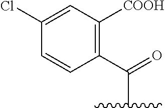 [SMILES: *C(=O)c1ccc(Cl)cc1C(=O)O]

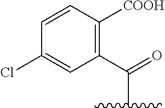 [SMILES: *C(=O)c1cc(Cl)ccc1C(=O)O]

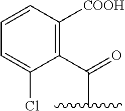 [SMILES: *C(=O)c1c(Cl)cccc1C(=O)O]

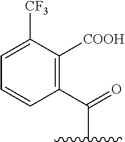 [SMILES: *C(=O)c1cccc(C(F)(F)F)c1C(=O)O]

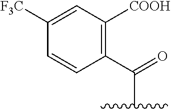 [SMILES: *C(=O)c1ccc(C(F)(F)F)cc1C(=O)O]

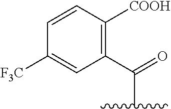 [SMILES: *C(=O)c1cc(C(F)(F)F)ccc1C(=O)O]

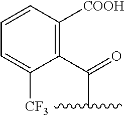 [SMILES: *C(=O)c1c(C(=O)O)cccc1C(F)(F)F]

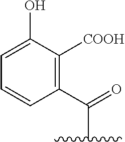 [SMILES: *C(=O)c1cccc(O)c1C(=O)O]

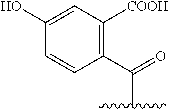 [SMILES: *C(=O)c1ccc(O)cc1C(=O)O]

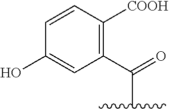 [SMILES: *C(=O)c1cc(O)ccc1C(=O)O]

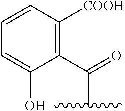 [SMILES: *C(=O)c1c(O)cccc1C(=O)O]

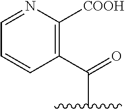 [SMILES: *C(=O)c1cccnc1C(=O)O]

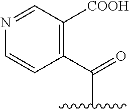 [SMILES: *C(=O)c1ccncc1C(=O)O]

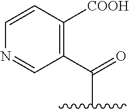 [SMILES: *C(=O)c1cnccc1C(=O)O]

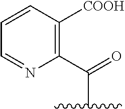 [SMILES: *C(=O)c1ncccc1C(=O)O]

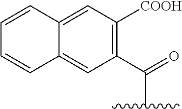 [SMILES: *C(=O)c1cc2ccccc2cc1C(=O)O]

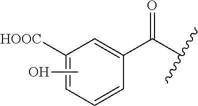

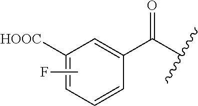

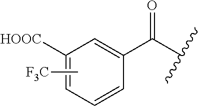

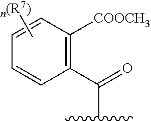

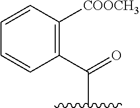 [SMILES: *C(=O)c1ccccc1C(=O)OC]

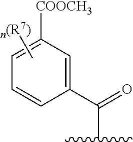

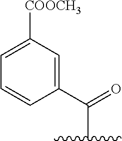 [SMILES: *C(=O)c1cccc(C(=O)OC)c1]

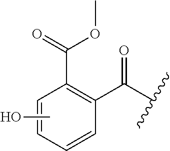

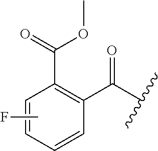

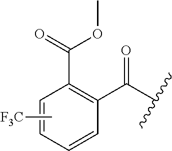

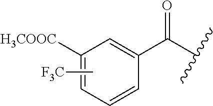

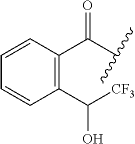 [SMILES: *C(=O)c1ccccc1C(O)C(F)(F)F]

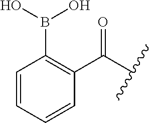 [SMILES: *C(=O)c1ccccc1B(O)O]

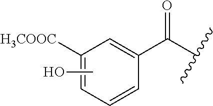

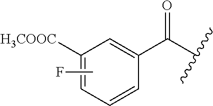

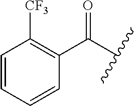 [SMILES: *C(=O)c1ccccc1C(F)(F)F]

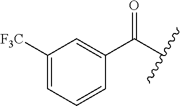 [SMILES: *C(=O)c1cccc(C(F)(F)F)c1]

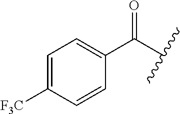 [SMILES: *C(=O)c1ccc(C(F)(F)F)cc1]

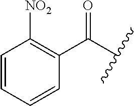 [SMILES: *C(=O)c1ccccc1[N+](=O)[O-]]

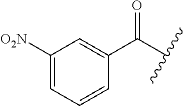 [SMILES: *C(=O)c1cccc([N+](=O)[O-])c1]

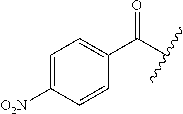 [SMILES: *C(=O)c1ccc([N+](=O)[O-])cc1]

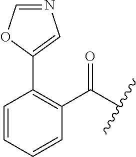 [SMILES: *C(=O)c1ccccc1-c1cnco1]

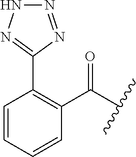 [SMILES: *C(=O)c1ccccc1-c1nn[nH]n1]

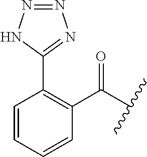 [SMILES: *C(=O)c1ccccc1-c1nnn[nH]1]

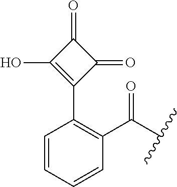 [SMILES: *C(=O)c1ccccc1-c1c(O)c(=O)c1=O]

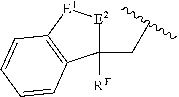

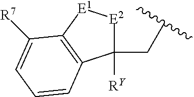

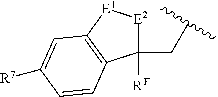

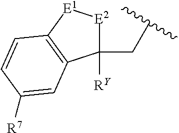

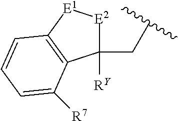

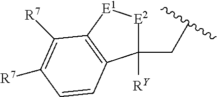

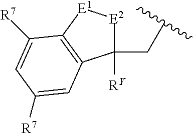

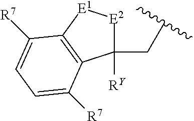

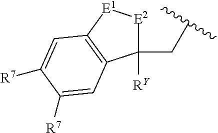

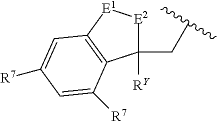

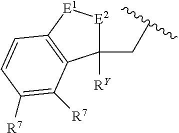

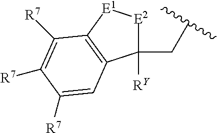

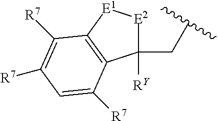

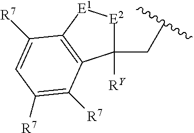

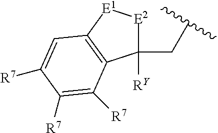

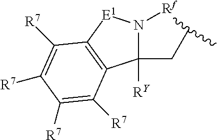

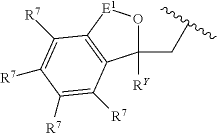

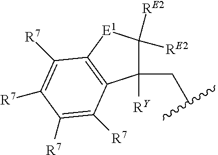

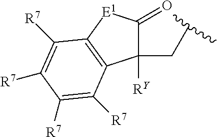

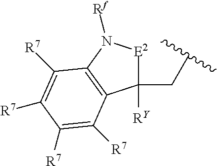

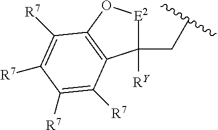

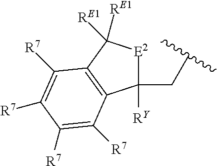

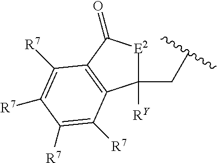

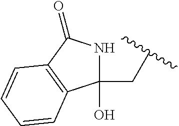 [SMILES: *CC1(O)NC(=O)c2ccccc21]

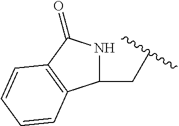 [SMILES: *CC1NC(=O)c2ccccc21]

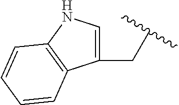 [SMILES: *Cc1c[nH]c2ccccc12]

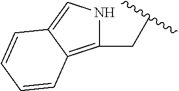 [SMILES: *Cc1[nH]cc2ccccc12]

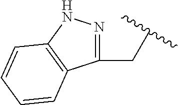 [SMILES: *Cc1n[nH]c2ccccc12]

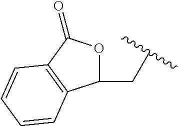 [SMILES: *CC1OC(=O)c2ccccc21]

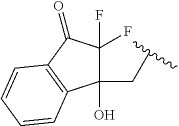 [SMILES: *CC1(O)c2ccccc2C(=O)C1(F)F]

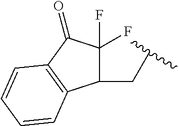 [SMILES: *CC1c2ccccc2C(=O)C1(F)F]

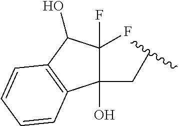 [SMILES: *CC1(O)c2ccccc2C(O)C1(F)F]

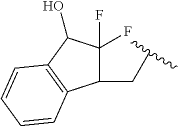 [SMILES: *CC1c2ccccc2C(O)C1(F)F]

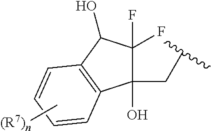

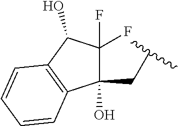 [SMILES: *C[C@@]1(O)c2ccccc2[C@H](O)C1(F)F]

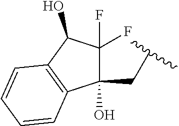 [SMILES: *C[C@@]1(O)c2ccccc2[C@@H](O)C1(F)F]

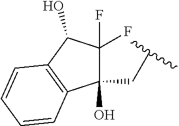 [SMILES: *C[C@]1(O)c2ccccc2[C@H](O)C1(F)F]

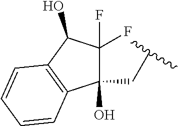 [SMILES: *C[C@]1(O)c2ccccc2[C@@H](O)C1(F)F]

### Pharmaceutical Compositions and Administration

The present disclosure also provides pharmaceutical compositions comprising a compound described herein (e.g., a compound of Formula (I)), or a pharmaceutically acceptable salt or tautomer thereof, and optionally a pharmaceutically acceptable excipient. In some embodiments, the pharmaceutical compositions comprises a compound described herein (e.g., a compound of Formula (I)), or a pharmaceutically acceptable salt, solvate, hydrate, polymorph, co-crystal, tautomer, stereoisomer, or prodrug thereof, and optionally a pharmaceutically acceptable excipient. In certain embodiments, the present disclosure also provides pharmaceutical compositions comprising a compound described herein (e.g., a compound of Formula (I)), or a pharmaceutically acceptable salt or tautomer thereof, and optionally a pharmaceutically acceptable excipient, and further comprising an additional pharmaceutical agent (e.g., antibiotic).

In certain embodiments, the pharmaceutical composition described herein comprises a compound of Formula (I), or a pharmaceutically acceptable salt, stereoisomer, or tautomer thereof, and a pharmaceutically acceptable excipient. In certain embodiments, the pharmaceutical composition described herein comprises a compound of Formula (I), or a pharmaceutically acceptable salt, stereoisomer, or tautomer thereof, a pharmaceutically acceptable excipient, and another pharmaceutical agent. In certain embodiments, the composition is useful for treating and/or preventing a disease. In certain embodiments, the composition is useful for treating a bacterial infection (e.g.. Mycobacterium tuberculosis infection, MRSA infection). In some embodiments, the composition is useful for treating and/or preventing E. coli, M. tuberculosis, B. anthracis, S. aureus, Y. pestis, and P. aeruginosa. In some embodiments, the composition is useful for treating and/or preventing fungal infections, viral infections, and parasitic infections.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating and/or preventing an infectious disease (e.g., bacterial infection (e.g., Mycobacterium tuberculosis infection, MRSA infection)) in a subject in need thereof. In certain embodiments, the effective amount is an amount effective for preventing an infectious disease (e.g., bacterial infection (e.g., Mycobacterium tuberculosis infection, MRSA infection)) in a subject in need thereof (e.g., at risk of contracting an infectious disease). In certain embodiments, the effective amount is an amount effective for reducing the risk of developing an infectious disease (e.g., bacterial infection (e.g., Mycobacterium tuberculosis infection, MRSA infection)) in a subject in need thereof.

In certain embodiments, the effective amount is an amount effective for inhibiting menaquinone biosynthesis. In certain embodiments, the effective amount is an amount effective for inhibiting o-succinylbenzoate-CoA synthetase (MenE) in an infection in a subject. In certain embodiments, the effective amount is an amount effective for inhibiting MenE in an infectious microorganism. In certain embodiments, the effective amount is an amount effective for inhibiting an acyl-CoA synthetase in an infectious microorganism in a subject. In certain embodiments, the effective amount is an amount effective for inhibiting an acyl-CoA synthetase in an infectious microorganism. In certain embodiments, the effective amount is an amount effective for inhibiting a adenylate-forming enzyme in an infectious microorganism in a subject. In certain embodiments, the effective amount is an amount effective for inhibiting an adenylate-forming enzyme in an infectious microorganism.

In certain embodiments, the effective amount is an amount effective for inhibiting MenE in an infectious microorganism in a subject. In certain embodiments, the effective amount is an amount effective for inhibiting MenE in an infectious microorganism. In certain embodiments, the effective amount is an amount effective for inhibiting an adenylate-forming enzyme in an infectious microorganism causing an infection in a subject. In certain embodiments, the effective amount is an amount effective for inhibiting an adenylate-forming enzyme in an infectious microorganism.

In certain embodiments, the subject being treated or at risk of contracting an infectious disease is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments. the subject is a zoo animal. In another embodiment, the subject is a research animal, such as a rodent (e.g., mouse, rat), dog, pig, or non-human primate. In certain embodiments, the animal is a genetically engineered animal. In certain embodiments. the animal is a transgenic animal (e.g., transgenic mice and transgenic pigs). In certain embodiments, the subject is a fish or reptile.

In certain embodiments, the effective amount is an amount effective for inhibiting menaquinone biosynthesis by at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for inhibiting cofactor (e.g., menaquinone) biosynthesis by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for a range of inhibition between a percentage described in this paragraph and another percentage described in this paragraph, inclusive.

In certain embodiments, the effective amount is an amount effective for inhibiting an adenylate-forming enzyme by at least about 10%, at least about 20%, at least about 30%, at least about 40%, at least about 50%, at least about 60%, at least about 70%, at least about 80%, at least about 90%, at least about 95%, at least about 98%, or at least about 99%. In certain embodiments, the effective amount is an amount effective for inhibiting adenylate-forming enzyme by not more than 10%, not more than 20%, not more than 30%, not more than 40%. not more than 50%, not more than 60%, not more than 70%, not more than 80%, not more than 90%, not more than 95%, or not more than 98%. In certain embodiments, the effective amount is an amount effective for a range of inhibition between a percentage described in this paragraph and another percentage described in this paragraph, inclusive.

In certain embodiments, the effective amount is an amount effective for inhibiting MenE by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting MenE by not more than 10%, not more than 20%, not more than 30%, not more than 40%, not more than 50%. not more than 60%, not more than 70%, not more than 80%, not more than 90%, not more than 95%, or not more than 98%. in certain embodiments, the effective amount is an amount effective for a range of inhibition between a percentage described in this paragraph and another percentage described in this paragraph, inclusive.

In certain embodiments, the effective amount is an amount effective for inhibiting an acyl-CoA synthetase by at least about 10%, at least about 20%, at least about 30%, at least about 40%, at least about 50%, at least about 60%, at least about 70%, at least about 80%, at least about 90%, at least about 95%, or at least about 99%. In certain embodiments, the effective amount is an amount effective for inhibiting an acyl-CoA synthetase by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for a range of inhibition between a percentage described in this paragraph and another percentage described in this paragraph. inclusive.

In certain embodiments, the effective amount is an amount effective for inhibiting respiration in a microorganism by at least about 10%, at least about 20%, at least about 30%, at least about 40%, at least about 50%, at least about 60%, at least about 70%, at least about 80%, at least about 90%, at least about 95%, or at least about 99%. In certain embodiments, the effective amount is an amount. effective for inhibiting respiration in a microorganism by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for a range of inhibition between a percentage described in this paragraph and another percentage described in this paragraph, inclusive.

In certain embodiments, the effective amount is an amount effective for killing a microorganism by at least about 10%, at least about 20%, at least about 30%, at least about 40%, at least about 50%, at least about 60%, at least about 70%, at least about 80%, at least about 90%, at least about 95%, or at least about 99%. In certain embodiments, the effective amount is an amount effective for killing a microorganism by not more than 10%, not more than 20%, not more than 30%, not more than 40%, not more than 50%, not more than 60%, not more than 70%, not more than 80%, not more than 90%, not more than 95%. or not more than 98%. In certain embodiments, the effective amount is an amount effective for a range of inhibition between a percentage described in this paragraph and another percentage described in this paragraph, inclusive.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping andlor packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size. and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inostol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked polyvinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutor), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Plutonic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.). natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(yinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g.. sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof. phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, iniidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A. vitamin C, vitamin E, beta-carotene, citric acid, acetic acid, dehydroacetic acid. ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine. sodium lauryl sulfate (SLS), sodium lauryl ether sulfate (SLES), sodium bisulfite, sodium metabisulfite, potassium sulfite, potassium metabisulfite, Glydane Plus, Phenonie, methylparaben, Germall® 115. Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop. isopropyl myristate, jojoba, kukui nut, lavandin, lavender, lemon, litsea cubeba, macademia nut, mallow, mango seed, meadowfoam seed, mink, nutmeg, olive, orange. orange roughy, palm. palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizinst agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfinyl alcohol, polyethylene glycols and fatty acid esters of sorbitan, and mixtures thereof, Besides inert diluents, the oral compositions can include adjuvants such as wetting agents, emulsifying and suspending agents, sweetening, flavoring, and perfuming agents. In certain embodiments for parenteral administration, the conjugates described herein are mixed with sotubilizing agents such as Crernophor®, alcohols, oils, modified oils, glycols, polysorbates, cyclodextrins, polymers, and mixtures thereof.

Injectable preparations. for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water. Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier, such as sodium citrate and dicalcium phosphate. andfor (a) fillers or extenders, such as starches, lactose, sucrose, glucose, mannitol, and silicic acid, (b) binders, such as, for example, carboxymethylcellulose, alginates, gelatin, polyvinylpyrrolidinone, sucrose, and acacia, (c) humectants, such as glycerol, (d) disintegrating agents, such as agar, calcium carbonate, potato or tapioca starch, alginic acid, certain silicates, and sodium carbonate, (e) solution retarding agents, such as paraffin, (f) absorption accelerators, such as quaternary ammonium compounds, (g) wetting agents, such as, for example, cetyl alcohol and glycerol monostearate, (h) absorbents such as kaolin and bentonite clay, and (i) lubricants, such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredients) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared. for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, andlor pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about I to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent, such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the infectious disease being treated and/or prevented, as well as the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment and/or prevention; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments. the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years. five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell,

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating an infectious disease in a subject in need thereof (e.g., tuberculosis), in preventing an infectious disease in a subject in need thereof, and/or in reducing the risk to develop an infectious disease in a subject in need thereof), improve bioavailability, improve safety, reduce drug resistance, reduce and/or modify metabolism, inhibit excretion, and/or modify distribution in a subject, microorganism, or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., infectious disease (e.g., tuberculosis), proliferative disease, hematological disease, or painful condition). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments. the levels utilized in combination will be lower than those utilized individually.

The additional pharmaceutical agents include, but are not limited to, anti-inflammatory agents, anti-bacterial agents, anti-viral agents, and pain-relieving agents.

In certain embodiments, the additional pharmaceutical agent inhibits cofactor biosynthesis. In certain embodiments, the additional pharmaceutical agent inhibits menaquinone biosynthesis. In certain embodiments, the additional pharmaceutical agent is a binder or inhibitor of MenE. In certain embodiments, the additional pharmaceutical agent is a binder or inhibitor of an adenylate-forming enzyme. In certain embodiments, the additional pharmaceutical agent is a binder or inhibitor of an acyl-CoA synthetase. In certain embodiments, the additional pharmaceutical agent is a binder or inhibitor of an AMP-producing synthetase. In certain embodiments, the additional pharmaceutical agent inhibits cellular respiration. In certain embodiments, the additional pharmaceutical agent inhibits protein synthesis. In certain embodiments, the additional pharmaceutical agent down-regulates the expression of PqsABCDE, PqsR, PqsH, or PhnAB. In certain embodiments, the additional pharmaceutical agent binds a ribosome. In certain embodiments, the additional pharmaceutical agent is an antibiotic. In certain embodiments, the additional pharmaceutical agent is an anti-bacterial agent.

In some embodiments, the additional pharmaceutical agent being used in combination with a compound of Formula (I) is an antibiotic. Exemplary antibiotics include, but are not limited to gentamicin, amikacin, tobramycin. ciprofloxacin, levofloxacin, ceftazidimine, cefepime, cefoperazone, cefpirome, ceftobiprole, carbenicllin, ticarcillin, mezlocillin, aziocillin, piperacillin, meropenern, imipenem, doripenem, polymyxin B, colistin, aztreonam, isoniazid, rifampicin (also called rifampin), pyrazinamide, ethambutol, streptomycin, moxifloxacin, gatifloxacin, amikacin, capremycin, kanamycin, ethionamide, prothionamide, cycloserine, terizidone, linezolide, clofazimine, pretomanid, bedaquiline, delamanid, or rifamycins. In certain embodiments, the additional pharmaceutical agent is isoniazid, rifampicin (also called rifampin), pyrazinamide, ethambutol, or streptomycin. In some embodiments, the additional pharmaceutical agent is levofloxacin, moxifloxacin, gatifloxacin, amikacin, capremycin, kanamycin, ethionamide, prothionamide, cycloserine, terizidone, linezolide, or clofazimine.

In certain embodiments, the additional pharmaceutical agent is a β-lactam antibiotic. Exemplary β-lactam antibiotics include, but are not limited to: β-lactamase inhibitors (e.g., avibactam, clavulanic acid, tazobactam, sulbactam); carbacephems (e.g., loracarbef); carbapenems (e.g., doripenem, imipenem, ertapenem, meropenem); cephalosporins (1^{st} generation) (e.g., cefacetrile, cefadroxil, cefalexin, cefaloglycin, cefalonium, cefaloridine, cefalotin, cefapirin, cefatrizine, cefazatlur. cefazedone, cefazolin, cefradine, cefroxadine, ceftezole, cephalosporin C); cephalosporins (2^{nd} generation) (e.g., cefaclor, cefamandole, cefbuperzone, ceftnetazole, cefonicid, ceforanide, cefotetan, cefotiam, cefoxitin, cefininox, cefprozil, cefuroxime, cefuzonam); cephalosporins (3^{rd} generation) (e.g., cefcapene, cefdaloxime, cefdinir, cefditorin, cefetamet, cefixime, cefmenoxime, cefodizime, cefoperazone, cefotaxime, cefovecin, cefpimizole, cefpiramide, cefpodoxime, ceftamere, ceftazidime, cefteram, ceftibuten, ceftiofur, ceftiolene, ceftizoxime, ceftriaxone, latamoxet); cephalosporins (4^{th} generation) (e.g., cefepime, cefluprenam, cefoselis, cefozopran. cefpirome, cefquinome, flomoxef); cephalosporins (5^{th} generation) (e.g., ceftaroline fosamil, ceftobiprole, ceftolozane); cephems (e.g., cefaloram, cefaparole, cefcanel, cefedrolor, cefempidone, cefetrizole, cefivitril, cefrnepidium cefoxazole, cefrotil, cefsulodin, cefsutnide, ceftioline, ceftioxime, cefuracetime, nitrocefin); monobactams (e.g., aztreonam, carumonam, norcadicin A, tabtoxinine ft-lactam, tigemonam?); penicillins/penams (e.g., amoxicillin, amoxicillin/clavulanate, ampicillin, ampicillin/flucloxacillin, ampicillin/sulbactam, azidocillin, azlocillin, bacampacillin, benzathine benzylpenicillin, benzathine phenoxymethylpenicillin, carbenicillin, carindacillin, clometocillin, cloxacillin, dicloxacillin, epicillin, flucloxacillin, hetactlin, mecillinam, mezlocillin, meticillin, metampiciillin, nafcillin, oxacillin, penamacillin, penicillin G, penicillin V, phenaticillin, piperacillin, piperacillinitazobactam, pivampicillin, pivmeclillinam, procaine benzylpenicillin, propicillin, sulbenicillin, talampicillin, temocllin, ticarcillin, ticarcillin/clavulanate); and penems/carbapenems (e.g., biapenem, doripenem, ertapenem, faropenem, imipenem, imipenem/cilastatin, lenapenem, meropenem, panipenem, razupenem, tebipenem, thienamycin, tomopenem).

In certain embodiments, the additional pharmaceutical agent is a non-β-lactam antibiotic. Exemplary non-β-lactam antibiotics include, but are not limited to: aminoglycosides (e.g., amikacin, dibekacin, gentamicin, kanamycin, neomycin, netilmicin, tobramycin, paromomycin, sisomicin, streptomycin, spectinomycin); ansamycins (e.g., geldanamycin, herbimycin); glycopeptides (e.g., belomycin, dalbavancin, oritavancin, ratnoplanin, teicoplanin, telavancin, vancomycin); glycylcyclines (e.g., tigecycline); lincosamides (e.g., clindamycin, lincomycin); lipopeptides (e.g., anidulafungin, caspofungin, cilofungin, daptomycin, echinocandin B, micafungin, mycosubtilin); macrolides (e.g., azithromycin, carbomycin A, clarithromycin, dirithromycin, erythromycin, josmycin, kitasamycin, midecamycin, oleandomycin, roxithromycin, solithromycin, spiramycin, troleandomycin, telithromycin, tylosin); nitrofurans (e.g., furazolidone, furylfurarnide, nitrofurantoin, nitrofitrazone, nifuratel, nifurquinazol, nifurtoinol, nifuroxazide, nifurtimox, nifurzide, ranbezolid); nitroimidazoles (e.g., metronidazole, nimorazole, tinadazole); oxazolidinones (e.g., cycloserine, linezolid, posizolid radezolid, tedizolid); polypeptides (e.g., actinomycin, bacitracin, colistin, polymyxin B); quinolones (e.g.. balofloxacin, besifloxacin, cinoxacin, ciprofloxacin, clinafloxacin, danofloxacin, delafloxacin, diflofloxacin, enoxacin, enrofloxacin, fleroxacin, flumequine, gatifloxacin, gemifloxacin, grepafloxacin, ibafloxacin, JNJ-Q2, levofloxacin, lomefloxacin, marbofloxacin, moxifloxacin, nadifloxacin, nalidixic acid, nemonoxacin, norfloxacin, ofloxacin, orbifloxacin, oxilinic acid, pazufloxacin, pefloxacin, piromidic acid, pipemidic acid, prulifloxacin, rosoxacin, rufloxacin, sarafloxacin, sparfloxacin, sitafloxacin, temafloxacin, tosufloxacin, trovafloxacin); rifamycins (e.g., rifamycin B, rifamycin S, rifamycin SV, rifampicin, rifabutin, rifapentine, rifalazil, rifaximin); sulfonamides (e.g., co-trimoxazole, mafenide, pediazole, sulfacetamide, sulfadiazine, silver sulfadiazine, sulfadimidine, sulfadimethoxine, sulfadoxine, sulfafurazole, sulfamethizole, sulfamethoxazote, sulfamethoxypyridazine, sulfametopyrazine, sulfametoxydiazine, sulfamoxole, sulfanilamide, sulfanitran, sulfasalazine, sulfisomidine, sulfonamidochrysoidine, trimethoprim); tetracyclines (e.g., 6-deoxytetracycline, aureomycin, chlortetracycline, demeclocycline, doxycycline, lymecycline, meclocyctine, methacycline, minocycline, oxytetracycline, PTK-0796, sancycline, rolitetracycline, tetracycline, tetramycin); tuberactinomycins (e.g., tuberactinomycin A, tuberactinomycin O, viomycin, enviomycin, capreomycin); arsphenamine; chloramphenicol; dalfoprisitin; fosfomycin; fusidic acid; fidaxomycin, gramicidin; lysozyme; mupirocin; platensimycin; pristinamycin; sparsomycin; quinupristin; quinupristinidalfopristin; teixobactin; and thiamphenicol.

In certain embodiments, the additional pharmaceutical agent is selected from the group consisting of allicin, amikacin, arginine, azlocillin, aztreonam, bedaquiline, capreomycin, carbenicllin, cefepime, cefoperazone, cefpirome, ceftaroline, ceftazidimine, ceftobiprole, ciprofloxacin, clarithromycin, clavulanic acid, clindamycin, clofazimine, co-amoxiclav, colistin, co-trimioxazole, cycloserine, dalfopristin, dapsone, daptomycin, delafloxacin, delamanid, doripenem, doxycycline, enviomycin, ethambutol, ethionamide, fosamil, gatifloxacin, gentamicin, imipenem, interferon-y, isoniazid, JNJ-Q2, kanamycin, levofloxacin, linezolid, meropenem, metronidazole, mezlocillin, minocycline, moxitloxacin, pretomanid, p-aminosalicyclic acid, perchlorperazine, piperacillin, polymyxin B, pretomanid, prothioamide, pyrazinamide, quinipristin, rifabutin, rifampicin, rifamycins, rifapentine, rifaximin. streptomycin, teicoplanin, terizidone, thioazetazeone, thioridazine, ticarcillin, tigecycline, tobramycin, vancomycin, viomycin, and vitamin D.

In certain embodiments, the additional pharmaceutical agent is isoniazid. In certain embodiments, the additional pharmaceutical agent is rifampicin (also called rifampin). In certain embodiments, the additional pharmaceutical agent is pyrazinamide. In certain embodiments, the additional pharmaceutical agent is ethambutol. In certain embodiments, the additional pharmaceutical agent is streptomycin. In certain embodiments, the additional pharmaceutical agent is a carbapenem. In some embodiments, the additional pharmaceutical agent is doripenem, imipenem, or meropenem. In certain embodiments, the additional pharmaceutical agent is a glycylcycline. In some embodiments, the additional pharmaceutical agent is tigecycline. In certain embodiments, the additional pharmaceutical agent is a aminoglycoside. In some embodiments, the additional pharmaceutical agent is gentamycin, amikacin, or tobramycin. In certain embodiments, the additional pharmaceutical agent is a quinolone. In some embodiments, the additional pharmaceutical agent is ciprofloxacin or levofloxacin. In certain embodiments, the additional pharmaceutical agent is a cephalosporin. In some embodiments, the additional pharmaceutical agent is ceftazidime, cefepime, cefoperazone, cefpirome, ceftobirprole, or ceftaroline fosamil. In certain embodiments, the additional pharmaceutical agent is a penicillin. In some embodiments, the additional pharmaceutical agent is an antipseudomonal penicillin or extended spectrum penicillin. In certain embodiments, the additional pharmaceutical agent is a carboxypenicillin or a ureidopenicillin. In some embodiments, the additional pharmaceutical agent is carbenicillin, ticarcillin, mezlocillin, aziocillin, piperacillin, or mecillinam. In certain embodiments, the additional pharmaceutical agent is a polymyxin. In some embodiments, the additional pharmaceutical agent is polymyxin B or colistin. In certain embodiments, the additional pharmaceutical anent is a monobactam. In some embodiments, the additional pharmaceutical agent is aztreonam. In certain embodiments, the additional pharmaceutical agent is a β-lactamase inhibitor. In some embodiments, the additional pharmaceutical agent is sulbactam.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating an infectious disease (e.g., bacterial infection (e.g., Mycobacterium tuberculosis infection. Staphylococcus aureus infection (e.g.. MRSA)) in a subject in need thereof In certain embodiments, the kits are useful for preventing an infectious disease (e.g., bacterial infection (e.g., Mycobacterium tuberculosis infection, Staphylococcus aureus infection)) in a subject in need thereof. In certain embodiments, the kits are useful for reducing the risk of developing an infectious disease (e.g., bacterial infection (e.g., Mycobacterium tuberculosis infection, Staphylococcus aureus infection)) in a subject in need thereof. In certain embodiments, the kits are useful for inhibiting the biosynthesis of cofactors in an infectious microorganims causing an infection in a subject or in an infectious microorganism. In certain embodiments. the kits are useful for inhibiting the biosynthesis of menaquinone in an infectious microorganims causing an infection in a subject or in an infectious microorganism. In certain embodiments, the kits are useful for inhibiting MenE. In certain embodiments, the kits are useful for inhibiting an adenylate-forming enzyme. In certain embodiments, the kits arc useful for inhibiting an acyl-CoA synthetase.

In certain embodiments, the kits are useful for treating a patient with tuberculosis. In certain embodiments, the kits are useful for treating a patient with a MRSA infection, In certain embodiments, the kits are useful for eradication of a biofilm in a patient. In certain embodiments, the kits are useful for preventing the formation of a biofilm in a patient. In certain embodiments, the kits are useful for preventing the formation of a biofilm on a surface.

In certain embodiments. a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating an infectious disease (e.g., bacterial infection (e.g., Mycobacterium tuberculosis infection, Staphylococcus aureus infection)) in a subject in need thereof. In certain embodiments, the kits and instructions provide for preventing an infectious disease (e.g., bacterial infection (e.g., Mycobacterium tuberculosis infection, Staphylococcus aureus infection)) in a subject in need thereof. In certain embodiments, the kits and instructions provide for reducing the risk of developing an infectious disease (e.g., bacterial infection (e.g., Mycobacterium tuberculosis infection, Staphylococcus aureus infection)) in a subject in need thereof. In certain embodiments, the kits and instructions provide for inhibiting biosynthesis of menaquinone in an infection in a subject or in an infectious microorganism. In certain embodiments, the kits and instructions provide for inhibiting MenE in an infection in a subject or in an infectious microorganism. In certain embodiments, the kits and instructions provide for inhibiting an adenylate-forming enzyme in an infection in a subject or in an infectious microorganism. In certain embodiments, the kits and instructions provide for inhibiting an acyl-CoA synthetase in an infection in a subject or in an infectious microorganism In certain embodiments, the kits and instructions provide for inhibiting menaquinone biosynthesis in an infection in a subject or in an infectious microorganism. A kit described herein may include one or more additional pharmaceutical agents described herein as a separate composition.

### Methods of Treatment and Uses

The present disclosure also provides methods that may be useful for the treatment and/or prevention of a disease. The present disclosure also provides compounds for use in methods that may be useful for the treatment and/or prevention of a disease. In certain embodiments, the disease is an infectious disease. In certain embodiments, the infectious disease is a bacterial infection. In certain embodiments, the infectious disease is a fungal infection. In certain embodiments, the infectious disease is a parasitic infection. in certain embodiments, the infectious disease is a viral infection. In certain embodiments, the infectious disease is associated with another disease or condition, for example, in subjects with a weakened immune system as a result of HIV infection, AIDS, lupus, cancer, cystic fibrosis, or diabetes, or subjects with burns. In certain embodiments, the bacterial infection is an infection caused by Gram-positive bacteria. In certain, embodiments, the bacterial infection is an infection caused by Gram-negative bacteria. In certain embodiments, the bacterial infection is a Staphylococcus infection, a Bacillus infection, or an Escherichia infection. In some embodiments, the bacterial infection is caused by a member of Mycobacteriacae. In certain embodiments, the bacterial infection is an infection caused by Mycobacterium tuberculosis. In some embodiments, the infectious disease is tuberculosis. In certain embodiments, the bacterial infection is a mycobacterial infection. In some embodiments the bacterial infection is an atypical mycobacterial infection. In some embodiments, the infectious disease is tuberculosis. In some embodiments, the infectious disease is multi-drug resistant tuberculosis (MDR-TB). In some embodiments, the infectious disease is extensively drug-resistant tuberculosis (XDR-TB). In some embodiments, the bacterial infection is caused by a member of Staphylococcaccae. In certain embodiments, the bacterial infection is a Staphylococcus infection. In some embodiments, the bacterial infection is a Staphylococcus aureus infection. In some embodiments, the bacterial infection is a methicillin-resistant Staphylococcus aureus (MRSA) infection. In some embodiments, the bacterial infection is healthcare-associated MRSA (HA-MRSA). In some embodiments. the bacterial infection is community-associated MRSA (CA-MRSA). In some embodiments, the bacterial infection is a vancomycin-intermediate Staphylococcus aureus (VISA) infection or a vancomycin-resistant Staphylococcus aureus (VRSA) infection. In some embodiments, the bacterial infection is B. anthraeis. In certain embodiments, the bacterial infection is E. coli.

Exemplary bacterial infections include, but are not limited to, infections with a Gram positive bacteria (e.g., of the phylum Actinobacteria, phylum Firmicutes, or phylum Tenericutes); Gram negative bacteria (e.g., of the phylum Aquificae, phylum Deinococcus-Thermus, phylum Fibrobacteres/Chlorobi/Bacteroidetes (FCB), phylum Fusobacteria, phylum Gemmainnonadest, phylum Ntrospirae, phylum Platictomycetes/Verrucomicrobial/Chlamydiae (PVC), phylum Proteobacteria, phylum Spirochaetes, or phylum Synergistetes); or other bacteria (e.g., of the phylum Acidabacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the bacteria is a member of the phylum Firmicutes and the genus Enterococcus, i.e., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus. In certain embodiments, the bacteria is a member of the phylum Firmicutes and the genus Staphylococcus, i.e., the bacterial infection is a Staphylococcus infection. Exemplary Staphylococci bacteria include, but are not limited to, S. arleuve, S. aureus, S. auricularis, S. capitis, S. caprae, S. carmous, S. chromogenes, S. cohii, S. condimenti, S. croceolyticus, S. delphini, S. devricsei, S. epidermis, S. equorum, S. felis, S. fluroeuil, S. gallinarum, S. haemolyticus, S. hominis, S. hyicus, S. intermedius, S. kloosii, S. leei, S. lemus, S. lugdunesis, S. lutrae, S. lyticans, S. massiliensis, S. microtic, S. muscae, S. nepalensis. S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succimus, S. vitulinus, S, warneri, and S. xylosus. In certain embodiments, the Staphylococcus infection is a S. aureus infection. In certain embodiments, the Staphylococcus infection is a methicillin-resistant Staphylococcus aureus (MRSA) infection. In some embodiments, the Staphylococcus infection is an vancomycin-intermediate Staphylococcus aureus (VISA) infection or a vancomycin-resistant Staphylococcus aureus (VRSA) infection.

In certain embodiments, the bacteria is a member of the phylum Firmicutes and the genus Bacillus, i.e., the bacterial infection is a Bacillus infection. Exemplary Bacillus bacteria include, but are not limited to, B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. lichenifirmis, B. megaterium, B. mesentericus, B. mucilaginocus, B. mycoides, B. natto, B. pantothenticus, B. polymyxa, B. pseudoanthracis, B. pumilus, B. schlegelii, B. sphaericus, B. sporothermochirans, B. stearothermophilus, B. subtilis, B. thermoglucosidasius, B. thuringiensis, B. vuligatis, and B. weihenstephanensis. In certain embodiments, the Bacillus infection is a B. subtilis infection. In certain embodiments, the B. subtilis has an efflux (e.g., mef, msr) genotype, In certain embodiments, the B. subtilis has a methylase (e.g., erm) genotype. In certain embodiments, the Bacillus infection is a B. anthracis infection. In certain embodiments, the bacteria is a member of the phylum Firmicutes and the genus Streptococcus, i.e., the bacterial infection is a Strepococcus infection. Exemplary Streptococcus bacteria include, but are not limited to, S. agalactiae, S. anginosus, S. bovis, S. canis. constellatus, S. dysgalactiae, S. equinus, S. iniae, S. intermedius, S. mitis, S. mutans, S. oralis, S. parasanguinis, S. peroris, S. pneumoniae, S. pyogenes, S. ratti, S. salivarius, S. thermophilus, S. sanguinis, S. sobrinus, S. suis, S. uberis, S. vestibularis, S. viridans, and S. zooepidemicus. In certain embodiments, the Streptococcus infection is an S. pyogenes infection. In certain embodiments, the Strepococcus infection is an S. pneumoniae infection. In certain embodiments, the S. pneumoniae has an efflux (e.g., mef, msr) genotype. In certain embodiments, the S. pneumoniae has a methylase (e.g., erm) genotype. In certain embodiments, the bacteria is a member of the phylum Firmicutes and the genus Clostridium, i.e., the bacterial infection is a Clostridium infection. Exemplary Clostridia bacteria include, but are not limited to, C. botulinum, C. difficile, C. perfringens, C. tetani, and C. sordellii.

In certain embodiments, the Gram negative bacteria is a bacteria of the phylum Proteobacteria and the genus Escherichia. i.e., the bacterial infection is an Escherichia infection. Exemplary Escherichia bacteria include, but are not limited to, E. alberiii, E. blattae, E. coli, E. jergusonii, E. hermannii, and E. vulneris. In certain embodiments, the Escherichia infection is an E. coli infection. In certain embodiments, the Gram negative bacteria is a bacteria of the phylum Proteobacteria and the genus Haemophilus. i.e., the bacterial infection is an Haemophilus infection. Exemplary Haemophilia bacteria include, but are not limited to, H. aegyptius, H. aphrophilus, H. avium, H. ducreyi, H. felis, H. haemolyticus, H. influenzae, H. paraiufluenzae, H. paracuniculus, K parahaemolyticus, H. pittmaniae, Haemophilia segnis, and H. somnus. In certain embodiments, the Haemophilus infection is an H. iufluenzae infection.

In certain embodiments, the Gram negative-bacteria is a bacteria of the phylum Proteobacteria and the genus Acinetobacter. i.e., the bacterial infection is an Acinetobacter infection. Exemplary Acinetobacter bacteria include, but are not limited to, A. baumanii, A. haemolyticus, and A. lwoffii. In certain embodiments, the Acinewbacter infection is an A. baumanii infection. In certain embodiments, the Gram-negative bacteria is a bacteria of the phylum Proteobacteria and the genus Klebsiella. i.e., the bacterial infection is a Klebsiella infection. Exemplary Klebsiella bacteria include, but are not limited to, K. granulomatis, K. oxytoca, K. michiganensis, K. pneumoniae, K. quasipneumoniae, and K. variicola. In certain embodiments, the Klebsiella infection is a K. pneumoniae infection. In certain embodiments, the Gram-negative bacteria is a bacteria of the phylum Proteobacteria and the genus Psendomonas. i.e., the bacterial infection is a Pseudomonas infection. Exemplary Pseudomonas bacteria include, but are not limited to, P. aeruginosa, P. oryzihabitans, P. plecoglissicida, P. syringae, P. putida, and P. fluoroscens. In certain embodiments, the Pseudomonas infection is a P. aeruginosa infection. In certain embodiments, the Gram-negative bacteria is a bacteria of the phylum Bacteroidetes and the genus Bacteroides. i.e., the bacterial infection is a Bacteroides infection. Exemplary Bacteroides bacteria include, but are not limited to, B. fragilis, B. distasomis, B. ovatus, B. thetaiotaomicron, and B. vulgatus. In certain embodiments, the Bacteroides infection is a B. fragilis infection. In certain embodiments, the Gram negative-bacteria is a bacteria of the phylum Proteobacteria and the genus Yersinia. i.e., the bacterial infection is an Yersinia infection. Exemplary Yersinia bacteria include, but are not limited to, Y. pestis, Y. entercolitica. and Y. pseudotuberculosis. In certain embodiments, the Acinetobacter infection is an Y. pestis infection.

In certain embodiments, the bacterial infection is caused by a bacteria of the phylum Actinobacteria. Exemplary bacteria of the phylum include, but are not limited to bacteria within Acidimicrohiaceae family, Actinomycetaceae family, Cotynebacteriaceae family, Gordoniaceae family, Mycobacteriaceae family, Nocardiaceae family, Tsukamureilaceae family, Williamslaceae family, Acidothermaceae family, Frankiaceae family, Geodermatophilaceae, Kineosporiaceae, Microsphaeraceae family, Sporichthyaceae family, Glycomycetaceae family, Bonenbergiaceae family, Bogorkliaceae family, Brevibacteriaceae family, Gellulomonadaceae family, Dermabacteraceae family, Dermaiophilaceae family, Dermacoccaceae family, Intrasporanglaceae family, Jonesiaceae family, Microbacteriaceae family, Micrococcaceae family, Promicrontonosporaceae family, Rarobacteraceae family, Sanguibacteraceae family, Micromonosporaceae family, Nocardioidaceae family, Propionibacteriaceae family, Actinosynnernataceae family, Pseudonocardiaceae family, Streptomycetaceae family, Nocardiopsaceae family, Streptosporangiaceae family, Thermomonosporaceae family, Bifidobacteriaceae family, Coriobacteriaceae family, Rubrobacteraceae family, and Sphaerobacteroceae family.

In certain embodiments, the bacteria is a member of the phylum Actinobacteria and the Mycobacterium. In some embodiments the bacteria is a baceteria associated with an atypical mycobacterial infection. Exemplary bacteria from genus Mycobacterium include, but are not limited to: M. abscessus, M. africanum, M. avium, M. bovis, M. caprae, M. canetti, M. chelonae, M. colombiense, M. flavescens, M. fortuitum, M. genavense, M. gordonae, M. haemophilum, M. intracellulare, M. kansasii, M. leprae, M. lepramatosis, M. malmoense, M. marinum, M. microti, M. parafortuitum, M. phlei, M. pinnipedii, M. scrofulaceum, M. simiae, M. smegmatis, M. szulgai, M. terrae, M. ulcerans, M. vaccae, and M. xenope. In some embodiments, the bacteria is a bacteria that can cause tuberculosis (e.g., a member of the Mycobacterium tuberculosis complex (e.g., M. tuberculosis, M. africanum, M. bovis, M. bovis BCG, M. microti, M. canetti, M. pinnipedii, M. suricattae, M. mungi). In some embodiments, the bacteria is M. tuberculosis. In some embodiments, the bacteria is a member of the Mycobacterium avium complex (e.g., M. avium, M. avium avium, M. avium paratuberculosis, M. avium silvaticum, M. avium hominissuis, M. colombiense, M. indicus pranii, M. intracellulare). In some embodiments, the bacteria is M. phlei. In some embodiments, the bacteria is M. smegmatis. In certain embodiments, the Mycobacterium infection is a M. tuberculosis infection. In certain embodiments, the Mycobacterium infection is a multi-drug-resistant tuberculosis (MDR-TB) infection or extensively drug-resistant tuberculosis (XDR-TB) infection. In certain embodiments, the M. tuberculosis infection is a multi-drug-resistant tuberculosis (MDR-TB) infection or extensively drug-resistant tuberculosis (XDR-TB) infection.

In certain embodiments, the bacterial infection is a Mycobacterium infection, a Staphylococcus infection, Pseudomonas infection, a Bacillus infection, or an Escherichia infection. In certain, embodiments, the bacterial infection is tuberculosis. In some embodiments, the bacterial infection is a Mycobacterium tuberculosis infection. In certain embodiments, the bacterial infection is a Pseudomonas infection. In some embodiments, the bacterial infection is Pseudomonas aeruginosa infection. In some embodiments, the bacterial infection is Yersinia infection. In some embodiments the bacterial infection is Yersinia pestis infection. In some embodiments the bacterial infection is E. coli infection. In some embodiments the bacterial infection is Bacillus anthracis infection. In some embodiments the bacterial infection is Bacillus anthracis infection. In some embodiments the bacterial infection is Vibrio cholera infection. In some embodiments, the bacterial infection is infection of multiple species of bacterium. In some embodiments, the bacterial infection is infection of multiple species of bacterium, one of which is P. aeruginosa. In some embodiments, the bacterial infection is infection of multiple species of bacterium, one of which is Mycobacterium tuberculosis.

In some embodiments, the infectious disease is a parasitic infection. Exemplary parasites causing the parasitic infection include, but are not limited to, Trypanosoma spp. (e.g., Trypanosorna Trtpansosoma brucei), Leishmania spp., Giardia spp., Trichomonas spp., Entamoeba spp., Naegleria spp., Acanthamoeba spp., Schistosoma spp., Plastmlitun spp. (e.g., P. flaciparum), Ciytosporidium spp., Isospora spp., Balantidium spp., Pneumocystis spp., Babesia, Loa Loa, Ascaris lumbricoides, Dirofilaria immitis, and Toxoplasina ssp. (e.g. T. gondii).

The present disclosure also provides methods that may be useful for the treatment and/or prevention of an infectious disease including, but not limited to pneumonic plague, septicemic plague, bubonic plague. gastroenteritis, urinary tract infections, neonatal meningitis, hemorrhagic colitis, Crohn's disease, pneumonia, septic shock, gastrointestinal infection, necrotizing enterocolitis, anthrax, and tuberculosis. The present disclosure also provides compounds for use in methods that may be useful for the treatment and/or prevention of an infectious disease including, but not limited to pneumonic plague, septicemic plague, bubonic plague, gastroenteiitis, urinary tract infections, neonatal meningitis, hemorrhagic colitis, Crohn's disease, pneumonia, septic shock, gastrointestinal infection, necrotizing enterocolitis, anthrax, and tuberculosis.

The compounds described herein (e.g., compounds of Formula (I)) may exhibit inhibitory activity towards an adenylate-forming enzyme (e.g., an acyl-CoA synthetase), may exhibit the ability to inhibit MenE, may exhibit the ability to inhibit an adenylate-forming enzyme, may exhibit the ability to inhibit menaquinone biosynthesis. may exhibit the ability to inhibit an acyl-CoA synthetase, may exhibit the ability to inhibit cofactor biosynthesis, may inhibit cellular respiration in a microorganism, may prevent biofilm formation, may exhibit a therapeutic effect and/or preventative effect in the treatment of infectious diseases (e.g., bacterial infections), and/or may exhibit a therapeutic and/or preventative effect superior to existing agents for treatment of an infectious disease.

The compounds described herein (e.g., compounds of Formula (I)) may exhibit selective inhibition of an acyl-CoA synthetase versus inhibition of other proteins. The compounds described herein (e.g., compounds of Formula (I)) may exhibit selective inhibition of MenE. The compounds described herein (e.g., compounds of Formula (I)) may exhibit selective inhibition of an adenylate-forming enzyme. In certain embodiments, the selectivity versus inhibition of another protein is between about 2 fold and about 10 fold. In certain embodiments, the selectivity is between about 10 fold and about 50 fold. In certain embodiments, the selectivity is between about 50 fold and about 100 fold. In certain embodiments, the selectivity is between about 100 fold and about 500 fold. In certain embodiments, the selectivity is between about 500 fold and about 1000 fold. In certain embodiments, the selectivity is between about 1000 fold and about 5000 fold. In certain embodiments. In certain embodiments, the selectivity is between about 5000 fold and about 10000 fold. In certain embodiments, or at least about 10000 fold.

The present disclosure provides methods that may be useful for the treatment and/or prevention of an infectious disease by administering a compound described herein, or pharmaceutically acceptable salt, solvate, hydrate. polymorph, co-crystal, tautomer, stereoisomer, or prodrug thereof, or pharmaceutical composition thereof, to a subject in need thereof. The present disclosure provides compounds for use in methods that may be useful for the treatment and/or prevention of an infectious disease by administering a compound described herein, or pharmaceutically acceptable salt, solvate, hydrate, polymorph, co-crystal, tautomer, stereoisomer, or prodrug thereof, or pharmaceutical composition thereof, to a subject in need thereof. In certain embodiments, the compound is administered as a pharmaceutically acceptable salt, stereoisomer, or tautomer thereof. In certain embodiments, the compound is administered as a pharmaceutically acceptable salt of the compound. In certain embodiments, the compound is administered as a specific stereoisomer or mixture of stereoisomers of the compound. In certain embodiments, the compound is administered as a specific tautomer or mixture of tautomers of the compound. In certain embodiments, the compound is administered as a pharmaceutical composition as described herein comprising the compound.

The present disclosure also provides uses of the inventive compounds, and pharmaceutically acceptable salts, solvates, hydrates, polymorphs, co-crystals, tautomers, stereoisomers, prodrugs, and pharmaceutical compositions thereof, in the manufacture of medicaments for the treatment and prevention of diseases. In certain embodiments, the disease is an infectious disease. In certain embodiments, the infectious disease is a bacterial infection. In certain embodiments, the disease is tuberculosis. In certain embodiments, the infectious disease is a parasitic infection. In certain embodiments, the infectious disease may be associated with another disease or condition, for example, in subjects with a weakened immune system as a result of HIV infection, AIDS, lupus, cancer, cystic fibrosis, or diabetes, or subjects with hums. In certain embodiments, the infectious disease may arise as complication of another disease or condition, for example. in subjects with a weakened immune system as a result of HIV infection, AIDS, lupus, cancer, cystic fibrosis or diabetes. In certain embodiments, the bacterial infection is an infection caused by Gram-positive bacteria, In certain, embodiments, the bacterial infection is an infection caused by Gram-negative bacteria. In certain embodiments, the bacterial infection is a Staphylococcus infection, a Bacillus infection, or an Escherichia infection. In certain embodiments, the bacterial infection is a Pseudontonas infection, a Mycobacterium infection, or a Yersinia infection. In some embodiments the bacterial infection is a Pseudonumas aeruginosa infection. In some embodiments the bacterial infection is a Mycobacterium tuberculosis infection. In some embodiments the bacterial infection is a Yersinia pestis infection. In some embodiments the bacterial infection is an E. coli infection. In some embodiments the bacterial infection is a S. aureus infection. In some embodiments the bacterial infection is a Bacillus subtilis infection. In some embodiments the bacterial infection is a Bacillus anthracis infection. In some embodiments the bacterial infection is a Vibrio cholera infection.

Certain methods described herein include methods of treating a bacterial infection, methods of treating an infection in a subject, preventing a bacterial infection, methods of preventing an infection in a subject, or methods of contacting an infectious microorganism with a compound described herein (e.g. a compound of Formula (I)). Any of these methods may involve a specific class of bacteria or type of bacteria. Certain compounds for use in methods described herein include methods of treating a bacterial infection, methods of treating an infection in a subject, preventing a bacterial infection, methods of preventing an infection in a subject, or methods of contacting an infectious microorganism with a compound described herein (e.g. a compound of Formula (I)). Any of these methods or uses may involve a specific class of bacteria or type of bacteria. In certain embodiments, the bacterial infection is caused by Gram-positive bacteria. In certain embodiments, the bacterial infection caused by Gram-negative bacteria. In certain embodiments the bacteria is from the genus Yersinia Staphylococcus, Escherichia, or Bacillus. In certain embodiments the bacteria is from the genus Pseudomonas. In certain embodiments the bacteria is from the genus Mycobacterium.

In certain embodiments, the microbial infection is an infection with a bacteria, i.e., a bacterial infection. In certain embodiments, the compounds of the disclosure exhibit anti-bacterial activity. For example, in certain embodiments, the compound has a mean inhibitory concentration, with respect to a particular bacterium, of less than 50 μg/mL, preferably less than 25 μg/mL, more preferably less than 5 g/mL, and most preferably less than 1 μg/mL.

Exemplary bacteria include, but are not limited to, Gram positive bacteria (e.g., of the phylum Actinobacieria, phylum Firmicutes, or phylum Tenericutes); Gram negative bacteria (e.g., of the phylum Aquificae, phylum Deinococcus—Thermos, phylum Fibrobacteres/Chlorobi/Bacteroidetes (FCB), phylum Fusobacteria, phylum Gemmatimonadest, phylum Ntrospirae, phylum Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

In certain embodiments, the bacteria is a member of the phylum Actinobacteria and the genus Mycobacterium, e.g., the bacterial infection is a Mycobacterium infection. Exemplary Mycobacterium bacteria include, but are not limited to, Mycobacterium tuberculosis, Mycobacterium leprae, Mycobacterium avium paratuberculosis, Mycobacterium ulcerans, Mycobacterium lepromatosis, and Mycobacterium marinum. in certain embodiments, the bacteria is Mycobacterium tuberculosis.

In certain embodiments, the bacteria is a member of the phylum Proteobacteria and the genus Pseudomonas, e.g., the bacterial infection is a Psuedomonas infection. Exemplary Psuedomonas bacteria include, but are not limited to, P. aeruginosa, P. anguilliseptica, P. agarici, P. luteola. P. oryzihabitans, P. plecoglossida, P. syringae, and P. tolaasii. In certain embodiments, the bacteria is P. aeruginosa.

In certain embodiments, the bacteria is a member of the phylum Proteobacteria and the genus Yersinia. e.g., the bacterial infection is a Yersinia infection. Exemplary Yersinia bacteria include, but are not limited to, Y. pestis, Y. entercolitica, and Y. pseudotuberculosis. In certain embodiments, the Acinetobacter infection is an Y. pestis infection.

In certain embodiments, the bacteria is a member of the phylum Proteobacteria and the genus Escherichia, e.g., the bacterial infection is a Escherichia infection. Exemplary Escherichia bacteria include, but are not limited to, E. albertii, E. blattae, E. coli, F. fergusonii, E. hermannii, and E. vulneris. In certain embodiments, the Escherichia infection is a E. coli infection.

In certain embodiments, the bacteria is a member of the phylum Firmicutes and the genus Staphylococcus, e.g., the bacterial infection is a Staphylococcus infection. Exemplary Staphylococcus bacteria include, but are not limited to, S. arlettae, S. aureus, S. auricularis, S. capitis, S. caprae, S. carnous, S. chromogenes, S. cohii, S. condimenti, S. crocealyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. linus, S. lugdunesis, S. hatrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae. S. simians, S. stepanovicii, S. succinus, S. vitulinus, S. warneri, and S. xylosus. In certain embodiments, the Staphylococcus infection is a S. aureus infection.

In certain embodiments. the bacteria is a member of the phylum Firmicutes and the genus Bacillus, e.g., the bacterial infection is a Bacillus infection. Exemplary Bacillus bacteria include, but are not limited to. B. alcalophilus, B. alvei, B. aminovorans, B. amyloliquefaciens, B. aneurinolyticus. B. anthracis, B. aquaemaris, B. atrophaeu s, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. suhtilis, B. thermoglucosidasius, B. thuringiensis, B. vulgaris, and B. weihenstephanensis. In certain embodiments, the Bacillus infection is a B. subtilis infection. In some embodiments, the Bacillus infection is a B. anthracis infection.

In certain embodiments, the methods of the disclosure include administering to the subject an effective amount of a compound described herein (e.g., a compound of Formula (I)), or a pharmaceutically acceptable salt, stereoisomer, or tautomer thereof, or a pharmaceutical composition thereof. In certain embodiments, the use of compounds of the disclosure include administering to the subject an effective amount of a compound described herein (e.g., a compound of Formula (I)), or a pharmaceutically acceptable salt, stereoisomer, or tautomer thereof, or a pharmaceutical composition thereof. In certain embodiments, the effective amount is a therapeutically effective amount. In certain embodiments, the effective amount is a prophylactically effective amount.

In one aspect the present disclosure provides methods for inhibiting cofactor biosynthesis (e.g., menaquinone). In one aspect the present disclosure provides compoudns for use in methods for inhibiting cofactor biosynthesis (e.g., menaquinone).

In certain embodiments, the disclosure provides methods for inhibiting cofactor biosynthesis. In some embodiments, the disclosure provides methods for inhibiting biosynthesis of menaquinone, a cofactor. In some embodiments, the disclosure provides methods for inhibiting biosynthesis of menaquinone by inhibiting MenE. In some embodiments, the disclosure provides methods for inhibiting biosynthesis of menaquinone by inhibiting an acyl-CoA synthetase. In another aspect, the present disclosure provides methods for inhibiting menaquinone biosynthesis in an infectious microorganims causing an infection in a subject by administering to the subject a compound described herein (e.g., a compound of Formula (I)), or a pharmaceutically acceptable salt, stereoisomer, or tautomer thereof, or a pharmaceutical composition thereof. In another aspect, the present disclosure provides methods for inhibiting menaquinone biosynthesis in an infectious microorganism, by contacting the sample with a compound described herein (e.g., a compound of Formula (I)), or a pharmaceutically acceptable salt, stereoisomer, or tautomer thereof, or a pharmaceutical composition thereof.

In certain embodiments, the disclosure provides compounds for use in inhibiting cofactor biosynthesis. In some embodiments, the disclosure provides compounds for use in inhibiting biosynthesis of menaquinone, a cofactor. In some embodiments, the disclosure provides compounds for use in methods for inhibiting biosynthesis of menaquinone by inhibiting MenE. In some embodiments, the disclosure provides compounds for use in methods for inhibiting biosynthesis of menaquinone by inhibiting an acyl-CoA synthetase. In another aspect, the present disclosure provides compounds for use in methods for inhibiting menaquinone biosynthesis in an infectious microorganims causing an infection in a subject by administering to the subject a compound described herein (e.g., a compound of Formula (I)), or a pharmaceutically acceptable salt, stereoisomer, or tautomer thereof, or a pharmaceutical composition thereof. In another aspect, the present disclosure provides compounds for use in methods for inhibiting menaquinone biosynthesis in an infectious microorganism, by contacting the sample with a compound described herein (e.g., a compound of Formula (I)), or a pharmaceutically acceptable salt, stereoisomer, or tautomer thereof, or a pharmaceutical composition thereof.

In another aspect, the present disclosure provides methods for inhibiting MenE in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present disclosure provides compounds for use in methods for inhibiting MenE in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present disclosure provides methods for inhibiting MenE in an infectious microorganism, by contacting the sample with a compound described herein (e.g., a compound of.Formula (I)), or a pharmaceutically acceptable salt, stereoisomer, or tautomer thereof, or a pharmaceutical composition thereof.

In another aspect, the present disclosure provides compounds for use in methods for inhibiting MenE in an infectious microorganism, by contacting the sample with a compound described herein (e.g., a compound of Formula (I)), or a pharmaceutically acceptable salt, stereoisomer, or tautomer thereof, or a pharmaceutical composition thereof.

In another aspect, the present disclosure provides methods for inhibiting an adenylate-forming enzyme (e.g., an acyl-CoA synthetase) in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present disclosure provides compounds for use in methods for inhibiting an adenylate-forming enzyme (e.g., an acyl-CoA synthetase) in an infection in a subject by administering to the subject a compound described herein (e.g., a compound of Formula (I)), or a pharmaceutically acceptable salt, stereoisomer. or tautomer thereof, or a pharmaceutical composition thereof.

In another aspect, the present disclosure provides methods for inhibiting an adenylate-forming enzyme (e.g., an acyl-CoA synthetase) in an infectious microorganism. by contacting the sample with a compound described herein (e.g., a compound of Formula (I)), or a pharmaceutically acceptable salt, stereoisomer, or tautomer thereof, or a pharmaceutical composition thereof.

In another aspect, the present disclosure provides compounds for use in methods for inhibiting an adenylate-forming enzyme (e.g., an acyl-CoA synthetase) in an infectious microorganism, by contacting the sample with a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting a ligase and/or adenylate-forming enzyme (e.g., o-succinylbenzoate-CoA synthetase (MenE)) in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present invention provides compounds for use in methods for inhibiting a ligase and/or adenylate-forming enzyme (e.g., o-succinylbenzoate-CoA synthetase (MenE)) in an infection in a subject by administering to the subject a compound described herein (e.g., a compound of Formula (I)), or a pharmaceutically acceptable salt, stereoisomer, or tautomer thereof, or a pharmaceutical composition thereof.

In another aspect, the present invention provides methods for inhibiting a ligase and/or adenylate-forming enzyme (e.g., o-succinylbenzoate-CoA synthetase (MenE)) in an infectious microorganism, by contacting the sample with a compound described herein (e.g., a compound of Formula (I)), or a pharmaceutically acceptable salt, stereoisomer, or tautomer thereof, or a pharmaceutical composition thereof. In another aspect, the present invention provides methods for killing a microorganism, by contacting the sample with a compound described herein (e.g., a compound of Formula (I)), or a pharmaceutically acceptable salt, stereoisomer, or tautomer thereof, or a pharmaceutical composition thereof. in another aspect, the present invention provides methods for inhibiting cellular respiration in a microorganism, by contacting the sample with a compound described herein (e.g., a compound of Formula (I)), or a pharmaceutically acceptable salt, stereoisomer, or tautomer thereof, or a pharmaceutical composition thereof. In another aspect, the present invention provides methods for inhibiting bioftlm formation, by contacting the sample with a compound described herein (e.g., a compound of Formula (I)), or a pharmaceutically acceptable salt, stereoisomer, or tautomer thereof, or a pharmaceutical composition thereof.

In another aspect, the present invention provides compounds for use in methods for inhibiting a ligase and/or adenylate-forming enzyme (e.g., o-succinylbenzoate-CoA synthetase (MenE)) in an infectious microorganism, by contacting the sample with a compound described herein (e.g., a compound of Formula (I)), or a pharmaceutically acceptable salt, stereoisomer, or tautomer thereof, or a pharmaceutical composition thereof. In another aspect, the present invention provides compounds for use in methods for killing a microorganism, by contacting the sample with a compound described herein (e.g., a compound of Formula (I)), or a pharmaceutically acceptable salt, stereoisomer, or tautomer thereof, or a pharmaceutical composition thereof. In another aspect, the present invention provides compounds for use in methods for inhibiting cellular respiration in a microorganism, by contacting the sample with a compound described herein (e.g., a compound of Formula (I)), or a pharmaceutically acceptable salt, stereoisomer, or tautomer thereof, or a pharmaceutical composition thereof. In another aspect, the present invention provides compounds for use in methods for inhibiting biofilm formation, by contacting the sample with a compound described herein (e.g., a compound of Formula (I)), or a pharmaceutically acceptable salt, stereoisomer, or tautomer thereof, or a pharmaceutical composition thereof.

The present disclosure also provides methods of using a compound described herein (e.g., a compound of Formula (I)), or a pharmaceutically acceptable salt, solvate, hydrate, polymorph, co-crystal, tautomer, stereoisomer, or prodrug thereof, or pharmaceutical compositions thereof, in research studies in the field of disease pathology, biochemistry, cell biology, and other fields associated with infectious diseases. The compounds of the disclosure can be used to study the roles of biomolecules (e.g., menaquinone, MenE, an adenylate-forming enzyme, o-succinylbenzoate-CoA synthetase, a Vitamin K, chorismate, o-succinyl benzoate, o-succinyl benzoate-AMP, o-succinylbenzoate-CoA, 1,4-clihydroxy-2-napthyol-CoA)). The compounds of the disclosure can be used to study quorum sensing in a microorganism. In certain embodiments, the use of a compound of Formula (I), or composition thereof, to inhibit MenE. In certain embodiments, the method comprises use of the compound or composition thereof to inhibit an adenylate-forming enzyme.

Certain methods or uses described herein, may comprise administering one or more additional pharmaceutical agent in combination with the compounds described herein. The additional pharmaceutical agents include, but are not limited to, anti-diabetic agents, anti-proliferative agents, anti-cancer agents, anti-angiogenesis agents, anti-inflammatory agents, anti-bacterial agents, anti-viral agents, cardiovascular agents, and pain-relieving agents. In certain embodiments, the additional pharmaceutical agent is an antibiotic. In certain embodiments, the additional pharmaceutical agent is an anti-bacterial agent. In certain embodiments, the additional pharmaceutical agent is a binder or inhibitor of an AMP-producing synthetase. In certain embodiments, the additional pharmaceutical agent is a binder or inhibitor of MenE. In certain embodiments, the additional pharmaceutical agent is a binder or inhibitor of an adenylate-forming enzyme.

In certain embodiments, the additional pharmaceutical agent is selected from the group consisting of allicin, amikacin, arginine, azlocillin, aztreonam, bedaquiline, capreomycin, carbenicilin, cefepime, cefoperazone, cefpirome, ceftaroline, ceftazidimine, ceilobiprole, ciprofloxacin, clarithromycin, clavulanic acid, clindamycin, clofazimine, co-amoxiclav, colistin, co-trimioxazole, cycloserine. dalfopristin. dapsone, daptomycin, delafloxacin, delamanid, doripenem, doxycycline, enviomycin, ethambutol, ethionamide, fosamil, gatifloxacin, gentamicin, imipenem, interferon-y, isoniazid, JNJ-Q2, kanamycin, levofloxacin, linezolid, meropenem, metronidazole, mezlocillin, minocycline, moxifloxacin, pretomanid, p-aminosalicyclic acid, perchlorperazine, piperacillin, polymyxin B, pretomanid, prothioamide, pyrazinamide, quinipristin, rifabutin, rifampicin, rifamycins, rifapentine, rifaximin, streptomycin, teicoplanin, terizidone, thiorizetazeone, thioridazine, ticarcillin, tigecycline, tobramycin, vancomycin, viomycin, and vitamin D.

In some embodiments, the additional pharmaceutical agent is isoniazid. In certain embodiments, the additional pharmaceutical agent is rifampicin (also called rifampin). In certain embodiments, the additional pharmaceutical agent is pyrazinamide. in certain embodiments, the additional pharmaceutical agent is ethambutol. In certain embodiments, the additional pharmaceutical agent is streptomycin. In certain embodiments, the additional pharmaceutical agent is a carbapenem. In some embodiments, the additional pharmaceutical agent is doripenem, imipenem, or meropenem. In certain embodiments, the additional pharmaceutical agent is a glycylcycline. In some embodiments, the additional pharmaceutical agent is tigecycline. In certain embodiments, the additional pharmaceutical agent is a aminoglycoside. In some embodiments, the additional pharmaceutical agent is gentamycin, amikacin, or tobramycin. In certain embodiments, the additional pharmaceutical agent is a quinolone. In some embodiments, the additional pharmaceutical agent is ciprofloxacin or levofloxacin. In certain embodiments, the additional pharmaceutical agent is a cephalosporin. In some embodiments, the additional pharmaceutical agent is ceftazidime, cefepime, cefoperazone, cefpirome, ceflobirprole, or ceftaroline fosamil. In certain embodiments, the additional pharmaceutical agent is a penicillin. In some embodiments. the additional pharmaceutical agent is an antipseudomonal penicillin or extended spectrum penicillin. In certain embodiments, the additional pharmaceutical agent is a carboxypenicillin or a ureidopenicillin. In some embodiments, the additional pharmaceutical agent is carbenicillin, ticarcillin, mezlocillin, aziocillin, piperacillin, or mecillinam. In certain embodiments, the additional pharmaceutical agent is a polymyxin. In some embodiments, the additional pharmaceutical agent is polymyxin B or colistin. In certain embodiments, the additional pharmaceutical agent is a monobactam. In some embodiments, the additional pharmaceutical agent is aztreonam. In certain embodiments, the additional pharmaceutical agent is a β-lactamase inhibitor. In some embodiments, the additional pharmaceutical agent is sulbactam.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al,, Enamiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N.Y., 1962); and Wilen, S. H. Tables of Resolving Agents and Optical Resolutions p. 268 (E. L. Eliel, Ed., Univ. of Notre Dame Press, Notre Dame, Ind. 1972). The disclosure additionally encompasses compounds as individual isomers substantially free of other isomers, and alternatively, as mixtures of various isomers.

In a formula, [symbol] is a single bond where the stereochemistry of the moieties immediately attached thereto is not specified, . . . is absent or a single bond, [symbol] or [symbol] is a single or double bond, and [symbol] is a single, double, or triple bond. If drawn in a ring, [symbol] indicates that each bond of the ring is a single or double bond, valency permitting. The precise of arrangement of single and double bonds will be determined by the number, type, and substitution of atoms in the ring, and if the ring is multicyclic or polycyclic. In general, any ring atom (e.g., C or N), can have a double bond with a maximum of one adjacent atom.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments. an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl. tert-butyl, sec-butyl, iso-butyl), peaty' (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-beptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted rt-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CH₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CF₃, —CF₂CF₃, —CF₂CF₂CF₃, —CCl₂, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl gaup is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having, 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”).

In some embodiments, an alkenyl group has 2 to 6 carbon atoms (“C₂₋₆ alkenyl”). In some embodiments, an alkenyl croup has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

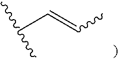

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a hetemalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl gaup has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms. at least one triple bond, and I or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms. at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cycloheptenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₄₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or Spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments. the 5-6 membered heterocyclyl has 1 ring heteroatom selected from nitrogen, oxygen, and sulfur.

Exemplary 3-membered heterocyclyl groups containing, 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyt, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered hetemcyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromertyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridirtyl, 4,5,6,7-tetrahydrofurop,2-cjpyridinyl, 4,5,6.7-tetrahydrothieno[3,2-b]pyridinyl, 1,2.3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n-i-2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitroeen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl. 10333) Exemplary 5-membered heteroaryl groups containing I heteroatom include, without limitation, pyrrolyl, (uranyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl. oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzismazolyl, benzoxadiazolyl, benzthiazolyl, benzisothiazolyl, benzthiadiazolyl, indolizinyl, and purinyl, Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, allcynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted'” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any one of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(—NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)R^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂. —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻. —P(OR^{cc})₃⁺X⁻, —P(R^{cc})₄, —P(OR^{cc})₄, —OP(R^{cc})₂, —OP(R^{cc})₃′X⁻, —OP(OR^{cc})₂, —OP(OR^{cc})₃′X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl. heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}—(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R¹¹ is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl. heteroC₂₋₁₀ alkynyl, C₃₋₁₀ carbocyclyl, 3-14 membered heterocyclyl, C₆₋₁₄ aryl, and 5-14 membered heteroaryl, or two R¹¹ groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} tooups;

each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃′X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{cc})₂, —OP(═O)(OR^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl. 3-10 membered heterocyclyl. and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0. 1, 2, 3, 4. or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl):, —N(C₁₋₆ alky))₂, —N(C₁₋₆ alkyl)₃⁺X⁻, —NH(C₁₋₆ alkyl)₂′X⁻, —NH₂(C₁₋₆ alkyl)⁺X⁻, —NH₃′X⁻, —N(OC₁₋₆ alkyl)(C₁₋₆ alkyl), —N(OH)C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl), —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl), —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alky))₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alky))₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alky))₂, —OP(═O)(C₁₋₆ alkyl)₂, —OP(═O)(OC₃₋₆ alkyl)₂, C₁₋₆ alkyl, C₁₋₆ perhaloalkyl, C₂₋₆ alkenyl, C₂₋₆ alkynyl, heteroCi-olkyl, heteroC₂₋₆alkenyl, heteroC₂₋₆alkynyl, C₃₋₁₀) carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (broino, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃′X⁻, —OP(OR^{ee})₂, —OP(OR^{ee})₃⁺X⁻, —OP(═O)(R^{aa})₂, —OP(═O)(OR^{ee})₂, and —OP(═O)(N(R^{bb}))₂, wherein X , R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb}, and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “disubstituted amino” refers to an amino group wherein the nitrogen atom directly attached to the parent molecule is substituted with three groups, and includes turoups selected from —N(R^{bb})₃ and —N(R^{bb})₃′X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-acylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any one of the substituents described herein, that result in the formation of a stable moiety (e.g., aliphatic, alkyl, alkenyl, alkynyl, heteroaliphatic, heterocyclic, aryl. heteroaryl, acyl. oxo, imino, thiooxo, cyano, isocyano, amino, azido, nitro, hydroxyl, thiol, halo, aliphaticamino, heteroaliphaticamino, alkylamino, heteroalkylamino, arytamino, heteroarylamino, alkylaryl, arylalkyl, aliphaticoxy, heteroaliphaticoxy, alkyloxy, heteroalkyloxy, acyloxy, heteroaryloxy, aliphaticthioxy, heteroaliphaticthioxy, alkylthioxy, heteroalkylthioxy, arylthioxy, heteroarylthioxy, acyloxy, and the like, each of which may or may nor be further substituted).

The term “carbonyl” refers a group wherein the carbon directly attached to the parent molecule is sp² hybridized, and is substituted with an oxygen, nitrogen, or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂. C₁₋₁₀ alkyl, C₁₋₁₀ perhaloalkyl, C₂₋₁₀ alkenyl, C₂₋₁₀ alkynyl, heteroCmoalkyl, heteroC₂₋₁₀alkenyl, heteroC₂₋₁₀alkynyl, C₃₋₁₀ carbocyclyl, 3-14 membered heterocyclyl, C₆₋₁₄ aryl. and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., C(═O)R^{aa}) include, but are not limited to, fonnamide, acetamide, chloroacetamide, trichloroacetamide, trifluoroacetamide, phenylacetamide, 3-phenylpropanamide, picolinamide, 3-pyridylcarboxainide, N-benzoylphenylalanyl derivative. benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate. ethyl carbamante, 9-fluorenylmethyl carbamate (Fmoc). 9-(2-sulfo)fluorenylmethyl carbatnate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Trot), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2.2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, i-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate. benzyl carbamate (Cbz), p-methoxybenzyl carbamate (Moz), p-nitobenzyl carbamate, p-bromobenzyl carbantate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Ppoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, piphenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methyIbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmetltyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaniinoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentarte adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyyidone, N-methylamine, N-allylamine, N-[2-(trimethylsilyl)ethoxy]methylamine (SEM), N-3-acetoxypropylarnine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-1(2-pyridyl)mesityllmethyleneamine, N-(N′,N′-dimethylaminomethylene)amine, N,N′-isopropylidenediamine, N-p-nitrobenzylideneamine, N-salicylideneamine, N-5-chlorosaticylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelateV-nitroamine, N-nitrosoamine, amine N-oxide, diphenylphosphinatnide (Dpp), dimethytthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenyltnethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}—, N(R^{bb}))₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), i-butylthiornethyl, (phenyldimethylsilyl)methoxyrnethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilypetboxymethyl (SEMOR), tetrahydropyranyl (THP), 3-bromotetrahydropyranyl, tetrahydrothiopyranyl, 1-inethoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl(CTMP), 1,4-dioxan-2-yl, tetrahydropyranyl, tetrahydrothiofuranyl, 2,3,3a,4,5,6,7,7a-octahydro-7,8,8-trimethyl-4.7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fiuoroethyl, 2,2,2-trichloroethyl, 2-trimethylsilylethyl, 2-(phenylselenyl)ethyl, butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlarophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-ditnetlioxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyt (TMS), triethylsilyl (TES), triisopropylsilyl (TIPS), dimethylisopropylsilyl (IPDMS), diethylisopropylsilyl (DEIPS), dimethylthexylsilyl, i-butyldimethylsilyl (TBDMS), t-butyldiphenylsityl (TBDPS), tribenzylsilyl, tri-p-xylylsilyl, triphenylsilyl, diphenylmethylsilyl (DPMS), i-butylmethoxyphenylsilyl (TBMPS), formate, benzoylformate, acetate, chloroacetate, dichloroacetate, trichloroacetate, tritluoroacetate, methoxyacetate, triphenylmethoxyacetate, phenoxyacetate, p-chlorophenoxyacetate 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, 1-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chiorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F , Cl , Br , I), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerite, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂ or (HCB₁₁Me₅Br₆)⁻). Exemplary counterions which may be multivalent include CO₃²⁻, HPO₄²⁻, PO₄³⁻, B₄O₇²⁻, SO₄^{2—}, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, Figures, and Claims. The invention is not intended to be limited in any manner by the above exemplary listing of substituents.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art, For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, non-toxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate. hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate. and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound, and x is a number greater than 0. A given compound may form more than one type of hydrate. including, e.g., monohydrates (x is 1), lower hydrates (x is a number greater than 0 and smaller than I, e.g., hemihydrates (R.0.5 H₂O)), and polyhydrates (x is a number greater than 1, e.g., dihydrates (R.2 H₂O) and hexahydrates (R.6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisorners that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure composed of at least two components. In certain embodiments, a co-crystal contains a compound of the present disclosure and one or more other component, including but not limited to, atoms, ions, molecules, or solvent molecules. In certain embodiments, a co-crystal contains a compound of the present disclosure and one or more solvent molecules. In certain embodiments, a co-crystal contains a compound of the present disclosure and one or more acid or base. In certain embodiments, a co-crystal contains a compound of the present disclosure and one or more components related to said compound, including not limited to. an isomer, tautomer, salt, solvate, hydrate, synthetic precursor, synthetic derivative, fragment or impurity of said compound.

The term “prodrugs” refers to compounds that have cleavable groups and become by solvolysis or under physiological conditions the compounds described herein, which are pharmaceutically active in vim. Such examples include, but arc not limited to, choline ester derivatives and the like, N-alkylmorpholine esters and the like. Other derivatives of the compounds described herein have activity in both their acid and acid derivative forms. but in the acid sensitive form often offer advantages of solubility, tissue compatibility, or delayed release in the mammalian organism (see, Bundgard, H., Design of Prodrugs. pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal “Disease,” “disorder,” and “condition” are used interchangeably herein.

The term “biological sample” refers to any sample including tissue samples (such as tissue sections and needle biopsies of a tissue): cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g.. obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “condition.” “disease,” and “disorder” are used interchangeably.

As used herein, and unless otherwise specified, the tenns “treat,” “treating” and “treatment” contemplate an action that occurs while a subject is suffering from the specified disease or condition, which reduces the severity of the disease or condition, or retards or slows the progression of the disease or condition (i.e., “therapeutic treatment”), and also contemplates an action that occurs before a subject begins to suffer from the specified disease or condition (i.e., “prophylactic treatment”).

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

As used herein the term “inhibit” or “inhibition” in the context of enzymes, for example, in the context of MenE, refers to a reduction in the activity of the enzyme. In some embodiments, the term refers to a reduction of the level of enzyme activity, e.g., MenE activity, to a level that is statistically significantly lower than an initial level, which may, for example, be a baseline level of enzyme activity. In some embodiments, the term refers to a reduction of the level of enzyme activity, e.g., MenE activity, to a level that is less than 75%, less than 50%, less than 40%, less than 30%, less than 25%, less than 20%, less than 10%, less than 9%, less than 8%. less than 7%, less than 6%, less than 5%. less than 4%, less than 3%, less than 2%, less than 1%, less than 0.5%, less than 0.1%, less than 0.01%, less than 0.001%, or less than 0.0001% of an initial level. which may, for example, be a baseline level of enzyme activity.

As used herein the term “infectious microorganism” refers to a species of infectious fungi, bacteria, or protista, or to a virus. In certain embodiments, the infectious microorganism is a fungi. In certain embodiments, the infectious microorganism is a bacteria. In certain embodiments, the infectious microorganism is a protista. In certain embodiments, the infectious microorganism is a virus.

An “infection” or “infectious disease” refers to an infection with a microorganism, such as a fungus, bacteria, or virus. In certain embodiments, the infection is an infection with a fungus, i.e., a fungal infection. In certain embodiments, the infection is an infection with a virus, i.e., a viral infection. In certain embodiments, the infection is an infection with bacteria. i.e., a bacterial infection. Various infections include, but are not limited to, skin infections, GI infections, urinary tract infections, _{B}enito-urinary infections, sepsis, pneumonia, lung infections, upper respiratory infections, lower respiratory infections, blood infections, and systemic infections. In some embodiments, the infectious disease is tuberculosis.

As used herein, the term “cofactor” refers to a non-protein chemical compound or metallic ion that is required for an enzyme's activity. Cofactors assist in biochemical transformations. Cofactors can be subclassified as either inorganic ions or complex organic molecules called coenzymes, the latter of which is mostly derived from vitamins and other organic essential nutrients in small amounts. Some enzymes or enzyme complexes require several cofactors. In some embodiments, the cofactor is menaquinone.

As used herein, the term “o-succinylbenzoate-CoA synthetase” or “MenE” refers to an enzyme of the menaquinone biosynthesis pathway. MenE may also refer to the encoding RNA and DNA sequences of the MenE protein. In some embodiments, a MenE inhibitor provided herein is specific for a MenE from a species. The term MenE further includes, in sonic embodiments, sequence variants and mutations (e.g., naturally occurring or synthetic MenE sequence variants or mutations), and different MenE isoforms. In some embodiments, the term MenE includes protein or encoding sequences that are homologous to a MenE protein or encoding sequence, for example, a protein or encoding sequence having at least 80%, at least 85%, at least 90%, at least 91%, at least 92%, at least 93%, at least 94%, at least 95%, at east 96%, at least 97%, at least 98%, at least 99%, or at least 99.5% sequence identity with a MenE sequence. MenE protein and encoding gene sequences are well known to those of skill in the art.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### General Materials and Methods

Reagents were obtained from Aldrich Chemical or Acros Organics and used without further purification. Optima or HPLC grade solvents were obtained from Fisher Scientific, degassed with argon (Ar), and purified on a solvent drying system. Reactions were performed in flame-dried glassware under positive Ar pressure with magnetic stirring.

TLC was performed on 0.25 mm E. Merck silica gel 60 F254 plates and visualized under UV light (254 um) or by staining with potassium permanganate (KMnO₄), cerium ammonium molybdenate (CAM), or iodine (I₂). Silica flash chromatography was performed on E. Merck 230-400 mesh silica gel 60. Preparative scale HPLC purification was carried out on a Waters 2545 HPLC with 2996 diode array detector using a Sunfire Prep C18 reverse phase column (10 Å˜150 mm, 5 μm) with UV detection at 254 nm. Samples were lyophilized using a Labconco Freezone 2.5 instrument.

IR spectra were recorded on a Bruker Optics Tensor 27 FTIR spectrometer with Pike technologies MIRacle ATR (attenuated total reflectance, ZnSe crystal) accessory and peaks reported in cm⁻¹. NMR spectra were recorded on a Bruker Avance III 500 instrument or Bruker Avance III 600 instrument at 24° C. in CDCl₃ unless otherwise indicated. Spectra were processed using Bruker TopSpin or nucleomatica iNMR (www.inmr.net) software, and chemical shifts are expressed in ppm relative to TMS (¹H, 0 ppm) or residual solvent signals: CDCl₃ (¹H, 7.24 ppm; ¹³C, 77.23 ppm), CD₃OD (¹H, 3.31 ppm; ¹³C. 49.15 ppm), D₂O (¹H, 4.80 ppm); coupling constants are expressed in Hz. Mass spectra were obtained at the MSKCC Analytical Core Facility on a Waters Acuity SQD If-MS by electrospray (ESI) ionization or atmospheric pressure chemical ionization (AP-CI).

### Example 1

### Design and Compinalional Docking of OSB-AMS Linker Analogues

The OSB-AMSIvienE co-crystal structure (PDB: 5C5H) was processed using the Protein Preparation Wizard in the Schrodinger suite (v2017.2). Bond orders were assigned, hydrogen's added, and waters beyond 5 Å were deleted. The protonation and tautomeric states of the protein-ligand complex were generated using EPIK at pH 7.4. Hydrogen bond assignment and optimization was performed with PROPKA to sample hydrogen bonding and orientation of water molecules. Non-bridging waters (<2 hydrogen bonds) were removed. Geometric refinement was performed using OPLS_3 force field restrained minimization to a heavy atom convergence of 0.3 Å.

Ligand preparation was performed using Ligprep in the Schrddinger suite (v2017.2). Lowest energy conformers were obtained using OPLS_3 force field optimization. Ionization and tautomeric states were generated using EPIK at pH 7.4.

Using the SchrOdinger suite (v2017.2) receptor grid generator, the receptor-binding site was defined as the area around the co-crystalized ligand with a cube grid of 10 Å side length. Nonpolar parts of the receptor were softened using Van der Waals radius scaling (factor 1.0 with partial cutoff of 0.25). No constraints were defined and rotations allowed for all hydroxyl groups in the defined binding pocket.

Using Glide (v7.2), ligands were docked to MenE using Glide XP docking precision. Flexible ligand sampling was used and EPIK state penalties applied to docking scores. Post-docking minimization was performed for all poses.

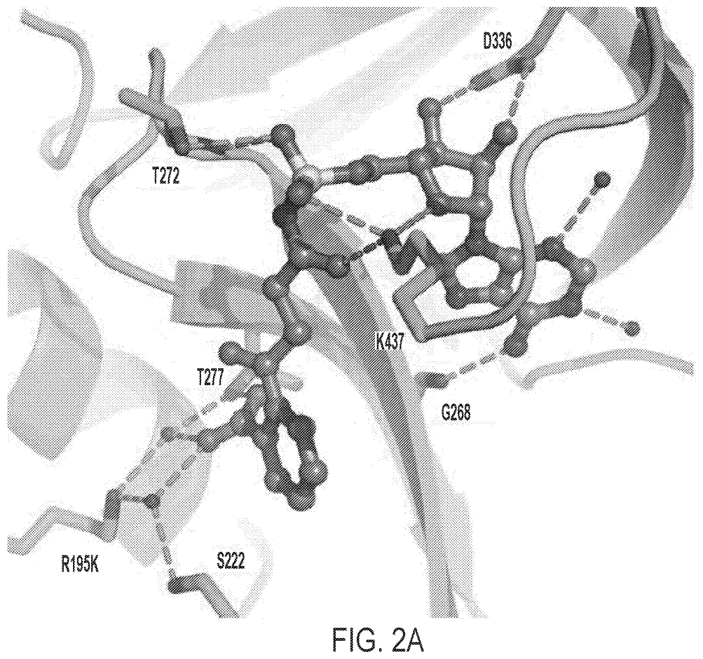

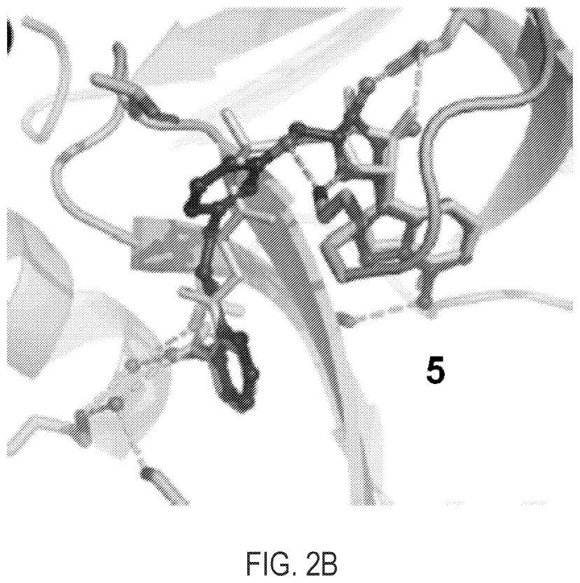

A virtual library of OSB-AMS analogues in which the acyl-sulfamate motif was replaced by a variety of other potential linkers was compiled. As this is the region of the molecule directly relevant to MenE catalytic activity, there are many crucial interactions between the inhibitor and the enzyme, making substitution at this position difficult. Thus, the virtual library focused on decreasing polarity while attempting to retain as many of these key interactions as possible, as well as the geometry and distance between OSB and adenosine observed in the cocrystal structure of OSB-AMS with E. coli MenE (R195K mutant) (Matarlo, J. S. et al. Biochemistry 2015, 54, 6514) (FIG. 2a).

The library members were docked with the E. coli MenE (R195K) crystal structure (SchrOdinger Glide) (Table 1) (Bisseret, P. et al. Tetrahedron Lett. 2007, 48. 6080). Briefly. the cocrystal structure (PDB entry: 5C₅H) (Matadi), J. S. et al. Biochemistry 2015, 54, 6514) was minimized (Protein Preparation Wizard), then OSB-AMS was emoved. Analogues were energy minimized (Ligprep) and probable tautomeric and protonation states predicted (EPIK), then docked into the binding pocket using a soft receptor model (Glide XP). Analogues were ranked by docking score, and those with scores above −10 kcal/mol removed from further consideration. Synthetic targets 5 and 8 were then selected based on combined considerations of docking score, reasonable docking pose that retained key interactions in the binding pocket (FIG. 2B), likelihood of improving overall physiochemical properties of the scaffold (e.g.. elimination of negative charge), synthetic accessibility, and ease of functional ization for further optimization. Analogue 6, which had a poor docking score, was selected for synthesis as a negative control.

TABLE 1
Computational docking scores for OSB-AMS (1), selected virtual library
members (5 and 6) and additional analogue (8).

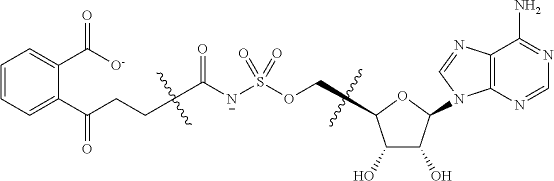

| Analogue | Linker | IC₅₀ (μM) | Docking Score (kcal/mol) |
|---|---|---|---|
| 1 | 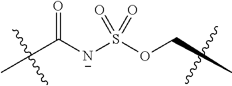 | 0.024 ± 0.003 | −13.78 |
| 5 | 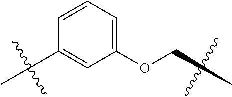 | 8.1 ± 0.9b | −14.03 |
| 6 | 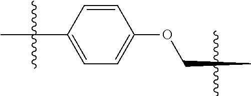 | >200d | >−10.00 |
| 8 | 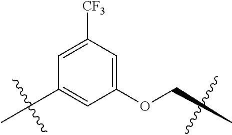 | 26.5 ± 2.3 | −12.80 |

### Example 2

### Synthesis of Selected OSB-AMS Linker Analogues

OSB-AMS (1) was synthesized according to literature procedures. Analogues 5, 6, and 8 were synthesized by following the general scheme below.

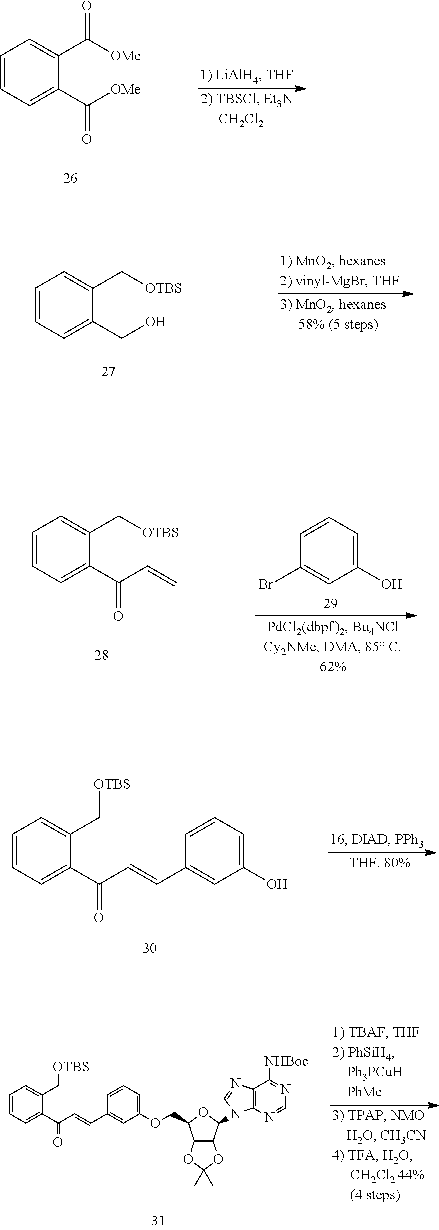 [SMILES: COC(=O)c1ccccc1C(=O)OC ; CC(C)(C)[Si](C)(C)OCc1ccccc1CO ; C=CC(=O)c1ccccc1CO[Si](C)(C)C(C)(C)C ; Oc1cccc(Br)c1 ; CC(C)(C)[Si](C)(C)OCc1ccccc1C(=O)/C=C/c1cccc(O)c1 ; CC(C)(C)OC(=O)Nc1ncnc2c1ncn2[C@@H]1O[C@H](COc2cccc(/C=C/C(=O)c3ccccc3CO[Si](C)(C)C(C)(C)C)c2)C2OC(C)(C)OC21]

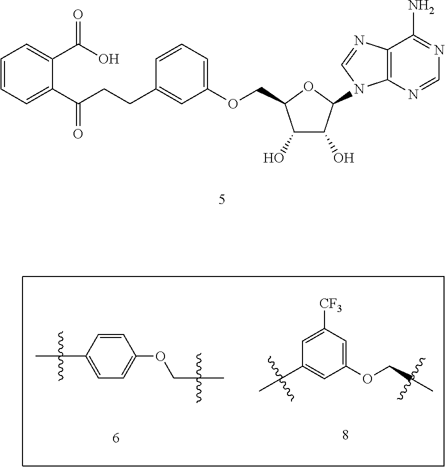

### Example 2A

### Synthesis of m-phenolic linker analogue (5)

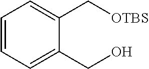 [SMILES: CC(C)(C)[Si](C)(C)OCc1ccccc1CO]

(2-((certButyldimethylsilyloxy)methyl)phenyl)methanol (50). Dimethyl phthalate (20 g, 102.9 mmol, 1 equiv.) in THF (50 mL) was added dropwise to a stirring solution of lithium aluminum hydride (4.880 g, 128.6 mmol, 1.25 equiv.) in ether (200 mL) at 0° C. before being allowed to return to room temperature. After 36 h the reaction was cooled to 0° C. before water (5 mL), aqueous NaOH (5 mL, 3.75 M), and water (15 mL) were added sequentially and the reaction stirred for 15 min. MgSO₄ (5 g) was then added and the reaction stirred for 15 min while returning to room temperature before being filtered through a pad of celite and the solvent removed by rotary evaporation. The crude diol (11.3 g, 81.78 mmol, 1 equiv.) and TBSCI (12.94 g, 85.86 mmol, 1.05 equiv) were dissolved in dichloromethane (150 mL) and cooled to 0° C. before triethylamine (45.64 mL, 327.1 mmol, 4 equiv) was added and the reaction returned to room temperature. After 14 h the reaction was quenched with 150 mL saturated ammonium chloride, the organic layer removed, the aqueous layer extracted with dichloromethane (3×150 mL), organics combined, dried (Na₂SO₄), filtered, and concentrated by rotary evaporation to give the crude alcohol 50 (17.6 g) which was used without further purification.

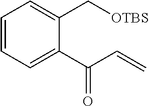 [SMILES: C=CC(=O)c1ccccc1CO[Si](C)(C)C(C)(C)C]

1-(2-((tort-Butyldimethylsilyloxy)methyl)phenyl)prop-2-en-1-one (52). Crude alcohol 50 (17.6 g, 69.72 mmol, 1 equiv) was suspended in hexanes (500 mL) before MnO₂ (90 g, 1.045 mol, 15 equiv.) was added and the reaction stirred for 14 h. The reaction was then filtered through a pad of celite and the solvent removed by rotary evaporation. The crude aldehyde (15.3 g, 61.10 nunol. 1 equiv.) was dissolved in THF (60 mL) and cooled to 0° C., before vinylmagnesium bromide (91.65 mL, 91.65 mmol, 1 M in THF, 1.5 equiv) was added dropwise over 30 min. After 1 h, the reaction was quenched with saturated ammonium chloride (200 mL), extracted with EtOAc (3×200 mL), organics combined, dried (Na₂SO₄), filtered, and concentrated by rotary evaporation. The crude alcohol was suspended in hexanes (500 mL) before MnO₂ (53 g, 610.4 mmol, 10 equiv.) was added and the reaction stirred for 8 h. The reaction was then filtered through a pad of celite and the solvent removed by rotary evaporation. Purification by silica flash chromatography (0%→20% EtOAc in hexanes) yielded the product (52) as a clear oil (16.2 g, 58% over 5 steps).

IR (ATR): 2957, 2932, 2888, 2859, 1674, 1609, 1575, 1474, 1404, 1364, 1298, 1258, 1230. 197, 1130, 1081, 994, 966, 840, 817, 779. 755, 671. ¹H-NMR (600 MHz; CDCl₃): δ 7.74 (dd, J=7.8, 0.7 Hz, 1H), 7.56 (dd, J=7.7, 1.2 Hz, IH), 7.51 (td, J=7.6, 1.3 Hz, 1H), 7.33-7.31 (m, 1H), 6.87 (dd. J=17.4, 10.6 Hz, 1H), 6.20 (dd, J=17.4, 1.4 Hz, 1H), 5.96 (dd, J=10.6, 1.4 Hz, 1H), 4.95 (s, 2H), 0.94 (s, 9H), 0.10 (s, 6H). ¹³C-NMR (151 MHz; CDCl₃): δ 195.2, 142.4, 135.7, 135.2, 131.5, 130.8, 128.8, 127.1, 126.3, 63.0, 26.0, 18.4, −5,4. HRMS (ESI) m/z calccl for C₁₆H₂₅O₂Si ([M+H]⁺) 277.1624; found 277.1631.

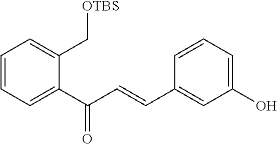 [SMILES: CC(C)(C)[Si](C)(C)OCc1ccccc1C(=O)/C=C/c1cccc(O)c1]

(E)-1-(2-((tert-Butyldimethylsilyloxy)methyl)phenyl)-3-(3-hydroxyphenyl) prop-2-en-1-one (54). Vinyl ketone 52 (300 mg, 1.085 mmol, 1.2 equiv.), 3-bromophenol (53) (156 mg, 0.904 mmol, 1 equiv.), NiBu₄Cl (25 mg, 0.0904 mmol, 0.1 equiv.), and PdCl₂(dtbpf) (59 mg, 0.0904 mmol. 0.1 equiv) were suspended in DMA (2.7mL) before NCy₂Me (265 mg, 1.356 mmol, 1.5 equiv.) was added and the reaction stirred vigorously at 85° C. in a sealed tube for 16 h. The reaction was cooled to room temperature before being diluted with 8 mL water and extracted with Et₂O (4×8 mL), organics combined, dried (Na₂SO₄), filtered and concentrated by rotary evaporation. Purification by silica flash chromatography (10% 30% EtOAc in hexanes) yielded the product (54) as a white solid (274 mg, 82%).

IR (ATR): 3354, 2955, 2929, 2892, 2857, 1625, 1600, 1472, 1452, 1361, 1257, 1160, 112, 1084, 1022, 997, 983, 839, 816, 778, 740, 677, 610. ¹H-NMR (600 MHz; CDCl₃): δ 7.75-7.74 (m, 1H), 7.60 (dd, J=7.6, 1.1 Hz, 1H), 7.53 (td, J=7.6, 1.2 Hz, 1H), 7.46 (d, J=16.0 Hz, 1H), 7.35 (t, J=7.3 Hz, 1H), 7.28-7.25 (m, 1H), 7.18 (d, J=16.0 Hz, 1H), 7.13 (d, J=7.7 Hz, 1H), 7.05 (t, J=2.0 Hz, 1H), 6.89 (ddd, J=8.1, 2.5, 0.7 Hz, 1H), 5.41 (s, 1H), 4.96 (s, 2H), 0.92 (s, 9H), 0.09 (s, 6H). ¹³C-NMR (151 MHz; CDCl₃): δ 195.4, 156.1, 145.6. 142.0, 136.30, 136.20, 131.3, 130.2, 128.4, 127.2, 126.4, 126.2, 121.3. 117.9, 114.7, 63.0, 26.0, 18.4, −5.3. HMIS (ESI) m/z calcd for C₂₂H₂₇O₃Si ([M+H]⁺) 367.1729; found 367.1743.

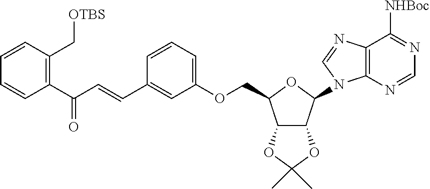 [SMILES: CC(C)(C)OC(=O)Nc1ncnc2c1ncn2[C@@H]1O[C@H](COc2cccc(/C=C/C(=O)c3ccccc3CO[Si](C)(C)C(C)(C)C)c2)[C@H]2OC(C)(C)O[C@H]21]

6-N-t-Butoxycarbonyl-2′,3-O-isopropylidene-5′-O-([E]-1-[2-([tert-butyldimethylsilyloxy]methyl)phenyl]-3-(3-hydroxyphenyl) prop-2-en-1-one)adenosine (55). DIAD (103 mg, 0.509 mmol, 1.5 equiv.) was added dropwise to a stirring solution of phenol 54 (125 mg, 0.339 mmol, 1 equiv.), protected adenosine 42 (138 mg, 0.339 mmol. 1 equiv.) (prepared as described in Lu, X.; Zhang, H.; Tonge, P. J.; Tan, D. S. Bioorg, Med. Chem. Lett. 2008, 18(22), 5963-5966. the contents of which are incorporated herein by reference in their entirety), and resin bound PPh₃ (417 mg, 0.509 mmol, 32% by weight, 1.5 equiv.) in THF (4 mL) at 0° C. before being allowed to return to room temperature. After 14 h, the reaction was quenched with water (0.2 mL) and filtered through a pad of celite, the pad washed with EtOAc, and solvent removed by rotary evaporation. Purification by silica flash chromatography (40%→60% EtOAc in hexanes) yielded the product (55) as a white solid (205 mg, 80%).

IR (AIR): 2932, 2858, 1752, 1700, 1620, 1586, 1528, 1464, 1370, 1326, 1303, 1232, 1213, 1146, 1083, 1012, 945, 911, 840, 776, 734, 670, 646. ¹H-NMR (600 MHz; CDCl₃): δ 8.78 (s, 1H), 8.09 (s, 1H), 7.92 (s, 1H), 7.75 (d, J=7.8 Hz, 1H), 7.61 (dd, J=7.6, 1.1 Hz, 1H), 7.53 (td, J=7.6, 1.2 Hz, 1H), 7.46 (t, J=13.3 Hz, 1H), 7.36 (td, J=7.5, 0.6 Hz, 1H), 7.27 (t, J=4.0 Hz, 2H), 7.19-7.15 (m, 2H), 7.01 (t, J=1.8 Hz, 1H), 6.79 (dd, J=8.1, 2.4 Hz, 1H), 6.26 (d, J=2.3 Hz, 1H), 5.48 (dd, J=6.2, 2.3 Hz, 1H), 5.18 (dd, J=6.2, 3.0 Hz, 1H), 4.96 (s, 2H), 4.75-4.66 (m, 2H), 4.29 (dd, J=10.2, 4.0 Hz, 1H), 4.18 (dd, J=10.1, 4.9 Hz, 1H), 1.67 (s, 3H), 1.55 (s, 9H), 1.49 (s, 3H), 1.43 (s, 3H), 0.93 (s, 9H), 0.09 (s, 6H). ¹³C-NMR (151 MHz; CDCl₃): δ 194.8, 158.4, 153.2, 150.4, 150.0, 149.5, 145.0, 142.1, 141.2, 136.3, 131.3, 130.2, 128.4, 127.2, 126.51, 126.40, 122.3, 122.0, 116.7, 114.7, 113.8, 113.3, 91.6, 85.5, 84.7, 82.4, 81.8, 68.1, 63.0, 28.21, 28.16, 27.3, 26.0, 25.4. 18.4, −5.3. HRMS (ESI) calcd for C₄₀H₅₂N₅O₈Si ([M+H]⁺) 758.3585; found 758.3561.

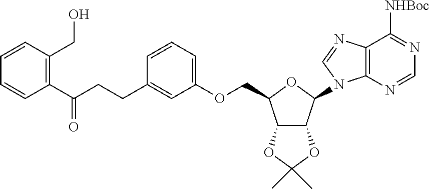 [SMILES: CC(C)(C)OC(=O)Nc1ncnc2c1ncn2[C@@H]1O[C@H](COc2cccc(CCC(=O)c3ccccc3CO)c2)[C@H]2OC(C)(C)O[C@H]21]

6-N-t-Butoxycarbonyl-2′,3′-O-isopropylidene-5′-O-(1-[2-(hydroxymethyl) phenyl]-3-(3-hydroxyphenyl) prop-2-en-1-one)adenosine (56). Phenylsilane (28.5 mg, 263.8 μmol, 2 equiv.) was added to a stirring solution of intermediate 55 (100 mg, 131.9 μmol, 1 equiv.) and Strykers catalyst (23 mg, 11.9 μmol, 0.09 equiv.) in toluene (2 mL). After 16 h, the reaction was quenched with saturated ammonium chloride (5 mL) and stirred for 5 min before 10% ammonium hydroxide (5 mL) was added and the reaction stirred for an additional 5 min. The reaction was then extracted with Et₂O (4×10 mL), organics combined, dried (Na₂SO₄), filtered, concentrated by rotary evaporation, and dried under high vacuum for 1 h. The reside was reconstituted in THF (3 mL) and cooled to 0° C. before TBAF (264 μL, 264 μmol, 1M in THF, 2 equiv.) was added and the reaction stirred for 1 h. CaCO₃ (132 mg, 1.315 mmol, 10 equiv.) and MeOH (3 mL) was added and the reaction stirred for 15 min before sulfonic acid resin (Dowex 50WX8, 200 mg) was added and the reaction stirred for an additional 10 min. The reaction was then filtered through a pad of celite and concentrated by rotary evaporation to give the crude product 56 (86 mg, 101% yield).

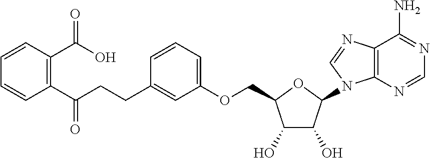 [SMILES: Nc1ncnc2c1ncn2[C@@H]1O[C@H](COc2cccc(CCC(=O)c3ccccc3C(=O)O)c2)[C@@H](O)[C@H]1O]

5′-O-([2-(Carboxyl)phenyl]3-(3-hydroxyphenyl)propanoyl)adenosine (5). Water (24 mg, 1.33 mmol, 10 equiv.). NMO (156 mg, 1.33 mmol, 10 equiv.) and TPAP (4.8 mg, 13 μmol, 0.1 equiv.) were added to a stirring solution of crude intermediate 56 (86 mg, 133.1 μmol, 1 equiv.) in MeCN (2 mL) before being stirred at room temperature for 14 h. The reaction was then quenched with isopropanol and 1 M KHSO₄ (1 mL) was added before the reaction was diluted with water (10 mL), extracted with EtOAc (4×10 mL), organics combined, dried (Na₂SO₄), filtered, concentrated by rotary evaporation. The residue was reconstituted in CH₂Cl₂ (5 mL) and cooled to 0° C. before TFA (5 mL) and water (0.1 mL) added and the reaction stirred for 4 h while returning to room temperature. Concentration by rotary evaporation, purification by preparative HPLC (5%→45% MeCN in H₂O with 0.1% TFA), and lyophilization yielded the product 5 as a fluffy white solid (30 mg, 44% over 4 steps).

IR (ATR): 3320, 2946, 2837, 1757, 1697, 1607, 1492, 1447, 1424, 1290, 1262, 1204, 1141, 1103, 1030, 900, 842, 802, 771, 726, 701, 644. ¹H-NMR (600 MHz; MeOD): δ 8.40 (s, 1H), 8.29 (s, 1H), 7.80-7.77 (m, 1H), 7.70-7.69 (m, 1H), 7.58-7.55 (m, 2H), 7.12-7.09 (m, 1H), 6.73-6.69 (m, 2H), 6.10 (d, =4.5 Hz, 1H), 4.67 (t, J=4.7 Hz, 1H), 4.44 (dd, J=5.9, 3.7 Hz, 1H), 4.35 (dt, J=4.7, 3.0 Hz. 1H), 4.28-4.26 (m, 1H), 4.17-4.14 (m, 1H), 2.69-2.28 (m. 4H). ¹³C-NMR (151 MHz; CDCl₃): δ 160.1, 153.1, 150.2, 147.36, 147.33, 144.27, 144.25, 144.25, 143.0, 130.74, 130.72, 122.45, 122.42, 122.37, 120.4, 115.66, 115.63, 113.2, 90.5, 85.1, 76.4, 71.9, 68.3, 49.9, 49.6, 31.1. HRMS (ESI) m/z calcd for C₂₆H26N₅O₇ ([M+H]⁺) 520.1832; found 520.1824.

### Example 2B

### Synthesis of p-phenolic Linker Analogue (6)

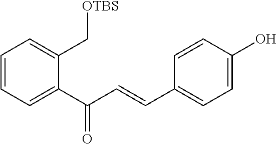 [SMILES: CC(C)(C)[Si](C)(C)OCc1ccccc1C(=O)/C=C/c1ccc(O)cc1]

(E)-1-(2-((tert-butyldimethylsilyloxy)methyl)phenyl)-3-(4-hydroxyphenyl) prop-2-en-1-one (S1). Vinyl ketone 52 (300 mg, 1.085 mmol, 1.2 equiv.), 4-bromophenol (156 mg, 0.904 mmol, 1 equiv.), NBu₄Cl (25 mg, 0.0904 mmol, 0.1 equiv.), and PdCl₂(dtbpf) (59 mg, 0.0904 mmol, 0.1 equiv) were suspended in DMA (2.7mL) before NCy₂Me (265 mg, 1.356 mmol, 1.5 equiv.) was added and the reaction stirred vigorously at 85° C. in a sealed tube for 16 h. The reaction was cooled to room temperature before being diluted with 8 mL water and extracted with Et₂O (4×8 mL), organics combined, dried (Na₂SO₄), filtered, and concentrated by rotary evaporation. Purification by silica flash chromatography (10%→30% EtOAc in hexanes) yielded the product (S1) as a white solid (270 mg, 81%).

IR (ATR): 3332, 2957, 2931, 2887, 2858, 1626, 1580, 1514, 1473, 1443, 1364, 1336, 1282, 1258, 1215, 1171, 1128, 1085, 1025, 985, 941, 911, 834, 777, 734, 671, 631. ¹H-NMR ((600 MHz; CDCl₃): δ 7.72 (d, J=7.8 Hz, 1H), 7.56 (dd, J=7.6, 0.9 Hz, 1H), 7.52 (td, J=7.6, 1.0 Hz, 1H), 7.47-7.43 (m, 3H), 7.35 (td, J=7.5, 0.4 Hz, 1H), 7.05 (d, J=15.9 Hz, 1H), 6.87-6.84 (m, 2H), 6.59 (s, 1H), 4.94 (s, 2H), 0.91 (s, 9H), 0.08 (s, 6H). ¹³C-NMR (151 MHz; CDCl₃): δ 196.5, 158.7, 146.7, 141.4, 136.7, 131.0, 130.6, 128.2, 127.3, 127.0. 126.5, 123.6, 116.1, 62.9, 26.0, 18.4, −5.4. HRMS (ESI) calcd for C₂₂H₂₇O₃Si ([M+H]⁺) 367.1729; found 367.1727.

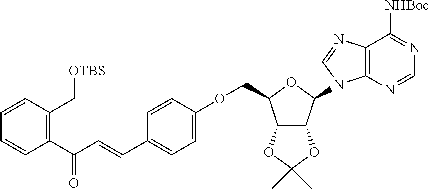 [SMILES: CC(C)(C)OC(=O)Nc1ncnc2c1ncn2[C@@H]1O[C@H](COc2ccc(/C=C/C(=O)c3ccccc3CO[Si](C)(C)C(C)(C)C)cc2)[C@H]2OC(C)(C)O[C@H]21]

6-N-t-Butoxycarbonyl-2′,3′-O-isopropylidene-5′-O-([E]-[2-([tert-butyldimethylsilyloxy]methyl)phenyl]-3-(4-hydroxyphenyl)prop-2-en-t-one)adenosine (S2). DIAD (189 mg, 0.936 mmol, 1.5 equiv.) was added dropwise to a stirring solution of phenol S1 (230 mg, 0.624 mmol, 1 equiv.), protected adenosine 42 (254 mg, 0.624 mmol, 1 equiv.) (prepared as described in Lu, X.; Zhang. H.; Tonₗe, P. J.; Tan, D. S. Bioorg. Med. Chem. Lett. 2008, 18(22), 5963-5966, the contents of which are incorporated herein by reference in their entirety), and resin bound PPh₃ (767 mg, 0.936 mmol, 32% by weigh, 1.5 equiv.) in THF (6 mL) at 0° C. before being allowed to return to room temperature. After 14 h, the reaction was quenched with water (0.2 mL) and filtered through a pad of celite, the pad washed with EtOAc, and solvent removed by rotary evaporation. Purification by silica flash chromatography (40% 60% EtOAc in hexanes) yielded the product (S2) as a white solid (310 mg, 66%).

IR (ATR): 2989, 2954, 2931, 2857, 2247, 1751, 1705, 1658, 1609, 1511, 1463, 1423, 1384, 1369, 1327, 1304, 1251, 1213, 1174, 1144. 1081, 1017, 982, 909, 836, 776. 729, 668, 645. ¹H-NMR ((600 MHz; CDCl₃): δ 8.79 (s, 1H), 8.09 (s, 1H), 8.05 (s, 1H), 7.74 (d, J=7.8 Hz, 1H), 7.60 (dd, J=7.6, 1.1 Hz, 1H), 7.52 (td, J 7.6, 1.2 Hz, 1H), 7.49-7.44 (m, 3H), 7.35 (t, J=7.5 Hz, 1H), 7.09 (d, J=15.9 Hz. 1H), 6.79-6.76 (m, 2H), 6.25 (d, J=2.2 Hz, 1H), 5.52 (dd. J=6.2, 2.2 Hz, 1H), 5.19 (dd, J=6.2, 2.9 Hz, 1H), 4.95 (s, 2H), 4.72 (dd, J=7.4, 4.3 Hz, 1H), 4.30 (dd, J=10.2, 4.1 Hz, 1H), 4.18 (dd. J=10.2. 4.9 Hz, 1H), 1.67 (s, 3H), 1.56 (s, 9H), 1.43 (s, 3H), 0.92 (s, 9H), 0.09 (s, 6H). ¹³C-NMR (151 MHz; CDCl₃): δ 195.1, 159.9, 153.2, 150.26, 150.06, 149.6, 145.1, 141.8, 141.3, 136.6, 131.0, 130.2, 128.25, 128.23, 127.1, 126.3, 124.2, 122.2, 114.74, 114.66, 91.7, 85.5, 84.6, 82.4, 81.8, 68.0, 62.9, 28.1, 27.2, 26.0, 25.4, 18.4, −5.3. HRMS (ESI) m/z calcd for C₄₀H₅₂N₅O₈Si ([M+H]⁺) 758.3585; found 758.3576.

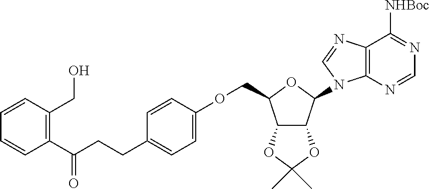 [SMILES: CC(C)(C)OC(=O)Nc1ncnc2c1ncn2[C@@H]1O[C@H](COc2ccc(CCC(=O)c3ccccc3CO)cc2)[C@H]2OC(C)(C)O[C@H]21]

6-N-t-Butoxycarbonyl-2′,3′-O-isopropylidene-5′-O-(1-[2-(hydroxymethyl) phenyl]-3-(3-hydroxyphenyl) prop-2-en-1-one)adenosine (S3). Phenylsilane (96 mg, 699.2 timol, 2 equiv.) was added to a stirring solution of intermediate S2 (265 mg, 349.6 μmol, 1 equiv.) and Strykers catalyst (62 mg, 31.5 μmol, 0.09 equiv.) in toluene (5 mL) and room temperature. After 16 h, the reaction was quenched with saturated ammonium chloride (10 mL) and stirred for 5 min before 10% ammonium hydroxide (10 mL) was added and the reaction stirred for an additional 5 min. The reaction was then extracted with Et₂O (4×20 organics combined, dried (Na₂SO₄). filtered, concentrated by rotary evaporation, and dried under high vacuum for 1 h. The reside was reconstituted in THF (5 mL) and cooled to 0° C. before TBAF (697 μM, 697 μmol, 1 M in THF, 2 equiv.) was added and the reaction stirred for 1 h. CaCO₃ (349 mg, 3.486 mmol, 10 equiv.) and MeOH (3 mL) was added and the reaction stirred for 15 min before sulfonic acid resin (dowex 50WX8, 500 mg) was added and the reaction stirred for an additional 10 min. The reaction was then filtered through a pad of celite and concentrated by rotary evaporation to give the crude product S3 (202 mg, 90% yield).

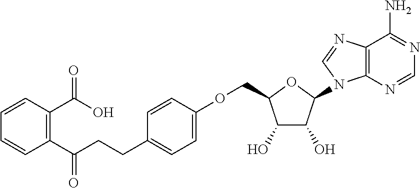 [SMILES: Nc1ncnc2c1ncn2[C@@H]1O[C@H](COc2ccc(CCC(=O)c3ccccc3C(=O)O)cc2)[C@@H](O)[C@H]1O]

5′-O-([2-(Carboxyl)phenyl]3-(3-hydroxyphenyl)propannyl)adenosine (6). Water (56 mg, 3.128 mmol, 10 equiv.), NMO (366 mg, 3.128 mmol, 10 equiv.) and TPAP (11 mg, 31 mol, 0.1 equiv.) were added to a stirring solution of crude intermediate S3 (202 mg, 312.8 μmol, 1 equiv.) in MeCN (5 mL) before being stirred at room temperature for 14 h. The reaction was then quenched with isopropanol and 1 M KHSO₄ (1 mL) was added before the reaction was diluted with water (20 mL), extracted with EtOAc (4×20 mL), organics combined, dried (Na₂SO₄), filtered, concentrated by rotary evaporation, and dried under high vacuum 1 h. The residue was reconstituted in CHCl₂ (8 mL) and cooled to 0° C. before TFA (8 mL) and water (0.8 mL) added and the reaction stirred for 4 h while returning to room temperature. Concentration by rotary evaporation, purification by preparative HPLC (5%→45% MeCN in H₂O with 0.1% TFA), and lyophilization yielded the product 6 as a fluffy white solid (65 mg, 36% over 4 steps).

IR (ATR): 3323, 2921, 2869, 1750, 1690, 1614, 1512, 1424, 1292, 1242, 1203, 1140, 1050, 980, 898, 827, 801, 769, 724, 699, 642. ¹H-NMR (600 MHz; MeOD): δ 8.45 (s, 1H), 8.33 (s, 1H), 7.84-7.83 (m, 1H), 7.77-7.76 (m, 1H), 7.64-7.63 (m, 2H), 7.04-7.03 (m, 2H), 6.87-6.85 (m, 2H), 6.14 (d, J=4.6 Hz, 1H), 4.72 (t, J=4.8 Hz, 1H), 4.49 (t, J =4.8 Hz, 1H), 4.40 (q. J=3.9 Hz, 1H), 4.32-4.30 (m, 1H), 4.20 (dd. J=10.9, 3.3 Hz, 1H), 2.72-2.65 (m, 1H), 2.48-2.29 (m, 3H). ¹³C-NMR (151 MHz: CDCl₃): δ 158.3, 153.38, 153.37, 150.2, 147.71, 147.70, 142.8, 136.7, 135.9, 135.20, 135.19, 135.19, 135.18, 131.7, 130.5, 120.46, 120.45, 115.7, 90.5, 85.1, 76.4, 71.9, 68.5, 50.0, 49.6, 30.2. HRMS (ESI) m/z calcd for C₂₆H₂₆N₅O₇ ([M+H]⁺) 520.1832; found 520.1809.

### Example 2C

### Synthesis of m-(3triflouromethyl)phenolic Linker Analogue (8)

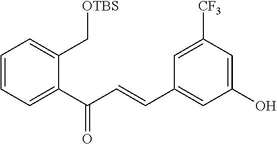 [SMILES: CC(C)(C)[Si](C)(C)OCc1ccccc1C(=O)/C=C/c1cc(O)cc(C(F)(F)F)c1]

(E)-1-(2-((tert-butyldimethylsilyloxy)methyl)phenyl)-3-(3-hydroxy-5-(trifluoromethyl) phenyl)prop-2-en-1-one (S4). Vinyl ketone 52 (300 mg, 1.085 mmol, 1.2 equiv.), 3-bromo-5-trifluoromethylphenol (218 mg, 0.904 mmol, 1 equiv.), NBu₄Cl (25 mg, 0.0904 mmol, 0.1 equiv.), and PdCl₂(dtbpf) (59 mg, 0.0904 mmol, 0.1 equiv) were suspended in DMA (2.7 mL) before NCy₂Me (265 mg, 1.356 mmol, 1.5 equiv.) was added and the reaction stirred vigorously at 85° C. in a sealed tube for 16 h. The reaction was cooled to room temperature before being diluted with 8 mL water and extracted with Et_{2O (}4×8 mL), organics combined, dried (Na₂SO₄), filtered, and concentrated by rotary evaporation. Purification by silica flash chromatography (10%→30% EtOAc in hexanes) yielded the product (S4) as a white solid (290 mg, 74%).

IR (ATR): 3372, 2955, 291, 2885, 2858, 1601, 1449, 1369, 1310, 1258, 1219, 1173, 1130, 1098, 1024, 981, 911, 839, 815, 778, 736, 690, 669, 620. ¹H-NMR ((500 MHz; CDCl₃): δ 7.73 (d, J=7.8 Hz, 1H),7.61 (d, J=7.6 Hz, 1H), 7.54 (t, J=7.5 Hz, 1H), 7.46 (d, J=16.0 Hz, 1H), 7.38-7.35 (m, 2H), 7.23 (d, J=16.0 Hz, 2H), 7.12 (s, 1H), 6.08 (s, 1H), 4.96 (s, 2H), 0.92 (s, 9H), 0.09 (s, 6H). ¹³C-NMR (126 MHz; CDCl₃): δ 195.1, 156.6, 143.8, 142.0, 137.0, 136.0, 132.8, 131.7, 128.5, 127.60, 127.52, 126.6, 123.5, 118.1, 117.3, 114.4, 63.1, 26.0, 18.4, −5.4. ¹⁹F-NMR (126 MHz; CDCl₃): δ 63.0. HRMS (ESI) m/z calcd for C₂₃H₂₈O₃F₃Si ([M+H]⁺) 437.1760; found 437.1739.

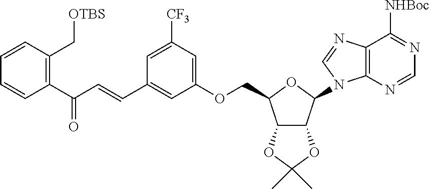 [SMILES: CC(C)(C)OC(=O)Nc1ncnc2c1ncn2[C@@H]1O[C@H](COc2cc(/C=C/C(=O)c3ccccc3CO[Si](C)(C)C(C)(C)C)cc(C(F)(F)F)c2)[C@H]2OC(C)(C)O[C@H]21]

6-N-t-Butoxycarbonyl-2′,3′-O-isopropylidene-5″-O-([E]-1-[2-([tert-butyldimethylsilyloxy]methyl)phenyl]-3-(3-hydroxy-5-(trifluoromethyl) phenyl) prop-2-en-1-one)adenosine (S5). DEAD (125 mg, 0.619 mmol, 1.5 equiv.) was added dropwise to a stirring solution of phenol S4 (270 mg, 0.619 mmol, 1 equiv.), protected adenosine 42 (126 mg, 0.309 mmol, 1 equiv.) (prepared as described in Lu, X.; Zhang, H.; Tonge, P. J.; Tan, D. S. Bioorg. Med. Chem. Lett. 2008, 18(22), 5963-5966, the contents of which are incorporated herein by reference in their entirety), and resin bound PPh₃ (507 mg, 0.619 mmol, 32% by weigh. 1.5 equiv.) in THF (6 mL) at 0° C. before being allowed to return to room temperature. After 14 h, the reaction was quenched with water (0.2 mL) and filtered through a pad of celite, the pad washed with EtOAc, and solvent removed by rotary evaporation. Purification by silica flash chromatography (40%→60% EtOAc in hexanes) yielded the product (S5) as a white solid (215 mg, 84%).

IR (ATR): 2982, 2955, 2934, 2857, 2244, 1753, 1717, 1666, 1610, 1521, 1464, 1359, 1326, 1300, 1233, 1173, 1133, 1104, 1080, 1020, 977, 911, 587, 839, 815, 778, 734, 689, 646. ¹H-NMR ((600 MHz; CDCl₃): δ 8.76 (s, 1H), 8.04 (s, 1H), 7.92 (s, 1H), 7.76 (d, J=7.7 Hz, 1H), 7.63 (d, J=7.6 Hz, 1H), 7.55 (td, J=7.6, 0.9 Hz, 1H), 7.44 (d, J=16.0 Hz, 1H), 7.39 (dd, J=13.8, 5.5 Hz, 2H), 7.26 (s, 1H), 7.22 (d, J=16.0 Hz, 1H), 7.15 (s, 1H), 7.03 (s, 1H), 6.22 (d, J=2.2 Hz, 1H), 5.52 (dd, J=6.2, 2.2 Hz, 1H), 5.21 (dd, J=6.2, 3.3 Hz, 1H), 4.96 (s, 2H), 4.70-4.68 (m, 1H), 4.29 (ddd, J=48.5, 10.0, 4.9 Hz. 2H), 1.67 (s. 3H), 1.55 (s, 9H), 1.44 (s, 3H), 0.93 (s, 9H), 0.09 (s, 614). ¹³C-NMR (126 MHz; CDCl₃): δ 194.1, 158,7, 153.2, 150.27, 150.16, 149,5, 142.9, 142.3, 141.4, 137.3, 135.9, 132.8, 131.6. 128.5, 127.9, 127.3, 126.5, 123.4, 122.4, 118.0, 117.1, 114.9, 113.0, 91.4, 85.3, 84.4, 82.4, 81.7, 68.5, 63.0, 28.1, 27.2, 26.0, 25.4, 18.4, −5.3. ¹⁹F-NMR (126 MHz; CDCl₃): δ-63.0. HRMS (ESI) m/z calcd for C₄₁H₅₁N₅O₈P₃Si ([M+H]⁺) 826.3459; found 826.3453.

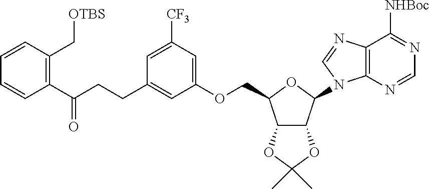 [SMILES: CC(C)(C)OC(=O)Nc1ncnc2c1ncn2[C@@H]1O[C@H](COc2cc(CCC(=O)c3ccccc3CO[Si](C)(C)C(C)(C)C)cc(C(F)(F)F)c2)[C@H]2OC(C)(C)O[C@H]21]

6-N-t-Butoxycarbonyl-2′,3′-O-isopropylidene-5′-O-(1-[2-(hydroxymethyl) phenyl]-3-(3-hydroxy-5-(trifluoromethyl)phenyl)prop-2-en-1-one) adenosine (56). Phenylsilane (56 mg, 521 μmol, 2 equiv.) was added to a stirring solution of intermediate S5 (215 mg, 260.3 mnol, 1 equiv.) and Strykers catalyst (51 mg, 26.0 μmol, 0.09 equiv.) in toluene (5 mL) and room temperature. After 16 h, the reaction was quenched with saturated ammonium chloride (10 mL) and stirred for 5 min before 10% ammonium hydroxide (10 mL) was added and the reaction stirred for an additional 5 min. The reaction was then extracted with Et₂O (4×20 mL), organics combined, dried (Na₂SO₄), filtered, concentrated by rotary evaporation. Purification by silica flash chromatography (50%→100% EtOAc in hexanes) yielded the product (S6) as a white solid (207 mg, 96%).

IR (ATR): 2982, 2956, 2933, 2902, 2858, 2245, 1753, 1681, 1610, 1587, 1522, 1463, 1359, 1329, 1233, 1172, 1144, 1128, 1105, 1080, 1107, 972, 911, 854, 839, 814, 777, 734, 702, 670, 646. ¹H-NMR ((600 MHz; CDCl₃): δ 8.78 (s, 1H), 8.05 (s, 1H), 7.95 (s, 1H), 7.82 (d, J=7.8 Hz, 1H), 7.73-7.71 (m, 1H), 7.55-7.52 (m, I H), 7.34-7.31 (m, 1H), 7.10 (s, 1H), 6.84 (s, 2H), 6.23 (d, J=2.2 Hz, 1H), 5.50 (dd, J=6.2, 2.3 Hz, 1H), 5.17 (dd, J=6.2, 3.1 Hz, 1H), 5.00 (s, 2H), 4.68 (q, J=3.9 Hz, 1H), 4.23 (ddd, J=55.1, 10.1, 4.6 Hz, 2H), 3.25 (t, J=7.6 Hz, 2H), 3.02 (t, J=7.5 Hz, 2H), 1.66 (s, 3H), 1.55 (s, 9H), 1.43 (s, 3H), 0.95 (s, 9H), 0.11 (s, 6H). ¹³C-NMR (126 MHz; CDCl₃): δ 201.4, 158.3, 153.2, 150.3, 150.1, 149.5, 144.2, 143.3, 141.3, 134.5, 132.3, 132.1, 128.7, 126.9, 126.4, 123.8, 122.3, 118.4, 118.2, 114.8, 109.0, 91.6, 85.3, 84.5, 82.4, 81.7, 68.2, 63.5, 41.8, 30.0, 28.1, 27.3, 26.0, 25.4, 18.4, −5.3. ¹⁹F-NMR (126 MHz; CDCl₃): δ 62.7. HRMS (ESI) calcd for C₄₁H₅₃N₅O₈F₃Si ([M+H]⁺) 828.3610; found 828.3616.

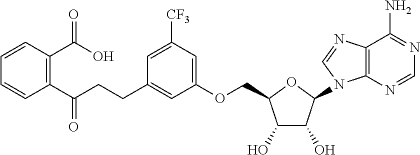 [SMILES: Nc1ncnc2c1ncn2[C@@H]1O[C@H](COc2cc(CCC(=O)c3ccccc3C(=O)O)cc(C(F)(F)F)c2)[C@@H](O)[C@H]1O]

5′-O-([2-(Carboxyl)phenyl]3-(3-hydroxy-5-(trifluoromethyl)phenyl)propanoyl)adenosine (8). TBAF (786 μL, 786.0 μmol, 1M in THF, 3 equiv.) was added to intermediate S6 (217 mg, 260.3 μmol, 1 equiv.) in THE (5 mL) at 0° C. before and stirred for 1 h. CaCO₃ (349 mg, 3.486 mmol, 10 equiv.) and MeOH (3 ml,) was added and the reaction stirred for 15 min before sulfonic acid resin (Dowex 50WX8, 500 mg) was added and the reaction stirred for an additional 10 min. The reaction was then filtered through a pad of celite and concentrated by rotary evaporation. The residue was reconstituted in MeCN (5 mL) before water (45 mg, 2.5 mmol, 10 equiv.), NMO (295 mg, 2.521 mmol, 10 equiv.), and TPAP (8.9 mg, 25 μmol, 0.1 equiv.) were added and stirred at room temperature for 14 h. The reaction was then quenched with isopropanol and 1 M KHSO₄ (1 mL) was added before the reaction was diluted with water (20 mL), extracted with EtOAc (4×20 mL), organics combined, dried (Na₂SO₄), filtered, concentrated by rotary evaporation, and dried under high vacuum 1 h. The residue was reconstituted in CH₂Cl₂ (8 mL) and cooled to 0° C. before TFA (8 mL) and water (0.8 mL) added and the reaction stirred for 4 h while returning to room temperature. Concentration by rotary evaporation, purification by preparative HPLC (5% 45% MeCN in H₂O with 0.1% TFA), and lyophilization yielded the product 8 as a fluffy white solid (46 mg, 31% over 4 steps).

IR (ATR): 3346, 2509, 2247, 2076, 1756, 1693, 1608, 1455, 1353, 1320, 1289, 1245, 1205, 1126, 1055, 982, 898. 842, 802, 767, 726. 703, 644. ¹H-NMR ((600 MHz; MeOD): δ 8.43 (s, 1H), 8.34 (s, 1H), 7.84-7.81 (m, 1H), 7.72-7.68 (m, 1H), 7.60-7.57 (m, 2H), 7.05-6.96 (m, 3H), 6.14 (d, J=4.4 Hz, 1H), 4,76 (t, J=4.7 Hz, 1H), 4.53 (t, J=5.0 Hz, 1H), 4.43-4.38 (m, 2H), 4.28 (dd, J=10.8, 3.5 Hz, 1H), 2.89-2.50 (m, 4H). ¹³C-NMR (126 MHz; MeOD): δ 160.4, 153.13, 153.05, 153.03, 150.1, 147.1, 145.77, 145.71, 145.65, 143.22, 143.18, 132.9, 131.58, 131.43, 125.4, 124.1, 120.5, 119.4, 118.9, 110.4, 90.7, 84.8, 76.0, 71.8, 68.8, 49.3, 31.0. ¹⁹F-NMR (126 MHz; MeOD): δ-64.07. HRMS (ESI) m/z calcd for C₂₇H₂₅N₅O₇F₃ ([M+H]⁺) 588.1706; found 588.1681.

### Example 3

### Synthesis of Selected Substituted m-Phenyl Ether Analogues

Certain analogues were synthesized by following the general scheme below. These analogs and others can also be prepared by the methods disclosed in Evans, C. E. et al. Biochem. 2019, 58, 1918-1930 and the accompanying Supporting Information, the contents of which are incorporated herein by reference in their entireties.

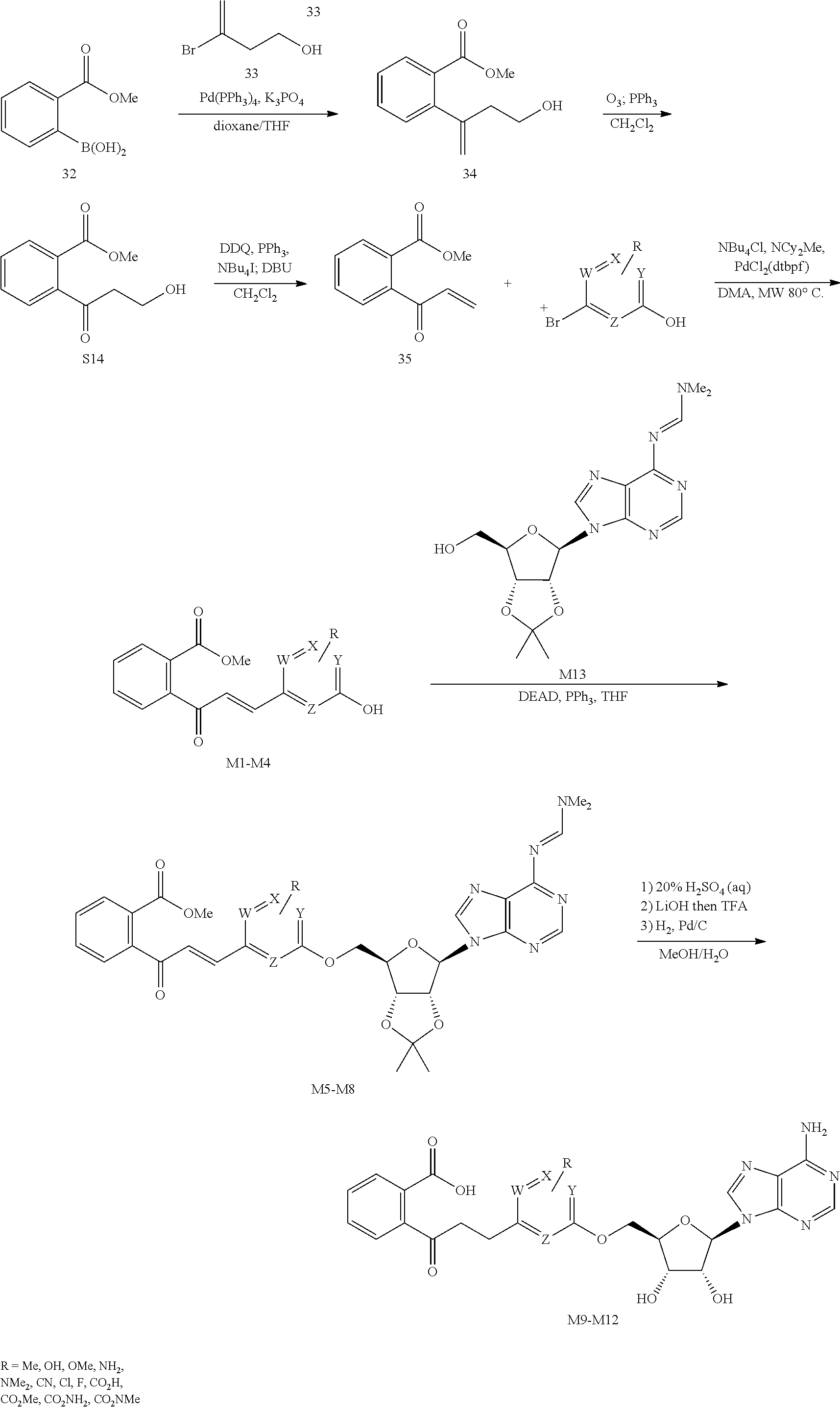

### Example 3A

### Synthesis of m-3-Pyridyl Ether Analogue (9)

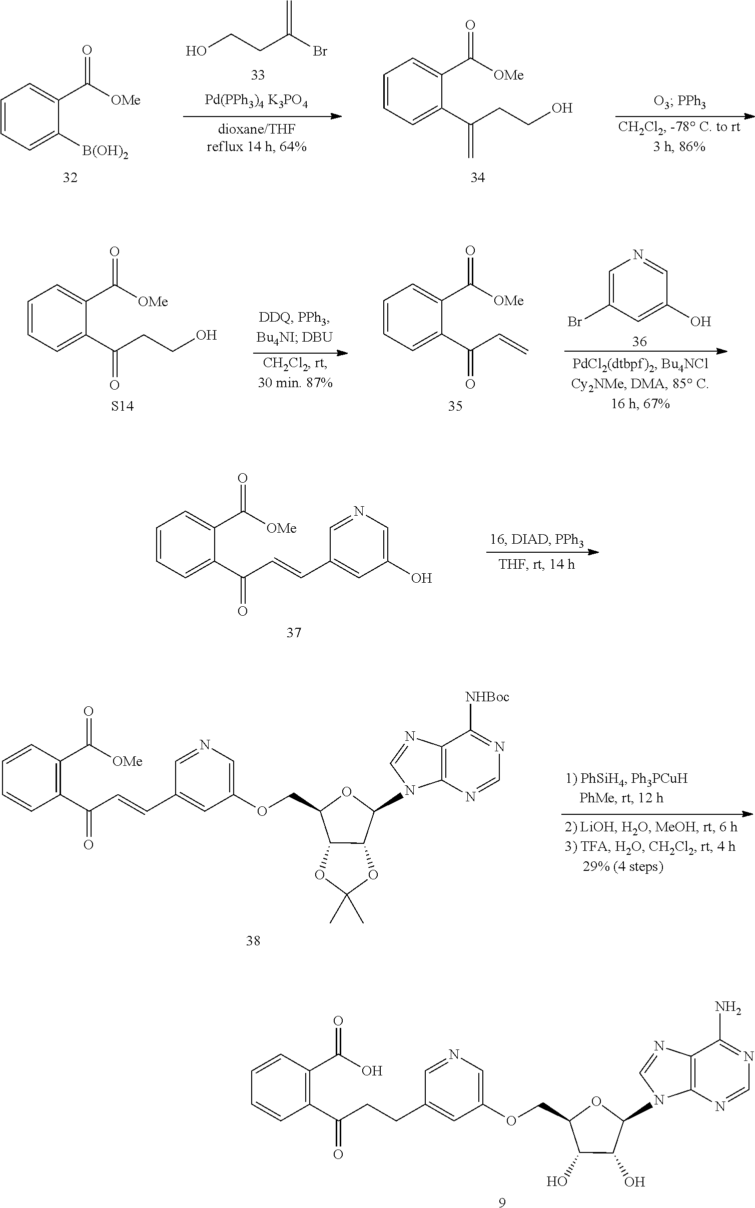

DBU, 1,8-diazabicyclo[5.4.0]undec-7-ene; DDQ, 2,3-dichloro-5,6-dicyano-1,4-benzoquinone; DIAD, diisopropyl azodicarboxylate; DMA, dimethylacetamide; dtbpf, 1,1′-bis(di-t-butylphosphino)ferroceue; TBSCI, t-butyldimethylsilyl chloride; TFA, trifluoroacetic acid; THF tetrahydrofuran.

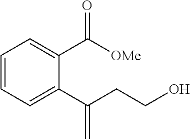

Methyl 2-(4-hydroxybut-1-en-2-Abenzaate (S13) In a 250-mL roundbottom flask, 3-bromobut-3-en- 1 -ol (33) (1 g, 6.622 mmol, 1 equiv), (methoxycarbonyl)phenyl)boronic acid (32) (1.787 g, 9.33 mmol, 1.5 equiv), Pd(PPh₃)₄ (765 mg, 0.662 mmol, 0.1 equiv) and K₃PO₄ (5.62 g, 26.48 mmol, 4.0 equiv) were suspended in THF (40 mL) and dioxane (40 mL) and heated to reflux for 14 h. The reaction was cooled to rt and filtered over celite. Solvent was concentrated by rotary evaporation. Purification by silica flash chromatography (25% 75% EtOAc in hexanes) yielded olefin intermediate 34 as a clear oil (863 mg, 63%). IR (ATR): 3412, 3078, 2952, 2884, 1716, 1636, 1598, 1571, 1484, 1434, 1292, 1259, 1192, 1164, 1123, 1076, 1050, 991, 964, 910, 860, 830, 772, 729, 658, 624. ¹ H-NMR ((600 MHz; CDCl₃): δ 7.76 (dd, J=7.8, 1.1 Hz, 1H), 7.47 (td, J=7.6, 1.3 Hz, 1H), 7.33 (td, J=7.6, 1.0 Hz, 1H), 7.27-7.26 (m, 1H), 5.25 (d, J=1.2 Hz, 1H), 4.94 (d, J=1.3 Hz, 1H), 3.88 (s, 3H), 3.62 (q, J=5.2 Hz, 2H), 3.34 (s, 1H), 2.72 (t, J=5.4 Hz, 2H). ¹³C-NMR (126 MHz; CDCl₃): δ 169.2 145.8, 143.2, 131.4, 130.6, 129.9, 128.9, 127.1, 117.1, 59.9, 52.5, 41.6. HRMS (ESI) calcd for C₁₂H₁₅O₃ ([M+H]⁺) 207.1021; found 207.1021.

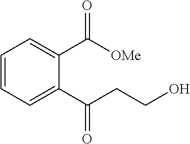 [SMILES: COC(=O)c1ccccc1C(=O)CCO]

Methyl 2-(3-hydroxypropanoyl)benzoate (S14). In a 100-mL roundbottom flask, methyl 2-(4- hydroxybut-1-en-2-yl)benzoate (34) (850 mg, 4.121 nunol, 1 equiv) was dissolved in CH₂Cl₂ (50 mL) and cooled to 78° C. Ozone was bubbled through the solution until a persistent blue color was observed, then nitrogen was bubbled through the solution until the blue color disappeared. The ozonide was quenched by addition of PPh₃ (1.167 g, 4.45 mmol, 1.08 equiv) and the reaction stirred for 3 h with warming to rt. The mixture was concentrated by rotary evaporation. Purification by silica flash chromatography (50% 100% EtOAc in hexanes) yielded keto alcohol intermediate S14 as a clear oil (732 mg, 85%). IR (ATR): 3428, 2956, 2894, 1719, 1599, 1576, 1488, 1347, 1390, 1362, 1285, 1211, 1195, 1167, 1137, 1098, 1048, 991, 962. 914, 867, 832, 766, 741, 710. 681, 648. ¹H-NMR ((600 MHz; CDCl₃): δ 8.40 (s, 1H), 8.34-8.32 (m, 2H), 8.22-8.22 (m, 1H), 7.87-7.86 (m, 2H), 7.74 (t, J=0.8 Hz, 1H), 7.61 (t, J=7.0 Hz, 2H), 6.11 (d, J=4.3 Hz. 1H), 4.79 (t, J=4.7 Hz, 1H), 4.58-4.54 (m, 2H), 4.48-4.42 (m, 2H), 3.03-2.94 (m, 2H), 2.68-2.36 (m, 2H). ¹³C-NMR (126 MHz; CDCl₃): δ 206.1, 167.2, 143.0, 132.5, 130.0, 128.2, 126.4, 126.1, 58.2, 52.8, 45.1. HRMS (ESI) m/z calcd for C₁₁H₁₃O₄ ([M+H]⁺) 209.0814; found 209.0809.

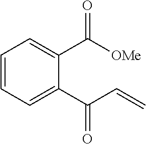

Methyl 2-acryloylbeazoate (S35). In a 25-mL roundbottom flask. DDQ (218 mg, 0.960 mmol, 2.0 equiv) and PPh₃ (264 mg, 1.008 mmol, 2.1 equiv) were dissolved in CH₂Cl₂ (10 mL). Tetrabutylammonium iodide (355 mg, 0.960 mmol. 2.0 equiv) was added and the reaction stirred for 10 min. Methyl 2-(3-hydroxypropanoyl)benzoate (S14) in CH₂Cl₂ (1 mL) was added and the reaction stirred at rt for 30 min. DBU (292 mg, 1.920 mtnol, 4.0 equiv) was added and the reaction stirred for 30 min. The mixture was poured into water (30 mL) and extracted with CH₂Cl₂ (4×30 mL). The combined organic extracts were dried (Na₂SO₄), filtered, and concentrated by rotary evaporation. Purification by silica flash chromatography (0%→20% EtOAc in hexanes) yielded vinyl ketone 35 as a clear oil (74 mg, 81%). IR (ATR): 2954, 1724, 1669, 1611, 1575, 1486, 1435, 1404, 1286, 1231, 1193, 1129, 1076, 1041, 997, 959. 912, 831, 801, 765, 746, 709, 678, 645. ¹H-NMR ((600 MHz; CDCl₃): 7.98 (dd, J=7.8, 0.9 Hz, 1H), 7.61 (td, J=7.5, 1.3 Hz, 1H), 7.54 (td, J=7.6, 1.3 Hz, 1H), 7.37 (dd, J=7.5, 0.9 Hz, 1H), 6.67 (dd, J=17.7, 10.6 Hz, 1H), 5.99 (dd, J=10.7, 0.7 Hz, 1H), 5.85 (dd, J=17.7, 0.7 Hz, 1H), 3.84 (s, 3H). ¹³C-NMR (126 MHz; CDCl₃): δ 197,1, 166.7, 141.1, 137.3, 132.3, 130.6, 130.1, 129.9, 129.2, 127.6, 52.5. HRMS (ESI) re: calcd for C₁₁H₁₁O₃ ([M+H]⁺) 191.0708; found 191.0709.

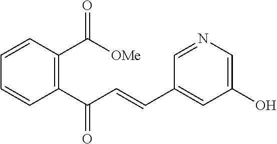 [SMILES: COC(=O)c1ccccc1C(=O)/C=C/c1cncc(O)c1]

Methyl (E)-2-(3-(5-hydroxypyridin-3-yl)acryloyl)benzoate (37). In a 15-mL sealed tube, vinyl ketone 35 (185 mg, 0.973 mmol, 1.2 equiv), aryl bromide 36 (141 mg, 0.811 mmol, 1 equiv), NBu₄Cl (23 mg, 0.081 mmol, 0.1 equiv), and PdCl₂(dtbpf) (53 mg, 0.081 mmol, 0.1 equiv) were suspended in DMA (2.4 mL). NCy₂Me (237 mg, 1.215 mmol, 1.5 equiv) was added, the reaction vessel sealed, and the reaction stirred vigorously at 85° C. for 16 h. The reaction was cooled to rt, diluted with water (8 mL), and extracted with Et₂O (4×8 mL). The combined organic extracts were dried (Na₂SO₄), filtered, and concentrated by rotary evaporation. Purification by silica flash chromatography (50%→100% EtOAc in hexanes) yielded phenol 37 as an off-white solid (154 mg, 67%). IR (ATR): 3066, 2953, 1719, 1653, 1609, 1576, 1486, 1434, 1282, 1217, 1186, 1138,1108, 1064, 1020, 977, 911, 859, 828, 792, 769, 729, 708, 690, 667, 648, 607. ¹H-NMR ((600 MHz; CDCl₃): δ 8.26 (d, J=2.3 Hz, 1H), 8.16 (d, J=0.9 Hz, 1H), 7.99 (dd, J=7.8, 1.0 Hz, 1H), 7.63 (td, J=7.5, 1.3 Hz, 1H), 7.57 (td, J=7.7. 1.3 Hz, 1H), 7.49 (d, J=1.8 Hz, 1H), 7.43 (dd, J=7.5. 1.0 Hz, 1H), 7.18 (d, J=16.3 Hz, 1H), 7.07 (d, J=16.3 Hz, 1H), 3.83 (s, 3H). ¹³C-NMR (126 MHz; CDCl₃): δ 196.0, 166.9, 155.0, 141.3, 140.2, 139.2, 137.8, 132.5, 132.3, 130.29, 130.23. 129.8, 129.2, 127.6, 122.7, 52.7. HRMS (EST) calcd for C₁₆H₁₄NO₄ ([M+H]⁺) 284.0923: found 284.0934.

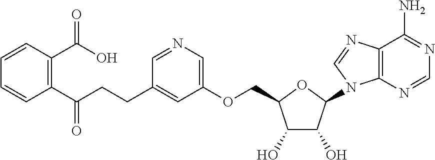 [SMILES: Nc1ncnc2c1ncn2[C@@H]1O[C@H](COc2cncc(CCC(=O)c3ccccc3C(=O)O)c2)[C@@H](O)[C@H]1O]

5′-O-([2-(Carboxyl)phenyl]3-(5-hydroxypyridin-3-yl)propanoyl)adenosine (9). In a 10-mL roundbottom flask, phenol 37 (90 mg, 0.317 mmol, 1 equiv), protected adenosine 16 (86 mg, 0.212 mmol, 1 equiv) (prepared as described in Lu, X.; Zhang, H.; Tonge, P. J.; Tan, D. S. Biourg. Med. Chem. Lett. 2008, 18(22), 5963-5966, the contents of which are incorporated herein by reference in their entirety), and resin-bound PPh₃ (260 mg, 0.317 mmol, 32 wt %. 1.5 equiv) were suspended in THF (5 mL) and cooled to 0° C. DIAD (62 mg, 0.317 mmol, 1.5 equiv) was added dropwise, then the reaction was stirred for 14 h with warming to rt. The reaction was quenched with water (0.2 mL), filtered through a pad of celite, and the pad washed with EtOAc. The combined filtrates were concentrated by rotary evaporation to afford the crude protected phenol adenosine intermediate 38, which was carried forward without further purification.

In a 10-mL roundbottom flask, the crude phenol-adenosine intermediate 38 above was dissolved in toluene (5 mL). Stryker's reagent (104 mg, 319 μmol, 1.5 equiv) was added and the reaction stirred for 12 h. The reaction was quenched with satd aq NH₄Cl (10 mL) and stirred for 5 min, then 10% aq NH₄OH (10 mL) was added and the reaction stirred for an additional 5 min. The reaction was then extracted with EtOAc (4×20 mL). The combined organic extracts were dried (Na₂SO₄), filtered, and concentrated by rotary evaporation to afford the crude reduced intermediate, which was carried forward without further purification.

In a 25-mL roundbottom flask, the crude reduced intermediate above was dissolved in MeOH (5 mL). Water (0.5 mL) and LiOH (10 mg, 0.423 mmol, 2 equiv) were added and the reaction stirred at rt for 6 h. Concentration by rotary evaporation afforded the crude carboxylate intermediate, which was carried forward without further purification.

In a 25-mL roundbottom flask, the crude carboxylate intermediate above was suspended in CH₂Cl₂. Water (0.5 mL) and TFA (5 mL) were added and the reaction stirred at rt for 4 h. The mixture was concentrated by rotary evaporation. Purification by preparative HPLC (5% 45% CH₃CN in H₂O with 0.1% TFA) yielded m-3-pyridyl ether analogue 9 as a fluffy white solid (32 mg, 29% over 4 steps). IR (ATR): 3397, 2521, 1681, 1577, 1428, 1290, 1202, 1138, 1046, 979, 899, 840, 800, 765, 723, 701, 680, 642, 611. ¹H-NMR ((600 MHz; CDCl₃): δ 8.40 (s, 1H), 8.34-8.32 (m, 2H), 8.22-8.22 (m, I H), 7.87-7.86 (m, 2H), 7.74 (t, J=0.8 Hz, 1H), 7.61 (t, J=7.0 Hz, 2H), 6.11 (d, J=4.3 Hz, 1H), 4.79 (t, J=4.7 Hz, 1H), 4.58-4.54 (m, 2H), 4.48-4.42 (m, 2H), 3.03-2.94 (m, 2H), 2.68-2.36 (m, 2H), ¹³C-NMR (126 MHz; CDCl₃): δ 162.5, 158.1, 153.82, 153.82, 153.80, 150.19, 150.17, 148.39, 148.37, 148.35, 143.06, 143.02, 137.69, 137.68, 131.7, 131.0, 129.9, 120.6, 90.8, 84.3, 75.4, 71.7, 70.0, 49.6, 28.0. HRMS (ESI) m/z: calcd for C₂₅H₂₅N₆O₇ ([M+H]⁺) 521.1785; found 521.1772.

### Example 3B

### Synthesis of m-2-Pyridyl Ether Analogue (M9)

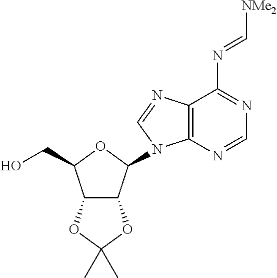 [SMILES: CN(C)/C=N/c1ncnc2c1ncn2[C@@H]1O[C@H](CO)[C@H]2OC(C)(C)O[C@H]21]

(E)-N′-(9-((3aR,4R,6R,6aR)-6-(hydroxymethyl)-2,2-dimethyltetrahydrofuro[3,4-d][1,3]dioxol-4-yl)-9H-purin-6-yl)-N,N-dimethylformimidamide (M13) 2′,3′-O-Isopropylideneadenosine (4.24 ,g 13.79 mmol, 1 equiv) was dissolved in 35 mlL anhydrous DMF in a flame-dried 100 mL round bottom flask. N,N-Dimethylformamide dimethyl acetal (8.22 g, 68.95 mmol, 5 equiv) was added and the reaction was allowed to stir at room temperature for 17 hr. The solvent was removed by rotary evaporation to afford the crude product as an off-white solid (4.98 g, 99%). The protected adenosine was used without further purification.

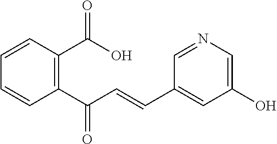 [SMILES: O=C(O)c1ccccc1C(=O)/C=C/c1cncc(O)c1]

Methyl (E)-2-(3-(2-hydroxypyridin-4-yl)acryloyl)benzaate (M1) In a 0.5-2 mL microwave vial, vinyl ketone 35 (270 mg, 0.893 mmol, 1 equiv), 4-brornopyridin-2-ol (233 mg, 1.340 mmol, 1.5 equiv), NBu₄Cl (24 mg, 0.089 mmol, 0.1 equiv), and PdCl₂(dtbpf) (58 mg, 0.089 mmol, 0.1 equiv) were suspended in DMA (2 mL). NCy₂Me (262 mg, 1.340 mmol, 1.5 equiv) was added, the reaction vessel sealed, and the reaction stirred vigorously at 140° C. in a Biotage Initiator microwave for 15 min. The reaction was cooled to rt and filtered over celite. Solvent was removed via rotary evaporation. Purification by silica flash chromatography (50%→100% EtOAc in hexanes) yielded pyridinol M1 as a yellow semi-solid (70 mg, 27%). ¹H NMR (600 MHz, CDCl₃) δ 8.02 (dd, J=7.8, 1.3 Hz, 1H). 7.66 (td, J=7.5, 1.3 Hz, 1H), 7.59 (td, J=7.6, 1.3 Hz, 1H). 7.47-7.40 (m, 1H), 7.28 (t, J=7.6 Hz, 1H), 7.06-6.95 (m, 2H), 6.56 (d, J=1.7 Hz, 1H), 6.40 (dd, J=6.9, 1.7 Hz, 1H), 3.85 (s, 314).

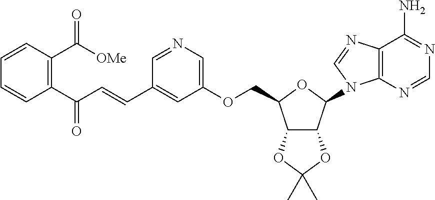 [SMILES: COC(=O)c1ccccc1C(=O)/C=C/c1cncc(OC[C@H]2O[C@@H](n3cnc4c(N)ncnc43)[C@@H]3OC(C)(C)O[C@@H]32)c1]

Methyl 2-((E)-3-(2-(((3aR,4R,6R,6aR)-6-(6-amino-9H-purin-9-yl)-2,2-dimethyltetrahydrofuro[3,4-d][1,3]dioxol-4-yl)methoxy)pyridin-4-yl)acryloyl)benzoate (M5) In a 10-mL round bottom flask, pyridinol M1 (78 mg, 0.275 mmol, 1 equiv), protected adenosine M13 (104 mg, 0.289 mmol, 1.05 equiv), and triphenylphosphine (75.8 mg, 0.289 mmol, 1.05 equiv) were suspended in a 1:1 mixture of DCM:THF (5 mL) and cooled to 0° C. DEAD (125.8 mg, 40% wt in toluene, 0.289 mmol, 1.0 equiv) was added dropwise, then the reaction was stirred for 22 h with warming to rt. After completion, the solvent was removed by rotary evaporation. Purification by silica flash chromatography (0→5% MeOH in DCM). Desired product co-eluted with starting material M13. The product mixture was purified using preparative HPLC (5→95% acetonitrile in water) to yield the DMF-deprotected product M5 as a waxy white solid (75 mg, 48%).

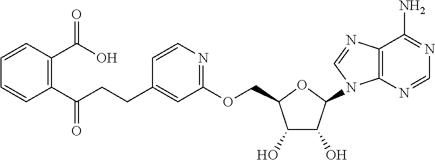 [SMILES: Nc1ncnc2c1ncn2[C@@H]1O[C@H](COc2cc(CCC(=O)c3ccccc3C(=O)O)ccn2)[C@@H](O)[C@H]1O]

2-(3-(2-(((3aR,4R,6R,6aR)-6-(6-ainino-9H-purin-9-yl)-2,2-dintethyltetrahydrofuro[3,4-d][1,3]dioxol-4-yl)methoxy)pyridin-4-yl)propanoyl)benzoic acid (M9) in a 1-dram vial the protected substrate M5 (17 mg, 0.027 mmol) was dissolved in a 0.5 mL MeOH and 0.5 mL of 20% aqueous H₂SO₄. The reaction stirred at room temperature was monitored by LCMS. At 14 hr the reaction mixture was basified to pH 14 with the addition of neat LiOH. After 5 minutes the ester was completely hydrolyzed and the reaction acidified to pH 2 with TFA. The reaction mixture was degassed and palladium on carbon (10% wt, 29 mg, 0.027 mmol, 1 equiv) was added. The reaction vial was sealed with a septum cap and placed under hydrogen at atmospheric pressure. After 4 hours, the reaction mixture was purged with argon and filtered over celite. The solvent was removed by rotary evaporation and the crude product was purified using preparative HPLC (5→95% acetonitrile in water) to yield desired product M9 as a white solid (2 mg, 14% over 3 steps).

### Example 3C

### Synthesis of m-3-Alethylpheityl Ether Analogue (M10)

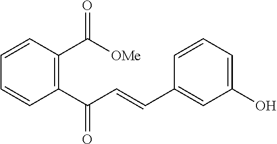 [SMILES: COC(=O)c1ccccc1C(=O)/C=C/c1cccc(O)c1]

Methyl (E)-2-(3-(3-hydroxy-5-methylphenyl)acryloyl)henzoate (M2) In a 0.5-2 mL microwave vial, vinyl ketone 35 (102 mg, 0.536 mmol, 1 equiv), 3-bromo-5-methylphenol (150 mg, 0.804 mmol, 1.5 equiv), NBu₄Cl (15 mg, 0.054 mmol, 0.1 equiv), and PdCl₂(dtbpf) (35 mg, 0.054 mmol, 0.1 equiv) were suspended in DMA (2 mL). NCy₂Me (157 mg, 0.804 mmol, 1.5 equiv) was added, the reaction vessel sealed, and the reaction stirred vigorously at 140° C. in a Biotage Initiator microwave for 15 min. The reaction was cooled to rt and filtered over celite. Solvent was removed via rotary evaporation. Purification by silica flash chromatography (50%→100% EtOAc in hexanes) yielded pyridinol M2 as a peach colored solid (34 mg, 22%). ¹H NMR (600 MHz, CDCl₃) δ 8.02 (dd, J=7.8, 1.3 Hz, 1H), 7.65 (td, J=7.5, 1.3 Hz, 1H), 7.58 (td, J=7.7, 1.3 Hz, 1H), 7.46 (dd, J=7.5, 1.3 Hz, 1H), 7.16 (d, J=16.2 Hz, 1H), 7.01 (d, J=16.2 Hz, 1H), 6.91 (s, 1H), 6.82 (t, J=1.9 Hz, 1H), 6.72 (s, 1H), 4.77 (s, 1H), 3.85 (s, 3H), 2.33 (s, 3H). ¹³C NMR (151 MHz, CDCl₃) δ 196.49, 166.89, 155.80, 145.28, 141.76, 140.41, 135.80, 132.25, 130.15. 129.31, 127.64, 122.36, 118.57, 111.70, 52.52, 21.25.

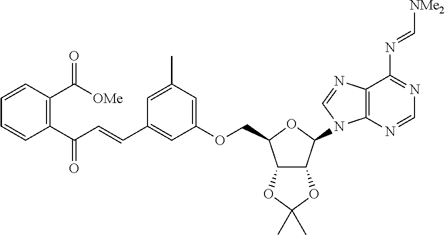 [SMILES: COC(=O)c1ccccc1C(=O)/C=C/c1cc(C)cc(OC[C@H]2O[C@@H](n3cnc4c(/N=C/N(C)C)ncnc43)[C@@H]3OC(C)(C)O[C@@H]32)c1]

Methyl 2-((E)-3-(3-(((3aR,4R,6R,6aR)-6-(6-(((E)-(dimethylamino)methylene)amino)-9H-purin-9-yl)-2,2-dimethyltetrahydrofuro[3,4-d][1,3]dioxol-4-yl)methoxy)-5-methylphenyl)acryloyl)benzonte (M6) In a 10-mL round bottom flask, phenol M2 (110 mg, 0.371 mmol, 1 equiv), protected adenosine M13 (141 mg, 0.389 mmol, 1.05 equiv), and triphenylphosphine (102 mg, 0.389 mmol, 1.05 equiv) were suspended in a 1:1 mixture of DCM:THF (5 mL) and cooled to 0° C. DEAD (170 mg, 40% wt in toluene, 0.389 mmol, 1.0 equiv) was added dropwise, then the reaction was stirred for 22 h with warming to rt. After completion, the solvent was removed by rotary evaporation. Purification by silica flash chromatography (0→10% MeOH in DCM) to yield product M6 as an orange, glassy solid (193 mg, 81%).

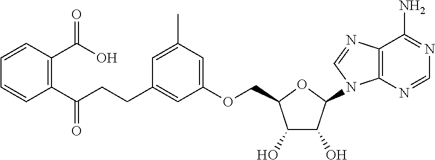 [SMILES: Cc1cc(CCC(=O)c2ccccc2C(=O)O)cc(OC[C@H]2O[C@@H](n3cnc4c(N)ncnc43)[C@H](O)[C@@H]2O)c1]

2-(3-(3-(((2R,3S,4R,5R)-5-(6-amino-9H-purin-9-yl)-3,4-dihydroxytetrahydrofuran-2-yl)methoxy)-5-methylphenyl)propanoyl)benzoic acid (M10) In a 1-dram vial the protected substrate M6 (10 mg, 0.016 mmol) was dissolved in a 0.5 mL MeOH and 0.5 mL of 20% aqueous H₂SO₄. The reaction stirred at room temperature was monitored by LCMS. At 14 hr the reaction mixture was basified to pH 14 with the addition of neat LiOH. After 5 minutes the ester was completely hydrolyzed and the reaction acidified to pH 2 with TFA. The reaction mixture was degassed and palladium on carbon (10% wt, 16.5 mg, 0.016 mmol. 1 equiv) was added. The reaction vial was sealed with a septum cap and placed under hydrogen at atmospheric pressure. After 4 hours, the reaction mixture was purged with argon and filtered over celite. The solvent was removed by rotary evaporation and the crude product was purified using preparative HPLC (5→95% acetonitrile in water) to yield desired product M10 as a white solid (3 mg, 35% over 3 steps).

### Example 3D

### Synthesis of m-2-Carboxyphenyl Ether Analogue (M11)

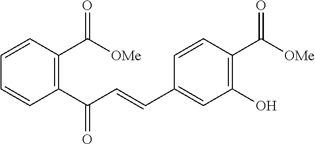 [SMILES: COC(=O)c1ccc(/C=C/C(=O)c2ccccc2C(=O)OC)cc1O]

Methyl (E)-2-hydroxy-4-(3-(2-(methoxycarbonyl)phenyl)-3-oxoprop-1-en-1-yl)benzoate (M3) In a 0.5-2 mL microwave vial, vinyl ketone 35 (150 mg, 0.788 mmol, 1 equiv), methyl 4-bromo-2-hydroxyhenzoate (273 mg, 1.182 mmol, 1.5 equiv), NBu₄Cl (22 mg, 0.079 mmol, 0.1 equiv). and PdCl₂(dtbpf) (52 mg, 0.079 mmol, 0.1 equiv) were suspended in DMA (2 NCy₂Me (231 mg, 1.182 mmol, 1.5 equiv) was added, the reaction vessel sealed, and the reaction stirred vigorously at 140° C. in a Biotage Initiator microwave for 15 min. The reaction was cooled to rt and filtered over celite. Solvent was removed via rotary evaporation. Purification by silica flash chromatography (50%→100% EtOAc in hexanes) yielded pyridinol M3 as a an off white solid (89 mg, 33%).

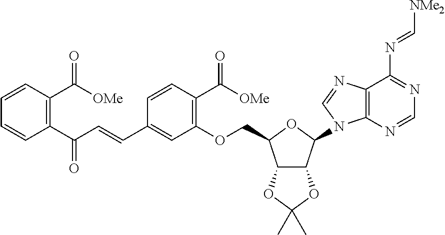 [SMILES: COC(=O)c1ccc(/C=C/C(=O)c2ccccc2C(=O)OC)cc1OC[C@H]1O[C@@H](n2cnc3c(/N=C/N(C)C)ncnc32)[C@@H]2OC(C)(C)O[C@@H]21]

Methyl 2-(((3aR,4R,6R,6aR)-6-(6-(((E)-(dimethylamino)methylene)amino)-9H-purin-9-yl)-2,2-dimethyltetrahydrefuro[3,4-d][1,3]dioxol-4-yl)methoxy)-4-((E)-3-(2-(methoxycarbonyl)phenyl)-3-oxoprop-1-en-1-yl)benzoute (M7) In a 10-ml, round bottom flask, phenol M3 (50 mg, 0.147 mmol, 1 equiv), protected adenosine M13 (58 mg, 0.162 mmol, 1.05 equiv). and triphenylphosphine (42 mg, 0.162 mmol, 1.05 equiv) were suspended in a 1:1 mixture of DCM:THF (5 mL) and cooled to 0° C. DEAD (70 mg, 40% wt in toluene, 0.162 mmol, 1.0 equiv) was added dropwise, then the reaction was stirred for 22 h with warming to rt. After completion, the solvent was removed by rotary evaporation. Purification by silica flash chromatography (0→5% MeOH in DCM) to yield product M7 as an off-white solid (66 mg, 66%).

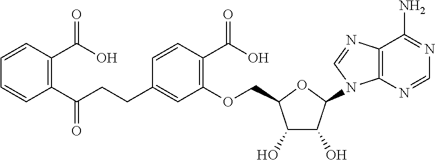 [SMILES: Nc1ncnc2c1ncn2[C@@H]1O[C@H](COc2cc(CCC(=O)c3ccccc3C(=O)O)ccc2C(=O)O)[C@@H](O)[C@H]1O]

2-(((2R,3S,4R,5R)-5-(6-amino-9H-purin-9-yl)-3,4-dihydroxytetrahydrofuran-2-yl)methoxy)-4-(3-(2-carboxyphenyl)-3-oxopropyl)benzoic acid (M11) In a 1-dram vial the protected substrate M7 (60 mg, 0.087 mmol) was dissolved in a 0.5 mL MeOH and 0.5 mL of 20% aqueous H₂SO₄. The reaction stirred at room temperature was monitored by LCMS. At 14 hr the reaction mixture was basified to pH 14 with the addition of neat LiOH. After 5 minutes the ester was completely hydrolyzed and the reaction acidified to pH 2 with TFA. The reaction mixture was degassed and palladium on carbon (10% wt, 92 mg, 0.097 mmol, 1 equiv) was added. The reaction vial was sealed with a septum cap and placed under hydrogen at atmospheric pressure. After 4 hours, the reaction mixture was purged with argon and filtered over celite. The solvent was removed by rotary evaporation and the crude product was purified using preparative HPLC (5→95% acetonitrile in water) to yield desired product M11 as a white solid (7 mg, 14% over 3 steps).

### Example 3E

### Synthesis of m-2-Carbmethoxyphenyl Ether Analogue (M14)

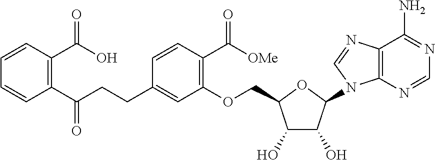 [SMILES: COC(=O)c1ccc(CCC(=O)c2ccccc2C(=O)O)cc1OC[C@H]1O[C@@H](n2cnc3c(N)ncnc32)[C@H](O)[C@@H]1O]

2-(3-(3-(((2R,3S,4R,5R)-5-(6-amina-9H-purin-9-yl)-3,4-dihydroxytetrahydrofuran-2-yl)methoxy)-4-(methoxycarbonyl)phenyl)propanoyl)benzoic acid (M14) is made by the general scheme below.

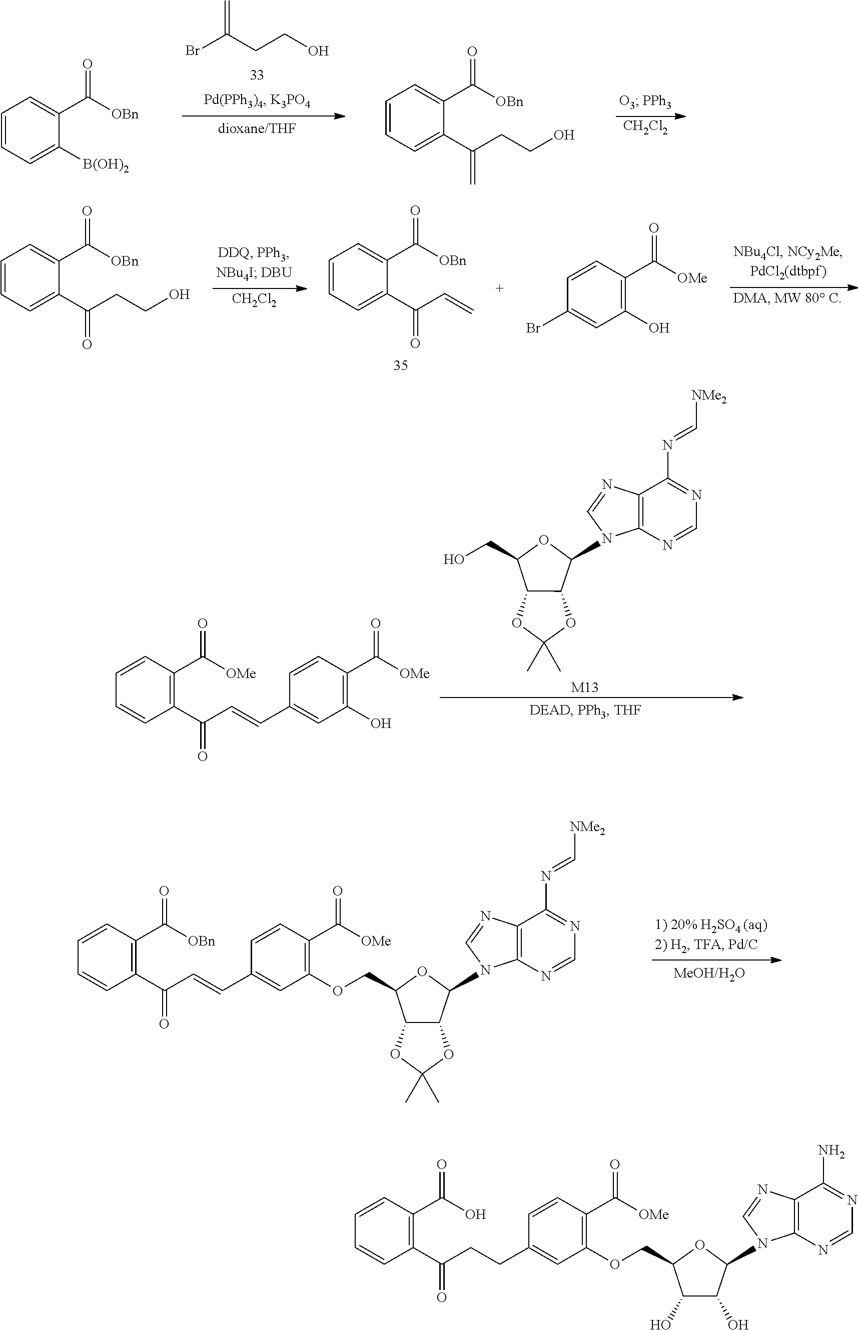

### Example 3E

### Synthesis of m-2-aminaphenyl Ether Analogue (M12)

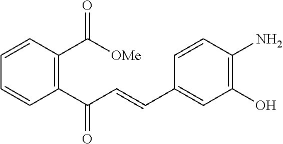 [SMILES: COC(=O)c1ccccc1C(=O)/C=C/c1ccc(N)c(O)c1]

Methyl (E)-2-(3-(4-amino-3-hydroxyphenyl)acryloyl)benzoate (M4) hi a 0.5-2 mL microwave vial, vinyl ketone 35 (170 mg, 0.894 mmol, 1 equiv), tert-butyl N-(4-bromo-2-hydroxyphenyl)carbamate (386 mg, 1.340 mmol, 1.5 equiv), NBu₄Cl (25 mg, 0.089 mmol, 0.1 equiv), and PdCl₂(dtbpf) (58 mg, 0.089 mmol, 0.1 equiv) were suspended in DMA (2 mL). NCy₂Me (262 mg, 1.340 nunol, 1.5 equiv) was added, the reaction vessel sealed, and the reaction stirred vigorously at 140° C. in a Biotage Initiator microwave for 15 min. The reaction was cooled to rt and filtered over celite. Solvent was removed via rotary evaporation. Purification by silica flash chromatography (50%→100% EtOAc in hexanes) yielded phenol M4 as an orange solid (62 mg, 33%).

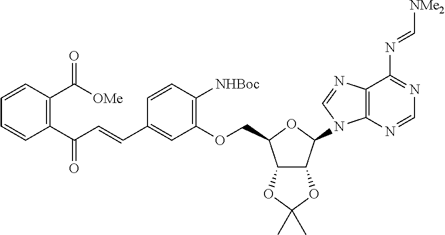 [SMILES: COC(=O)c1ccccc1C(=O)/C=C/c1ccc(NC(=O)OC(C)(C)C)c(OC[C@H]2O[C@@H](n3cnc4c(/N=C/N(C)C)ncnc43)[C@@H]3OC(C)(C)O[C@@H]32)c1]

Methyl 2-((E)-3-(4-((tert-butoxycarbonyl)amino)-3-(((3aR,4R,6R,6aR)-6-(6-(((E)-(dimethylainino)methylene)amino)-9H-purin-9-yl)-2,2-dimethyltetrahydrofuro[3,4-d][1,3]dioxol-4-yl)methoxy)phenyl)acryloyl)benzoate (M8) In a 10-mL round bottom flask, phenol M4 (34 mg, 0.087 mmol, 1 equiv), protected adenosine M13 (33 mg, 0.091 mmol, 1.05 equiv), and triphenylphosphine (24 mg, 0.091 mmol, 1.05 equiv) were suspended in a 1:1 mixture of DCM:THF (5 mL) and cooled to 0° C. DEAD (40 mg, 40% wt in toluene, 0.091 mmol, 1.0 equiv) was added dropwise, then the reaction was stirred for 22 h with warming to rt. After completion, the solvent was removed by rotary evaporation. Purification by silica flash chromatography (0→5% MeOH in DCM) to yield product M9 as a yellow semi-solid (18 mg, 28%).

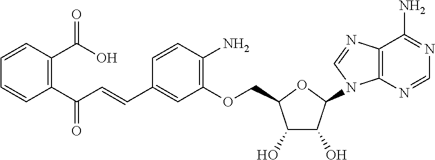 [SMILES: Nc1ccc(/C=C/C(=O)c2ccccc2C(=O)O)cc1OC[C@H]1O[C@@H](n2cnc3c(N)ncnc32)[C@H](O)[C@@H]1O]

2-((E)-3-(4-amino-3-(((2R,3S,4R,5R)-5-(6-amino-9H-purin-9-yl)-3,4-dihydroxytetrahydrofuran-2-yl)methoxy)phenyl)acryloyl)benzoic acid (M12) In a 1-dram vial the protected substrate M8 (58 mg, 0.078 mmol) was dissolved in a 0.5 mL MeOH and 0.5 mL of 20% aqueous H₂SO₄. The reaction stirred at room temperature was monitored by LCMS. At 22 hr the reaction mixture was basifted to pH 14 with the addition of neat LiOH. After 5 minutes the ester was completely hydrolyzed and the reaction acidified to pH 2 with TFA. The reaction mixture was degassed and palladium on carbon (10% wt, 16.5 mg, 0.016 mmol, 0.2 equiv) was added. The reaction vial was sealed with a septum cap and placed under hydrogen at atmospheric pressure. After 4 hours, the reaction mixture was purged with argon and filtered over celite. The solvent was removed by rotary evaporation and the crude product was purified using preparative HPLC (5 95% acetonitrile in water) to yield desired product M12 as a white solid (10 mg, 24% over 3 steps).

### Example 4

### Purification of Wild-Type and K437.4 E. coil MenE

E. coli MenE was purified as described previously (Lu, X. et al. ChemBioChem 2012, 13, 129). Briefly, a pET15b plasmid containing E. coli MenE with an N-terminal His6 tag was transformed into E. coli BL21(DE3) pLysS cells that were then grown overnight in 10 mL LB media containing 200 μg/mL ampicillin. Next, the overnight culture was inoculated into 1 L of LB media containing same concentration of ampicillin and the cells were grown until the 0D600 reached 0.6. Protein expression was induced by addition of 1 mM IPTG and the culture was shaken overnight at 20° C. Cells were harvested by centrifugation at 5,000 rpm for 10 min at 4° C. The cell pellet was resuspended in 30 mL of His-binding buffer (20 mM TrisHCl, 100 mM NaCl, 5 mM imidazole, pH 8.0) and the bacteria were disrupted by sonication. Cell debris was removed by centrifugation at 40,000 rpm for 60 min at 4° C. and the clear supernatant was loaded onto a Hisbind column (1.5 cm×15 cm) containing 4 mL His-bind resin (Novagen) that had been charged with 10 mL of charge buffer (Ni²⁺). The column was washed with washing buffer containing 60 mM imidazole and the protein was eluted from the column with elution buffer containing 500 mM imidazole. Fractions containing protein were loaded onto a size-exclusion column (Superdex 75, GE Healthcare) and eluted using 20 mM TrisHCl, pH 8.0 buffer containing 100 mM NaCl in order to remove imidazole. The purified protein was >97% pure by SDS-PAGE and was stored at 80° C. in storage buffer consisting of 20 mM TrisHCl, pH 8.0, containing 100 mM NaCl.

### Example 5

### Site-Directed Mutagenesis

Based on the nucleotide sequence of MenE (from E. coli strain K-12), the forward primer with the sequence of 5′-AACGGCGGTATTGCGATITCACG-3′ and T7 reverse primer were designed, synthesized, and used to introduce the K437A mutation in E. coli MenE. High-fidelity Phusion polymerase (New England BioLabs) was used for standard polymerase chain reaction amplification and mutagenesis.

### Example 6

### MenE Biochemical Assay

Enzyme inhibition studies were performed in 20 mM NaHPO₄ buffer (pH 7.4) containing 150 mM NaCl and 1 mM MgCl₂ using a MenE-MenD coupled assay in which MenE is rate-limiting. IC₅₀ values were determined in reaction mixtures containing OSB (60 μM), ATP (240 μM), CoA (240 μM), mtMenB (2.5 μM), and varying inhibitor concentrations (5-250 μM). Reactions were initiated by addition of ecMenE (50 nM), and the production of DHNA-CoA was monitored at 392 nm (ε392=4000 M⁻¹ cm⁻¹).

With these linker analogues in hand, their inhibition of E. coli MenE using the previously reported MenE-Meng coupled assay was evaluated (Lu, X. et al. Bioorg. Med. Chem. Lett. 2008, 18, 5963). Consistent with predictions from the docking experiments, all but one of the analogues inhibited MenE, albeit with modest IC₅₀ values compared to OSB-AMS (1) (Table 2). The m-phenyl ether analogue 5 was the most potent inhibitor, with an IC₅₀ of 8.1±0.9 μM, while the corresponding 3-trifluoromethyl-substituted analogue 8 exhibited slightly weaker inhibition. In contrast, the p-phenyl ether analogue 6 showed no inhibition of MenE, consistent with its docking score of >−10 kcal/mol. Taken together, these results demonstrated that computational docking could be used effectively to prioritize OSB-AMS analogues for synthesis and biochemical evaluation against MenE.

TABLE 2
Enzyme Inibition and Docking Scores

| Analogue | Linker | IC₅₀ (μM) | Docking Score (kcal/mol) |
|---|---|---|---|
| 1 | 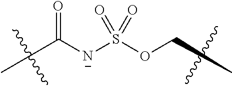 | 0.024 ± 0.003 | −13.78 |
| 5 | 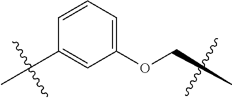 | 8.1 ± 0.9b | −14.03 |
| 6 | 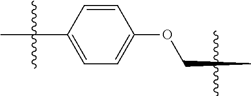 | >200d | >−10.00 |
| 8 | 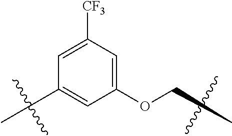 | 26.5 ± 2.3 | −12.80 |

### Example 7

### Isothermal Titration Calorimetry

ITC was performed using VP-ITC instrument at 22° C. Inhibitor (1 mM) and E. coli MenE(wt) (30 μM) were prepared in 20 mM NaHPO₄, pH 7.4 buffer containing 150 mM NaCl and 1 mM MgCl₂. From the injection syringe, the inhibitor was titrated into the sample cell containing 1.8 mL solution of MenE. The data were fit to a single-binding site model with the Origin software package.

Next. the binding affinity of the 5 with E. coli MenE was deteremined using isothermal titration calorimetry (ITC). The measured Kd values of m-phenyl ether analogue 5 (244±11 nM) was generally consistent with IC₅₀ value determined in the biochemical assay. In contrast, p-phenyl ether analogue 6, which did not inhibit MenE, also showed no binding in the ITC assay.

### Example 8

### Minimum Inhibitory Concentration

Minimum inhibitory concentrations (MICs) were determined using visual growth inspection of cells grown in transparent 96-well plates. E. coli (MG1655), MRSA (ATCC BAA-1762), and Bacillus subtilis (ATCC 6057) were grown to mid-log phase (OD600 0.6-0.8) in cation-adjusted Miller Hinton (CAMH) media at 37° C. in an orbital shaker. A final inoculum of 100 μL of 10⁶ CFU/mL cells was treated with inhibitor with final concentrations ranging from 0.2-100 μg/mL. The MIC was defined as the minimum concentration at which a well showed no obvious growth by visual inspection.

All of the synthesized analogues were evaluated for antibacterial activity against E. coli, MRSA, and B. subtilis. Unfortunately, none of the compounds exhibited an MIC lower than 100 μg/mL, compared to the MIC of 31.25 μg/mL observed for OSB-AMS (1) against MRSA (ATCC BAA1762) (Matarlo, J. S. et al. Biochemistry 2015, 54, 6514). This lack of antimicrobial activity may be due to the decreased biochemical potency of these linker analogues in comparison to OSB-AMS (1), which may be improved in the future through further optimization of these newly discovered chemotypes.

### Example 9

### X-ray Codystal Structure of m-Phenyl Ether Analogue 5 Bound to E. coli MenE

Wild-type E. coli MenE was prepared as previously described (Lu, X. et al. ChemBioChem 2012, 13, 129). Cocrystallization of analogue 5 with MenE was achieved using the hanging drop diffusion technique, in which 2 μL of the complex solution (10 mg/mL E. coli MenE and 600 μM analogue 5 dissolved in 20 mM Tris-HCl, pH 8.0 buffer containing 100 mM NaCl) was mixed with 2 μL of reservoir solution (0.1 M TrisHCl, 0.2 M MgCl₂, 20% PEG at pH 6.5) and equilibrated against 300 μL of reservoir solution. Crystals formed at 293.15 K after 4-5 days. A single crystal was selected for diffraction by flash freezing directly from the crystallization solution. Diffraction data were collected at 100 K using beamline 17-ID-1 (AMX) at the National Synchrotron Light Source II at Brookhaven National Laboratory. Data were integrated using XDS (Kabsch, W. Acta Crystallogr. D Biol. Crystallogr. 2010, 66, 125) and scaled using Aimless (Evans, P. R. et al. Acta Crystallogr. D Biol. Crystallogr. 2013, 69, 1204). The structure was solved by molecular replacement with MolRep (Vagin, A. et al. Acta Crytstallogr. D Biol. Crystallogr. 2010, 66, 2), using a previously solved E. coli MenE (R195K) structure (PDB entry 5C₅H) as a search model. The model was refined through successive rounds of manual model building using COOT (Emsley, P. Acta Crystallogr. D Biol. Crystallogr. 2004, 60, 2126) and restrained refinement using REFMAC5 (Murshudov, G. N. et al. Acta Crystallogr. D Biol. Crystallogr. 1997, 53, 240). Electron density for analogue 5 bound in the active site was clearly visible and was added directly to the difference Fourier map after refinement converged. Ligand restraints were generated using the PRODRG server (Schuttelkopf, A. W et al. Acta Crystallogr. D Biol. Crystallogr. 2004, 60, 1355). To compare the binding poses of cocrystallized OSB-AMS (1) (PDB entry: 5C5H) and docked and cocrystallized m-phenyl ether analogue 5, the N-terminal domains (residues 1-351) of the structures were aligned using PyMol (align command, default parameters) (Schrodinger, L. “The PyMOL molecular graphics system, Version 1.8. 2015.” 2017).

To understand the binding of m-phenyl ether analogue 5 and to provide guidance for further inhibitor design, the cocrystal structure of 5 bound to wildtype E. coli MenE was solved at 1.8 Å resolution. The structure was determined by molecular replacement using a previously reported structure of OSB-AMS (1) bound E. coli MenE (R195K) (PDB entry: 5C₅H) (Matadi), J. S. et al. Biochemistry 2015, 54, 6514) as a search model.

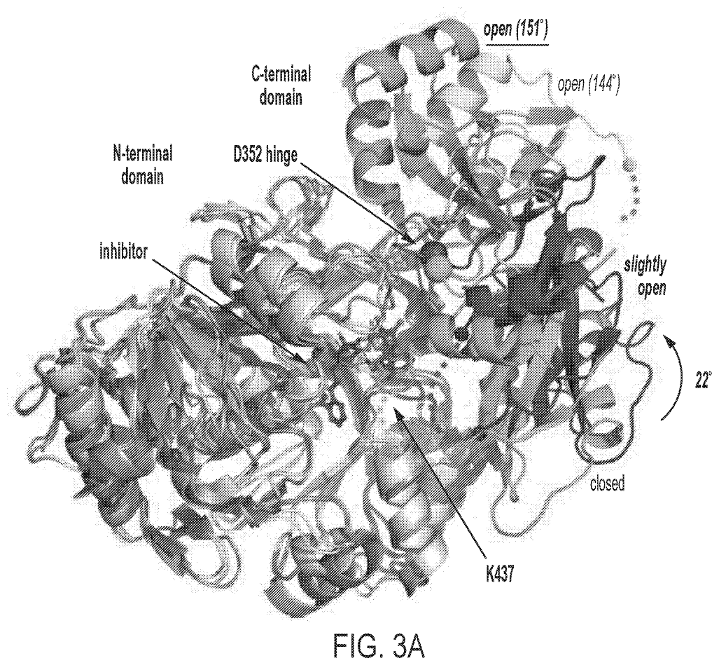

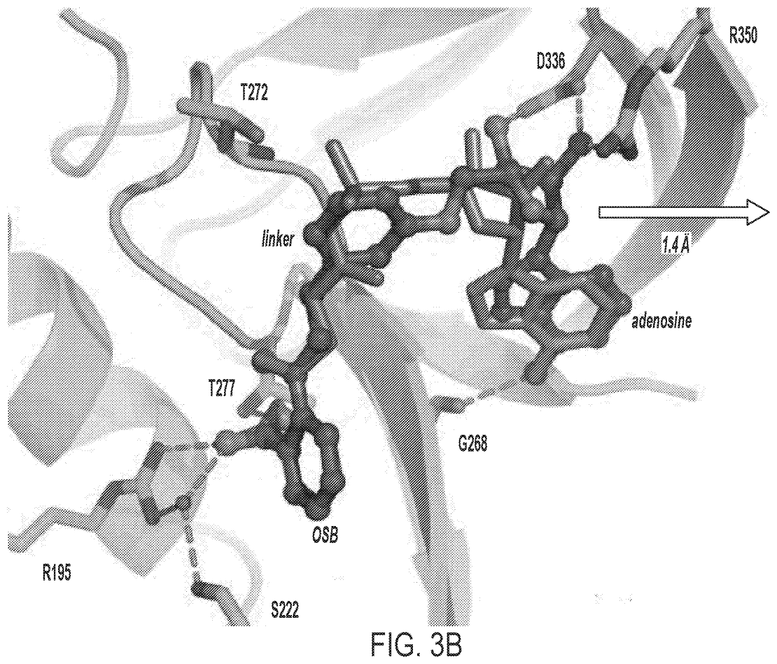

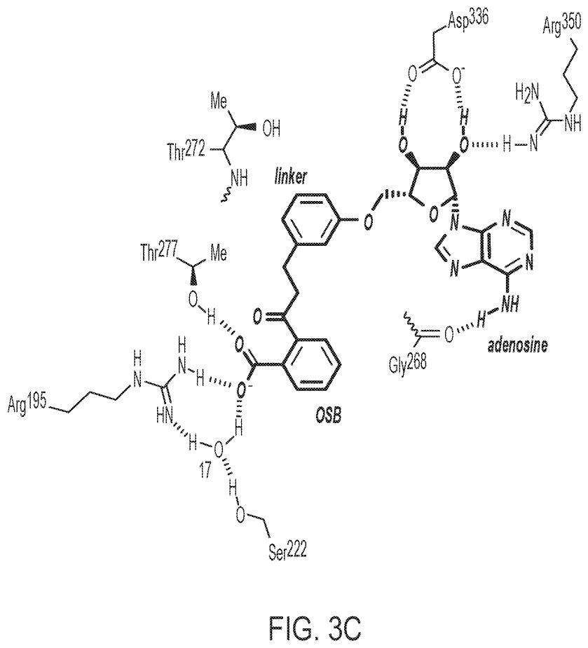

Comparison of the structures of m-phenyl ether analogue 5 bound to wild-type MenE and OSB-AMS (1) bound to MenE (R195K) revealed differences in the orientation of the small C-terminal domain relative to the large N-terminal domain (FIG. 3a). In the structure with m-phenyl ether analogue 5, the C-terminal domain is rotated by 22° (determined using dynDoni53) about a hinge residue D352, away from the active site, making it “slightly open” relative to the “closed” conformation seen in the structure with OSB-AMS (1). Notably, apo structures of S. aureus MenE have been reported previously (Patskovsky, Y.; Protein Data Bank, 31PL; doi: 10.2210/pdb3IPL/pdb) and the C-terminal domains are rotated much more dramatically, by 144° and 151°, in a fully “open” conformation. In contrast, both apo and liganded structures of Bacillus subtilis MenE have also been reported, all in the “closed” conformation (Chen, Y. et al. J. Biol. Chem. 2015, 290, 23971; Chen, Y. et al. Biochemistry 2016, 55, 6685). Both “open” and “closed” states have been observed previously for other members of the ANL family (Gulick, A. M. ACS Chem. Biol. 2009, 4, 811; ANL Super Family Solved Structures, http://www.acsu.buffalo. edu/˜amgulick/RANLChart.html (accessed Nov. 24, 2018)) with fully open states typically observed only in untiganded structures (Conti, E. et al. Structure 1996, 4, 287; Nakatsu, T. et al. Nature 2006, 440, 372; Hisanaga, Y. et al. J. Biol. Chem. 2004, 279, 31717; Andersson, C. S. et al. Structure 2012. 20, 1062; Vergnolle, O. et al. J. Biol. Chem. 2016, 291, 22315; Liu, Z. et al. J Biol. Chem. 2013, 288, 18473). In the closed conformations, a highly conserved lysine residue (K437 in E. coli MenE, K471 in B. subtilis MenE) (Stachelhaus, T. et al. Chem. Biol. 1999, 6, 493) serves as a lynchpin that coordinates the acyl-adenylate intermediate via its acyl group carbonyl, phosphate pro-S oxygen, ribose 4′-oxygen, and/or ribose 5′-oxygen. (Matarlo, J. S. et al. Biochemistry 2015, 54. 6514; Chen. Y. et al. Biochemistry 2016, 55, 6685; Nakatsu, T. et al. Nature 2006, 440, 372; Hisanaga, Y. et al. J. Biol. Chem. 2004, 279, 31717; Conti, E. et al. EMBO J. 1997, 16, 4174) In the OSB-AMS (1) MenE (R195K) complex, K437 is observed in the active site and binds OSB-AMS through these interactions. In contrast, in the complex of 5 bound to wild-type MenE, the loop region from residues 434-438 that includes K437 is disordered. The loss of the K437-ligand interaction may result in decreased force holding the C-terminal domain in the closed conformation, resulting in a slight rotation of the C-terminal domain away from the active site. Further, without the interaction of K437 with the ligand, the loop region becomes highly dynamic and disordered (Patskovsky, Y.; Protein Data Bank, 3IPL; doi: 10.2210/pdb3IPL/pdb; Chen, Y. et al. J. Biol. Chem. 2015, 290, 23971).

Comparison of these structures reveals how the C- and N-terminal domains may move relative to each other in the course of binding substrates or inhibitors. The ligand may bind to the protein in an open conformation and, subsequently, the small C-terminal domain rotates towards the active site of the large N-terminal domain, leading to an interaction between the ligand and K437 that stabilizes the closed state. Notably, Gulick has reported a distinct, ≈140° rotation about the hinge residue in coerystal structures of acetyl-CoA synthetase with the adenylate mimic propyl-AMP and the cosubstrate CoA, as well as of 4-chlorobenzoate-CoA synthetase with a product analogue 4-chlorophenacyl-CoA, which generates a second, distinct “closed” conformation (Gulick, A. M. et al. Biochemistry 2003, 42, 2866; Reger, A. S. el al. Biochemistry 2007, 46, 6536). This conformation has also been observed recently in the structure of B. suhtilis MenE with a stable OSB-CoA analogue (Chen, Y. et al. J. Biol. Chem. 2017, 292, 12296). This “domain alternation” introduces new residues into the active site to catalyze thioesterification in the second half-reaction. Thus, rotation of the C-terminal domain about the hinge residue may be involved in both substrate binding and catalysis of the first half-reaction as well as CoA binding and catalysis of the second half-reaction.

### Example 10

### Active Site Interactions Between M-Phenyl Ether Analogue 5 and E. coli MenE

The difference in the relative orientation of the C-terminal domain does not appear to impact the overall orientation of m-phenyl ether analogue 5 in the MenE active site compared to that of OSB-AMS (1) (FIG. 3b). However, the geometric constraints of the phenyl ether linker force the ribose moiety ≠1.4 Å deeper into the adenine region of the binding pocket.

Enzymeinhibitor interactions are highlighted in FIGS. 3b and 3c. Residues R195, S222, T277, G268 (carbonyl), D336 and R350 form hydrogen-bonding interactions with m-phenyl ether analogue S and these residues are conserved in E. coli, S. aureus, and M. tuberculosis MenE (Matarlo, J. S. et al. Biochemistry 2015, 54, 6514). In B. subtilis MenE, R195 is replaced by K205, T277 by Q294, G268 by S285 (Chen, Y. et al. J. Biol. Chem. 2015, 290, 23971). E. coli residues R195, S222, and T277 are clustered around the OSB aromatic carboxylate. Residues R195 and T277 interact directly with the carboxylate and R195 also engages in a second, water-mediated interaction via water-17, which is coordinated by S222. It had previously been proposed that a direct interaction with R195 should be present based on the analysis of the OSB-AMS (1) MenE (R 195K) crystal structure, in which the OSB carboxylate interacts with K195 via two water-mediated interactions (FIG. 2a). In contrast, in the structure of wild-type MenE complexed with 5 reported here, the R195 guanidinium group engages the OSB carboxylate via one direct interaction and one water-mediated interaction. In the linker region, no direct interactions are observed with the m-phenyl ether. Residue T272, which binds to the pro-S oxygen of the sulfarnate in OSB-AMS, remains within 4 Å of the aromatic ring of m-phenyl ether analogue 5. However, the lynchpin K437, which interacts with multiple atoms in the linker region of OSB-AMS, is disordered and not observed in the structure of m-phenyl ether analogue 5. In the ribose region, D336 interacts with the 2′- and 3′-hydroxyl groups of m-phenyl ether analogue 5 in the same way as OSB-AMS (1).

### Example 11

### Binding of m-Phenyl Ether Analogue 5 to a MenE (K437A) Lynchpin Mutant

Crucially, none of the predicted interactions between K437 and the linker region of m-phenyl ether analogue 5 were observed, because the residue 434-438 loop was disordered. While analogue 5 lacks the carbonyl and phosphate pro-S oxygen of the cognate intermediate OSB-AMP, K437 might still hydrogen bond with the ribose 4′- and/or 5′-oxygens and could also engage in a cation-it interaction with the phenyl ether ring, based on earlier docking predictions (FIG. 2b).

To probe for these interactions experimentally. the binding affinity of m-phenyl ether analogue 5 to wild-type E. coli MenE and the K437A mutant were compared. ITC titration curves show very similar K_{d} values of 249 and 244 nM, respectively, consistent with the lack of a productive binding interaction of K437 with m-phenyl ether analogue 5.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of die group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in hack verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any one of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (I): 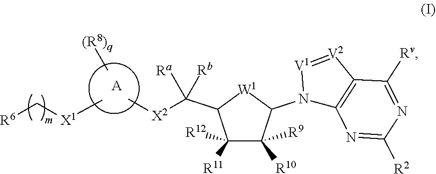
or a pharmaceutically acceptable salt, stereoisomer, or tautomer thereof, wherein:
V¹ is ═CR³or ═N—;
V² is ═CH or ═N;
R^{v} is N(R¹)₂, —OR¹, halogen, or hydrogen;
R¹ is hydrogen, optionally substituted alkyl, optionally substituted heteroalkyl, optionally substituted carbocyclyl, optionally substituted heterocyclyl, optionally substituted aryl, optionally substituted heteroaryl, or optionally substituted acyl, or two R¹ are joined to form an optionally substituted heterocyclic ring;
each of R² and R³ is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR^{e}, —N(R^{e})₂, —N₃, —SO₂H, —SO₃H, —SR^{e}, —SSR^{e}, —OC(═O)R^{e}, —OCO₂R^{e}, —OC(═O)N(R^{e})₂, —C(═O)N(R^{e})₂, —NC(═O)N(R^{e})₂, —OC(═O)O(R^{e})₂, —SO₂R^{e}, —SO₂OR^{e}, —OSO₂R^{e}, —S(═O)R^{e}, or —OS(═O)R^{e};
W¹ is —O—, —CR^{e}₂, —NR^{e}—, or —S—;
each of R⁹, R¹⁰, R¹¹, and R¹² is hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —NO₂, —CN, —OR⁴, —OR⁵, —OR^{e}, N(R^{e})₂, —N₃, —SO₂H, —SO₃H; —SR^{e}, —OC(═O)R^{e}, —C(═O)OR^{e}, —C(═O)N(R^{e})₂, —N(R^{e})C(═O)R^{e}, or —SO₂R^{e}, or two occurrences of any R⁹, R¹⁰, R¹¹, and R¹² are joined to form an optionally substituted carbocyclic ring or an optionally substituted heterocyclic ring;
each of R⁴ and R⁵ is independently hydrogen, optionally substituted C₁₋₆ alkyl, optionally substituted acyl, or an oxygen protecting group, or R⁴ and R⁵ are joined to form an optionally substituted heterocyclic ring;
each of R^{a} and R^{b} is independently hydrogen, halogen, optionally substituted C₁₋₆ alkyl, —OR^{e}, or N(R^{e})₂;
X¹ is a bond, —C(R^{e})₂, —O—, or —NR^{e}—;
X² is a bond, —C(R^{e})₂—, —O—, or —NR^{e}—;
R⁶ is of the formula: 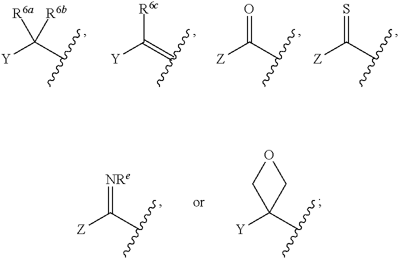
Ring A is phenylene, pyridinylene, or pyrimidinylene;
each of Y and Z is independently optionally substituted alkyl, optionally substituted alkenyl, optionally substituted alkynyl, optionally substituted heteroalkyl, optionally substituted heteroalkenyl, optionally substituted heteroalkynyl, optionally substituted alkoxy, optionally substituted amino, —OR^{e}, —N(R^{e})₂, optionally substituted carbocyclyl, optionally substituted heterocyclyl, optionally substituted aryl, or optionally substituted heteroaryl;
each of R^{6a}, R^{6b}, and R^{6c} is independently hydrogen, halogen, optionally substituted C₁₋₆ alkyl, —OR^{e}, or —N(R^{e})₂;
each occurrence of R^{e} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, an oxygen protecting group when attached to an oxygen atom, a nitrogen protecting group when attached to a nitrogen atom, or a sulfur protecting group when attached to a sulfur atom, or two R^{e} are joined to form an optionally substituted carbocyclic, an optionally substituted aryl, an optionally substituted heterocyclic, or optionally substituted heteroaryl ring;
each occurrence of R⁸ is independently halogen, optionally substituted alkyl, haloalkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —COOR^{e}, —CON(R^{e})₂, —NO₂, —CN, —OR^{e}, or —N(R^{e})₂, or two R⁸ are joined to form an optionally substituted carbocyclyl ring, optionally substituted heterocyclyl ring, optionally substituted aryl, or optionally substituted heteroaryl ring; and
m is 0, 1, 2, 3, 4, 5, or 6; and
q is 0, 1, 2, 3, or 4.

2. The compound of claim 1, wherein the compound is of: (a) Formula (I-A): 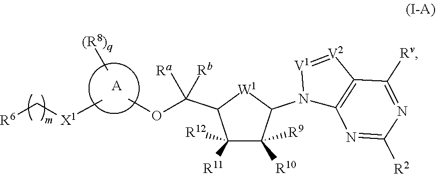
or a pharmaceutically acceptable salt, stereoisomer, or tautomer thereof; (b) Formula (I-B): 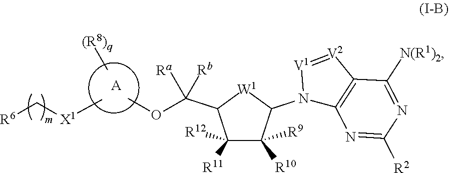
or a pharmaceutically acceptable salt, stereoisomer, or tautomer thereof; (c) Formula (II): 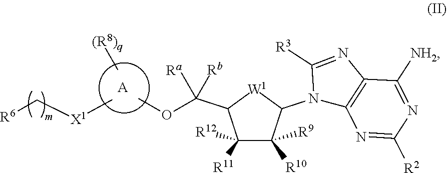
or a pharmaceutically acceptable salt, stereoisomer, or tautomer thereof; (d) Formula (III): 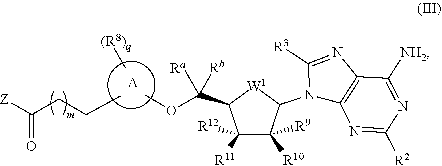
or a pharmaceutically acceptable salt, stereoisomer, or tautomer thereof; (e) Formula (IV): 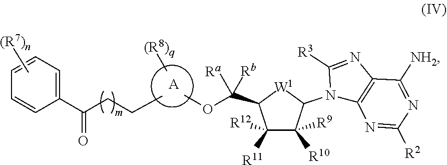
or a pharmaceutically acceptable salt, stereoisomer, or tautomer thereof, wherein:
each occurrence of R⁷ is independently is halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —COOR^{e}, —CON(R^{e})₂, —NO₂, —CN, —OR^{e}, or —N(R^{e})₂, or two R⁷ are joined to form an optionally substituted carbocyclyl, optionally substituted heterocyclyl, optionally substituted aryl, or optionally substituted heteroaryl ring; and
n is 0, 1, 2, 3, 4, or 5;
(f) Formula (V): 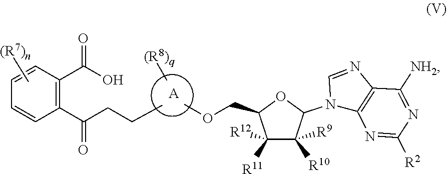
or a pharmaceutically acceptable salt, stereoisomer, or tautomer thereof, wherein;
each occurrence of R⁷ is independently is halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —COOR^{e}, —CON(R^{e})₂, —NO₂, —CN, —OR^{e}, or —N(R^{e})₂, or two R⁷ are joined to form an optionally substituted carbocyclyl, optionally substituted heterocyclyl, optionally substituted aryl, or optionally substituted heteroaryl ring; and
n is 0, 1, 2, 3, 4, or 5; or
(g) Formula (VI): 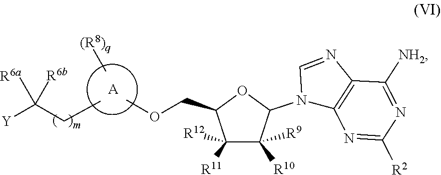
or a pharmaceutically acceptable salt, stereoisomer, or tautomer thereof.

3-11. (canceled)

12. The compound of claim 1, wherein the compound is of the formula (VI-B): 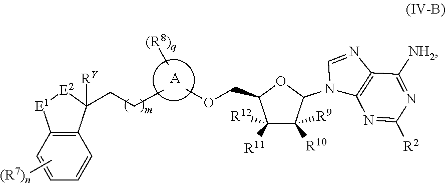
or a pharmaceutically acceptable salt, stereoisomer, or tautomer thereof, wherein:
E¹ is —C(═O)—, —C(═S)—, —C(═NR^{f})—, —C(R^{E1})₂—, —O—, or —NR^{f}—; and each R^{E1} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, —SR^{e}, or —N(R^{e})₂;
E² is —C(═O—, —C(═S)—, —C(═NR^{f})—, —C(R^{E2})₂—, —O—, or —NR^{f}—; and each R^{E2} is independently hydrogen, halogen, optionally substituted alkyl, optionally substituted alkenyl, optionally substituted alkynyl, optionally substituted carbocyclyl, optionally substituted heterocyclyl, optionally substituted aryl, optionally substituted heteroaryl, optionally substituted acyl, —OR^{e}, —SR^{e}, or —N(R^{e})₂;
each R^{f} is independently hydrogen, optionally substituted C₁₋₆ alkyl, optionally substituted acyl, or a nitrogen protecting group; and
R^{Y} is hydrogen, halogen, optionally substituted alkyl, optionally substituted alkenyl, optionally substituted alkynyl, —OR^{e}, or —N(R^{e})₂.

13-30. (canceled)

31. The compound of claim 1, wherein Y or Z is of formula

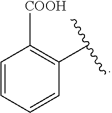 [SMILES: *c1ccccc1C(=O)O]

32-33. (canceled)

34. The compound of any one of claim 1, wherein at least one of Y or Z is of the formula

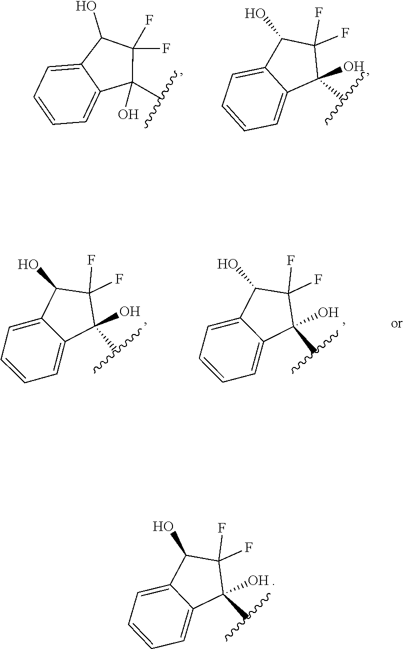 [SMILES: *C1(O)c2ccccc2C(O)C1(F)F ; *[C@]1(O)c2ccccc2[C@H](O)C1(F)F ; *[C@]1(O)c2ccccc2[C@@H](O)C1(F)F ; *[C@@]1(O)c2ccccc2[C@H](O)C1(F)F ; *[C@@]1(O)c2ccccc2[C@@H](O)C1(F)F]

35-39. (canceled)

40. The compound of claim 1, wherein X¹ is attached to Ring A meta to X²; or X¹ is attached to Ring A para to X².

41. (canceled)

42. The compound of claim 1, wherein Ring A is phenylene or pyridinylene.

43-46. (canceled)

47. The compound of claim 1, wherein the compound is of the formula: 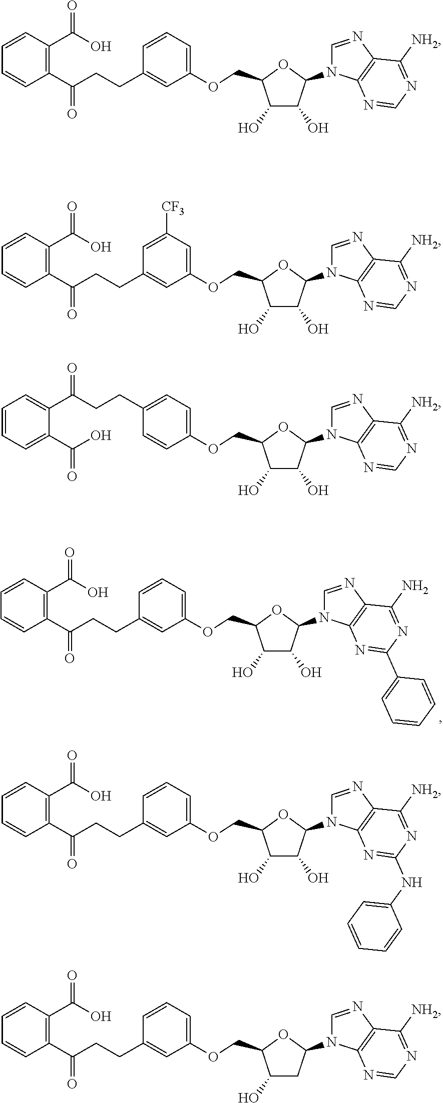 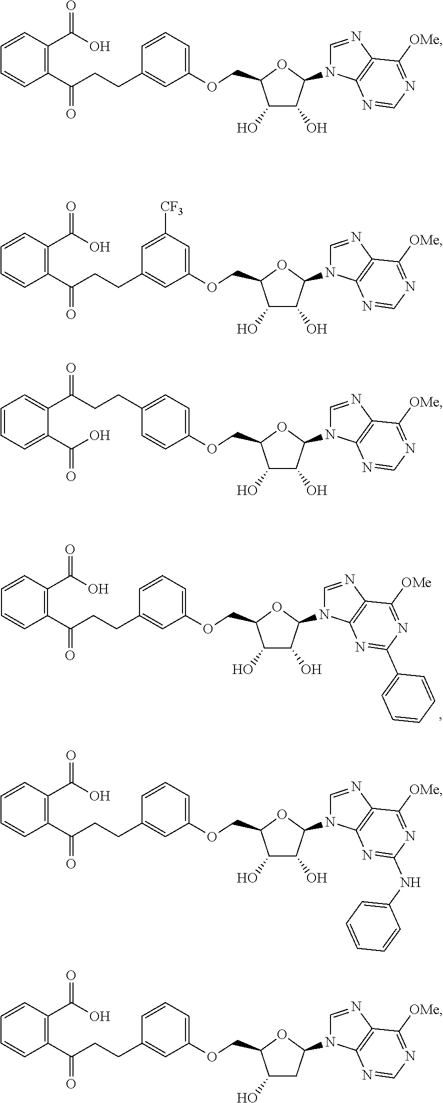 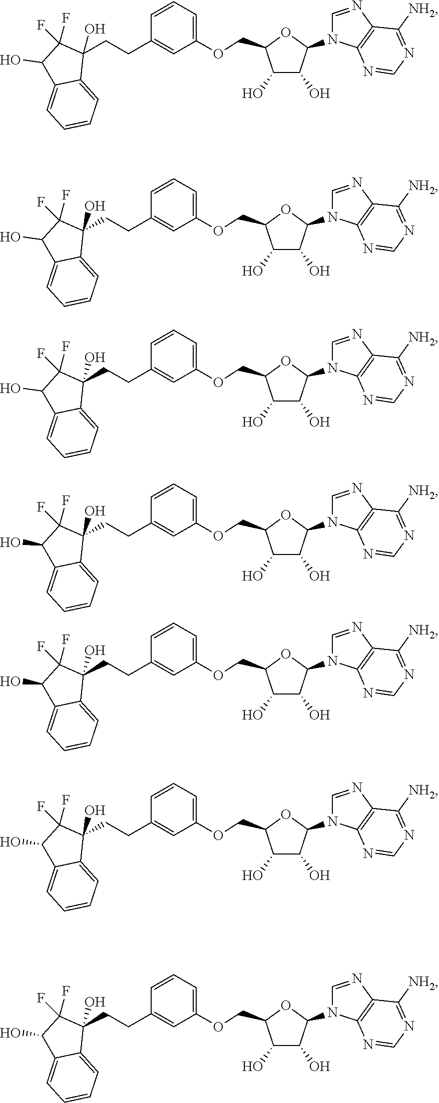 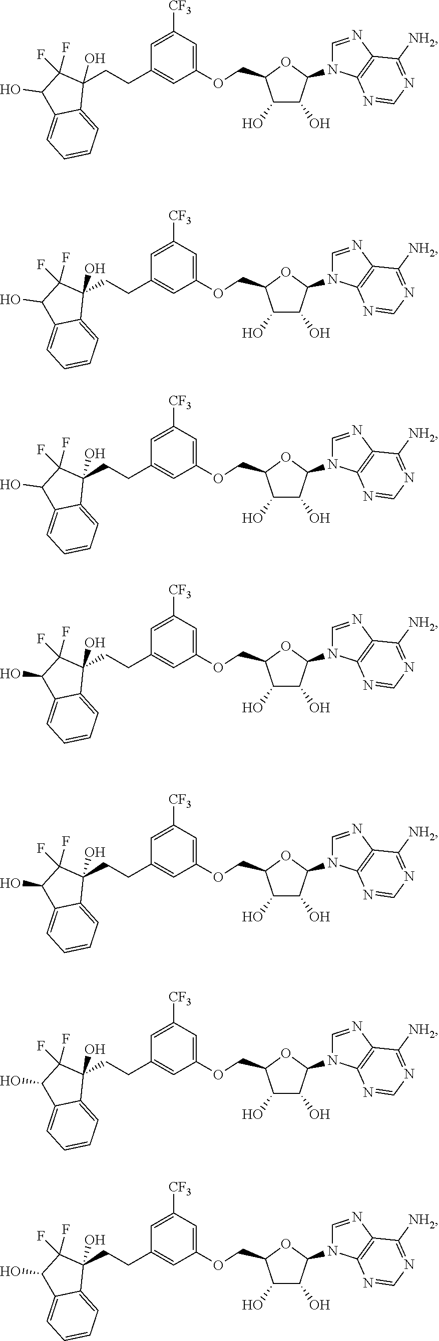 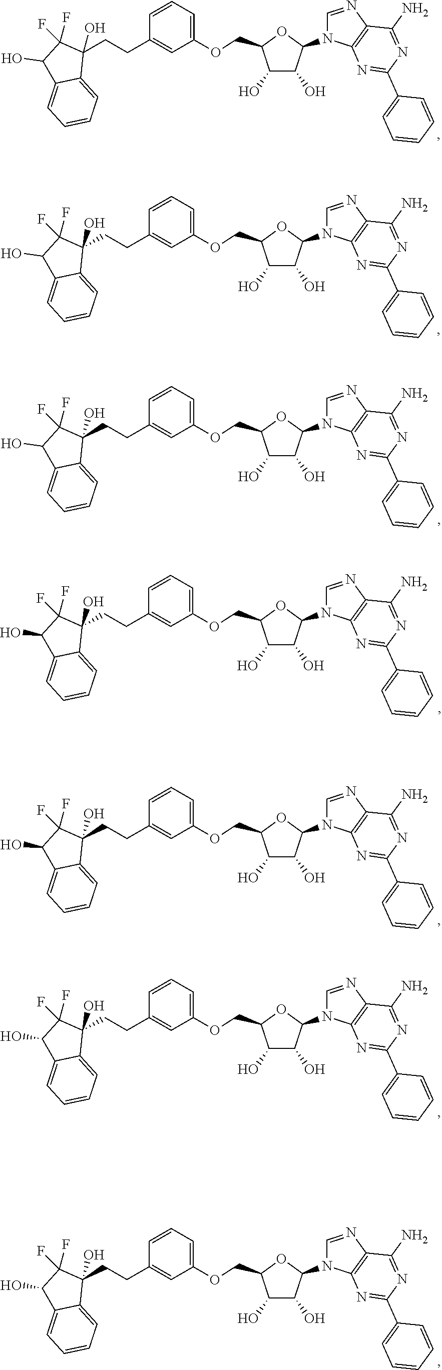 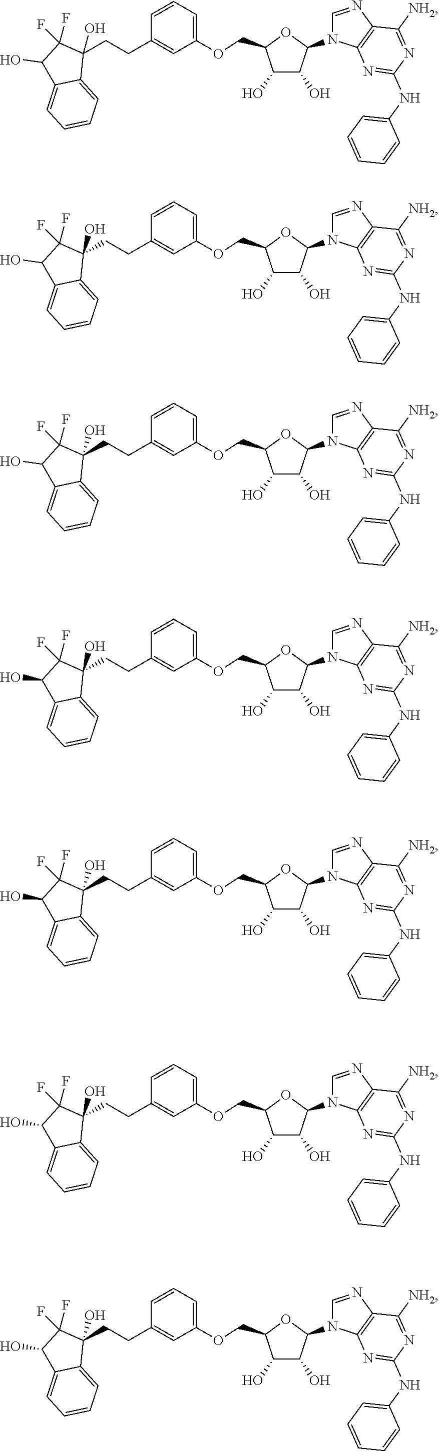 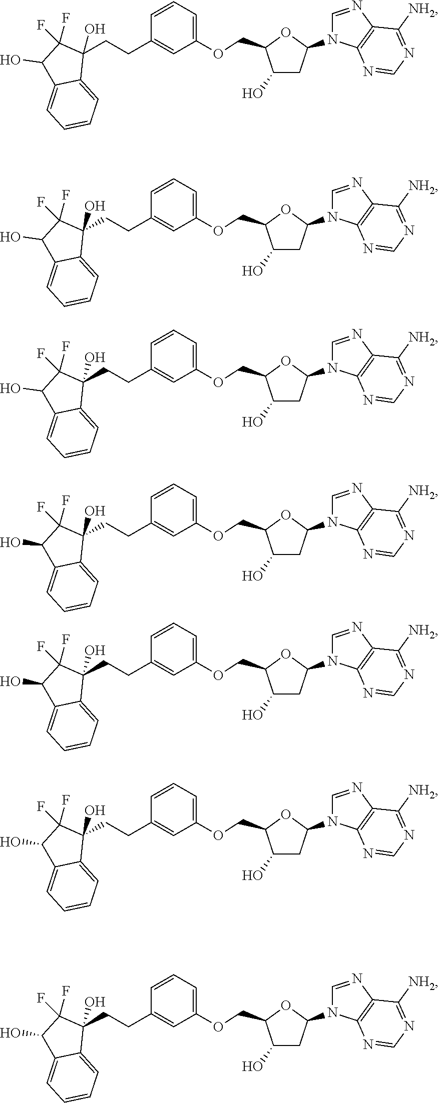 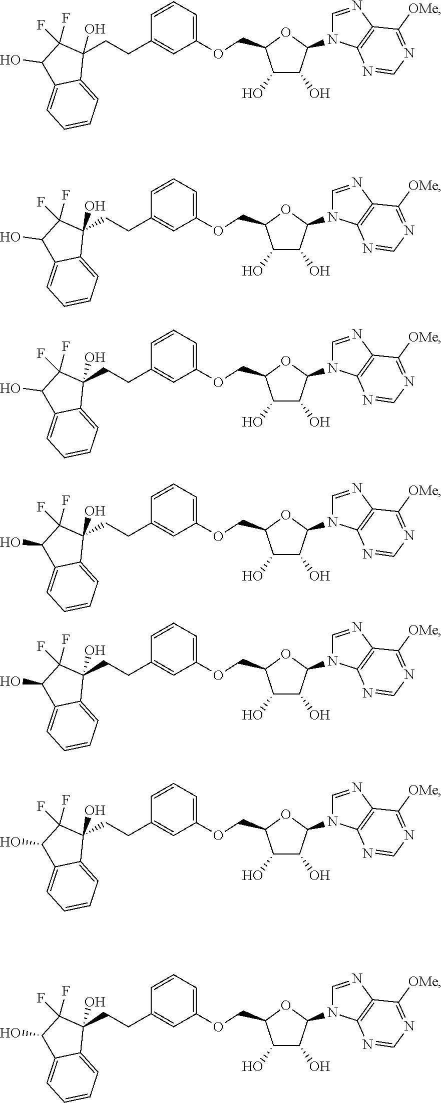 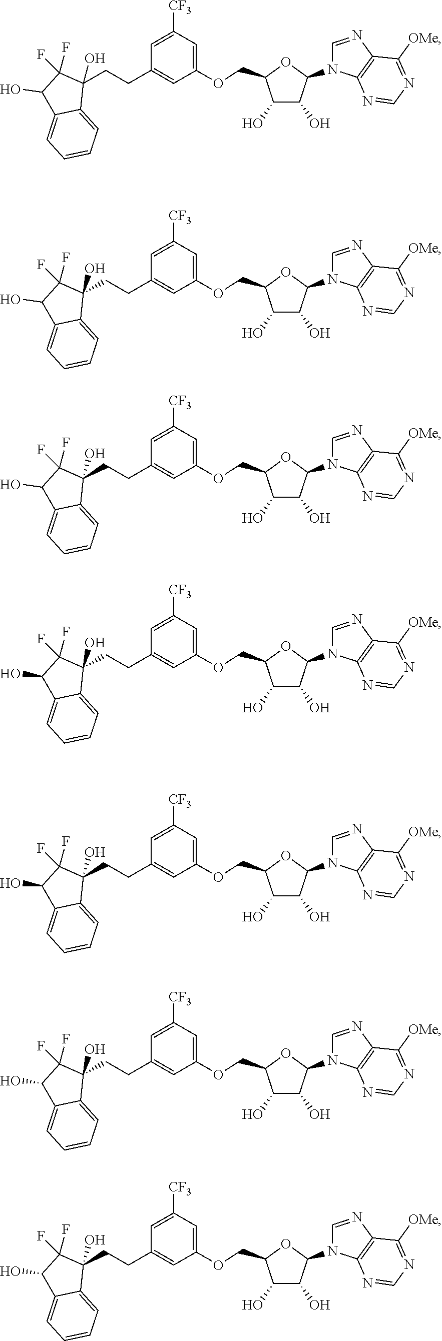 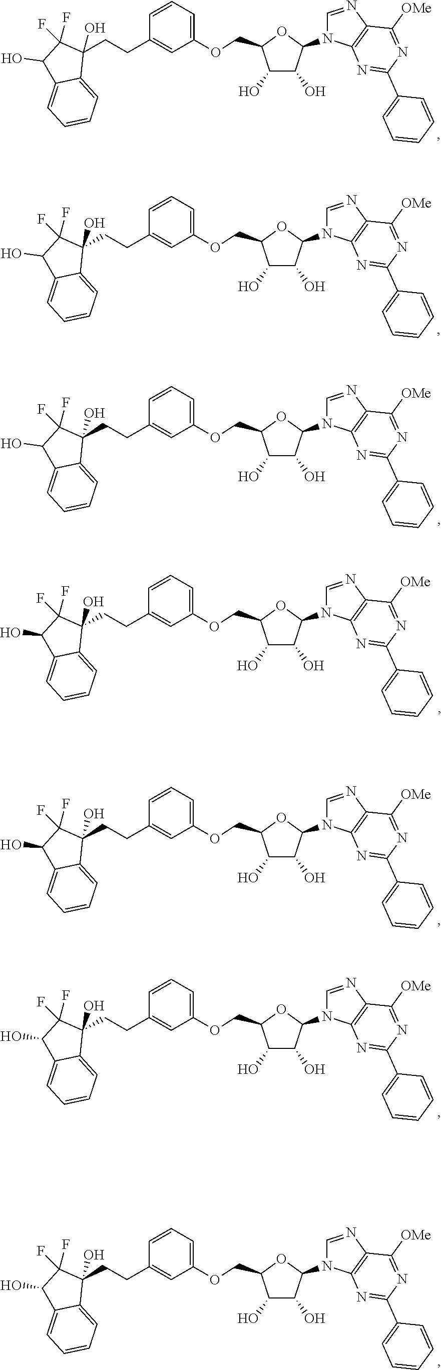 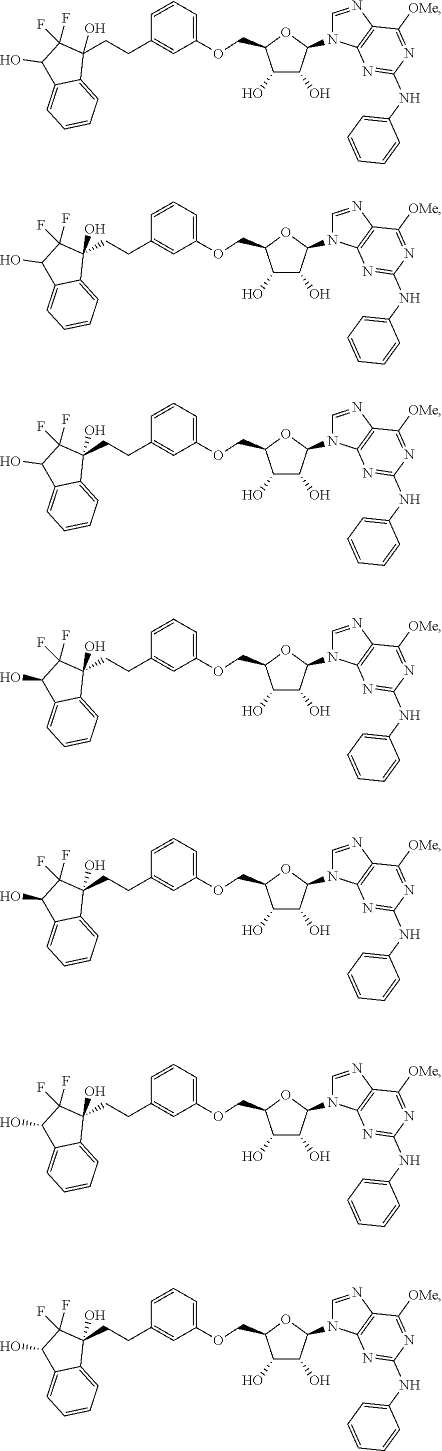 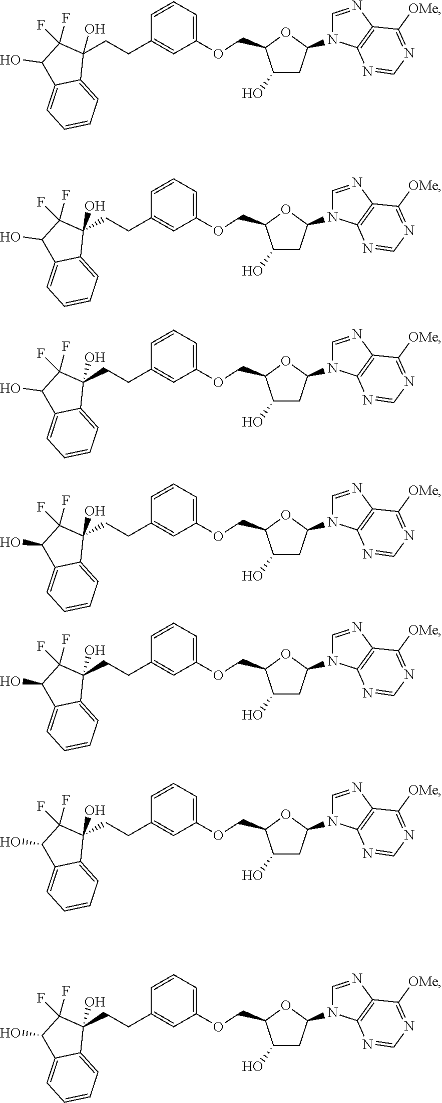 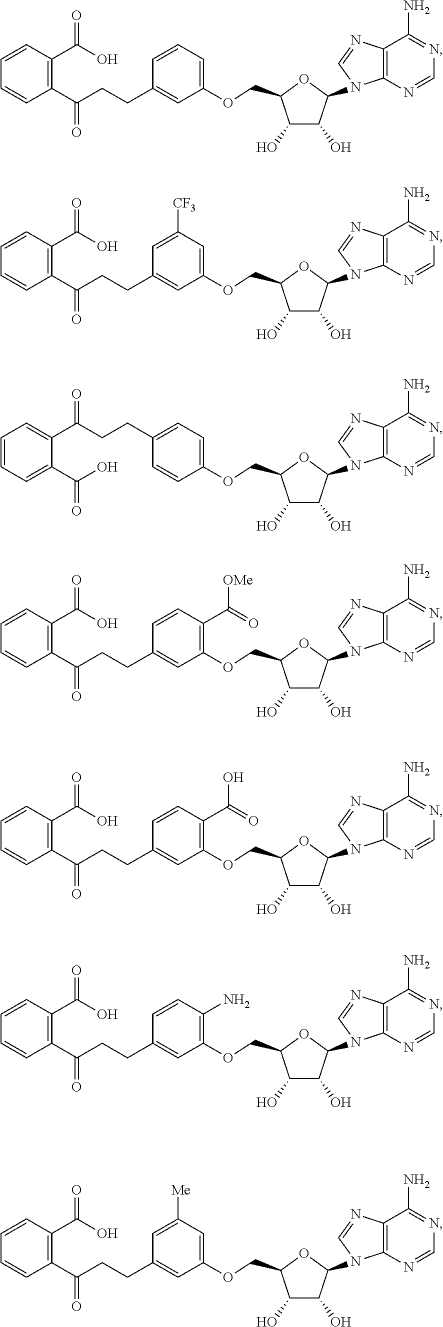 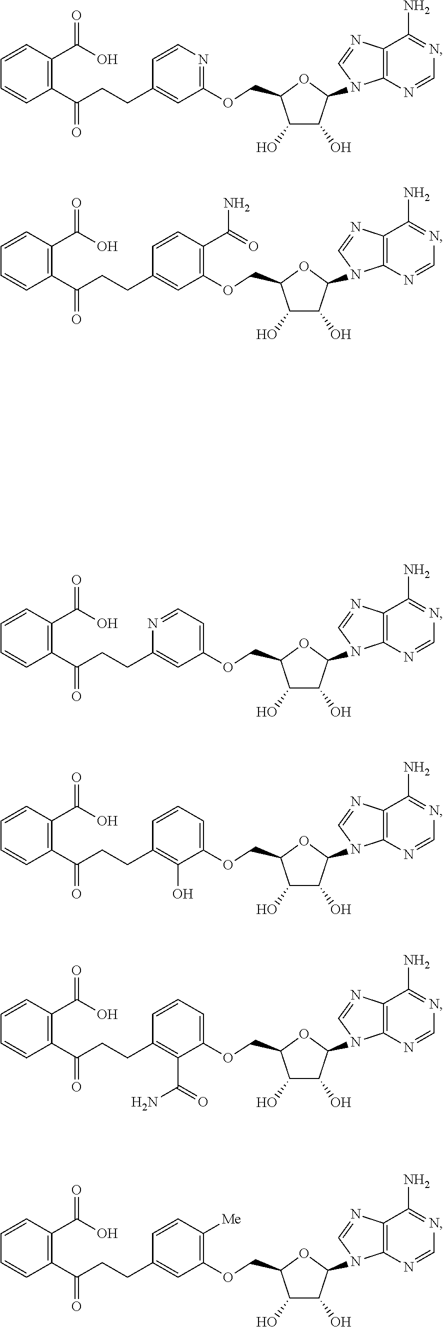
or a pharmaceutically acceptable salt, stereoisomer, or tautomer thereof.

48. The compound of claim 1, wherein the compound is of the formula: 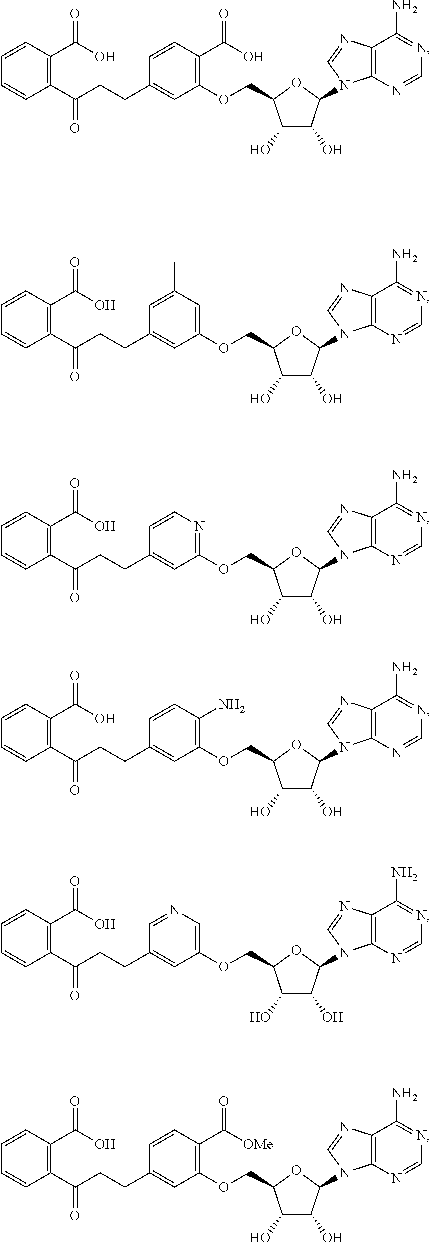
or a pharmaceutically acceptable salt, stereoisomer, or tautomer thereof.

49. A pharmaceutical composition comprising a compound of claim 1, and a pharmaceutically acceptable excipient.

50-52. (canceled)

53. A method of treating or preventing an infectious disease comprising administering an effective amount of a compound of claim 1 to a subject in need thereof.

54. (canceled)

55. The method of claim 53, wherein the infectious disease is a bacterial infection.

56-57. (canceled)

58. The method of claim 55, wherein the bacterial infection is:
(a) a Mycobacterium infection:,
(b) a Staphylococcus infection;
(c) a Escherichia infection; or
(d) a Bacillus infection.

59-67. (canceled)

68. A method of inhibiting menaquinone biosynthesis in a microorganism, the method comprising contacting the microorganism with a compound of claim 1.

69. A method of inhibiting o-succinylbenzoate CoA ligase (MenE) in a microorganism, the method comprising contacting the microorganism with a compound of claim 1.

70. A method of inhibiting an acyl-CoA synthetase in a microorganism, the method comprising contacting the microorganism with a compound of claim 1.

71. A method of inhibiting menaquinone biosynthesis in an infection in a subject, the method comprising administering to the subject a compound of claim 1.

72. A method of inhibiting o-succinylbenzoate CoA ligase (MenE) in an infection in a subject, the method comprising administering to the subject a compound of claim 1.

73. A method of inhibiting an acyl-CoA synthetase in an infection in a subject, the method comprising administering to the subject a compound of claim 1.

74. (canceled)

75. A kit for treating an infectious disease comprising a container, a compound of claim 1, and instructions for administering the compound or composition to a subject in need thereof.

76. (canceled)


SEQUENCE LISTING (1 sequence)

SEQ ID NO: 1 - DNA, 23 bp, Artificial Sequence
  Synthetic Polynucleotide
aacggcggtattgcgatttcacg